SEQUENCE LISTING (64 sequences)

SEQ ID NO: 1 - DNA, 636 bp, Homo sapiens
  source 1..636: mol_type = genomic DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 2 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 3 - DNA, 795 bp, synthetic construct
  source 1..795: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 4 - DNA, 810 bp, synthetic construct
  source 1..810: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 916 bp, synthetic construct
  source 1..916: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 6 - DNA, 931 bp, synthetic construct
  source 1..931: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 7 - DNA, 617 bp, synthetic construct
  source 1..617: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 8 - DNA, 632 bp, synthetic construct
  source 1..632: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcactgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 9 - DNA, 701 bp, synthetic construct
  source 1..701: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 10 - DNA, 716 bp, synthetic construct
  source 1..716: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 11 - DNA, 792 bp, synthetic construct
  source 1..792: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 12 - DNA, 686 bp, synthetic construct
  source 1..686: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggccgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 13 - DNA, 807 bp, synthetic construct
  source 1..807: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 14 - DNA, 701 bp, synthetic construct
  source 1..701: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcctgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 15 - DNA, 913 bp, synthetic construct
  source 1..913: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 16 - DNA, 928 bp, synthetic construct
  source 1..928: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 17 - DNA, 529 bp, synthetic construct
  source 1..529: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatgagctctccccatagctgggctgcggcccaaccccaccccctcaggctatgccagggggtgttgccaggggcacccgggcatcgccagtctagcccactccttcataaagccctcgcatcccaggagcgagcagagccagagc

SEQ ID NO: 18 - DNA, 646 bp, synthetic construct
  source 1..646: mol_type = other DNA
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacccgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 19 - DNA, 614 bp, synthetic construct
  source 1..614: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 20 - DNA, 696 bp, synthetic construct
  source 1..696: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgcttctg

SEQ ID NO: 21 - DNA, 709 bp, synthetic construct
  source 1..709: mol_type = other DNA
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggcgggctgggcataaaagtcagggcagagccatctattgcttacatttgcttctg

SEQ ID NO: 22 - DNA, 768 bp, synthetic construct
  source 1..768: mol_type = other DNA
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggggggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 23 - DNA, 736 bp, synthetic construct
  source 1..736: mol_type = other DNA
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcccgagcggagaggcggcccgggagcaggggggcggcccccactccggccgggtgcccggcccctggcccctgcctgccctctagatcgccgccgcagccgccgctactgggagtctg

SEQ ID NO: 24 - DNA, 1744 bp, synthetic construct
  source 1..1744: mol_type = other DNA
ttgtaatgggaataagggcaggactcctgggtataagtagctcagctgatcccaccctgcttctatgtgttaattcatttattcattcattcaacaagcatttgttgaaatgctctttgtgtcaggctcagcaggaagcagtggcaataaaatggtgaacaagaaagactcggggtttcttcatctatgttgatgtctgcagagaacagtatcagccttctaggaagtttgtaatcagatacattgttagagagatacttatctagtaaattcctactcatcctataaggctcaaaacaaatgcctctatgaaaccttccgtgattccctcaggcagagttaagagcttcctttcctgggcctctatctccttccattagtattataactgtttaccagtttcccctctagactaaatttctcaaaagagagaatgaggtctctttcagtcttctttgcatctttaaactagcctgggtcccagcctgtttgatgaaagaaacaagaacactgatacaagccacagccccttggcaaaaaagatacccaatagcaatggcaatgtaaaatcagttttagtaaatgaatcaagaattctgatgctttagggaaagtaatgtgaacctggcaccattaacaaattcagaactcttcttcttaggagctctctaactgaacagacagagggatgtcaacccctaattcagcttgatcgtatctcagcaactacatttaatgagacagtgggaaaaagagagctgtccacttttaaatcagcatatttctaactaaacaatggcaatggctaaatctttaaaatgcctatttctctcaagaacactgcaatggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaaacatgagatgccaggaaaacacacttaaaaaaaaaattcccactttaagctttagactgaatgtgagaaaggagatgataaaaagagtatcacaagagaatcttcaggctgtggg

SEQ ID NO: 25 - DNA, 1104 bp, synthetic construct
  source 1..1104: mol_type = other DNA
tttggcactgtgagcagtttacttgacaaattctgtcaaatatttgctttctgaaatctcgagaattggttgaatataattgtacttaatgtttgcaaaataaataaatatgggactaaggacgttctatcattaatttgtcagaaaagagagttgtcatttctgaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatttccagcagagttctattaaagggaggacagaatctgtctcttccaaggtggaaaatcgtgaatattccctgcattaatgaaccaagttaacactttaattgcttatagaaccgagttctccaatgacagcattaaaagatagggaggctctgatttatggtcaacacagatttgtaaccc

SEQ ID NO: 26 - DNA, 1910 bp, synthetic construct
  source 1..1910: mol_type = other DNA
tcaacatggataaccaaagttcttaaaactacgctttcaatgaacacatatcctttgagcaagactaataatgaggaatgggagccagctcctgtgatatttatgcaactactaaattctcactgaagtcaatgggagtttgcttacgtaagggctgcaaactttagcctccagagattaaaggggaaaaaaatccttaaactctttcaacattaatattgcctgtaaggaatccagccatgacctaagccatggagctttctgaacctagcaagtagaagggtaaacagtaaacaccagttattttaagcacaatctaatcagagttcaatgagaagcaatattatatttgatctctaaggtattaatacttgtatatcactattagacatctttatgtagtccattatccaaacaatggcttaagtctgtggtatttaataaatcaagtttccatggccgtgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagtgatatttaaaaaagaaactttggcaaatgcttgcaaaaaacacaccttttacaattttaaatgtgatttactgatggccagaacttgttaaacatagtaggaaattaaatatttattcatcttatttcattttcagggccgtaaacgctccttctgagtcattcccaataacaagaatttctaccagtaaagctattaacaggcatcaaaataggggagtgctaaattaagatgagattgtaaaagcaaataagaacatacgcagactcgcataggagtgcaaatgatcgtttctgattgaaatgtttatagctaaatgagtttggctgaattaaacacaaatgttccaaaagataagccgtagctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgctatgcttcaattttcatctaggtgatcaagattcattttcttgttctgtcatccaaataggcagacagaaaagtgattgaaatacattatggagatgtgtcattgcaca

SEQ ID NO: 27 - DNA, 540 bp, synthetic construct
  source 1..540: mol_type = other DNA
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgc

SEQ ID NO: 28 - DNA, 696 bp, synthetic construct; identical to SEQ ID NO: 20
  source 1..696: mol_type = other DNA

SEQ ID NO: 29 - DNA, 709 bp, synthetic construct; identical to SEQ ID NO: 21
  source 1..709: mol_type = other DNA

SEQ ID NO: 30 - DNA, 768 bp, synthetic construct; identical to SEQ ID NO: 22
  source 1..768: mol_type = other DNA

SEQ ID NO: 31 - DNA, 736 bp, synthetic construct; identical to SEQ ID NO: 23
  source 1..736: mol_type = other DNA

SEQ ID NO: 32 - DNA, 1744 bp, synthetic construct; identical to SEQ ID NO: 24
  source 1..1744: mol_type = other DNA

SEQ ID NO: 33 - DNA, 1104 bp, synthetic construct; identical to SEQ ID NO: 25
  source 1..1104: mol_type = other DNA

SEQ ID NO: 34 - DNA, 1910 bp, synthetic construct; identical to SEQ ID NO: 26
  source 1..1910: mol_type = other DNA

SEQ ID NO: 35 - DNA, 540 bp, synthetic construct; identical to SEQ ID NO: 27
  source 1..540: mol_type = other DNA

SEQ ID NO: 36 - DNA, 982 bp, synthetic construct
  source 1..982: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 37 - DNA, 876 bp, synthetic construct
  source 1..876: mol_type = other DNA
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 38 - DNA, 835 bp, synthetic construct
  source 1..835: mol_type = other DNA
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaagggctgggcataaaagtcagggcagagccatctattgcttacatttgcttctg

SEQ ID NO: 39 - DNA, 812 bp, synthetic construct
  source 1..812: mol_type = other DNA
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatgggctgggcataaaagtcagggcagagccatctattgcttacatttgcttctg

SEQ ID NO: 40 - DNA, 487 bp, synthetic construct
  source 1..487: mol_type = other DNA
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagggctgggcataaaagtcagggcagagccatctattgcttacatttgcttctg

SEQ ID NO: 41 - DNA, 593 bp, synthetic construct
  source 1..593: mol_type = other DNA
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgcgggctgggcataaaagtcagggcagagccatctattgcttacatttgcttctg

SEQ ID NO: 42 - DNA, 53 bp, synthetic construct
  source 1..53: mol_type = other DNA
gggctgggcataaaagtcagggcagagccatctattgcttacatttgcttctg

SEQ ID NO: 43 - DNA, 553 bp, synthetic construct
  source 1..553: mol_type = other DNA
gggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 44 - DNA, 359 bp, synthetic construct
  source 1..359: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgcca

SEQ ID NO: 45 - DNA, 463 bp, synthetic construct
  source 1..463: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 46 - DNA, 386 bp, synthetic construct
  source 1..386: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacat

SEQ ID NO: 47 - DNA, 418 bp, synthetic construct
  source 1..418: mol_type = other DNA
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacc

SEQ ID NO: 48 - DNA, 502 bp, synthetic construct
  source 1..502: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 49 - DNA, 324 bp, synthetic construct
  source 1..324: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcac

SEQ ID NO: 50 - DNA, 121 bp, synthetic construct
  source 1..121: mol_type = other DNA
agcgcgcagagtctgcatgcgtgaggaagctcctgggcgcgtcacagccgcgctattctcagcgtctctccttttatggctccggaagtgagctggggttgctggcagcctggctggcact

SEQ ID NO: 51 - DNA, 393 bp, synthetic construct
  source 1..393: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcc

SEQ ID NO: 52 - DNA, 408 bp, synthetic construct
  source 1..408: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaa

SEQ ID NO: 53 - DNA, 499 bp, synthetic construct
  source 1..499: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaa

SEQ ID NO: 54 - DNA, 643 bp, synthetic construct
  source 1..643: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatg

SEQ ID NO: 55 - DNA, 656 bp, synthetic construct
  source 1..656: mol_type = other DNA
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggc

SEQ ID NO: 56 - DNA, 215 bp, synthetic construct
  source 1..215: mol_type = other DNA
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggg

SEQ ID NO: 57 - DNA, 782 bp, synthetic construct
  source 1..782: mol_type = other DNA
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaa

SEQ ID NO: 58 - DNA, 759 bp, synthetic construct
  source 1..759: mol_type = other DNA
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgat

SEQ ID NO: 59 - DNA, 434 bp, synthetic construct
  source 1..434: mol_type = other DNA
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccata

SEQ ID NO: 60 - DNA, 540 bp, synthetic construct; identical to SEQ ID NO: 27
  source 1..540: mol_type = other DNA

SEQ ID NO: 61 - DNA, 467 bp, synthetic construct
  source 1..467: mol_type = other DNA
gagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcg

SEQ ID NO: 62 - DNA, 636 bp, synthetic construct
  source 1..636: mol_type = other DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaattggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 63 - DNA, 636 bp, synthetic construct
  source 1..636: mol_type = other DNA
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatgtga

SEQ ID NO: 64 - DNA, 9678 bp, synthetic construct
  source 1..9678: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctagtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattgcaccatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaccatcgatagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaacgaccgagcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaactccgtcgagaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtctaaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctgcttccggtattcgcttaattcagcacaacggaaagagcactggctaaccaggctcgccgactcttcacgattatcgactcaatgctcttacctgttgtgcagatataaaaaatcccgaaaccgttatgcaggctctaactattacctgcgaactgtttcgggattgcattttgcagacctctctgcctgcgatggttggagttccagacgatacgtcgaagtgaccaactaggcggaatcggtagtaagcgccgcctcttttcatctcactaccacaacgagcgaattaacccatcgttgagtcaaatttacccaattttattcaataagtcaatatcatgccgttaatatgttgccatccgtggcaatcatgctgctaacgtgtgaccgcattcaaaatgttgtctgcgattgactcttctttgtggcattgcaccaccagagcgtcatacagcggcttaacagtgcgtgaccaggtgggttgggtaaggtttgggattagcatcgtcacagcgcgatatgctgcgcttgctggcatccttgaatagccgacgcctttgcatcttccgcactctttctcgacaactctcccccacagctctgttttggcaatatcaaccgcacggcctgtaccatggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatg

(19) United States
(12) Patent Application Publication
(10) Pub. No.: US 2025/0222072 A1
(43) Pub. Date: Jul. 10, 2025

(54) METHODS AND COMPOSITIONS FOR THE TREATMENT OF PARKINSON'S DISEASE

(71) Applicant: ASKLEPIOS BIOPHARMACEUTICAL, INC., Research Triangle Park, NC (US)

(72) Inventors: Krystof S. BANKIEWICZ, Oakland, CA (US); Adrian P. KELLS, San Francisco, CA (US); Amber VAN LAAR, Research Triangle Park, NC (US); Massimo S. FIANDACA, Castro Valley, CA (US)

(73) Assignee: ASKLEPIOS BIOPHARMACEUTICAL, INC., Research Triangle Park, NC (US)

(21) Appl. No.: 18/850,414

(22) PCT Filed: Mar. 24, 2023
(86) PCT No.: PCT/US2023/016270

§ 371 Date: Sep. 24, 2024

Related U.S. Application Data
(60) Provisional application No. 63/438,164, filed on Jan. 10, 2023.
(60) Provisional application No. 63/393,196, filed on Jul. 28, 2022.
(60) Provisional application No. 63/341,841, filed on May 13, 2022.
(60) Provisional application No. 63/326,236, filed on Mar. 31, 2022.
(60) Provisional application No. 63/323,830, filed on Mar. 25, 2022.

(51) Int. Cl. A61K 38/18; A61K 48/00; A61P 25/16; C07K 14/475; C12N 15/86

(52) U.S. Cl. CPC A61K 38/185; A61K 48/0058; A61K 48/0075; A61P 25/16; C07K 14/475; C12N 15/86; C12N 2750/14143

(57) ABSTRACT

Aspects of the disclosure relate to compositions and methods useful for treating Parkinson's disease. In some embodiments, the disclosure provides a method for treating Parkinson's disease comprising administration of a viral vector comprising a GDNF nucleic acid sequence. In some embodiments, administration is locally to the subject putamen. In some embodiments, administration is systemically, e.g., via the viral vector comprising a modified viral capsid, such as for preferentially targeting cells in the CNS or PNS.

### CROSS-REFERENCE TO RELATED APPLICATIONS

This application claims benefit under 35 U.S.C. § 119(e) of U.S. Provisional Application No. 63/323,830 filed Mar. 25, 2022; U.S. Provisional Application No. 63/326,236 filed Mar. 31, 2022; U.S. Provisional Application No. 63/341,841 filed May 13, 2022; U.S. Provisional Application No. 63/393,196 filed Jul. 28, 2022; and U.S. Provisional Application No. 63/438,164 filed Jan. 10, 2023, the contents of each of which are incorporated herein by reference in their entireties.

### GOVERNMENT SUPPORT

This invention was created in the performance of a Cooperative Research and Development Agreement with the Department of Veterans Affairs, an agency of the U.S. Government, which has certain rights in this invention.

### TECHNICAL FIELD

The technology described herein relates to methods for treating neurological diseases or disorders, e.g., Parkinson's disease.

### BACKGROUND

Parkinson's disease (PD) is a progressive neurodegenerative disease that advances inexorably over a period of 10 to 30 years to disability and death. Medications, generally those aimed at ameliorating the known striatal dopamine deficiency, can provide substantial clinical benefits for the cardinal motor signs of PD, namely rest tremor, rigidity, bradykinesia and postural instability. However, disease progression continues since dopamine replacement and other medical therapies do not impact the underlying neurodegenerative process. Clinical responses to anti-parkinsonian medications wane over time and a variety of drug-related complications ensue, including motor fluctuations, dyskinesias, and neuropsychiatric manifestations.

Deep brain stimulation (DBS) is a rational and efficacious symptomatic treatment option for specific cardinal motor signs. However, the use of DBS has been limited due partially to risks and complexities of surgical implantation and device programing, as well as hardware-related complications and maintenance. More recently, Duopa has been approved for more advanced patients with severe motor fluctuations. Duopa is a levodopa/carbidopa intestinal gel administered via a gastrostomy tube connected to an external portable pump to provide consistent dosing. Though this circumvents intracranial surgery, Duopa requires the need to maintain stoma site and the inconvenience of carrying external components. Due to oxidation of Duopa, this therapy is approved for 16 hr/day and therefore leaves some patients inadequately treated overnight.

Research efforts have pointed to a number of potential mechanisms that might underlie the neurodegenerative process in PD. Oxidative stress, mitochondrial dysfunction and intracellular protein processing abnormalities are commonly posited mechanisms. Experimental therapeutic studies have been designed to correct such pathobiological disturbances, with the intent to slow, prevent or reverse neurodegenerative processes. Neurotrophic factors such as GDNF have the potential to alter the course of PD rather than only treating specific clinical features.

PD is a progressive, multicentric neurodegenerative disease characterized by tremor at rest, rigidity, bradykinesia and postural instability. The majority of PD is an idiopathic disease and the second most common neurodegenerative disorder after Alzheimer's disease. Patients struggle with emotional symptoms including depression and anxiety and with characteristic motor features and movement disturbances. There is currently no cure for PD; therapeutic options are limited to ameliorating disease symptoms.

### SUMMARY

One aspect provided herein describes a method of slowing or inhibiting progression of Parkinson's disease (PD) in a subject in need thereof comprising introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the GDNF gene (sometimes referred to as a transgene), and wherein the subject does not exhibit an increase in PD-associated symptoms for a least 6 months following the introducing as compared to prior to introducing.

In one embodiment of any aspect herein, the rAAV is introduced via systemic introduction.

In one embodiment of any aspect herein, the rAAV is introduced via local introduction.

In one embodiment of any aspect herein, local introduction is introduction directly to the subject's putamen.

In one embodiment of any aspect herein, the local introduction comprises directly introducing the rAAV to each of the subject's putamen.

In one embodiment of any aspect herein, the local introduction is performed in simultaneously with non-invasive imaging. Exemplary the non-invasive imaging techniques include intraoperative magnetic resonance image (iMRI)-guided convection enhanced delivery (CED), ultrasound, computed tomography (CT); functional magnetic resonance imaging (fMRI); positron emission tomography (PET); electroencephalography (EEG); magnetoencephalography (MEG); functional near-infrared spectroscopy (fNIRS); and combinations thereof.

In one embodiment of any aspect herein, the local introduction comprises introducing about half of the total delivered dose of rAAV vector to each putamen via intraoperative magnetic resonance image (iMRI)-guided convection enhanced delivery (CED).

In one embodiment of any aspect herein, local introduction further comprises introducing an MRI contrast agent at substantially the same time as the AAV vector.

In one embodiment of any aspect herein, the MRI contrast agent is gadoteridol.

In one embodiment of any aspect herein, the MRI contrast agent is introduced to the subject in the same composition as the rAAV. In one embodiment of any aspect herein, the MRI contrast agent is introduced to the subject in a different composition as the rAAV.

In one embodiment of any aspect herein, the rAAV is introduced via systemic (e.g., intravenous) introduction.

In one embodiment of any aspect herein, the transduction and/or coverage of the putamen is assessed via Magnetic-resonance imaging. In one embodiment of any aspect herein, at least 40%, 50%, 60%, 70%, 80%, 90%, 95% or more of the volume of the subject's putamen is transduced with the GDNF gene.

In one embodiment of any aspect herein, the subject does not exhibit a substantial increase in PD-associated symptoms for at least 12 months immediately following the introducing as compared to prior to the introducing.

In one embodiment of any aspect herein, the subject exhibits a decrease in PD-associated symptoms for at least 6 months or more immediately following the introducing as compared to prior to introducing.

In one embodiment of any aspect herein, the subject exhibits a decrease in PD-associated symptoms for a least 12 months or more immediately following the introducing as compared to prior to introducing.

In one embodiment of any aspect herein, the subject has an initial Movement Disorder Society-Unified Parkinson Disease Rating Scale (MDS-UPDRS) score, prior to introduction, that is less than 32.

In one embodiment of any aspect herein, the slowing or inhibiting the progression of Parkinson's disease in the subject is characterized by a second MDS-UPDRS score 6 months immediately following the introducing that is not substantially higher than the initial MDS-UPDRS score.

In one embodiment of any aspect herein, the slowing or inhibiting the progression of Parkinson's disease in the subject is characterized by a second MDS-UPDRS score about 12 months immediately following the introducing that is not substantially higher than the initial MDS-UPDRS score.

In one embodiment of any aspect herein, the subject has an initial MDS-UPDRS score, prior to introduction, that is greater than or equal to 32.

In one embodiment of any aspect herein, the subject exhibits a decrease in the initial MDS-UPDRS score for at least 6 months immediately following the introducing as compared to prior to introducing.

In one embodiment of any aspect herein, the slowing or inhibiting the progression of Parkinson's disease in the subject is characterized by a second MDS-UPDRS score about 6 months immediately following the introducing that is at least about 20% lower than the initial MDS-UPDRS score.

In one embodiment of any aspect herein, the slowing or inhibiting the progression of Parkinson's disease in the subject is characterized by a second MDS-UPDRS score about 12 months immediately following the introducing that is at least about 30% lower than the initial MDS-UPDRS score

In one embodiment of any aspect herein, the method further comprises, prior to introducing, determining an initial MDS-UPDRS score for the subject.

In one embodiment of any aspect herein, the method further comprises, prior to introducing, receiving results of an assay that provides an initial MDS-UPDRS score for the subject.

In one embodiment of any aspect herein, slowing or inhibiting the progression of PD in the subject is characterized by a reduction of an initial MDS-UPDRS score following introduction.

In one embodiment of any aspect herein, the reduction is an at least 20%, 30%, 40%, 50%, 60%, 70%, 80%, 90%, 95% or greater reduction of the initial MDS-UPDRS score 6 months following introduction.

In one embodiment of any aspect herein, the reduction is an at least 20%, 30%, 40%, 50%, 60%, 70%, 80%, 90%, 95% or greater reduction of the initial MDS-UPDRS score 12 months following introduction.

In one embodiment of any aspect herein, slowing or inhibiting the progression of PD in the subject is characterized stabilization of an initial MDS-UPDRS score following introduction.

In one embodiment of any aspect herein, the stabilization is characterized by no more than a 10% increase or decrease of the initial MDS-UPDRS score. In one embodiment of any aspect herein, stabilization occurs for at least 6 months or longer.

In one embodiment of any aspect herein, the subject is mildly affected by PD. In one embodiment of any aspect herein, the subject mildly affected by PD has an initial MDS-UPDRS score less than 32 prior to the introduction of rAAV and was diagnosed with PD less than 5 years prior to the introduction.

In one embodiment of any aspect herein, the method further comprises, prior to the introduction, diagnosing the subject as being mildly affected by PD.

In one embodiment of any aspect herein, the method further comprises, prior to the introduction, receiving the results of an assay that diagnoses the subject as being mildly affected by PD.

In one embodiment of any aspect herein, the subject is moderately affected by PD. In one embodiment of any aspect herein, the subject moderately affected by PD has an initial MDS-UPDRS score equal to or greater than 32 prior to the introduction of rAAV and was diagnosed with PD less than 4 years prior to the introduction.

In one embodiment of any aspect herein, the method further comprises, prior to the introduction, diagnosing the subject as being moderately affected by PD.

In one embodiment of any aspect herein, the method further comprises, prior to introduction, receiving the results of an assay that diagnoses the subject as being moderately affected by PD.

In one embodiment of any aspect herein, the promoter is a cytomegalovirus (CMV) promoter.

In one embodiment of any aspect herein, the promoter is a nervous system (NS) or central nervous system (CNS) specific promoter. In one embodiment of any aspect herein, the NS specific promoter is selected from the NS specific promoters in Table 1. In one embodiment of any aspect herein, the CNS specific promoter is selected from the CNS specific promoters in Table 2.

In one embodiment of any aspect herein, the nucleic acid comprises a sequence of SEQ ID NO: 1, or a functional variant that is at least 85, 86, 87, 88, 89, 90, 91, 92, 93, 94, 95, 96, 97, 98, 99% or more identical to SEQ ID NO: 1.

In one embodiment of any aspect herein, the rAAV is AAV1, AAV2, AAV3, AAV3b, AAV4, AAV5, AAV6, AAV7, AAV8, AAV9, AAV10, AAV11, AAV12, Rh10, or a rational haploid thereof. In one embodiment of any aspect herein, the rAAV is AAV2.

In one embodiment of any aspect herein, the rAAV exhibits brain-specific tropism. In one embodiment of any aspect herein, the rAAV comprises a modification that increases its brain-specific tropism. In one embodiment of any aspect herein, brain-specific tropism is increased by at least 20%, 30%, 40%, 50%, 60%, 70%, 80%, 90%, 95% or greater as compared to an unmodified AAV.

In one embodiment of any aspect herein, the rAAV is introduced at a total dose within the range of 5×10¹² vg to about 1.5×10¹³ vg.

In one embodiment of any aspect herein, about one half of the total dose is administered to each of the subject's putamen.

In one embodiment of any aspect herein, introducing is performed at a flow rate of from about 1 μL/min to about 30 μL/min.

In one embodiment of any aspect herein, the rAAV is introduced as a liquid composition comprising the rAAV and a pharmaceutically acceptable carrier.

In one embodiment of any aspect herein, the liquid composition has an rAAV concentration of from about 3×10¹² vg/mL to about 4×10¹² vg/mL.

In one embodiment of any aspect herein, the subject is administered at least one anti-PD therapeutic prior to the introduction of the rAAV.

In one embodiment of any aspect herein, the subject is administered at least one anti-PD therapeutic prior to and following the introduction of the rAAV. In one embodiment of any aspect herein, the at least one anti-PD therapeutic is selected from the group consisting of levodopa, Sinemet, Rytary, Stalevo, amantadine, pramipexole, rotigotine, ropinirole, apomorphine, entacapone.

In one embodiment of any aspect herein, the subject maintains or decreases the dose of the at least one anti-PD therapeutic following introduction. In one embodiment of any aspect herein, the dose of the at least one anti-PD therapeutic is decreased by at least 5%, 10%, 15%, 20%, 25%, 30%, 35%, 40%, 45%, 50% or more.

Another aspect provided herein describes a method of slowing or inhibiting a progression of Parkinson's disease (PD) in a subject in need thereof comprising locally introducing to the subject's putamen a recombinant adeno-associated virus (rAAV) vector comprising a nucleic acid encoding glial cell line-derived neurotrophic factor (GDNF) operably linked to a promoter, wherein at least 30% of the volume of the subject's putamen is transduced with the GDNF gene.

Another aspect provided herein describes a method of slowing or inhibiting a progression of PD in a subject in need thereof comprising transducing greater than or equal to about 30% of the volume of the subject's putamen with a glial cell line-derived neurotrophic factor (GDNF) gene, wherein the subject does not exhibit a substantial increase in PD-associated symptoms for a least 6 months following the transducing. In one embodiment of any aspect herein, the transducing is performed by administering a rAAV comprising the GDNF gene to each of the subject's putamen.

Another aspect provided herein describes a method of reducing or stabilizing an initial Movement Disorder Society-Unified Parkinson's Disease Rating Scale Part (MDS-UPDRS) score in a subject having Parkinson's disease (PD) comprising administering to the subject's putamen a recombinant adeno-associated virus (rAAV) comprising a nucleic acid encoding glial cell line-derived neurotrophic factor (GDNF) operably linked to a promoter, wherein the subject has a second MDS-UPDRS score at 6 months following the administration is decreased or stabilized as compared to the initial MDS-UPDRS score of the subject prior to administering.

In one embodiment of any aspect herein, the method further comprises the step of, prior to administering, obtaining or receiving an initial MDS-UPDRS score from the subject. In one embodiment of any aspect herein, the second MDS-UPDRS score is decreased by at least 20%, 30%, 40%, 50%, 60%, 70%, 80%, 90%, 95% or greater as compared to the initial MDS-UPDRS score 12 months following administering.

In one embodiment of any aspect herein, stabilization is no more than a 10% increase or decrease of the initial MDS-UPDRS score.

Another aspect provided herein describes a method of treating a subject mildly affected by Parkinson's disease (PD) comprising administering to each of the subject's putamen a recombinant adeno-associated virus (rAAV) comprising a nucleic acid encoding glial cell line-derived neurotrophic factor (GDNF) operably linked to a promoter, wherein at least 30% of the subject's putamen is transduced with GDNF, and wherein the subject has a second MDS-UPDRS score at 6 months post-administering that is stabilized as compared to the initial MDS-UPDRS score.

In one embodiment of any aspect herein, the subject has a MDS-UPDRS score at 12 month post-administering that is stabilized as compared to the initial MDS-UPDRS score prior to administering.

Another aspect provided herein describes a method of treating a subject moderately affected by Parkinson's disease (PD) comprising administering to each of the subject's putamen a recombinant adeno-associated virus (AAV) comprising a nucleic acid encoding glial cell line-derived neurotrophic factor (GDNF) operably linked to a promoter, wherein at least 30% of the subject's putamen is transduced with the transgene, and wherein the subject has a second MDS-UPDRS score at 6 months post-administering that is at least about 20% lower than the initial MDS-UPDRS score. In one embodiment of any aspect herein, the reduction is an at least 30%, 40%, 50%, 60%, 70%, 80%, 90%, 95% or greater as compared to the initial MDS-UPDRS score.

Another aspect provided herein describes a method of slowing or inhibiting progression of Parkinson's disease (PD) in a subject in need thereof comprising locally introducing to each of the subject's putamen a recombinant adeno-associated virus (rAAV) vector comprising a nucleic acid encoding glial cell line-derived neurotrophic factor (GDNF) operably linked to a promoter; and locally introducing an MRI contrast agent to each of the subject's putamen at substantially the same time as the rAAV, wherein at least 30% of the volume of the subject's putamen is transduced with the nucleic acid, and wherein the subject does not exhibit a substantial increase in PD-associated symptoms for a least 6 months immediately following the introducing as compared to prior to introducing.

Another aspect provided herein describes a method of slowing or inhibiting progression of Parkinson's disease (PD) in a subject in need thereof comprising introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the GDNF gene, and wherein the subject does not exhibit a substantial increase in PD-associated symptoms for a least 6 months immediately following the introducing as compared to prior to introducing.

Another aspect provided herein describes a composition for slowing or inhibiting a progression of Parkinson's disease (PD) in a subject comprising a recombinant adeno-associated virus (rAAV) comprising a genome comprising a glial cell line-derived neurotrophic factor (GDNF) gene operably linked to a promoter; and a pharmaceutically acceptable carrier.

In one embodiment of any aspect herein, the composition has a rAAV concentration of 3×10¹² vg to 4×10¹² vg per mL.

In one embodiment of any aspect herein, the composition comprises an rAAV concentration of 3.3×10¹² vg per mL.

Another aspect provided herein describes a formulation for slowing or inhibiting a progression of Parkinson's disease (PD) in a subject comprising an adeno-associated virus (AAV) at a concentration of 3×10¹² vg to 4×10¹² vg per mL of a pharmaceutically acceptable carrier, wherein the rAAV comprises a genome comprising a glial cell line-derived neurotrophic factor (GDNF) gene operably linked to a promoter.

### BRIEF DESCRIPTION OF THE DRAWING

FIG. 1 shows a schematic of the clinical study schedule. The subject will cycle “ON” and “OFF” their prescribed anti-Parkinson's therapeutic as indicated by “OFF” (off medication) and “ON” (on medication) arrows. MRIs, FDG, and DaT scans are administered as indicated by crosses. Blood work is taken as indicated by the droplet. The subject's activity is monitored as indicated by the hexagon. For visits requiring evaluation in the defined OFF medication state, participants are asked to stop all PD medications (e.g. carbidopa/levodopa, Sinemet, Rytary, Stalevo, amantadine, pramipexole, rotigotine, ropinirole, apomorphine, entacapone) from the evening prior to the visit, and should be withheld for at least 12 hours.

FIG. 2 shows a summary of the subject's included in Cohort A (mildly affected by PD).

FIG. 3 shows a summary of the subject's included in Cohort B (moderately affected by PD).

FIG. 4 shows a summary of the percent volume of a subject's putamen that is transduced with GDNF following local administration of AAV2-GDNF. The average volume of putamen transduced with GDNF is 63%.

FIG. 5 presents a bar graph showing the percent volume of a subject's putamen that is transduced with GDNF following local administration of AAV2-GDNF operatively linked to CMV promoter. The average volume of putamen transduced with GDNF is ˜63%.

FIGS. 6A and 6B show representative post-surgical MRI of subjects in Cohort A. FIG. 6A shows MRI T1 (pre-contrast) images a period of time following administration of the therapeutic. FIG. 6B shows MRI T2 images a period of time following administration of the therapeutic. Variable appearance of putaminal hyperintensities are observed.

FIGS. 7A and 7B show representative post-surgical MRI of subjects in Cohort B. FIG. 7A shows MRI T1 (pre-contrast) images a period of time following administration of the therapeutic. FIG. 7B shows MRI T2 images a period of time following administration of the therapeutic. Variable appearance of putaminal hyperintensities are observed.

FIGS. 8A-8H present data that assess PD progression at various time points post administration. FIG. 8A-8D present line graphs showing the change in MDS-UPDRS aggregate motor skills either on an anti-Parkinson's therapeutic (FIG. 8A) or off an anti-Parkinson's therapeutic (FIG. 8B). A stabilized MDS-UPDRS score is observed in cohort A (represented by “mild PD cohort”) for 12 months. A marked decrease in MDS-UPDRS score is observed in cohort B (represented by “moderate PD cohort”). MDS-UPDRS scores were assessed when screening prior to administration, to establish a baseline, and then 3, 6, 9 and 12 months post-surgery. Total UPDRS scores (FIG. 8C) and MDS-UPDRS II score (FIG. 8D) follow the same trend over the indicated time post surgery. FIGS. 8E-8H show stable motor measures over 18 months post AAV2-GDNF dosing in the mild PD cohort (FIGS. 8E and 8F) and motor improvement over 18 months post AAV2-GDNF dosing in moderate PD cohort (FIGS. 8G and 8H). In FIGS. 8E and 8F, (A) indicates stability demonstrated over 18 months in mild PD. (B) indicates limited window to measure large magnitude of functional improvements in the mild PD. (C) indicates one outlier identified as having a TH mutation.

FIG. 9A-9D present data showing PD motor diary data. (FIG. 9A-9B) Bar graphs showing PD motor diary data for cohort A (FIG. 9A) and cohort B (FIG. 9B). The diary “on/off” times have been normalized to 16-hour waking times. (FIG. 9C) Pie charts illustrating marked improvement of subjects in cohort B 12-months and 18-months post administration. Good ON time was improved by 27% from baseline and OFF time was improved by 52% from baseline. (FIG. 9D) Pie charts illustrating marked improvement of subjects in cohort A 12-months and 18-months post administration. Good ON time was decreased by 12% from baseline and OFF time was increased by 46% from baseline.

FIGS. 10A and 10B present plot graphs showing NMSS (circles) and PDG-39 (squares) for cohort A (mildly affected; FIG. 10A) and cohort B (moderately affected; FIG. 10B). The NMSS and PDQ-39 scores were assessed when screening prior to administration, 6 months post-surgery, 12 months post-surgery, 18 months post-surgery, and 24 months post-surgery.

FIG. 11 presents a line graph showing the dose response of AAV2-GDNF in cohort B (moderately affected) versus phase 1 of the trial. The data indicate that there is a dose and putamen coverage (>50%) correlation with clinical response in moderate-stage PD. The magnitude of functional motor improvement in moderate-stage PD exceeded expectations of anticipated placebo effect (−5 pt).

FIG. 12 presents a graph showing volumetric distribution of putaminal infusions, including for gene therapy, are highly dependent on the infusion volume delivered, as predicted in animal models. For clinical PD cases, the average unilateral putaminal volume is approximately 4200 cubic mm. As depicted below the dashed horizontal line in this figure, early gene therapy products infused within the putamen, provided limited volumes of distribution, much less than 50% of the total putaminal volume. Initial limitations in distribution volumes were primarily a result of the small infusion volumes delivered and utilizing the standard bi-frontal trajectories to the putamen. The standard bi-frontal approach provides trajectories that are nearly perpendicular to the long axis of the putamen; such trajectories volumetric coverage of the putamen is limited by the small dorsoventral putaminal dimension, requiring multiple trajectories to expand volumetric coverage. The evolution of gene therapy infusions that parallel the long axis of the putamen has provided for much larger infusion volumes (up to 1800 microliters/putamen) and achieving putaminal coverage of >50%.

FIGS. 13A and 13B show schematics of bi-frontal and bi-occipital trajectory techniques. (FIG. 13A) Bi-frontal Trajectories-One or more frontal burr hole(s) is made bilaterally. Minimum of 2 trajectories per putamen to cover pre- and post-commissural putamen. Trajectories are nearly perpendicular to long axis of putamen and volumetric coverage primarily limited by short dorsoventral dimension of putamen and number of trajectories used. Putaminal volumetric coverage typically achieved is <50%. (FIG. 13B) Bi-occipital Trajectories-A single occipital burr hole is made per putamen. This technique requires a single trajectory per putamen to cover pre- and post-commissural putamen. Trajectories parallel to long axis of putamen and volumetric coverage primarily limited by perivascular leakage from within putamen. Putaminal volumetric coverage typically achieved is >50%.

FIGS. 14A and 14B present a summary of a previous, completed trial and the current ongoing trial. FIG. 14A presents a chart showing the clinical experience with both the bi-frontal and bi-occipital delivery methods for AAV2-GDNF gene therapy to the putamen in Parkinson's disease. FIG. 14A provides details from previous, completed Phase 1 and ongoing clinical trials testing the safety and tolerability of differing vector doses and putaminal coverage in advanced, moderate, and earlier stages of PD. The current clinical trial (as described in Examples 1-3 herein below) is the first human gene therapy trial to be approved for testing the safety of a gene therapy product in participants with earlier stage PD. As of the end of March 2022, the clinical study has enrolled and treated 11 of the 12 planned participants. The Phase 1 study delivered 450 microliters of infusion volume (at 9×10¹⁰ vg to 9×10¹¹ vg) to each putamen of 13 participants, resulting in a mean putaminal coverage of 26%. The bi-occipital delivery in the clinical trial described herein, so far in 11 participants, provided up to 1800 microliters of infusate in each putamen and has provided a mean putaminal distribution of 62.5%. FIG. 14B presents a summary of putaminal coverage achieved in the previous, completed Phase 1 trial and the current clinical trial (Phase 1b; as described in Examples 1-3 herein below) for indicated cohort.

FIG. 15 presents a schematic of AAV-GDNF. CMV, cytomegalovirus; hGDNF, human glial cell line-derived neurotrophic factor; hGH, human growth hormone; ITR, inverted terminal repeat.

FIG. 16 presents a schematic of the study design. AAV2-GDNF was administered via one-time, MRI-monitored CED to bilateral putamina (up to 1.8 mL per putamen with maximum dose of 1.2×10¹³ vg) and contrast agent to visualize distribution (2 mM gadoteridol).

FIG. 17 present a summary of postoperative adverse effects (i.e., treatment emergent adverse events (TEAEs)) observed more than 1 month after surgery.

FIGS. 18A and 18B present a summary of individual post-treatment changes across motor and non-motor assessments for mild cohort (FIG. 18A) and moderate cohort (FIG. 18B).

FIGS. 19A-19F present data showing expression of AAV2-GDNF 3.5 years post-administration. (FIG. 19A) MRI image of participant of intraputaminal administration of AAV2-GDNF. (FIG. 19B-19D) Tyrosine hydroxylase staining of putamen biopsy sample showing enrichment of dopaminergic neurons in the putamen. FIG. 19B shows area in FIG. 19A as indicated by arrow. FIG. 19C shows enhanced, zoomed-in image of area in FIG. 19B as indicated by arrow.

FIG. 19D shows enhanced, zoomed-in image of area in FIG. 19C as indicated by arrow. (FIG. 19E) Locations of 6 biopsies performed in sample. Biopsy locations #1 and 5 are the infusion sites used during surgery. Biopsy location #6 is located outside the putamen in white matter tract. (FIG. 19F) Level of GDNF transgene (pg GDNF/mg protein) in indicated biopsy location. The highest levels of GDNF were found in locations #1 and 5. No expression of GDNF identified in biopsy location #6.

FIG. 20 presents data showing longitudinal MRI monitoring for safety reads. T1 (top row) and T2 (bottom row) weighted MRI brain scans in the left column show gadoteridol distribution (bright white signal from T1 image) following bilateral infusion into the putamen (outlines). Matched MRI scans acquired at 6 and 18 month time points demonstrate no remaining gadoteridol signal or tissue abnormalities in the putamen or other brain structures.

FIG. 21 presents a chart depicting response of moderate PD cohort. A strong and more progressive restoration and motor function was found as compared to previous CGTs. 18 months clinical data shows (1) stronger improvements than previous neurotrophic CGTs, (2) AAV2-GDNF effects are more progressive than previous neurotrophic factor GTx with continuous improvement after six months, unlike brief improvement in other CGTs, and (3) clinically meaningful improvements beyond six months consistent with anticipated Mechanism of action, e.g., terminal sprouting and progressive restoration of dopamine function.

FIGS. 22A and 22B present charts showing unified dyskinesia rating scale historical, objective, and total scores up to 18 months post treatment for mild (FIG. 22A) and moderate (FIG. 22B) cohorts.

FIGS. 23A and 23B present charts showing levadopa equivalent daily dose (LEDD) average values for mild (FIG. 23A) and moderate (FIG. 23B) cohorts up to 18 months post treatment.

FIGS. 24A-24C present data showing preliminary analysis of functional imaging with DaT Scan in mild and moderate cohorts. FIG. 24A present bar graph of values. FIGS. 24B and 24C show tables presenting values depicted in FIG. 24A for mild (FIG. 24B) and moderate (FIG. 24C) cohorts. Preliminary analysis of change in DAT binding overtime is shown. Reductions in binding in caudate in both mild and moderate cohorts is observed. Relatively stable or increased put him in DaT signal in both cohorts is shown.

FIGS. 25A-25D present data showing change in F-dopa uptake at the infusion site 6 and 18 months after gene therapy administration. (FIGS. 25A-25C) MRI images show gadoteridol distribution in the axial (left column) and coronal (right column) planes following bilateral infusion into the interior (precommissural) and posterior (postcommissural) putamina (FIG. 25A). F-dopa Ki parametric maps in axial and coronal planes from one patient at baseline (FIG. 25B) and 18 months after surgery (FIG. 25C) showing increased Ki in the areas corresponding to the infusion sites as visualized as gadoteridol signal in the MRIs.

FIG. 26 presents a schematic of a plasmid used to generate the AAV2-GDNF vector, e.g., SEQ ID NO: 64.

### DETAILED DESCRIPTION

Aspects of the technology disclosed herein relate to administration, e.g., local or systemic, of the glial cell line-derived neurotrophic factor (GDNF) gene such that at least 30% of the subject's putamen is covered and/or transduced with the gene. This level of coverage and/or transduction is shown to be effective for reducing, slowing, or inhibiting the progression of symptoms related to PD. Accordingly, methods and compositions described by the disclosure are useful, in some embodiments, for the treatment of PD.

### Parkinson's Disease (PD)

Methods provided herein relate to slowing or inhibiting the progression of Parkinson's disease. As used herein, “Parkinson's disease”, or “PD”, refers to a neurodegenerative disease characterized by progressively worsening shaking and stiffness and increasing problems with balance, walking, and coordination. Although the majority of PD is considered idiopathic in nature, typically a combination of genetic predisposition and environmental influences acting on epigenetic controls, and a number of genes mutations can contribute to or increase the risk of PD, including those mutations with the synuclein alpha (SNCA; NCBI Gene ID: 6622), leucine rich repeat kinase 2 (LRRK2/PARK8; NCBI Gene ID 120892), glucosylceramidase beta (GBA1; NCBI Gene ID 2629), parkin RBR E3 ubiquitin (PRKN; NCBI Gene ID 5071), PTEN induced kinase 1 (PINK1; NCBI Gene ID 65018), Parkinsonism associated deglycase (DJ1/PARK7; NCBI Gene ID 11315), VPS35 retromer complex component (VPS35; NCBI Gene ID 55737), eukaryotic translation initiation factor 4 gamma 1 (EIF4G1; NCBI Gene ID 1981), DnaJ heat shock protein family member C13 (DNAJC13; NCBI Gene ID 23317), coiled-coil-helix-coiled-coil-helix domain containing 2 (CHCHD2; NCBI Gene ID 51142), and/or ubiquitin C-terminal hydrolase L1 (UCHL1; NCBI Gene ID 7345) genes. The sequences of such PD-associated genes are known in a number of species, e.g., human mRNAs and protein sequences are available in the NCBI database using the provided Gene ID numbers. These PD-associated genes and others, as well as PD-associated alleles thereof (e.g. mutations, duplications, SNPs, etc.) are known in the art and described further in, e.g., D'Souza et al. Acta Neuropsychiatrica 2020 32:10-22; Sardi et al. Parkinsonism & Related Disorders 2019 59:32-38; Hardy et al. Current Opinion in Genetics & Development 2009 19:254-65; Ferreria et al. Neurologica 2017 135:273-84; Jain et al. Clinical Science 2005 109:355-64; Fagan et al. European Journal of Neurology 2017 24:561-e20; Campelo et al. Parkinson's Disease 2017 4318416; and Porter et al. Chapter 15 of “Neurodegeneration and Alzheimer's Disease” 2019; each of which is incorporated by reference herein in its entirety.

Risk factors for developing PD include, but are not limited to, age, heredity, exposure to certain toxins, and sex. Diagnosis of PD as a juvenile and young adult is rare. The risk of developing Parkinson's increases with age, beginning at middle to late age; subjects typically develop the disease around age 60 or older. Having a close relative (e.g., an immediate family member, uncle, aunt, or grandparent) with PD increases the chances that a subject will develop the disease. However, the risk is still considered small unless multiple relatives have been diagnosed as having PD. Ongoing exposure to certain herbicides and pesticides has been shown to slightly increase the risk of PD in a subject. And finally, males are more likely to develop PD than females.

Symptoms of PD are well documented and known to one skilled in the art. Early symptoms of PD include, but are not limited to, tremors (e.g., shaking that usually begins in a limb, often in hands or fingers, when one's body is at rest); pilling-rolling tremor (e.g., rubbing a thumb and forefinger back and forth when one's body is at rest); slowed movement (bradykinesia); rigid muscles (i.e., muscle stiffness in any part of the body that can be painful and limit one's range of motion); impaired posture and balance (e.g., posture may become stooped, or one may have balance problems); loss of automatic movements (e.g., decreased ability to perform unconscious movements, including blinking, smiling or swinging arms when walking); speech changes (e.g., one may speak more softly and quickly, slur or hesitate before talking; or change in tone and loss of inflections); and writing changes (e.g., writing may appear smaller and more crowded).

Complications of PD include, but are not limited to, cognitive problems (dementia) and thinking difficulties in the later stages of PD; depression and emotional changes (i.e., fear, anxiety or loss of motivation) in early and late stages of Parkinson's; swallowing problems as the condition progresses (e.g., difficulties with swallowing, saliva accumulation and drooling); chewing and eating problems in late stage PD that can lead to choking and poor nutrition; sleep problems and sleep disorders (i.e., frequent waking, waking up early, and falling asleep during the day); rapid eye movement sleep behavior disorder; bladder problems (i.e., inability to control urine or having difficulty urinating); constipation; orthostatic hypotension (i.e., sudden drop in blood pressure); smell dysfunction (e.g., loss of smell or difficulty identifying certain odors or the difference between odors); fatigue; pain, i.e., either in specific areas of their bodies or throughout their bodies; and sexual dysfunction.

No one specific test for diagnosing Parkinson's exist, rather, a skilled clinician will diagnose a subject via the subject's medical record, family history, signs and/or symptom present, and a neurological and physical examination. A specific single-photon emission computerized tomography (SPECT) scan called a dopamine transporter scan (DaTscan) can be performed to support the diagnosis, but is not likely to be the key determinant for the diagnosis. Most patients do not require a DaTscan. Non-invasive imaging, e.g., MRI, ultrasound of the brain, and PET scans, can be performed to rule out other neurological disorders, but are not helpful in diagnosing PD. Further, a subject suspected of having Parkinson's can be administered a sufficient (i.e., high) dose of an anti-Parkinson's therapeutic (e.g., carbidopa-levodopa) and monitor for improvement of symptom(s); an improvement following administration would indicate/confirm a diagnosis of PD.

Treatment for PD include, but are not limited to, therapeutics designed to treat the ongoing symptoms of the disease. These therapeutics include, but are not limited to, carbidopa-levodopa; Inhaled carbidopa-levodopa; Carbidopa-levodopa infusion; Dopamine agonists; MAO B inhibitors; catechol O-methyltransferase (COMT) inhibitors; anticholinergics; and amantadine.

Levodopa, the most effective PD medication, is a natural chemical that passes into the brain and is converted to dopamine. Levodopa is typically combined with carbidopa (e.g., Lodosyn® carbidopa), which protects levodopa from early conversion to dopamine outside the brain, preventing or lessening side effects such as nausea. As the disease progresses to later stages, the benefit from levodopa may become less stable, with a tendency to wax and wane (i.e., “wearing off”). Involuntary movements (dyskinesia) is associated with higher doses of levodopa. Inbrija® levodopa inhalation powder is a therapeutic drug delivering levodopa in an inhaled form. Duopa™ carbidopa/levodopa suspension is a brand-name medication made up of carbidopa and levodopa administered via a feeding tube such that the medication is delivered via a gel form directly to the small intestine. Duopa™ carbidopa/levodopa suspension is for patients with more-advanced Parkinson's who still respond to carbidopa-levodopa, but who have significant fluctuations in their response. Because Duopa™ is continually infused, blood levels of the two drugs (carbidopa and levodopa) remain constant.

Unlike levodopa, dopamine agonists do not change into dopamine, but rather mimic dopamine effects in the patient's brain. Dopamine agonists are less effective than levodopa in treating PD symptoms; however, they last longer and may be used with levodopa to support the off-and-on effect of levodopa. Exemplary dopamine agonists include pramipexole (e.g., Mirapex® pramipexole), ropinirole (e.g., Requip® ropinirole), rotigotine (e.g., Neupro® rotigotine transdermal system, given as a patch), and apomorphine (e.g., Apokyn® apomorphine), which is a short-acting injectable dopamine agonist.

MAO B inhibitors help prevent the breakdown of brain dopamine by inhibiting the brain enzyme monoamine oxidase B (MAO B), which metabolizes brain dopamine. Exemplary MAO B inhibitors include selegiline (e.g., Zelapar® selegiline hydrochloride), rasagiline (e.g., Azilect® rasagiline) and safinamide (e.g., Xadago® safinamide). Administration of the selegiline with levodopa has been shown to help prevent wearing-off.

Catechol O-methyltransferase (COMT) inhibitors mildly prolongs the effect of levodopa therapy by blocking an enzyme that breaks down dopamine. Exemplary COMT inhibitors include entacapone (e.g., Comtan® entacapone), opicapone (e.g., Ongentys® opicapone), and tolcapone (e.g., Tasmar® tolcapone). Tolcapone is rarely prescribed due to a risk of serious liver damage and liver failure.

Anticholinergics were previously administered to be help control the tremor associated with PD. Exemplary anticholinergic include Antipsychotics (clozapine, quetiapine); Atropine; Benztropine (e.g., Cogentin® benztropine mesylate); Biperiden; Chlorpheniramine; Certain SSRIs (Paroxetine); Dicyclomine (Dicycloverine); Dimenhydrinate; Diphenhydramine; Doxepi; Doxylamine; Flavoxate; Glycopyrrolate; Glycopyrronium; Hyoscyamine; Ipratropium; Orphenadrine; Oxitropium; Oxybutynin; Promethazine; Propantheline bromide; Scopolamine; Solifenacin; Tolterodine; Tiotropium; Tricyclic antidepressants; Trihexyphenidyl; Tropicamide; and Umeclidinium.

Amantadine (e.g., Gocovri® amantadine) is an anti-dyskinesia medication prescribed as a mono-therapy to provide short-term relief of symptoms of mild, early-stage PD. It is further prescribed with carbidopa-levodopa therapy during the later stages of PD to control involuntary movements (dyskinesia) induced by carbidopa-levodopa.

Patients with PD can further undergo surgery to implant a deep brain stimulation (DBS) to reduce disease-related symptoms. DBS involves implanting electrodes into a specific part of a patient's brain; the electrodes are connected to a generator implanted in the patient's chest near the collarbone and the generator sends electrical pulses to the patient's brain. DBS is effective in controlling erratic and fluctuating responses to levodopa or for controlling dyskinesia that doesn't improve with medication adjustments. DBS is more commonly used in later stage patients that exhibit unstable responses to medication, e.g., levopoda.

### Treatment Methods

Methods for delivering a nucleic acid and/or a transgene (e.g., a nucleic acid encoding GDNF) to a subject are provided by the disclosure. The methods typically involve administering to a subject an effective amount of a nucleic acid encoding GDNF. In some embodiments, administration is systemic administrations. In some embodiments, administration is local administration. In some embodiments, the nucleic acid is provided in a viral vector and/or in a viral particle, e.g., a rAAV.

One aspect provided herein relates to a method of slowing or inhibiting progression of PD in a subject in need thereof comprising introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the GDNF gene, and wherein the subject does not exhibit an increase in PD-associated symptoms for a least 6 months immediately following the introducing as compared to prior to introducing.

One aspect provided herein relates to a method of slowing or inhibiting progression of PD in a subject in need thereof comprising introducing to the subject a recombinant adeno-associated virus (rAAV) comprising a nucleic acid encoding glial cell line-derived neurotrophic factor (GDNF) operably linked to a promoter, wherein the introducing the rAAV results in at least 30% coverage of the subject's putamen with the rAAV, and wherein the subject does not exhibit an increase in PD-associated symptoms for a least 6 months immediately following the introducing as compared to prior to introducing.

The putamen comprises two bilaterally symmetrical, oblong, ovular subcortical lobes that extend longitudinally about an anterior-posterior (A-P) axis. As used herein, and as can be determined through context, the term “putamen” can refer to either a single putamen (i.e., the left putamen or right putamen) or both putamen collectively. The putamen are located within the paraventricular deep white matter of the forebrain of each brain hemisphere (telencephalon) and comprise a plurality of nerve cell (neuronal) bodies. The putamen form the striatum together with the adjacent caudate nucleus. The striatum is additionally one component of many that form the basal ganglia of each brain hemisphere. Through various pathways, the putamen are connected to the substantia nigra (including the pars compacta and pars reticulata), the globus pallidus, the claustrum, and the thalamus, in addition to many regions of the cerebral cortex. A primary function of the putamen is to regulate the preparation and execution of physical movements and plays a role in various types of learning. The putamen also plays a role in the development of degenerative neurological disorders, such as PD. Retrograde axonal transport of the GDNF protein and/or AAV2 vector from the putamen to substantia nigra is possible; however, anterograde axonal transport of the GDNF protein and/or AAV2 vector to the pars reticulata is more probable in a PD state. The direction of axonal transport can be determined by the vector used to deliver the GDNF transgene.

One aspect provided herein relates to a method of slowing or inhibiting progression of PD in a subject in need thereof comprising introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the GDNF gene and/or wherein at least 30% of the subject's putamen volume is covered by the rAAV.

In one embodiment, the rAAV is introduced via local introduction. In one embodiment, the AAV capsid is a rational haploid, e.g., the capsid is AAV8, AAV9, and contains at least one capsid protein from a Rhesus AAV strain.

In various embodiments, local introduction is introduction directly to the subject's putamen. For example, local introduction can comprise directly introducing the rAAV to one or both of the subject's putamen.

In various embodiments, local introduction is performed simultaneously with non-invasive imaging. For example, the local introduction comprises introducing about half of the total rAAV vector dose to each putamen via intraoperative magnetic resonance image (iMRI)-guided convection enhanced delivery (CED).

In one embodiment, local introduction further comprises introducing an MRI contrast agent at the same time or substantially the same time as the AAV vector. Exemplary MRI contrast agents include gadoterate; gadobutro; gadoteridol; gadopentetate; gadobenate; gadopentetic acid dimeglumine; gadoxentate; gadoversetamide; gadodiamide; gadofosveset; gadocoletic acid; gadomelitol and gadomer.

In one embodiment, the MRI contrast agent is introduced to the subject in the same composition as the rAAV. In one embodiment, the MRI contrast agent is introduced to the subject in a different composition as the rAAV, but are administered at substantially the same time.

In one embodiment, the rAAV is introduced via systemic introduction.

Another aspect provided herein relates to a method of slowing or inhibiting a progression of PD in a subject in need thereof, the method comprising locally introducing to the subject's putamen a recombinant adeno-associated virus (rAAV) vector comprising a nucleic acid encoding glial cell line-derived neurotrophic factor (GDNF) operably linked to a promoter, wherein at least 30% of the volume of the subject's putamen is transduced with the GDNF nucleic acid. In one embodiment, transducing the putamen is transducing the putaminal neuron population. In one embodiment, at least 30% of the volume of the subject's putaminal neuron population are transduced with the GDNF nucleic acid

Another aspect provided herein relates to a method of slowing or inhibiting a progression of PD in a subject in need thereof comprising transducing greater than or equal to about 30% of the volume of the subject's putamen with a glial cell line-derived neurotrophic factor (GDNF) gene, wherein the subject does not exhibit a substantial increase in PD-associated symptoms for a least 6 months following the transducing. In one embodiment, transducing is performed by administering a rAAV comprising the GDNF gene to each putamen of the subject's brain hemisphere. In one embodiment, transducing the putamen is transducing the putaminal neuron population. In one embodiment, at least 30% of the volume of the subject's putaminal neuron population are transduced with the GDNF nucleic acid

Another aspect provided herein relates to a method of reducing or stabilizing an initial Movement Disorder Society-Unified PD Rating Scale Part III (MDS-UPDRS III) score in a subject having PD comprising administering to the subject's putamen a recombinant adeno-associated virus (rAAV) comprising a nucleic acid encoding glial cell line-derived neurotrophic factor (GDNF) operably linked to a promoter, wherein the subject has a second MDS-UPDRS III score at 6 months following the administration is decreased or stabilized as compared to the initial MDS-UPDRS III score of the subject prior to administering.

Another aspect provided herein relates to a method of treating a subject mildly affected by PD comprising administering to each of the subject's putamen a recombinant adeno-associated virus (rAAV) comprising a nucleic acid encoding glial cell line-derived neurotrophic factor (GDNF) operably linked to a promoter, wherein at least 30% of the subject's putamen is transduced with GDNF and/or wherein at least 30% of the subject's putamen volume is covered by the rAAV, and wherein the subject has a second MDS-UPDRS III score at 6 months post-administering that is stabilized as compared to the initial MDS-UPDRS III score.

Another aspect provided herein relates to a method of treating a subject moderately affected by PD comprising administering to each of the subject's putamen a recombinant adeno-associated virus (AAV) comprising a nucleic acid encoding glial cell line-derived neurotrophic factor (GDNF) operably linked to a promoter, wherein at least 30% of the subject's putamen is transduced with the transgene and/or wherein at least 30% of the subject's putamen volume is covered by the rAAV, and wherein the subject has a second MDS-UPDRS III score at 6 months post-administering that is at least about 20% lower than the initial MDS-UPDRS III score.

Another aspect provided herein relates to a method of slowing or inhibiting progression of PD in a subject in need thereof comprising locally introducing to each of the subject's putamen a recombinant adeno-associated virus (rAAV) vector comprising a nucleic acid encoding glial cell line-derived neurotrophic factor (GDNF) operably linked to a promoter; and locally introducing an MRI contrast agent to each of the subject's putamen at substantially the same time as the rAAV, wherein at least 30% of the volume of the subject's putamen is transduced with the nucleic acid and/or wherein at least 30% of the subject's putamen volume is covered by the rAAV, and wherein the subject does not exhibit a substantial increase in PD-associated symptoms for a least 6 months immediately following the introducing as compared to prior to introducing.

Another aspect provided herein relates to a method of slowing or inhibiting progression of PD in a subject in need thereof comprising introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the GDNF gene and/or wherein at least 30% of the subject's putamen volume is covered by the rAAV, and wherein the subject does not exhibit a substantial increase in PD-associated symptoms for a least 6 months immediately following the introducing as compared to prior to introducing.

In various embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the volume of the subject's putamen is transduced with the GDNF gene. In one embodiment, transduction of the subject's putamen is assessed via non-invasive imaging, for example, via MRI. One skilled in the art can assess the transduction of the rAAV by measuring the total volume of the putamen comprising the rAAV (e.g., as assessed by the infused MRI contrast agent) as compared to the total volume that does not comprise the rAAV.

In one embodiment, transducing the putamen is transducing intrinsic medium spiny neurons (MSNs) of the putamen. In one embodiment, at least 5%, at least 10%, at least 15%, at least 20%, at least 25%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 85%, at least 90%, at least 95%, at least 99% or more of the MSNs are transduced with the nucleic acid, e.g., GDNF.

In one embodiment, transducing the putamen is transducing the putaminal neuron population. In one embodiment, at least 30% of the volume of the subject's putaminal neuron population are transduced with the GDNF nucleic acid. In various embodiments, the coverage is at least 5%, at least 10%, at least 15%, at least 20%, at least 25%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 85%, at least 90%, at least 95%, at least 99% or more of the subject's putaminal neuron population are transduced with the GDNF nucleic acid.

In various embodiments, the coverage is at least 5%, at least 10%, at least 15%, at least 20%, at least 25%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 85%, at least 90%, at least 95%, at least 99% or more of the volume of the subject's putamen is covered with the rAAV. In some embodiments, coverage of the subject's putamen is assessed via non-invasive imaging, for example, via MRI. One skilled in the art can assess the coverage of the rAAV by measuring the total volume of the putamen comprising the rAAV (e.g., as assessed by the infused MRI contrast agent) as compared to the total volume of the putamen.

In one embodiment, the MRI contrast agent is co-administered or co-introduced with any of the rAAVs described herein to provide enhanced real-time intraoperative MRI monitoring of the CED distribution and to assess the volume of transduction.

### Non-Invasive Imaging

In one embodiment, local administration is performed simultaneously with non-invasive imaging, e.g., to guide local delivery to a preferred or predetermined location or example, the putamen, and/or to visualize transduction following administration. In one embodiment, the non-invasive imaging is intraoperative magnetic resonance image (iMRI)-guided convection enhanced delivery (CED). As used herein, “intraoperative magnetic resonance image (iMRI)” refers to an MRI image, for example, of the brain, acquired during a neurosurgical procedure. iMRI technology can be relied upon to create accurate, real time pictures of the brain for guidance during a neurosurgical procedure, e.g., removal of a tumor or placement of a therapeutic to a desired location (e.g., the putamen). As used herein, “Convection-enhanced delivery (CED)” refers to a drug-delivery technique that uses positive hydrostatic pressure to deliver a fluid containing a therapeutic substance by bulk flow directly into the interstitial space within a localized region of the brain parenchyma. Direct intracerebral CED circumvents the blood-brain barrier and provides a wider, more homogenous distribution than bolus deposition (focal injection) or other diffusion-based direct delivery approaches. CED is further described in, e.g., Rogawski M A, Neurotherapeutics. 2009 April; 6(2): 344-351 and Mehta A M, et al. Neurotherapeutics. 2017 April; 14(2):358-371, the contents of each of which are incorporated herein by reference in their entireties.

In on embodiment, iMRI will be used to monitor administration of the rAAV or composition thereof described herein using T1-weighted sequences to visualize the MRI contrast agent, e.g., the gadolinium-based contrast agent, that is co-infused with the rAAV or composition thereof.

In one embodiment, local introduction further comprises introducing an MRI contrast agent at substantially the same time as the AAV vector. In this case, MRI contrast agents are utilized to improve the visibility of internal brain structures captured in an MRI image. Preferred are paramagnetic contrast agents comprising gadolinium(III), known in the art as gadolinium-based MRI contrast agents (GBCAs), see “Gadolinium(III) Chelates as MRI Contrast Agents Structure, Dynamics, and Applications” by P. Caravan et al. Chem. Rev. 99, 2293-2352 (1999), incorporated herein in its entirety by reference. Other contrast agents that may be used include gadoxetate disodium (e.g., Eovist™ gadoxetate disodium; Schering AG); the contrast agents disclosed in U.S. Pat. Nos. 5,798,092 and 5,695,739; gadobenate dimeglumine (e.g., MultiHance™ gadobenate dimeglumine, Bracco SpA); and the contrast agents disclosed in U.S. Pat. No. 5,733,528. Particularly preferred are “blood pool” MRI contrast agents, see “Blood pool Contrast Agents for Cardiovascular MR Imaging” by L. J. M. Kroft et al. JMRI 10, 395-403 (1999), incorporated herein by reference, and “The Future of Contrast-Enhanced Magnetic Resonance Angiography: Are Blood Pool Agents Needed?” by A. Muhler Invest. Radiol. 33, 709-714 (1998), also incorporated herein by reference. Examples of blood pool contrast agents include MP-2269 (Mallinckrodt, Inc.); the contrast agents disclosed in U.S. Pat. No. 5,888,576; MS-325 (EPIX Medical, Inc.); the contrast agents disclosed in PCT publication WO 96/23526; P760 (Geurbet); gadolinium-diethylene triamine pentaacetic acid (GD-DTPA; e.g., Gadomer-17™, Schering AG); the contrast agents disclosed in U.S. Pat. Nos. 5,876,698, 5,820,849, 5,681,543, 5,650,136, and 5,364,614; gadoterate meglumine (e.g., Clariscan™ gadoterate meglumine, Nycomed Amersham); the contrast agents disclosed in PCT publications WO 96/09840 and WO 9725073; B22956/1 (Bracco SpA); and the contrast agents disclosed in PCT publications WO 00/30688, WO 98/05625, WO 98/05626, WO 95/32741, WO 98/38738, WO 95/32741, and U.S. Pat. No. 5,649,537. Other examples of such blood pool agents, include but are not limited to, ferucarbotran (e.g., Resovist™ ferucarbotran) or SHU 555 A and C (Schering). The contents of all patents and patent applications noted herein above are explicitly incorporated herein by reference in their entireties.

Exemplary MRI contrast agents include gadoterate; gadobutro; gadoteridol; gadopentetate; gadobenate; gadopentetic acid dimeglumine; gadoxentate; gadoversetamide; gadodiamide; gadofosveset; gadocoletic acid; gadomelitol and gadomer.

In one embodiment, the MRI contrast agent is gadoteridol (e.g., ProHance® gadoteridol). In one embodiment, gadoteridol (e.g., ProHance® gadoteridol) is administered in a 2 mM solution.

MRI contrast agents may be administered by injection into the blood stream (intravenously) or orally, depending on the subject of interest. Oral administration is well suited to G.I. tract scans, while intravascular administration proves more useful for most other scans. In one embodiment, the MRI contrast agent is administered in the same composition as the rAAV. In one embodiment, the MRI contrast agent is administered in a separate composition as the rAAV, but is administered concurrently with the separate rAAV composition. When administered in a separate composition, the MRI contrast agent need not be administered in the same manner as the rAAV. For example, if the rAAV is locally administered, e.g., to the putamen, the MRI contrast agent can be administered intravenously or orally.

MRI brain scans can be performed pre-operatively as part of the screening process, as well as during the gene therapy infusion procedure and at 6- and 18-months after dosing. Scans may be obtained at other time points if deemed necessary by the investigators. MRI brain scans can be obtained, for example, on a 1.5 or 3T scanner and sequences may include T1, T2, turbo FLAIR, T2 gradient echo and diffusion. Optional imaging at screening 18-months may also include expanded diffusion weighted sequences, resting state, and functional assessments with image acquisition while participants perform simple tasks (i.e. finger tapping or hand grasping). Total imaging time is 90 minutes per session, with the inclusion of functional and resting state imaging.

Exemplary non-invasive imaging techniques that can be utilized in the methods described herein include ultrasound, computed tomography (CT); functional magnetic resonance imaging (fMRI); iMRI; positron emission tomography (PET); electroencephalography (EEG); magnetoencephalography (MEG); functional near-infrared spectroscopy (fNIRS); DaTscan Dopamine Transporter Imaging; FDG imaging and combinations thereof.

Ioflupane 1-123 (e.g., DaTscan™ ioflupane I23) selectively binds to presynaptic dopamine transporters and provides a method for imaging nigrostriatal terminals in the striatum. DaTscanrm ioflupane I23 is an FDA-approved radiopharmaceutical used in conjunction with single photon emission computed tomography (SPECT) scan for use in adults. Iodine-123 is a cyclotron-produced radionuclide that decays to ¹²³Te by electron capture and has a physical half-life of 13.2 hours. The recommended dose is 111 to 185 MBq (3 to 5 mCi) administered intravenously in adults. The Effective Dose resulting from a DaTscan administration with an administered activity of 185 MBq (5 mCi) is 3.94 mSv in an adult. DaTscan injection may contain up to 6% of free iodide (iodine 123). To decrease thyroid accumulation of iodine-123, a dose up to 100 mg of Potassium Iodide Oral Solution or Lugol's Solution will be administered.

Fluoro-2-Deoxyglucose (FDG) is a common FDA-approved radiopharmaceutical tracer used with positron emission topography (PET) imaging to measure glucose metabolism in the brain and other organs. Brain metabolic patterns specific to PD, and not present in other parkinsonian-like diseases, have been characterized. FDG PET will be utilized to confirm PD diagnosis during screening. FDG is F¹⁸ labeled with a half-life of 110 minutes. The recommended dose is 111 to 185 MBq (3 to 5 mCi) administered intravenously in adults. The Effective Dose resulting from an FDG scan with an administered activity of 185 MBq (5 mCi) is 3.51 mSv in an adult.

In one embodiment, the coverage or transduction of the putamen is assessed via non-invasive imaging, for example, intraoperative MRI. Volume of the putamen that is transduced with GDNF is indirectly determined by measuring the percent of the putamen showing CED-infused MRI contrast within the putamen as compared to the total volume of the putamen. In one embodiment, the volume of the putamen transduced is assessed via F-DOPA PET imaging that is correlated to the intraoperative CED-infused MRI contrast localization.

As used herein, “coverage” refers to the volume of the putamen occupied by the infused rAAV relative to the total volume of the putamen. The coverage provides that putamen cells are exposed to the rAAV. There are many ways to determine coverage by the therapeutic agent delivered following administration. For example, in one embodiment, the coverage of the putamen can be assessed via non-invasive imaging, for example, via co-infusion with a MRI contrast agent that can be visualized. For example, the coverage of the putamen following introduction/administration can be determined by measuring the area or volume of the putamen that displays the co-infused MRI agent and comparing it to the area or volume of the putamen that does not display the agent. Similarly, the coverage is assessed via F-DOPA PET imaging. Further, in one embodiment, the rAAV can be further comprise a reporter gene, e.g., a fluorescent tag, that can be visualized postmortem using standard histological methods, e.g., microscopy.

As used herein, “transduction” or “transduced” refers to a cell within the putamen that comprises the administered rAAV or composition thereof. In one embodiment, the transduced cell comprises the genome of the rAAV, and has the potential to express the GDNF transgene. In one embodiment, the transduced cell comprises the genome of the rAAV and does not need to comprise the ability to express the GDNF transgene. In one embodiment, the transduced cell transiently expresses the GDNF transgene. In one embodiment, the transduced cell stably expresses the GDNF transgene.

There are many ways to determine transduction following administration; these are typically second hand, inferential (or indirect) techniques. For example, transduction can be assessed via co-infusion with a MRI contrast agent that can be visualized. The percentage of transduced cells of the putamen can be determined, e.g., by measuring the percent of the cells in the putamen that comprises the rAAV or composition, as compared to the total volume of the putamen. The transduction can be accessed via non-invasive imaging, for example, via co-infusion with a MRI contrast agent that can be visualized. In one embodiment, the rAAV can be further comprise a reporter gene, e.g., a fluorescent tag, that can be visualized using standard methods, e.g., microscopy; such reporter gene can be utilized to determine the percent transduction. Probes designed to target the rAAV (e.g., a capsid protein) can be used to assess whether the cell is transduced with the rAAV. Further, probes designed to target the GDNF nucleic action can be used to assess whether the cell expresses the GDNF transgene. In one embodiment, the volume of the putamen transduced is assessed via F-DOPA PET imaging. Further, in one embodiment, the rAAV can be further comprise a reporter gene, e.g., a fluorescent tag) that can be visualized using standard methods, e.g., microscopy.

### Diagnostic Assays for Parkinson's Disease and Disease Progression

The progression and severity of PD is often measured using a various clinical surveys which assess various symptoms and the mental status of a subject having or thought to have PD. These clinical surveys can be completed by the subject, the subject's caretaker, and/or a skilled physician. Often, these surveys are used by clinicians and researchers to assess the longitudinal course of PD during the course of treatment or in a clinical study. The results of such surveys can, e.g., aid the clinician or researcher in determining the best course of action for treating a subject, i.e., altering the type of a therapeutic, the dosage of a therapeutic, or frequency of administration for a therapeutic.

In one embodiment, the methods described herein further comprise the step of determining an initial score of at least one diagnostic assay described herein for a subject prior to introducing an rAAV described herein. In one embodiment, the diagnostic assay can be Movement Disorder Society-Unified Parkinson Disease Rating Scale (MDS-UPDRS), Non-Motor Symptoms Scale (NMSS), Parkinson's Disease Questionnaire (PDQ-39) score; MDS-UPDRS Part III; Modified Hoehn and Yahr; Stand-Walk-Sit; 9-Hole Pegboard Dexterity Test; and Standing Balance Test; Global Impression (CGI & PGI); Brief Smell Identification Test (BSIT); Parkinson's Disease Sleep Scale (PDSS-2); Scales for Outcomes in Parkinson's Disease-Autonomic (SCOPA-AUT); Global Cognitive Assessment via Montreal Cognitive Assessment (MoCA); 30-Item Boston Naming Test (BNT); Verbal Fluency Test; Cambridge Neuropsychological Test Automated Battery (CANTAB); Beck Depression Inventory-II (BDI-II); Beck Anxiety Inventory (BAI); and Questionnaire for Impulsive-Compulsive Disorders in Parkinson's (QUIP-RS), or any combination thereof.

In one embodiment, the methods described herein further comprises the step of receiving an initial score of at least one diagnostic assay described herein for a subject prior to introducing any of the rAAVs described herein.

### Movement Disorder Society-Unified Parkinson Disease Rating Scale (MDS-UPDRS)

The Unified Parkinson Disease Rating Scale (UPDRS) is a rating scale to assess the short term (less than 1 year) and long term (greater than 1 year) progression of PD. The UPDRS is a uniform and accepted assay utilized in a clinical setting that allows a clinician to follow the progression of patients' symptoms in an objective manner. This test is made up of six parts and is both self-administered and clinican/researcher-administered. The six parts include-Part I: evaluation of mentation, behavior, and mood, including intellectual impairment, thought disorder, motivation/initative, depression; Part II: self-evaluation of the activities of daily life (ADLs) speech, salivation, swallowing, handwriting, cutting food, dressing, hygiene, turning in bed, falling, freezing, walking, tremor, sensory complaints; Part III: clinician-scored monitored motor evaluation, including speech, facial expression, tremor at rest, action tremor, rigidity, finger taps, hand movements, hand pronation and supination, leg agility, arising from chair, posture, gait, postural stability, body bradykinesia; Part IV: complications of therapy, including dyskinesia-duration, dyskinesia-disability, dyskinesia-pain, early morning dystonia, OFF-predictable, OFF-unpredictable, OFF-sudden, OFF-duration, anorexia-nausea-vomiting, sleep disturbance, symptomatic orthostasis; Part V: Hoehn and Yahr staging of severity of PD; and Part VI: Schwab and England ADL scale.

The Movement Disorder Society-Unified Parkinson Disease Rating Scale (MDS-UPDRS) is a rating scale (i.e., from 0-272) to assess the short term (less than 1 year) and long term (greater than 1 year) progression of PD. Historically, researchers use the UPDRS to measure therapeutic benefits from a given therapy in a unified and accepted rating system. The MDS-UPDRS is an updated version of the UPDRS took aspects of nonmotor functioning out of each subcategory. MDS-UPDRS Part I is titled “Non-Motor Experiences of Daily Living” and includes Part IA (concerning a number of behaviors that are assessed by an investigator with all pertinent information from patients and caregivers) and Part IB (completed by the patient with or without the aid of a caregiver, but independently of an investigator). MDS-UPDRS Part II is identical to the second part of the original UPDRS, but has been renamed “Motor Experiences of Daily Living” to separate it from the new title of Part I. MDS-UPDRS Part III is titled “Motor Examination.” MDS-UPDRS Part IV has been condensed relative to UPDRS to include only “Motor Complications.” A total MDS-UPDRS score is a sum of Parts I, II, III (in Off state), and IV, which provides a score of disease severity and progression as it provides both functional and rater-derived subscores. A person of ordinary skill in the art would be able to properly administer the MDS-UPDRS survey and guide a subject to self-administer portions of the MDS-UPDRS survey to determine, e.g., an initial MDS-UPDRS score (i.e., prior to administration of an rAAV described herein), and a second MDS-UPDRS score (i.e., a MDS-UPDRS score subsequent to the administration of rAAV, e.g., at least 6 months or at least 12 months immediately following administration of an rAAV described herein). It is understood that additional MDS-UPDRS scores (e.g., third, fourth, fifth, and so on) can be determined as deemed necessary by, for example, a clinician. It is further understood that the initial and second MDS-UPDRS scores can be any individual part of the MDS-UPDRS survey (e.g., Part I, Part II, Part III, Part IV) or atotal MDS-UPDRS score.

In one embodiment, the methods described herein further comprise the step of determining an initial MDS-UPDRS score for a subject prior to introducing any of the rAAVs described herein. In one embodiment, the methods described herein further comprise the step of receiving an initial MDS-UPDRS score for a subject prior to introducing any of the rAAVs described herein, i.e., receiving an in initial MDS-UPDRS score that was previously determined by a skilled practitioner that is not performing the administration of the rAAV.

In one embodiment, the subject is mildly affected by PD. As used herein, “mildly affected” refers to a subject having an initial MDS-UPDRS score that is less 32.

In one embodiment, the subject mildly affected by PD has an initial MDS-UPDRS score less than 32 prior to the introduction of rAAV and was diagnosed with PD less than 5 years prior to the introduction of rAAV.

In one embodiment, the MDS-UPDRS refers to the MDS-UPDRS III score.

In one embodiment, the subject is moderately affected by PD. As used herein, “moderately affected” refers to a subject having an initial MDS-UPDRS score that is equal to or greater than 32.

In one embodiment, the subject moderately affected by PD has an initial MDS-UPDRS score (e.g., MDS-UPDRS III score) equal to or greater than 32 prior to the introduction of rAAV and was diagnosed with PD less than 4 years prior to the introduction of rAAV.

In one embodiment, the methods described herein further include the step of determining a second MDS-UPDRS score at least 6 months or at least 12 months immediately following administration/introduction of the rAAV.

In one embodiment, the methods described herein further include the step of determining a second MDS-UPDRS score at least 6 months immediately following administration/introduction of the rAAV to the subject who is moderately affected by PD (i.e., the subject having an initial MDS-UPDRS score that is equal to or greater than 32).

In one embodiment, the methods described herein further include the step of determining a second MDS-UPDRS score at least 12 months immediately following administration/introduction of the rAAV to the subject who is mildly affected by PD (i.e., the subject having an initial MDS-UPDRS score that is less 32).

In one embodiment, the subject who is mildly affected by PD does not exhibit an increase of their initial MDS-UPDRS score for at least 12 months immediately following introducing any of the rAAVs described herein. In one embodiment, the subject who is mildly affected by PD does not exhibit an increase of their initial MDS-UPDRS score for at least 1 month; 2 months; 3 months; 4 months; 5 months; 6 months; 7 months; 8 months; 9 months; 10 months; 11 months; 13 months; 14 months; 15 months; 16 months; 17 months; 18 months; 19 months; 20 months; 21 months; 22 months; 23 months; 24 months; or longer immediately following introducing any of the rAAVs described herein.

In one embodiment, the subject who is mildly affected by PD, does not exhibit a substantial increase of their initial MDS-UPDRS score for at least 12 months immediately following introducing any of the rAAVs described herein. As used herein, “substantial increase” refers to an increase that is no more than 10% of the initial MDS-UPDRS score. In one embodiment, the subject who is mildly affected by PD does not exhibit a substantial increase of their initial MDS-UPDRS score for at least 1 month; 2 months; 3 months; 4 months; 5 months; 6 months; 7 months; 8 months; 9 months; 10 months; 11 months; 13 months; 14 months; 15 months; 16 months; 17 months; 18 months; 19 months; 20 months; 21 months; 22 months; 23 months; 24 months; or longer immediately following introducing any of the rAAVs described herein.

In one embodiment, the subject who is mildly affected by PD exhibit a stabilization of their initial MDS-UPDRS score for at least 6 months immediately following introducing any of the rAAVs described herein. As used herein, “stabilization” refers to an initial MDS-UPDRS score that does not increase or decrease by greater than 10%, i.e., the second MDS-UPDRS score is no more than +/−10% of the initial MDS-UPDRS score. In one embodiment, the subject who is mildly affected by PD exhibits a stabilization of their initial MDS-UPDRS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein.

In one embodiment, the subject who is mildly affected by PD exhibits a reduction of their initial MDS-UPDRS score for at least 12 months immediately following introducing any of the rAAVs described herein. In one embodiment, the subject who is mildly affected by PD exhibits a reduction of their initial MDS-UPDRS score for at least 1 month; 2 months; 3 months; 4 months; 5 months; 6 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein. In one embodiment, the reduction of the initial MDS-UPDRS score is by at least 1 point, 2 points; 3 points; 4 points; 5 points; 6 points; 7 points; 8 points; 9 points; 10 points; 11 points; 12 points; 13 points; 14 points; 15 points; 16 points; 17 points; 18 points; 19 points; 20 points; 21 points; 22 points; 23 points; 24 points; 25 points; 26 points; 27 points; 28 points; 29 points; 30 points; or 31 points; or by at least 5%; 10%; 15%; 20%; 25%; 30%; 35%; 40%; 45%; 50%; 55%; 60%; 65%; 70%; 75%; 80%; 85%; 90%; 95%; or greater.

In one embodiment, the subject who is moderately affected by PD exhibits a reduction of their initial MDS-UPDRS score for at least 6 months immediately following introducing any of the rAAVs described herein. In one embodiment, the subject who is moderately affected by PD exhibits a reduction of their initial MDS-UPDRS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein. In one embodiment, the reduction of the initial MDS-UPDRS score is by at least 20%. In one embodiment, the reduction of the initial MDS-UPDRS score is by at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater, or by at least 1 point; 2 points; 3 points; 4 points; 5 points; 6 points; 7 points; 8 points; 9 points; 10 points; 11 points; 12 points; 13 points; 14 points; 15 points; 16 points; 17 points; 18 points; 19 points; 20 points; 21 points; 22 points; 23 points; 24 points; 25 points; 26 points; 27 points; 28 points; 29 points; 30 points; 31 points; 32 points; 33 points; 34 points; 35 points; 36 points; 37 points; 38 points; 39 points; 40 points; 41 points; 42 points; 43 points; 44 points; 45 points; 46 points; 47 points; 48 points; 49 points; 50 points; 51 points; 52 points; 53 points; 54 points; 55 points; 56 points; 57 points; 58 points; 59 points; 60 points; 61 points; 62 points; 63 points; 64 points; 65 points; 66 points; 67 points; 68 points; 69 points; 70 points; 71 points; 72 points; 73 points; 74 points; 75 points; 76 points; 77 points; 78 points; 79 points; 80 points; 81 points; 82 points; 83 points; 84 points; 85 points; 86 points; 87 points; 88 points; 89 points; 90 points; 91 points; 92 points; 93 points; 94 points; 95 points; 96 points; 97 points; 98 points; 99 points; 100 points; 101 points; 102 points; 103 points; 104 points; 105 points; 106 points; 107 points; 108 points; 109 points; 110 points; 111 points; 112 points; 113 points; 114 points; 115 points; 116 points; 117 points; 118 points; 119 points; 120 points; 121 points; 122 points; 123 points; 124 points; 125 points; 126 points; 127 points; 128 points; 129 points; 130 points; 131 points; 132 points; 133 points; 134 points; 135 points; 136 points; 137 points; 138 points; 139 points; 140 points; 141 points; 142 points; 143 points; 144 points; 145 points; 146 points; 147 points; 148 points; 149 points; 150 points; 151 points; 152 points; 153 points; 154 points; 155 points; 156 points; 157 points; 158 points; 159 points; 160 points; 161 points; 162 points; 163 points; 164 points; 165 points; 166 points; 167 points; 168 points 169 points; 170 points; 171 points; 172 points; 173 points; 174 points; 175 points; 176 points; 177 points; 178 points; 179 points; 180 points; 181 points; 182 points; 183 points; 184 points; 185 points; 186 points; 187 points; 188 points; 189 points; 190 points; 191 points; 192 points; 193 points; 194 points; 195 points; 196 points; 197 points; 198 points; 199 points; 200 points; 201 points; 202 points; 203 points; 204 points; 205 points; 206 points; 207 points; 208 points; 209 points; 210 points; 211 points; 212 points; 213 points; 214 points; 215 points; 216 points; 217 points; 218 points; 219 points; 220 points; 221 points; 222 points; 223 points; 224 points; 225 points; 226 points; 227 points; 228 points; 229 points; 230 points; 231 points; 232 points; 233 points; 234 points; 235 points; 236 points; 237 points; 238 points; 239 points; 240 points; 241 points; 242 points; 243 points; 244 points; 245 points; 246 points; 247 points; 248 points; 249 points; 250 points; 251 points; 252 points; 253 points; 254 points; 255 points; 256 points; 257 points; 258 points; 259 points; 260 points; 261 points; 262 points; 263 points; 264 points; 265 points; 266 points; 267 points; 268 points; 269 points; 270 points; 271 points; or 272 points.

In one embodiment, the subject who is moderately affected by PD does not exhibit an increase of their initial MDS-UPDRS score for at least 6 months immediately following introducing any of the rAAVs described herein. In one embodiment, the subject who is moderately affected by PD does not exhibit an increase of their initial MDS-UPDRS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein.

In one embodiment, the subject who is moderately affected by PD does not exhibit a substantial increase of their initial MDS-UPDRS score for at least 6 months immediately following introducing any of the rAAVs described herein. As used herein, “substantial increase” refers to an increase that is no more than 10% of the initial MDS-UPDRS score. In one embodiment, the subject who is moderately affected by PD does not exhibit a substantial increase of their initial MDS-UPDRS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein.

### Non-Motor Symptoms Scale (NMSS)

The Non-Motor Symptoms Scale (NMSS) is a 30-item self-administered survey scale to assess a wide range of non-motor symptoms in subjects with PD. Non-motor symptoms in PD generally include neuropsychiatric symptoms, sleep disorders, autonomic dysfunction, gastrointestinal symptoms and sensory symptoms, and can significantly reduce quality of life. The NMSS measures the severity and frequency of non-motor symptoms across nine dimensions: cardiovascular, sleep/fatigue, mood/cognition, perceptual problems, attention/memory, gastrointestinal, urinary, sexual function, and miscellaneous. The scale can be used for patients at all stages of PD. The scores for each item are based on a combination of severity (from 0 to 3) and frequency scores (from 1 to 4), to capture symptoms that are severe but relatively infrequent, or that are less severe but persistent. The total NMSS score ranges from 0 to 360

In one embodiment, the methods described herein further comprises the step of determining an initial NMSS score for a subject prior to introducing any of the rAAVs described herein. In one embodiment, the methods described herein further comprise the step of receiving an initial NMSS score for a subject prior to introducing any of the rAAVs described herein, i.e., receiving an in initial NMSS score that was previously determined by a clinician/research that is not performing the administration of the rAAV.

In one embodiment, the methods described herein further include the step of determining a second NMSS score at least 6 months or at least 12 months immediately following administration/introduction of the rAAV.

In one embodiment, the subject exhibits a decrease of their initial NMSS score for at least 6 months following introducing any of the rAAVs described herein. In one embodiment, the subject exhibits a decrease of their initial NMSS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein. In one embodiment, the decrease of the initial NMSS score is by at least 20%. In one embodiment, the decrease of the initial NMSS score is by at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater, or by at least 1 point; 2 points; 3 points; 4 points; 5 points; 6 points; 7 points; 8 points; 9 points; 10 points; 11 points; 12 points; 13 points; 14 points; 15 points; 16 points; 17 points; 18 points; 19 points; 20 points; 21 points; 22 points; 23 points; 24 points; 25 points; 26 points; 27 points; 28 points; 29 points; 30 points; 31 points; 32 points; 33 points; 34 points; 35 points; 36 points; 37 points; 38 points; 39 points; 40 points; 41 points; 42 points; 43 points; 44 points; 45 points; 46 points; 47 points; 48 points; 49 points; 50 points; 51 points; 52 points; 53 points; 54 points; 55 points; 56 points; 57 points; 58 points; 59 points; 60 points; 61 points; 62 points; 63 points; 64 points; 65 points; 66 points; 67 points; 68 points; 69 points; 70 points; 71 points; 72 points; 73 points; 74 points; 75 points; 76 points; 77 points; 78 points; 79 points; 80 points; 81 points; 82 points; 83 points; 84 points; 85 points; 86 points; 87 points; 88 points; 89 points; 90 points; 91 points; 92 points; 93 points; 94 points; 95 points; 96 points; 97 points; 98 points; 99 points; 100 points; 101 points; 102 points; 103 points; 104 points; 105 points; 106 points; 107 points; 108 points; 109 points; 110 points; 111 points; 112 points; 113 points; 114 points; 115 points; 116 points; 117 points; 118 points; 119 points; 120 points; 121 points; 122 points; 123 points; 124 points; 125 points; 126 points; 127 points; 128 points; 129 points; 130 points; 131 points; 132 points; 133 points; 134 points; 135 points; 136 points; 137 points; 138 points; 139 points; 140 points; 141 points; 142 points; 143 points; 144 points; 145 points; 146 points; 147 points; 148 points; 149 points; 150 points; 151 points; 152 points; 153 points; 154 points; 155 points; 156 points; 157 points; 158 points; 159 points; 160 points; 161 points; 162 points; 163 points; 164 points; 165 points; 166 points; 167 points; 168 points; 169 points; 170 points; 171 points; 172 points; 173 points; 174 points; 175 points; 176 points; 177 points; 178 points; 179 points; 180 points; 181 points; 182 points; 183 points; 184 points; 185 points; 186 points; 187 points; 188 points; 189 points; 190 points; 191 points; 192 points; 193 points; 194 points; 195 points; 196 points; 197 points; 198 points; 199 points; 200 points; 201 points; 202 points; 203 points; 204 points; 205 points; 206 points; 207 points; 208 points; 209 points; 210 points; 211 points; 212 points; 213 points; 214 points; 215 points; 216 points; 217 points; 218 points; 219 points; 220 points; 221 points; 222 points; 223 points; 224 points; 225 points; 226 points; 227 points; 228 points; 229 points; 230 points; 231 points; 232 points; 233 points; 234 points; 235 points; 236 points; 237 points; 238 points; 239 points; 240 points; 241 points; 242 points; 243 points; 244 points; 245 points; 246 points; 247 points; 248 points; 249 points; 250 points; 251 points; 252 points; 253 points; 254 points; 255 points; 256 points; 257 points; 258 points; 259 points; 260 points; 261 points; 262 points; 263 points; 264 points; 265 points; 266 points; 267 points; 268 points; 269 points; 270 points; 271 points; 272 points; 273 points; 274 points; 275 points; 276 points; 277 points; 278 points; 279 points; 280 points; 281 points; 282 points; 283 points; 284 points; 285 points; 286 points; 287 points; 288 points; 289 points; 290 points; 291 points; 292 points; 293 points; 294 points; 295 points; 296 points; 297 points; 298 points; 299 points; 300 points; 301 points; 302 points; 303 points; 304 points; 305 points; 306 points; 307 points; 308 points; 309 points; 310 points; 311 points; 312 points; 313 points; 314 points; 315 points; 316 points; 317 points; 318 points; 319 points; 320 points; 321 points; 322 points; 323 points; 324 points; 325 points; 326 points; 327 points; 328 points; 329 points; 330 points; 331 points; 332 points; 333 points; 334 points; 335 points; 336 points; 337 points; 338 points; 339 points; 340 points; 341 points; 342 points; 343 points; 344 points; 345 points; 346 points; 347 points; 348 points; 349 points; 350 points; 351 points; 352 points; 353 points; 354 points; 355 points; 356 points; 357 points; 358 points; 359 points; or 360 points.

In one embodiment, the subject does not exhibit an increase of their initial NMSS score for at least 6 months following introducing any of the rAAVs described herein. In one embodiment, a subject does not exhibit an increase of their initial NMSS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein.

In one embodiment, the subject does not exhibit a substantial increase of their initial NMSS score for at least 6 months following introducing any of the rAAVs described herein. As used herein, “substantial increase” refers to an increase that is no more than 10% of the initial NMSS score. In one embodiment, a subject does not exhibit a substantial increase of their initial NMSS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein.

In one embodiment, the subject exhibits a stabilization of their initial NMSS score for at least 6 months immediately following introducing any of the rAAVs described herein. As used herein, “stabilization” refers to an initial NMSS score that does not increase or decrease by greater than 10%. In one embodiment, the subject exhibits a stabilization of their initial NMSS score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein.

### Parkinson's Disease Questionnaire (PDQ-39) Score

The Parkinson's Disease Questionnaire (PDQ-39) is a 39-item self-administered questionnaire with eight subscales: mobility, activities of daily living (ADLs), emotional well-being, stigma, social support, cognitive impairment, communication, and physical discomfort. The questionnaire is scored on a scale from 0 to 100 with lower scores indicating a better perception of health status, and higher scores indicating a more severe state of the disease. The PDQ-39 can be used as a reliable tool for measuring quality of life for individuals with PD. Its inclusion in comprehensive assessment is especially important due to the tendency of treatment to focus on motor deficits and cardinal features rather than other clinical features including depression, cognitive impairment, and fall risk which can significantly impact quality of life.

In one embodiment, the methods described herein further comprises the step of determining an initial PDQ-39 score for a subject prior to introducing any of the rAAVs described herein. In one embodiment, the methods described herein further comprise the step of receiving an initial PDQ-39 score for a subject prior to introducing any of the rAAVs described herein, i.e., receiving an in initial PDQ-39 score that was previously determined by a clinician/research that is not performing the administration of the rAAV.

In one embodiment, the methods described herein further include the step of determining a second PDQ-39 score at least 6 months or at least 12 months immediately following administration/introduction of the rAAV.

In one embodiment, the subject exhibits a decrease of their initial PDQ-39 score for at least 6 months following introducing any of the rAAVs described herein. In one embodiment, a subject who is a decrease of their initial PDQ-39 score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein. In one embodiment, the decrease of the initial PDQ-39 score is by at least 20%. In one embodiment, the decrease of the initial PDQ-39 score is by at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater, or by at least 1 point; 2 points; 3 points; 4 points; 5 points; 6 points; 7 points; 8 points; 9 points; 10 points; 11 points; 12 points; 13 points; 14 points; 15 points; 16 points; 17 points; 18 points; 19 points; 20 points; 21 points; 22 points; 23 points; 24 points; 25 points; 26 points; 27 points; 28 points; 29 points; 30 points; 31 points; 32 points; 33 points; 34 points; 35 points; 36 points; 37 points; 38 points; 39 points; 40 points; 41 points; 42 points; 43 points; 44 points; 45 points; 46 points; 47 points; 48 points; 49 points; 50 points; 51 points; 52 points; 53 points; 54 points; 55 points; 56 points; 57 points; 58 points; 59 points; 60 points; 61 points; 62 points; 63 points; 64 points; 65 points; 66 points; 67 points; 68 points; 69 points; 70 points; 71 points; 72 points; 73 points; 74 points; 75 points; 76 points; 77 points; 78 points; 79 points; 80 points; 81 points; 82 points; 83 points; 84 points; 85 points; 86 points; 87 points; 88 points; 89 points; 90 points; 91 points; 92 points; 93 points; 94 points; 95 points; 96 points; 97 points; 98 points; 99 points; or 100 points.

In one embodiment, the subject does not exhibit an increase of their initial PDQ-39 score for at least 6 months following introducing any of the rAAVs described herein. In one embodiment, a subject does not exhibit an increase of their initial PDQ-39 score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein.

In one embodiment, the subject does not exhibit a substantial increase of their initial PDQ-39 score for at least 6 months following introducing any of the rAAVs described herein. As used herein, “substantial increase” refers to an increase that is no more than 10% of the initial PDQ-39 score. In one embodiment, a subject does not exhibit a substantial increase of their initial PDQ-39 score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer following introducing any of the rAAVs described herein.

In one embodiment, the subject exhibits a stabilization of their initial PDQ-39 score for at least 6 months immediately following introducing any of the rAAVs described herein. As used herein, “stabilization” refers to an initial PDQ-39 score that does not increase or decrease by greater than 10%. In one embodiment, the subject exhibits a stabilization of their initial PDQ-39 score for at least 1 month; 2 months; 3 months; 4 months; 5 months; 7 months; 8 months; 9 months; 10 months; 11 months; 12 months; 13 months; 14 months; 15 months; 16 months; 17 months; 18 months; 19 months; 20 months; 21 months; 22 months; 23 months; 24 months; or longer immediately following introducing any of the rAAVs described herein.

In one embodiment, the subject is administered at least one standard clinical test to assess PD patients' motor symptoms and function prior to and after administration/introduction of any of the rAAVs described herein. Such assessments include MDS-UPDRS Part III; Modified Hoehn and Yahr; Stand-Walk-Sit; 9-Hole Pegboard Dexterity Test; and Standing Balance Test.

The Modified Hoehn & Yahr scale is a 5-stage scale that measures the overall level of disability due to PD (Hoehn and Yahr, 1967).

Stand-Walk-Sit (SWS) is a postural stability and gait assessment that involves standing up from a chair, walking 7 meters (23 feet) in a straight line, turning around and walking back to the chair and sitting down. Timer will be stopped when subjects back contacts back of chair. Subjects will perform this test in practically defined OFF and ON medication states. The SWS test will be video recorded and can be combined with the MDS-UPDRS assessment.

The 9-Hole Pegboard Dexterity Test is a simple test of manual dexterity; it records the time required for the participant to accurately place and remove nine plastic pegs into a plastic pegboard.

The Standing Balance Test assesses a person's ability to orient their body in space, maintain an upright posture under both static and dynamic conditions, and move and walk without falling. It involves the participant assuming and maintaining up to five poses for 50 seconds each. The sequence of poses includes: eyes open on a solid surface, eyes closed on solid surface, eyes open on foam surface, eyes closed on foam surface, and eyes open in tandem stance on solid surface. Detailed stopping rules are in place to ensure participant safety with these progressively demanding poses. Postural sway is recorded for each pose using an accelerometer that the participant wears at waist level. This test takes approximately seven minutes to administer.

In one embodiment, the subject is administered any of the standard clinical tests described herein prior to administration/introduction, and again at least 3, 6, 9, 12 months or later immediately following administration/introduction. In one embodiment, the subject exhibits an improved score on any of the standard clinical test at least 3, 6, 9, 12 months or later immediately following administration/introduction. In one embodiment, the improvement is at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater as compared to the score prior to administration/introduction.

In one embodiment, dyskinesia severity is measured with the Unified Dyskinesia Rating Scale (UDysRS). This scale evaluates involuntary movements often associated with treated PD. It includes two primary sections: Historical [Part 1 (ON-Dyskinesia) and Part 2 (OFF-Dystonia)] and Objective [Part 3 (Impairment) and Part 4 (Disability)]. ON-Dyskinesia refers to the choreiform and dystonic movements described to the patient as jerking or twisting movements that occur when PD medication is working. OFF-Dystonia refers to spasms or cramps that can be painful and occur when PD medications are not taken or are not working. Throughout the assessment, the focus is on these two forms of movements and a continual emphasis must be placed on excluding from the evaluation the impact of parkinsonism itself and tremor from the ratings (Goetz 2008).

In one embodiment, the subject completes a subject-reported PD Motor Diary. Hauser and colleagues have developed a paper motor diary to assess PD motor symptoms over a 24-hour period (Hauser 2004). The diary captures the duration of time, in half-hour intervals, the participant is in the ON state without dyskinesia, ON with non-troublesome dyskinesia, ON with troublesome dyskinesia, in the OFF state, or asleep. Participants are required to record this information at half hour intervals throughout the day.

In one embodiment, the subject is administered UDysRS and/or PD Motor diary prior to administration/introduction, and again at at least 3, 6, 9, 12 months or later immediately following administration/introduction. In one embodiment, the subject exhibits an improved score on the UDysRS and/or PD Motor at at least 3, 6, 9, 12 months or later immediately following administration/introduction. In one embodiment, the improvement is at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater as compared to the score prior to administration/introduction.

In one embodiment, the subject is provided a wearable activity monitor (e.g. Fitbit®) to assess daily activity for at least 18 months immediately following administration/introduction.

In one embodiment, the subject undergoes a Global Disability and Quality of Life Assessment prior to and/or at at least 3, 6, 9, 12 months or later immediately following administration/introduction. Exemplary Global Disability and Quality of Life Assessments include Global Impression (CGI & PGI); Brief Smell Identification Test (BSIT); Parkinson's Disease Sleep Scale (PDSS-2); and Scales for Outcomes in Parkinson's Disease-Autonomic (SCOPA-AUT).

The Clinical Global Impression (CGI) provides an overall clinician-determined summary measure that takes into account all available information, including a knowledge of the patient's history, psychosocial circumstances, symptoms, behavior, and the impact of the symptoms on the patient's ability to function. The CGI actually comprises 2 companion 1-item measures evaluating the following: (a) severity of illness from 1 to 7 and (b) change from the initiation of treatment on a similar 7-point scale. The Patient Global Impression (PGI) is the same as the CGI but is completed by the patient. The PGI and CGI will be completed separately. The PGI is a self-rating tool and will be completed independently by the participant at home with either paper assessment or by answering on a Sponsor provided tablet.

The Brief Smell Identification Test (BSIT) is a 12-item test of olfactory system function using “scratch and sniff” strips. After each scent is released by scratching with a pencil, the participant smells the odor and then answers a four-option multiple choice question related to the scent. This is a self-directed assessment.

The Parkinson's Disease Sleep Scale (PDSS-2) uses visual analogue scales to address 15 commonly reported symptoms associated with sleep disturbance in PD. Subject complete the 15 questions based on their experiences over the previous week. This is a self-directed measure.

The Scales for Outcomes in Parkinson's Disease-Autonomic (SCOPA-AUT) is a 26 item self-report questionnaire of autonomic function. Questions cover upper and lower gastro-intestinal function, urinary function, cardio-circulatory function, sexuality, and other miscellaneous autonomic problems. This is a self-directed symptom scale.

In one embodiment, the subject is administered any of the Global Disability and Quality of Life Assessments described herein prior to administration/introduction, and again at at least 3, 6, 9, 12 months or later immediately following administration/introduction. In one embodiment, the subject exhibits an improved score on the Global Disability and Quality of Life Assessment at at least 3, 6, 9, 12 months or later immediately following administration/introduction. In one embodiment, the improvement is at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater as compared to the score prior to administration/introduction.

In one embodiment, the subject undergoes a neuropsychological testing prior to and/or at at least 3, 6, 9, 12 months or later immediately following administration/introduction. Exemplary neuropsychological tests include Global Cognitive Assessment via Montreal Cognitive Assessment (MoCA); 30-Item Boston Naming Test (BNT); Verbal Fluency Test; Cambridge Neuropsychological Test Automated Battery (CANTAB); Beck Depression Inventory-II (BDI-II); Beck Anxiety Inventory (BAI); and Questionnaire for Impulsive-Compulsive Disorders in Parkinson's (QUIP-RS).

The Montreal Cognitive Assessment (MoCA) was designed as a rapid screening instrument for mild cognitive dysfunction. It assesses different cognitive domains: attention and concentration, executive functions, memory, language, visuoconstructional skills, conceptual thinking, calculations, and orientation. The BNT evaluates confrontation naming and language deficits that can be present in PD. Participants are presented with stimuli of line drawings of objects with increasing naming difficulty and required to provide a response within 20 seconds. The score is based on number of spontaneously provided correct responses, number of cues given, and number of responses after cuing. This brief verbal assessment will be completed with remote guidance from the study team via a video call.

In a verbal fluency test, subjects are asked to produce as many words as possible from a pre-selected letter and category within 60 seconds, with instructions not to use proper nouns or words only changed by a suffix. The number of correct answers will be scored for each category.

The Cambridge Neuropsychological Test Automated Battery (CANTAB) was developed to include sensitive and objective measures of cognitive function in the evaluation of neurologic disorders. The cognitive assessments have been developed to detect changes in neuropsychological performance over time and as an effect of an intervention. Several assessments have been validated in PD with a focus on domains of working memory, episodic memory, executive function, planning, and information processing. All tasks will be completed by subjects using a tablet that includes the collection of response times. CANTAB allows for electronically captured outcome measures that have been validated in a variety of neurodegenerative diseases. CANTAB can be completed by self-direction or guided remotely with the study coordinator or investigator on a Sponsor provided tablet.

Reaction Time (RTI) assesses mental response times as well as a measure of movement time, reaction time, response accuracy and impulsivity.

Motor Screening Task (MOT) provides a general assessment of whether a sensorimotor deficit or lack of comprehension may limit the ability to collect valid data from a participant. This task measures the participant's speed of response and accuracy of pointing to the center of an object on the screen.

One Touch Stockings of Cambridge (OTS) is an assessment of executive function through evaluation of spatial planning and working memory subdomains. Participants are asked to create a 3-D arrangement on screen in a prescribed number of moves. This is measured by number of problems solved on first choice, mean choices correct, meant latency of response, and mean latency to correct.

Paired Associates Learning (PAL) is a visual memory and new learning ability are evaluated by this task. Participants are asked to select boxes in a predesignated pattern with increased difficulty levels. Outcomes are measured by number of errors made, number of trials needed to correctly identify the pattern, stages completed, and memory scores. Estimated completion time: 8 minutes.

Pattern Recognition Memory (PRM) is a test of visual pattern recognition memory in a 2-choice forced discrimination paradigm. Unrelated words presented via audio recording and participant recalls as many as possible immediately or after a delay. This is measured by number and percentage of correct trials and latency of responses.

Multitasking Test (MTT) is an assessment of an individual's ability to interpret and manage conflicting information and to correctly ignore task-irrelevant information. Changing rules between trials places a higher cognitive demand on a participant to reveal underlying deficits of executive dysfunction, a domain often affected in PD patients. This is measured by latency of response and number of errors.

The Beck Depression Inventory-II (BDI-II) can be performed, e.g., at screening as part of the eligibility evaluation, and participants with a score >20 at screening will be excluded from the study and referred to their primary care physician for psychiatric evaluation and treatment. Score guidelines for the BDI-II are provided with the recommendation that thresholds be adjusted based on the characteristics of the sample and the purpose for using the BDI-II. In general, total BDI-II scores of 0-13 indicate minimal depression, scores of 1419 indicate mild depression, scores of 20-28 indicate moderate depression, and scores of 29-63 indicate severe depression. If post-treatment BDI-II scores are greater than 28, then the participant will continue the study but will be referred to their primary care physician for psychiatric evaluation and treatment. This assessment is a self-reported measure.

Beck Anxiety Inventory (BAI) is a 21-question multiple-choice self-report inventory that is used for measuring the severity of anxiety in children and adults. This assessment is a self-reported measure.

Compulsive Disorders Questionnaire for PD Rating Scale (QUIP-RS) is a rating scale designed to measure severity of symptoms and support a diagnosis of impulse control disorders and related disorders in PD. This rating scale covers impulse control behaviors on a 5-point Likert scale to assess the frequency of the following behaviors: gambling, shopping, eating, hypersexuality, simple (punding) and/or complex (hobbyism) repetitive behaviors, and compulsive overuse of medication (dopamine dysregulation syndrome). This assessment is a self-reported measure.

In one embodiment, the subject is administered any neuropsychological tests described herein prior to administration/introduction, and again at at least 3, 6, 9, 12 months or later immediately following administration/introduction. In one embodiment, the subject exhibits an improved score on the neuropsychological test at at least 3, 6, 9, 12 months or later immediately following administration/introduction. In one embodiment, the improvement is at least 1%; 2%; 3%; 4%; 5%; 6%; 7%; 8%; 9%; 10%; 11%; 12%; 13%; 14%; 15%; 16%; 17%; 18%; 19%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99%; or greater as compared to the score prior to administration/introduction.

In one embodiment, the subject does not exhibit any serious adverse event for a least 6 months immediately following the introducing or administering. In one embodiment, the subject does not exhibit any serious adverse event for a least 12 months immediately following the introducing or administering. In one embodiment, the subject does not exhibit any serious adverse event for a for at least 1 month; 2 months; 3 months; 4 months; 5 months; 7 months; 8 months; 9 months; 10 months; 11 months; 13 months; 14 months; 15 months; 16 months; 17 months; 18 months; 19 months; 20 months; 21 months; 22 months; 23 months; 24 months; or longer immediately following introducing or administering. Serious adverse events include, but are not limited to Blood and lymphatic system disorders (e.g., Anemia, Decreased lymphocyte count, and Leukocytosis); Gastrointestinal disorders (e.g., Dyspepsia, Dysphagia, and Constipation); Localized edema; Fatigue; Fall; Bruising of the neck; Aspartate aminotransferase increased; Platelet count decrease; Activated partial thromboplastin time prolonged; Weight loss; Blood lactate dehydrogenase; Metabolism and nutrition disorders (e.g., Hyperglycemia, Hypocalcemia, Hypoalbuminemiaa, Hypophosphatemia, Hypernatremia, Hypoglycemia, Hyperkalemia, and Hyponatremia); Musculoskeletal and connective tissue disorders (e.g., pain, neck pain, back pain, chest wall pain and extremity pain); Nervous system disorders (e.g., Headache, Involuntary Movements, Memory impairment, Sleep disorder—increased dreams, Sensory neuropathy and Hypersomnia); Psychiatric disorders (e.g., Hallucinations, Depression, Insomnia, and Impulse control disorder); Renal and urinary disorders (e.g., Urinary incontinence); Respiratory, thoracic and mediastinal disorders (e.g., cough and productive cough); Skin and subcutaneous tissue disorders (e.g., Skin ulceration); Surgical and medical procedures (e.g., Carpal tunnel release surgery); transient paresthesia; transient tremor; hypotension; and Vascular disorders (e.g., Hypertension).

### Nucleic Acids

In some aspects, the disclosure provides isolated nucleic acids that are useful for expressing the GDNF gene or GDNF gene product. A “nucleic acid” sequence refers to a DNA or RNA sequence. In some embodiments, nucleic acids, and proteins translated therefrom, of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The ordinarily skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequences of the proteins and polypeptides disclosed herein.

The isolated nucleic acids described herein may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid comprised of capsid proteins and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. In various aspects, left and right ITRs are independently 145 bp or fewer or 130 bp or fewer. By “independently,” it is meant that the left and right ITRs can be the same length or different lengths. As non-limiting examples, the left and right ITRs can independently be 145 bp, 130 bp, 128 bp, 124 bp, or 119 bp. Preferably, substantially the entire sequences encoding the ITRs are used in the nucleic acid sequence, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a nucleic acid molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656.

In addition to the elements identified above for the recombinant AAV vector, the vector also includes conventional control elements which are operably linked with elements of the transgene in a manner that permits its transcription, translation and/or expression in a cell transfected with the vector or infected with the virus produced by methods described herein. As used herein, “operably linked” sequences include both expression control sequences that are contiguous with the gene of interest (i.e., GDNF) and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency {i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the regulatory sequence (e.g., a 5′ regulatory sequences) results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably linked to a nucleic acid sequence when the promoter region is capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein.

### Promoter

In various embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene (e.g., a coding sequence of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences, i.e., downstream of the transgene sequences or 3′ of the transgene sequences, and before the 3′ AAV ITR sequence. A rAAV construct useful in the present disclosure may also contain an intron, desirably located between the promoter/enhancer sequence and the transgene. One exemplary intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1 a promoter [Invitrogen]. In some embodiments, a promoter is an enhanced chicken R-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionein (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268: 1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. As used herein, “native promoter” refers to the endogenous promoter of the transgene.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter.

Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11:654-64 (1996)), CD2 promoter (Hansal et al., J. Immunol., 161: 1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

Nervous system (NS)-specific promoters contemplated for use in the present methods and compositions also include those described in International Patent Application Numbers WO/2022/049385 and WO/2021/214443, which are incorporated by reference herein in their entireties. In some embodiments, the NS-specific promoter is a promoter of Table 1, or a promoter having at least 80%, at least 85%, at least 90%, at least 95%, at least 98% identity to a promoter of Table 1. In some embodiments, the NS-specific promoter is a promoter of Table 1, or a promoter having at least 80%, at least 85%, at least 90%, at least 95%, at least 98% identity to a promoter of Table 1 and retaining the NS-specific promoter activity of the promoter of Table 1.

CNS-specific promoters contemplated for use in the present methods and compositions also include those described in International Patent Application WO/2021/214443, the contents of which are incorporated by reference herein in their entireties. In some embodiments, the CNS-specific promoter is a promoter of Tables 2-4, or a promoter having at least 80%, at least 85%, at least 90%, at least 95%, at least 98% identity to a promoter of Tables 2-4. In some embodiments, the CNS-specific promoter is a promoter of Tables 2-4, or a promoter having at least 80%, at least 85%, at least 90%, at least 95%, at least 98% identity to a promoter of Tables 2-4 and retaining the CNS-specific promoter activity of the promoter of Tables 2-4.

In some embodiments, the nucleic acid comprises one or more cis-regulatory elements (CREs). In some embodiments, the nucleic acid comprises one or more NS-specific CREs or CNS-specific CREs. In some embodiments, the nucleic acid comprises one or more CREs of Tables 4-6, or a CRE having at least 80%, at least 85%, at least 90%, at least 95%, at least 98% identity to a CRE of Tables 4-6. In some embodiments, the CRE is a CRE of Tables 4-6, or a CRE having at least 80n, at least 85%, at least 90%, at least 95%, at least 98% identity to a CRE of Tables 4-6 and retaining the activity of the CRE of Tables 4-6.

In some embodiments, the CRE can comprise one or more CREs known in the art. For example, in one embodiment, the one or more CREs may be selected from SEQ ID NOs: 19-24, 27, 28, 37, 38 in International Patent Application Number WO/2022/049385. For example, in one embodiment, the one or more CREs may be selected from: SEQ ID NOs: 1-8 from WO 2019/199867A1, SEQ ID NOs: 1-7 from WO2020/076614A1 and SEQ ID NOs: 25-51, 177-178, 188 from WO2020/097121. The foregoing references are incorporated by reference herein in their entireties.

TABLE 1
NS-specific promoters

| NAME | SEQUENCE | Length |
|---|---|---|
| SP0013 | AACATATCCTGGTGTGGAGTAGGGGACGCTGCTCTGACAGAGGCTCGGGGGCCTGAGCTG | 795 |
| (SEQ ID | GCTCTGTGAGCTGGGGAGGAGGCAGACAGCCAGGCCTTGTCTGCAAGCAGACCTGGCAGC | |
| NO: 3) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACAGCCCCCTAGGGGCTGCCCTTGCTGTGTGGCGCCAC | |
| | CGGCGGTGGAGAACAAGGCTCTATTCAGCCTGTGCCCAGGAAAGGGGATCAGGGGATGCC | |
| | CAGGCATGGACAGTGGGTGGCAGGGGGGGAGAGGAGGGCTGTCTGCTTCCCAGAAGTCCA | |
| | AGGACACAAATGGGTGAGGGGAGGGCTAGGCCTGCGCACCCACCCACCGACCCCTCACCC | |
| | ACCGACCCGTCACCCACCGACCAAGGGGCACCCTGGCCTAGAGGGGATGCTGAGCGGGAC | |
| | CCGCCTCCTGCCTCTGGCAGTCCCAGATGGGACTTGGACCCCGCAGTTGCTCTCTCGGAC | |
| | CCTAAGTTTCTACCCCTGGATCTAAGGCGGAGCTGGGTTTGCGGATCCCACGGTTCCCGG | |
| | CGGGGCGGGGCCCGGTCGCCCCTCCCCCTCCCCGCCCTCCTGCGCCGGGAGCAGTGCATT | |
| | GTGGGAAACTCCCGA | |
| SP0014 | AACATATCCTGGTGTGGAGTAGGGGACGCTGCTCTGACAGAGGCTCGGGGGCCTGAGCTG | 810 |
| (SEQ ID | GCTCTGTGAGCTGGGGAGGAGGCAGACAGCCAGGCCTTGTCTGCAAGCAGACCTGGCAGC | |
| NO: 4) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACAGCCCCCTAGGGGCTGCCCTTGCTGTGTGGCGCCAC | |
| | CGGCGGTGGAGAACAAGGCTCTATTCAGCCTGTGCCCAGGAAAGGGGATCAGGGGATGCC | |
| | CAGGCATGGACAGTGGGTGGCAGGGGGGGAGAGGAGGGCTGTCTGCTTCCCAGAAGTCCA | |
| | AGGACACAAATGGGTGAGGGGATGCGGCGAGGCGCGTGCGCACTGCCAGCTTCAGCACCG | |
| | CGGACAGTGCCTTCGCCCCCGCCTGGCGGCGCGCGCCACCGCCGCCTCAGCACTGAAGGC | |
| | GCGCTGACGTCACTCGCCGGTCCCCCGCAAACTCCCCTTCCCGGCCACCTTGGTCGCGTC | |
| | CGCGCCGCCGCCGGCCCAGCCGGACCGCACCACGCGAGGCGCGAGATAGGGGGGCACGGG | |
| | CGCGACCATCTGCGCTGCGGCGCCGGCGACTCAGCGCTGCCTCAGTCTGCGGTGGGCAGC | |
| | GGAGGAGTCGTGTCGTGCCTGAGAGCGCAG | |
| SP0026 | AACATATCCTGGTGTGGAGTAGGGGACGCTGCTCTGACAGAGGCTCGGGGGCCTGAGCTG | 916 |
| (SEQ ID | GCTCTGTGAGCTGGGGAGGAGGCAGACAGCCAGGCCTTGTCTGCAAGCAGACCTGGCAGC | |
| NO: 5) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACAGCCCCCTAGGGGCTGCCCTTGCTGTGTGGCGCCAC | |
| | CGGCGGTGGAGAACAAGGCTCTATTCAGCCTGTGCCCAGGAAAGGGGATCAGGGGATGCC | |
| | CAGGCATGGACAGTGGGTGGCAGGGGGGGAGAGGAGGGCTGTCTGCTTCCCAGAAGTCCA | |
| | AGGACACAAATGGGTGAGGGGAAGCGCGCAGAGTCTGCATGCGTGAGGAAGCTCCTGGGC | |
| | GCGTCACAGCCGCGCTATTCTCAGCGTCTCTCCTTTTATGGCTCCGGAAGTGAGCTGGGG | |
| | TTGCTGGCAGCCTGGCTGGCACTGGGCTAGGCCTGCGCACCCACCCACCGACCCCTCACC | |
| | CACCGACCCGTCACCCACCGACCAAGGGGCACCCTGGCCTAGAGGGGATGCTGAGCGGGA | |
| | CCCGCCTCCTGCCTCTGGCAGTCCCAGATGGGACTTGGACCCCGCAGTTGCTCTCTCGGA | |
| | CCCTAAGTTTCTACCCCTGGATCTAAGGCGGAGCTGGGTTTGCGGATCCCACGGTTCCCG | |
| | GCGGGGCGGGGCCCGGTCGCCCCTCCCCCTCCCCGCCCTCCTGCGCCGGGAGCAGTGCAT | |
| | TGTGGGAAACTCCCGA | |
| SP0027 | AACATATCCTGGTGTGGAGTAGGGGACGCTGCTCTGACAGAGGCTCGGGGGCCTGAGCTG | 931 |
| (SEQ ID | GCTCTGTGAGCTGGGGAGGAGGCAGACAGCCAGGCCTTGTCTGCAAGCAGACCTGGCAGC | |
| NO: 6) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACAGCCCCCTAGGGGCTGCCCTTGCTGTGTGGCGCCAC | |
| | CGGCGGTGGAGAACAAGGCTCTATTCAGCCTGTGCCCAGGAAAGGGGATCAGGGGATGCC | |
| | CAGGCATGGACAGTGGGTGGCAGGGGGGGAGAGGAGGGCTGTCTGCTTCCCAGAAGTCCA | |
| | AGGACACAAATGGGTGAGGGGAAGCGCGCAGAGTCTGCATGCGTGAGGAAGCTCCTGGGC | |
| | GCGTCACAGCCGCGCTATTCTCAGCGTCTCTCCTTTTATGGCTCCGGAAGTGAGCTGGGG | |
| | TTGCTGGCAGCCTGGCTGGCACTTGCGGCGAGGCGCGTGCGCACTGCCAGCTTCAGCACC | |
| | GCGGACAGTGCCTTCGCCCCCGCCTGGCGGCGCGCGCCACCGCCGCCTCAGCACTGAAGG | |
| | CGCGCTGACGTCACTCGCCGGTCCCCCGCAAACTCCCCTTCCCGGCCACCTTGGTCGCGT | |
| | CCGCGCCGCCGCCGGCCCAGCCGGACCGCACCACGCGAGGCGCGAGATAGGGGGGCACGG | |
| | GCGCGACCATCTGCGCTGCGGCGCCGGCGACTCAGCGCTGCCTCAGTCTGCGGTGGGCAG | |
| | CGGAGGAGTCGTGTCGTGCCTGAGAGCGCAG | |
| SP0030 | AACATATCCTGGTGTGGAGTAGGGGACGCTGCTCTGACAGAGGCTCGGGGGCCTGAGCTG | 617 |
| (SEQ ID | GCTCTGTGAGCTGGGGAGGAGGCAGACAGCCAGGCCTTGTCTGCAAGCAGACCTGGCAGC | |
| NO: 7) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACGGGCTAGGCCTGCGCACCCACCCACCGACCCCTCAC | |
| | CCACCGACCCGTCACCCACCGACCAAGGGGCACCCTGGCCTAGAGGGGATGCTGAGCGGG | |
| | ACCCGCCTCCTGCCTCTGGCAGTCCCAGATGGGACTTGGACCCCGCAGTTGCTCTCTCGG | |
| | ACCCTAAGTTTCTACCCCTGGATCTAAGGCGGAGCTGGGTTTGCGGATCCCACGGTTCCC | |
| | GGCGGGGCGGGGCCCGGTCGCCCCTCCCCCTCCCCGCCCTCCTGCGCCGGGAGCAGTGCA | |
| | TTGTGGGAAACTCCCGA | |
| SP0031 | AACATATCCTGGTGTGGAGTAGGGGACGCTGCTCTGACAGAGGCTCGGGGGCCTGAGCTG | 632 |
| (SEQ ID | GCTCTGTGAGCTGGGGAGGAGGCAGACAGCCAGGCCTTGTCTGCAAGCAGACCTGGCAGC | |
| NO: 8) | ATTGGGCTGGCCGCCCCCCAGGGCCTCCTCTTCATGCCCAGTGAATGACTCACCTTGGCA | |
| | CAGACACAATGTTCGGGGTGGGCACAGTGCCTGCTTCCCGCCGCACCCCAGCCCCCCTCA | |
| | AATGCCTTCCGAGAAGCCCATTGAGCAGGGGGCTTGCATTGCACCCCAGCCTGACAGCCT | |
| | GGCATCTTGGGATAAAAGCAGCACTGCGGCGAGGCGCGTGCGCACTGCCAGCTTCAGCAC | |
| | CGCGGACAGTGCCTTCGCCCCCGCCTGGCGGCGCGCGCCACCGCCGCCTCAGCACTGAAG | |
| | GCGCGCTGACGTCACTCGCCGGTCCCCCGCAAACTCCCCTTCCCGGCCACCTTGGTCGCG | |
| | TCCGCGCCGCCGCCGGCCCAGCCGGACCGCACCACGCGAGGCGCGAGATAGGGGGGCACG | |
| | GGCGCGACCATCTGCGCTGCGGCGCCGGCGACTCAGCGCTGCCTCAGTCTGCGGTGGGCA | |
| | GCGGAGGAGTCGTGTCGTGCCTGAGAGCGCAG | |
| SP0032 | TTCAGAGGTCGGATCTGAATCCAGCTCCAAGGCCCCAGCACCCAAGCCCTGACCCCATGG | 701 |
| (SEQ ID | CTGCCTGCTGGCTGGGAGTGGCATTCTTTAGAACAGGAAAGGCAGTGAGTTCTCATTGCA | |
| NO: 9) | TCAATACTTGCATTTGCTACAACAGAAGCTTTTTGTCTCTTCCTCACATTCCTTTAGAAC | |
| | ACAAGCCTCCTTTTCTGCCTGTGGCCATCGGTCACTGGAGTCAGCCTCGTGAGTGGCTTG | |
| | GTGGCGGATGGCACCGTCCTTTGTGGGAGAAAACAATGTTGCTGCCCAGGCCTTTCTGGA | |
| | ATGACCCCTTCCCACCCCCTGACCAGCCCCAGCAAGGCCCGGGGCTGGCTGCCTAGTTGA | |
| | TTGGAGCCAAGAGTTTGCTGAATGGATGAAGGGAGAAGGGACATCCAAGGGCTAGGCCTG | |
| | CGCACCCACCCACCGACCCCTCACCCACCGACCCGTCACCCACCGACCAAGGGGCACCCT | |
| | GGCCTAGAGGGGATGCTGAGCGGGACCCGCCTCCTGCCTCTGGCAGTCCCAGATGGGACT | |
| | TGGACCCCGCAGTTGCTCTCTCGGACCCTAAGTTTCTACCCCTGGATCTAAGGCGGAGCT | |
| | GGGTTTGCGGATCCCACGGTTCCCGGCGGGGCGGGGCCCGGTCGCCCCTCCCCCTCCCCG | |
| | CCCTCCTGCGCCGGGAGCAGTGCATTGTGGGAAACTCCCGA | |
| SP0033 | TTCAGAGGTCGGATCTGAATCCAGCTCCAAGGCCCCAGCACCCAAGCCCTGACCCCATGG | 716 |
| (SEQ ID | CTGCCTGCTGGCTGGGAGTGGCATTCTTTAGAACAGGAAAGGCAGTGAGTTCTCATTGCA | |
| NO: 10) | TCAATACTTGCATTTGCTACAACAGAAGCTTTTTGTCTCTTCCTCACATTCCTTTAGAAC | |
| | ACAAGCCTCCTTTTCTGCCTGTGGCCATCGGTCACTGGAGTCAGCCTCGTGAGTGGCTTG | |
| | GTGGCGGATGGCACCGTCCTTTGTGGGAGAAAACAATGTTGCTGCCCAGGCCTTTCTGGA | |
| | ATGACCCCTTCCCACCCCCTGACCAGCCCCAGCAAGGCCCGGGGCTGGCTGCCTAGTTGA | |
| | TTGGAGCCAAGAGTTTGCTGAATGGATGAAGGGAGAAGGGACATCCAATGCGGCGAGGCG | |
| | CGTGCGCACTGCCAGCTTCAGCACCGCGGACAGTGCCTTCGCCCCCGCCTGGCGGCGCGC | |
| | GCCACCGCCGCCTCAGCACTGAAGGCGCGCTGACGTCACTCGCCGGTCCCCCGCAAACTC | |
| | CCCTTCCCGGCCACCTTGGTCGCGTCCGCGCCGCCGCCGGCCCAGCCGGACCGCACCACG | |
| | CGAGGCGCGAGATAGGGGGGCACGGGCGCGACCATCTGCGCTGCGGCGCCGGCGACTCAG | |
| | CGCTGCCTCAGTCTGCGGTGGGCAGCGGAGGAGTCGTGTCGTGCCTGAGAGCGCAG | |
| SP0019 | TCAGGGGTGCAGCTTTTTTTCTGTCTTTTACTCAGCCTGAGAAAGGTTGTCGTTTGACAA | 792 |
| (SEQ ID | GGTTTGTTCAGAGGTCGGATCTGAATCCAGCTCCAAGGCCCCAGCACCCAAGCCCTGACC | |
| NO: 11) | CCATGGCTGCCTGCTGGCTGGGAGTGGCATTCTTTAGAACAGGAAAGGCAGTGAGTTCTC | |
| | ATTGCATCAATACTTGCATTTGCTACAACAGAAGCTTTTTGTCTCTTCCTCACATTCCTT | |
| | TAGAACACAAGCCTCCTTTTCTGCCTGTGGCCATCGGTCACTGGAGTCAGCCTCGTGAGT | |
| | GGCTTGGTGGCGGATGGCACCGTCCTTTGTGGGAGAAAACAATGTTGCTGCCCAGGCCTT | |
| | TCTGGAATGACCCCTTCCCACCCCCTGACCAGCCCCAGCAAGGCCCGGGGCTGGCTGCCT | |
| | AGTTGATTGGAGCCAAGAGTTTGCTGAATGGATGAAGGGAGAAGGGACATCCAAGTGACT | |
| | GAAGAAGATCTTAACAGAAGGGCTAGGCCTGCGCACCCACCCACCGACCCCTCACCCACC | |
| | GACCCGTCACCCACCGACCAAGGGGCACCCTGGCCTAGAGGGGATGCTGAGCGGGACCCG | |
| | CCTCCTGCCTCTGGCAGTCCCAGATGGGACTTGGACCCCGCAGTTGCTCTCTCGGACCCT | |
| | AAGTTTCTACCCCTGGATCTAAGGCGGAGCTGGGTTTGCGGATCCCACGGTTCCCGGCGG | |
| | GGCGGGGCCCGGTCGCCCCTCCCCCTCCCCGCCCTCCTGCGCCGGGAGCAGTGCATTGTG | |
| | GGAAACTCCCGA | |
| SP0020 | CTTCCTCTTATATTTCACCAAGACTCAGTTCCTGAGCAAGAAACCACAGGCACAGCAAGT | 686 |
| (SEQ ID | GCCATGAAAAGCGGCTTTGTGTGGGGTGGGCTCTTCACACTCCAATCTCCACTTCCTTCT | |
| NO: 12) | CAAGGCCTCAAAAAAAGTTGAAAAATGAAAACAAAAGCCCTGCTGTGTTGAGCTGGGCTC | |
| | TGGCGTTGCCATGGACCCAGGGCAAACAGCGGTGCTCCTGCTCTGCCCCCGGCTCAGCTC | |
| | ATGCTGGGCCTGCACTTCTGGAAGGGAGCATGGACTTTGGAATGACTGGTTAGAACCCAA | |
| | ATGAATTAATGGAATTTGACATAGTTCAAAAATAATAAAATGTGATACCCATGAAATGCT | |
| | GATATTCTGCCTTAATTTGCCAGATTGGGGGCCGGGCTAGGCCTGCGCACCCACCCACCG | |
| | ACCCCTCACCCACCGACCCGTCACCCACCGACCAAGGGGCACCCTGGCCTAGAGGGGATG | |
| | CTGAGCGGGACCCGCCTCCTGCCTCTGGCAGTCCCAGATGGGACTTGGACCCCGCAGTTG | |
| | CTCTCTCGGACCCTAAGTTTCTACCCCTGGATCTAAGGCGGAGCTGGGTTTGCGGATCCC | |
| | ACGGTTCCCGGCGGGGCGGGGCCCGGTCGCCCCTCCCCCTCCCCGCCCTCCTGCGCCGGG | |
| | AGCAGTGCATTGTGGGAAACTCCCGA | |
| SP0021 | TCAGGGGTGCAGCTTTTTTTCTGTCTTTTACTCAGCCTGAGAAAGGTTGTCGTTTGACAA | 807 |
| (SEQ ID | GGTTTGTTCAGAGGTCGGATCTGAATCCAGCTCCAAGGCCCCAGCACCCAAGCCCTGACC | |
| NO: 13) | CCATGGCTGCCTGCTGGCTGGGAGTGGCATTCTTTAGAACAGGAAAGGCAGTGAGTTCTC | |
| | ATTGCATCAATACTTGCATTTGCTACAACAGAAGCTTTTTGTCTCTTCCTCACATTCCTT | |
| | TAGAACACAAGCCTCCTTTTCTGCCTGTGGCCATCGGTCACTGGAGTCAGCCTCGTGAGT | |
| | GGCTTGGTGGCGGATGGCACCGTCCTTTGTGGGAGAAAACAATGTTGCTGCCCAGGCCTT | |
| | TCTGGAATGACCCCTTCCCACCCCCTGACCAGCCCCAGCAAGGCCCGGGGCTGGCTGCCT | |
| | AGTTGATTGGAGCCAAGAGTTTGCTGAATGGATGAAGGGAGAAGGGACATCCAAGTGACT | |
| | GAAGAAGATCTTAACAGAATGCGGCGAGGCGCGTGCGCACTGCCAGCTTCAGCACCGCGG | |
| | ACAGTGCCTTCGCCCCCGCCTGGCGGCGCGCGCCACCGCCGCCTCAGCACTGAAGGCGCG | |
| | CTGACGTCACTCGCCGGTCCCCCGCAAACTCCCCTTCCCGGCCACCTTGGTCGCGTCCGC | |
| | GCCGCCGCCGGCCCAGCCGGACCGCACCACGCGAGGCGCGAGATAGGGGGGCACGGGCGC | |
| | GACCATCTGCGCTGCGGCGCCGGCGACTCAGCGCTGCCTCAGTCTGCGGTGGGCAGCGGA | |
| | GGAGTCGTGTCGTGCCTGAGAGCGCAG | |
| SP0022 | CTTCCTCTTATATTTCACCAAGACTCAGTTCCTGAGCAAGAAACCACAGGCACAGCAAGT | 701 |
| (SEQ ID | GCCATGAAAAGCGGCTTTGTGTGGGGTGGGCTCTTCACACTCCAATCTCCACTTCCTTCT | |
| NO: 14) | CAAGGCCTCAAAAAAAGTTGAAAAATGAAAACAAAAGCCCTGCTGTGTTGAGCTGGGCTC | |
| | TGGCGTTGCCATGGACCCAGGGCAAACAGCGGTGCTCCTGCTCTGCCCCCGGCTCAGCTC | |
| | ATGCTGGGCCTGCACTTCTGGAAGGGAGCATGGACTTTGGAATGACTGGTTAGAACCCAA | |
| | ATGAATTAATGGAATTTGACATAGTTCAAAAATAATAAAATGTGATACCCATGAAATGCT | |
| | GATATTCTGCCTTAATTTGCCAGATTGGGGGCCTGCGGCGAGGCGCGTGCGCACTGCCAG | |
| | CTTCAGCACCGCGGACAGTGCCTTCGCCCCCGCCTGGCGGCGCGCGCCACCGCCGCCTCA | |
| | GCACTGAAGGCGCGCTGACGTCACTCGCCGGTCCCCCGCAAACTCCCCTTCCCGGCCACC | |
| | TTGGTCGCGTCCGCGCCGCCGCCGGCCCAGCCGGACCGCACCACGCGAGGCGCGAGATAG | |
| | GGGGGCACGGGCGCGACCATCTGCGCTGCGGCGCCGGCGACTCAGCGCTGCCTCAGTCTG | |
| | CGGTGGGCAGCGGAGGAGTCGTGTCGTGCCTGAGAGCGCAG | |
| SP0028 | TCAGGGGTGCAGCTTTTTTTCTGTCTTTTACTCAGCCTGAGAAAGGTTGTCGTTTGACAA | 913 |
| (SEQ ID | GGTTTGTTCAGAGGTCGGATCTGAATCCAGCTCCAAGGCCCCAGCACCCAAGCCCTGACC | |
| NO: 15) | CCATGGCTGCCTGCTGGCTGGGAGTGGCATTCTTTAGAACAGGAAAGGCAGTGAGTTCTC | |
| | ATTGCATCAATACTTGCATTTGCTACAACAGAAGCTTTTTGTCTCTTCCTCACATTCCTT | |
| | TAGAACACAAGCCTCCTTTTCTGCCTGTGGCCATCGGTCACTGGAGTCAGCCTCGTGAGT | |
| | GGCTTGGTGGCGGATGGCACCGTCCTTTGTGGGAGAAAACAATGTTGCTGCCCAGGCCTT | |
| | TCTGGAATGACCCCTTCCCACCCCCTGACCAGCCCCAGCAAGGCCCGGGGCTGGCTGCCT | |
| | AGTTGATTGGAGCCAAGAGTTTGCTGAATGGATGAAGGGAGAAGGGACATCCAAGTGACT | |
| | GAAGAAGATCTTAACAGAAAGCGCGCAGAGTCTGCATGCGTGAGGAAGCTCCTGGGCGCG | |
| | TCACAGCCGCGCTATTCTCAGCGTCTCTCCTTTTATGGCTCCGGAAGTGAGCTGGGGTTG | |
| | CTGGCAGCCTGGCTGGCACTGGGCTAGGCCTGCGCACCCACCCACCGACCCCTCACCCAC | |
| | CGACCCGTCACCCACCGACCAAGGGGCACCCTGGCCTAGAGGGGATGCTGAGCGGGACCC | |
| | GCCTCCTGCCTCTGGCAGTCCCAGATGGGACTTGGACCCCGCAGTTGCTCTCTCGGACCC | |
| | TAAGTTTCTACCCCTGGATCTAAGGCGGAGCTGGGTTTGCGGATCCCACGGTTCCCGGCG | |
| | GGGCGGGGCCCGGTCGCCCCTCCCCCTCCCCGCCCTCCTGCGCCGGGAGCAGTGCATTGT | |
| | GGGAAACTCCCGA | |
| SP0029 | TCAGGGGTGCAGCTTTTTTTCTGTCTTTTACTCAGCCTGAGAAAGGTTGTCGTTTGACAA | 928 |
| (SEQ ID | GGTTTGTTCAGAGGTCGGATCTGAATCCAGCTCCAAGGCCCCAGCACCCAAGCCCTGACC | |
| NO: 16) | CCATGGCTGCCTGCTGGCTGGGAGTGGCATTCTTTAGAACAGGAAAGGCAGTGAGTTCTC | |
| | ATTGCATCAATACTTGCATTTGCTACAACAGAAGCTTTTTGTCTCTTCCTCACATTCCTT | |
| | TAGAACACAAGCCTCCTTTTCTGCCTGTGGCCATCGGTCACTGGAGTCAGCCTCGTGAGT | |
| | GGCTTGGTGGCGGATGGCACCGTCCTTTGTGGGAGAAAACAATGTTGCTGCCCAGGCCTT | |
| | TCTGGAATGACCCCTTCCCACCCCCTGACCAGCCCCAGCAAGGCCCGGGGCTGGCTGCCT | |
| | AGTTGATTGGAGCCAAGAGTTTGCTGAATGGATGAAGGGAGAAGGGACATCCAAGTGACT | |
| | GAAGAAGATCTTAACAGAAAGCGCGCAGAGTCTGCATGCGTGAGGAAGCTCCTGGGCGCG | |
| | TCACAGCCGCGCTATTCTCAGCGTCTCTCCTTTTATGGCTCCGGAAGTGAGCTGGGGTTG | |
| | CTGGCAGCCTGGCTGGCACTTGCGGCGAGGCGCGTGCGCACTGCCAGCTTCAGCACCGCG | |
| | GACAGTGCCTTCGCCCCCGCCTGGCGGCGCGCGCCACCGCCGCCTCAGCACTGAAGGCGC | |
| | GCTGACGTCACTCGCCGGTCCCCCGCAAACTCCCCTTCCCGGCCACCTTGGTCGCGTCCG | |
| | CGCCGCCGCCGGCCCAGCCGGACCGCACCACGCGAGGCGCGAGATAGGGGGGCACGGGCG | |
| | CGACCATCTGCGCTGCGGCGCCGGCGACTCAGCGCTGCCTCAGTCTGCGGTGGGCAGCGG | |
| | AGGAGTCGTGTCGTGCCTGAGAGCGCAG | |
| SP0011 | CTAGCCCACAGGAAATGTCTGTCTATATCCAGGCAAGTACCTTGCTCATTGGACCAACCC | 529 |
| (SEQ ID | GAAACTGTTCAGGGAAGATCAGGGAAATCAACTCAGTTACAAATGGGATAATCATGCCCA | |
| NO: 17) | GTAAAAACTACCTGTGGTGAATAAAGAGTTAACCCCTGTTCCATCTTAGGTCACTATGCA | |
| | GAGTACCAATGAGTACAAGAGATGGTGCCAAAGAGGGTGGCCCCTCCCTAGCTGGGAACA | |
| | GTCAACCCTTAGGAACTAGACTGTCAACACATCAGCCAGCCAGAGACAAGGGAAACCGTG | |
| | GCAACCAAGTGTTGCTGGCACATTGTGAGGTGGTGATGGGAACTGCAGAGGCCCTGCACA | |
| | GCATGCTAATGAGCCCAGGCAAACATGAGCTCTCCCCATAGCTGGGCTGCGGCCCAACCC | |
| | CACCCCCTCAGGCTATGCCAGGGGGTGTTGCCAGGGGCACCCGGGCATCGCCAGTCTAGC | |
| | CCACTCCTTCATAAAGCCCTCGCATCCCAGGAGCGAGCAGAGCCAGAGC | |
| SP0034 | TCCAAAGAAAAGCCAGATAAGTAGCTGATTATTGCATAGAGCTGACAGTATCACAGGAAG | 646 |
| (SEQ ID | ATCAGTAGTAGCAGCTCAAGTACAAAAAGGTTAATTAGCAATACTTAATAAGAAAAACTA | |
| NO: 18) | CCTCTGGCAGGTGAAGAGTTAATCCCTGGTCAATTTTAAGCTACTCTGCTGAGAGTACTA | |
| | ATAAGTGTAGGGGTTGGAGCCAATGAGGGTGACCCCTTCCTTGATGGGAACAGTCATCCC | |
| | TTAGGAACTGCCCTGGAAAGCATCAGCCAGCCAGAAAACAGGGAAAGAGGCTGAGAAACC | |
| | GTGGTAACCAAGTTTTGCTGGCACTTTGTAAAATGGTAACTGCAACTGCCGAGGCTGTGC | |
| | AGAGAATGCTAATAAGCCTAGGACAACCTGTAAAGAGTGGACCTAGAAAATGTCCACCCG | |
| | CTAGAGAGAGGGAGCGAGCATGTGCGATGAGCAATAGCTGTGGACCTTACAGTTGCTGCT | |
| | AACTGCCCTGGTGTGTGTGAGGGAGAGAGAGGGAGGGAGGGAGAGAGAGCGCGCTAGCGC | |
| | GAGAGAGCGAGTGAGCAAGCGAGCAGAAAAGAGGTGGAGAGGGGGGGAATAAGAAAGAGA | |
| | GAGAAGGAAAGGAGAGAAGGCAGGAAGAAGGCAAGGGACGAGACAA | |
| SP0035 | CTAGCCCACAGGAAATGTCTGTCTATATCCAGGCAAGTACCTTGCTCATTGGACCAACCC | 614 |
| (SEQ ID | GAAACTGTTCAGGGAAGATCAGGGAAATCAACTCAGTTACAAATGGGATAATCATGCCCA | |
| NO: 19) | GTAAAAACTACCTGTGGTGAATAAAGAGTTAACCCCTGTTCCATCTTAGGTCACTATGCA | |
| | GAGTACCAATGAGTACAAGAGATGGTGCCAAAGAGGGTGGCCCCTCCCTAGCTGGGAACA | |
| | GTCAACCCTTAGGAACTAGACTGTCAACACATCAGCCAGCCAGAGACAAGGGAAACCGTG | |
| | GCAACCAAGTGTTGCTGGCACATTGTGAGGTGGTGATGGGAACTGCAGAGGCCCTGCACA | |
| | GCATGCTAATGAGCCCAGGCAAACATCGCTAGAGAGAGGGAGCGAGCATGTGCGATGAGC | |
| | AATAGCTGTGGACCTTACAGTTGCTGCTAACTGCCCTGGTGTGTGTGAGGGAGAGAGAGG | |
| | GAGGGAGGGAGAGAGAGCGCGCTAGCGCGAGAGAGCGAGTGAGCAAGCGAGCAGAAAAGA | |
| | GGTGGAGAGGGGGGGAATAAGAAAGAGAGAGAAGGAAAGGAGAGAAGGCAGGAAGAAGGC | |
| | AAGGGACGAGACAA | |
| CNS-1 | CTGGGCAGAGAGGGGGCATCGGGGGCATGGCTAGGGGCCAGCACTGTGCTTCCTGGGCGC | 696 |
| (SEQ ID | CTCACCTCCTCCCTGACTCCTGGAGACTCCCAGCCCCTGTCTGGGAGATGAGCATTTAGG | |
| NO: 20) | AATCTGCTTGTGCAGGGGTGGTGGGAGGGGCCGGGGTGGAGGGCGCATCCCCACGGGGAG | |
| | ATTGGATGGAAATGGCCTGCCAGTGTGTGTGTGAGTGTGCGCCTGTGGCAGCAGCAGAGT | |
| | AAACAGCCGCTGCCCTGTCCTCTCTGCGGCCGTGGCCAGGTACACAGGCCTGTTTGGACA | |
| | GCTGCCTTGTCTGTCCGTCTGTTTGGGAGATGCTGGCTGATAGATGGGGATGGGCGGACT | |
| | GTTAACCCCTCGTTGCCTGCACTGCTATGTGCTTCCTGCCTCATCCATGGGGTAGAAGGT | |
| | AGCCAGAAGGTGGTCCTGGCTGTGCCCCCAGCTCCTCTCTAGGGGGGAAACCTCTAGTTC | |
| | TGAGTCAGGGACAGAGTGAGGAGGGCTCCAGGGCATCAAGAGCTTGCTCCTCCCCGCACC | |
| | AGGGAGCCAAGGACAGAGGAGAAGGGGGTCTTCCCCAGTGGTGACTAGGGGCAGAATATG | |
| | TCTCTGAGTGAGTGTCTGGAGCCCTCCTCACCCCAACACCATGGGGCTGGGCATAAAAGT | |
| | CAGGGCAGAGCCATCTATTGCTTACATTTGCTTCTG | |
| CNS-2 | GGTGTGTGGAAGGGTGAGAGGCACACACACAGACACTGAAAGAATCCTAGGCCTGGTAGG | 709 |
| (SEQ ID | CACTTAACAAATGTCTGTTACAGACCAGAATTTTATTGCTGTTAGAGACCCAAGCCCCTC | |
| NO: 21) | ATAGGAACAGTGAGAAACAGGTGCAGAAAGGCGGAGTAACTTTATCTAAAGTCATAGGCT | |
| | CCCTGAATAGCAGAGCTGACACCTACAAGGAAGCGTTGGAGACCAGATCTACCAGCTAGC | |
| | CTCCCTGAGACCACGAGGTGGCGCCGCAGCACCGGCTGTGGCCGATGCCAGCCAGGTAGC | |
| | CGGTTTCCCACGTCCCCCGCACGCACGCACCTCTTTGCTGCAGGAATCCCGGGCTGCCCC | |
| | GACCTGGAGTAGGGGGGGTGGTGAGTGGGACTGAGTCCCTAGAAGCCTGGACCCTCACTT | |
| | CGTTCCTGTACATCCAGCTCGCCTGTAGACAGTGGGGGAGGATGAAGGGAAGAGGACTCA | |
| | AGCGCAACTTTGAATCATCACGCCTTCGACAGTCCGCGCACGTTTATTTCATTTATCTTT | |
| | GAAAACGAGGGAGGGGAAGCCTGGAGAAGGCGGGATGGGCCAAGGGTGAGTTGGCCCCCG | |
| | GGGAGCTGGTCCCTGTTCCTGGCTTTAGTCCCAGGGGCGCGGTCTGTGTGTAGGGCGGGC | |
| | TGGGCATAAAAGTCAGGGCAGAGCCATCTATTGCTTACATTTGCTTCTG | |
| CNS-3 | TGCTACCAGAGCCGGGAGAGCTGCTCGGAGACGCCTCCGGGGTGCGGGCTGGACATGAGC | |
| (SEQ ID | AGCGGCTGCCGGTCCTGGGACTAGGCCCCGCCATTTTGGATCCGCTGACAGGTCAGCGAA | |
| NO: 22) | GTCTCTTCCTAGAGTTCCGGTGTCGTGAAGGCCGCCCTGACATCGCAATAGGGAATTAGT | |
| | GGGAAGGGCCCTTAAATTGGGCGAGCCAAGGTGGGGGGAGGATTGGAACAGAGACAAAAG | |
| | GGAGGAGAGACGGACAGCGACAAGTGGAGAAAATCGGCGAAACTTGAGTGGCAGAGAAGT | |
| | CTGAGCGCTGAGACCCGGCGGCCCCGTGCGCCTTCCCACCTGGCGCCGATCCACTTTCCT | |
| | CGGGGTAGCGGCCCAACCCACTTCGCTGCCAGCCGATCCCTTTTACCCGTGGCTACCGGG | |
| | ACCACTCTACTCTCGCCCACTTGGCTCTGCCTAAGCGTCCTAGCCGGAGCGCGGTCTCTG | |
| | CCACGTGGGGAGGGGCGCGGCCGAGTTGCTGAAGAGCGCTTCTGATTGGCCAGAGGGCGG | |
| | GGTTCTTGGCGTCTCGCCGGCCAGACCCCTCCCTCAAAGGCGGGGCCTGGAGATCCACAG | |
| | CTGGAAAGGGCGGAGCCCCAGCAGGGCAGCTGGAAAGGGGCGGGGCCTGACGCGCGCGGC | |
| | TCGCCGCGGCGGGCTGGGGGCGCCCTGGTCTGCCATAAAGTGAATGGGCGCCGGCTGGGG | |
| | GTGGCAGTACGCGGTGAGGCTCACTCCCTCCGAGAGTCCAGGAGCGCC | |
| CNS-4 | AAGGAGAATGGTAAACAGCAGGAGCGAAGCGGCTGAGGAGAAAGAAGAGGAAAGAAAGGC | |
| (SEQ ID | GAGACGTGGGAGGATTGGAACAGAGACAAAAGGGAGGAGAGACGGACAGCGACAAGTGGA | |
| NO: 23) | GAAAATCGGCGAAACTTGAGTGGCAGAGAAGTCTGAGCGCTGAGACCCGGCGGCCCCGTG | |
| | CGCCTTCCCACCTGGCGCCGATCCACTTTCCTCGGGGTAGCGGCCCAACCCACTTCGCTG | |
| | CCAGCCGATCCCTTTTACCCGTGGCTACCGGGACCACTCTACTCTCGCCCACTTGGCTCT | |
| | GCCTAAGCGTCCTAGCCGGAGCGCGGTCTCTGCCACGTGGGGAGGGGCGCGGCCGAGTTG | |
| | CTGAAGAGCGCTTCTGATTGGCCAGAGGGCGGGGTTCTTGGCGTCTCGCCGGCCAGACCC | |
| | CTCCCTCAAAGGCGGGGCCTGGAGATCCACAGCTGGAAAGGGCGGAGCCCCAGCAGGGCA | |
| | GCTGGAAAGGGGCGGGGCCTGACGCGCGCGGCTCGCCGCGGCGGGCTGGGGGCGCCCTGG | |
| | TCTGCCATAAAGTGAATGGGCGCCGGCTGGGGGTGGCAGTACGCGGTGAGGCTCACTCCC | |
| | TCCGAGAGTCCAGGAGCGCCCGAGCGGAGAGGCGGCCCGGGAGCAGGGGGGCGGCCCCCA | |
| | CTCCGGCCGGGTGCCCGGCCCCTGGCCCCTGCCTGCCCTCTAGATCGCCGCCGCAGCCGC | |
| | CGCTACTGGGAGTCTG | |
| CNS-5 | ttgtAATGGGAATAAGGGCAGGACtcctgggtataagtagctcagctgatcccaccctgc | 1744 |
| (SEQ ID | ttctatgtgttaattcatttattcattcattcaacaagcatttgttgaaatgctctttgt | |
| NO: 24) | gtcaggctcagcaggaagcagtggcaataaaatggtgaacaagaaagactcggggtttct | |
| | tcatctatgttgatgtctgcagagaacagtatcagccttctaggaagtttgtaatcagat | |
| | acattgttagagagatacttatctagtaaattcctactcatcctataaggctcaaaacaa | |
| | atgcctctatgaaaccttccgtgattccctcaggcagagttaagagcttcctttcctggg | |
| | cctctatctccttccattagtattataactgtttaccagtttcccctctagactaaattt | |
| | ctcaaaagagagaatgaggtctctttcagtcttctttgcatctttaaactagcctgggtc | |
| | ccagcctgtttgatgaaagaaacaagaacactgatacaagccacagccccttggcaaaaa | |
| | agatacccaatagcaatggcaatgtaaaatcagttttagtaaatgaatcaagaattctga | |
| | tgctttagggaaagtaatgtgaacctggcaccattaacaaattcagaactcttcttctta | |
| | ggagctctctaactgaacagacagagggatgtcaacccctaattcagcttgatcgtatct | |
| | cagcaactacatttaatgagacagtgggaaaaagagagctgtccacttttaaatcagcat | |
| | atttctaactaaacaatggcaatggctaaatctttaaaatgcctatttctctcaagaaca | |
| | ctgcaatggaacatttagactttgggaaagagattagtgatttacattgctatctcactg | |
| | atttaatttaaatgctcttccaaaccaaacacacatgtgccgaagaggctactaagaaac | |
| | ccaacatgcagagttctctataagtgcagccgacagtgttgactgaaactaaacttggaa | |
| | atccagggcactaatgcacaatatcaagcaataaaacggcatctctttggcaatatttaa | |
| | tttaaaaaagaagaaagagacaggcgaagatcaggcactgtctgttttggaggatcaacc | |
| | attctgcatttcaaagcattggtccctgcaatatccaggttactgtgctagaatctcgac | |
| | tattatatcgcagttgtgagagggagggcaaagatgtgtttactcagtgattaggccctt | |
| | agaataagcctctagctcctagagagacagctcaccacttattcatttgggccaattcac | |
| | aaagcctaggaagattaaacatccatgctgagaagacaagcgaatgcagacggtgaaaaa | |
| | gaaataaaaattctttaaaaactctgagatgacttcattatttttccacaaggaaacttt | |
| | aggaaagtgtttagttagagaaaaacccacattgacctctctctaaacccttaatctttc | |
| | ctttgtggtggcactgctttgtggtaagcgactggctcgcctcgcccctcttttcactgg | |
| | aagctgagagaaaaaagactctggagaaacagttttcgttccagggacacaaacccctga | |
| | cactgttaaacatgagatgccaggaaaacacacttaaaaaaaaaattcccactttaagct | |
| | ttagactgaatgtgagaaaggagatgataaaaagagtatcacaaGAGAATCTTCAGGCTG | |
| | TGGG | |
| CNS-6 | TTTGGCACTGTGAGCAGTTTACttgacaaattctgtcaaatatttgctttctgaaatctc | 1104 |
| (SEQ ID | gagaattggttgaatataattgtacttaatgtttgcaaaataaataaatatgggactaag | |
| NO: 25) | gacgttctatcattaatttgtcagaaaagagagttgtcatttctgaaaatttaatgtcat | |
| | tgaagctctatttccaatagcaaaggagcactattgctaatagacttcagagcttgaaat | |
| | aaataaatctttggaatcctgttgcatctcttggggtgtgacatttgacagtcttttata | |
| | gcacagaacgaaacaagtttgtgagctggaattcaattgtggcgtattgattccttgcat | |
| | cagtcattattccctgctgattgacaggtgaaaattggttacgttaagtatttcatatgt | |
| | tatattggctgacatttgcttgcctgctcttgtgtcaatattgttgtaaagatctccagc | |
| | tttatgagatagcaatagacactgactgtggcttttgtgtgatgttccagtgtttttcct | |
| | gacataatttaagacatattaaaaaccagcagcatcttccctcttgagaagcttaatgcc | |
| | aatattattgtcttccaggggaagatcatgtatgctcataatcgggtgctaatttccacc | |
| | agtacgctcatgtttaggcattaggcactataactgtaaaattgagccttcttgattgat | |
| | tcatgtcaagcctcatctcggctcctgcaggggaagtcatccggctgaccctttttacac | |
| | taaaagaagagatttgtgttcctttctttcacctggaaccatcaaattgactgaataatc | |
| | tgtaatacattagtgctgacatttgttagggagaattaaacaagacacagtaatcattcc | |
| | ccagaataaaaattgtgtttgatttccagcagagttctattaaagggaggacagaatctg | |
| | tctcttccaaggtggaaaatcgtgaatattccctgcattaatgaaccaagttaacacttt | |
| | aattgcttatagaaccgagttctccaatgacagcattaaaagatagggaggctctgattt | |
| | aTGGTCAACACAGATTTGTAACCC | |
| CNS-7 | TCAACATGGATAACCAAAGTTCTtaaaactacgctttcaatgaacacatatcctttgagc | 1941 |
| (SEQ ID | aagactaataatgaggaatgggagccagctcctgtgatatttatgcaactactaaattct | |
| NO: 26) | cactgaagtcaatgggagtttgcttacgtaagggctgcaaactttagcctccagagatta | |
| | aaggggaaaaaaatccttaaactctttcaacattaatattgcctgtaaggaatccagcca | |
| | tgacctaagccatggagctttctgaacctagcaagtagaagggtaaacagtaaacaccag | |
| | ttattttaagcacaatctaatcagagttcaatgagaagcaatattatatttgatctctaa | |
| | ggtattaatacttgtatatcactattagacatctttatgtagtccattatccaaacaatg | |
| | gcttaagtctgtggtatttaataaatcaagtttccatggccgtgagactgagtgggagtg | |
| | gggatgaagccttttttcttcatttttttttcctcaggtgcaattctgtgttaatataag | |
| | agaagtgtggccttccttctcatagcactaaaagtgagataatccctgtgtaagaaatca | |
| | gtaagtacggtctgcttaatctagtcccagtgtgaaactgttgacatttgttcttttttc | |
| | tatcattatgtgactgggcctgttttgtgctggattaggcacaaatctcctatgcagcac | |
| | atttggcatgttactagtagtttaacttcattaataatgtatgaagaaaatgtaatccat | |
| | gacaaggaagcaaagaaaagtatttttttttttttttgcttctcccaaatcctttggaat | |
| | gagtaattattcaacattttatgtttgatgttatattttacaattcaacttccatagtga | |
| | tatttaaaaaagaaactttggcaaatgcttgcaaaaaacacaccttttacaattttaaat | |
| | gtgatttactgatggccagaacttgttaaacatagtaggaaattaaatatttattcatct | |
| | tatttcattttcagggccgtaaacgctccttctgagtcattcccaataacaagaatttct | |
| | accagtaaagctattaacaggcatcaaaataggggagtgctaaattaagatgagattgta | |
| | aaagcaaataagaacatacgcagactcgcataggagtgcaaatgatcgtttctgattgaa | |
| | atgtttatagctaaatgagtttggctgaattaaacacaaatgttccaaaagataagccgt | |
| | agctggtgcttcttttttctgttttttaagctgctttacagacgaaaatggaactatatt | |
| | tggaacaatgctttctgtttttccatactattgatatttgtggaaagtcacaaaatggcc | |
| | taaggaagctaagctcgccccaagcagtggtcacttacaagtacttttgtactctgtact | |
| | cctgtcacatttgggcgatcagagcaacagctggggagactttttcaacaaagatgagtg | |
| | tcagataatcctgatgagattccacatccaacatcttttgtaattatgtcacattcagct | |
| | gtaatggaataattcaagctgaaagaacaagctttgatcctttcttaaacctttccctgt | |
| | ggactggctatctaaaagatttaaagatatttctgttacaagatctagtgtttcctcaga | |
| | gaagtcatgcttctgaagcatcgtgatctacaagaacaatatcaagtttgccaaacacat | |
| | ttctgaaagcatcgtgttttggggggaggggttgtatttaatgaagatatcaataatatg | |
| | ctatgcttcaattttcatctaggtgatcaagattcattttcttgttctgtcatccaaata | |
| | ggcagacagaaaagtgattgaaatacattaTGGAGATGTGTCATTGCACA | |
| CNS-8 | GCTGGTGCTTCTTTTTTCTGTTTTTTAAGCTGCTTTACAGACGAAAATGGAACTATATTT | 540 |
| (SEQ ID | GGAACAATGCTTTCTGTTTTTCCATACTATTGATATTTGTGGAAAGTCACAAAATGGCCT | |
| NO: 27) | AAGGAAGCTAAGCTCGCCCCAAGCAGTGGTCACTTACAAGTACTTTTGTACTCTGTACTC | |
| | CTGTCACATTTGGGCGATCAGAGCAACAGCTGGGGAGACTTTTTCAACAAAGATGAGTGT | |
| | CAGATAATCCTGATGAGATTCCACATCCAACATCTTTTGTAATTATGTCACATTCAGCTG | |
| | TAATGGAATAATTCAAGCTGAAAGAACAAGCTTTGATCCTTTCTTAAACCTTTCCCTGTG | |
| | GACTGGCTATCTAAAAGATTTAAAGATATTTCTGTTACAAGATCTAGTGTTTCCTCAGAG | |
| | AAGTCATGCTTCTGAAGCATCGTGATCTACAAGAACAATATCAAGTTTGCCAAACACATT | |
| | TCTGAAAGCATCGTGTTTTGGGGGGAGGGGTTGTATTTAATGAAGATATCAATAATATGC | |

TABLE 2
CNS-specific promoters

| NAME | SEQUENCE | Length |
|---|---|---|
| CNS-1 | CTGGGCAGAGAGGGGGCATCGGGGGCATGGCTAGGGGCCAGCACTGTGCTTCCTGGGCGC | 696 |
| (SEQ | CTCACCTCCTCCCTGACTCCTGGAGACTCCCAGCCCCTGTCTGGGAGATGAGCATTTAGG | |
| ID NO: | AATCTGCTTGTGCAGGGGTGGTGGGAGGGGCCGGGGTGGAGGGCGCATCCCCACGGGGAG | |
| 28) | ATTGGATGGAAATGGCCTGCCAGTGTGTGTGTGAGTGTGCGCCTGTGGCAGCAGCAGAGT | |
| | AAACAGCCGCTGCCCTGTCCTCTCTGCGGCCGTGGCCAGGTACACAGGCCTGTTTGGACA | |
| | GCTGCCTTGTCTGTCCGTCTGTTTGGGAGATGCTGGCTGATAGATGGGGATGGGCGGACT | |
| | GTTAACCCCTCGTTGCCTGCACTGCTATGTGCTTCCTGCCTCATCCATGGGGTAGAAGGT | |
| | AGCCAGAAGGTGGTCCTGGCTGTGCCCCCAGCTCCTCTCTAGGGGGGAAACCTCTAGTTC | |
| | TGAGTCAGGGACAGAGTGAGGAGGGCTCCAGGGCATCAAGAGCTTGCTCCTCCCCGCACC | |
| | AGGGAGCCAAGGACAGAGGAGAAGGGGGTCTTCCCCAGTGGTGACTAGGGGCAGAATATG | |
| | TCTCTGAGTGAGTGTCTGGAGCCCTCCTCACCCCAACACCATGGGGCTGGGCATAAAAGT | |
| | CAGGGCAGAGCCATCTATTGCTTACATTTGCTTCTG | |
| CNS-2 | GGTGTGTGGAAGGGTGAGAGGCACACACACAGACACTGAAAGAATCCTAGGCCTGGTAGG | 709 |
| (SEQ | CACTTAACAAATGTCTGTTACAGACCAGAATTTTATTGCTGTTAGAGACCCAAGCCCCTC | |
| ID NO: | ATAGGAACAGTGAGAAACAGGTGCAGAAAGGCGGAGTAACTTTATCTAAAGTCATAGGCT | |
| 29) | CCCTGAATAGCAGAGCTGACACCTACAAGGAAGCGTTGGAGACCAGATCTACCAGCTAGC | |
| | CTCCCTGAGACCACGAGGTGGCGCCGCAGCACCGGCTGTGGCCGATGCCAGCCAGGTAGC | |
| | CGGTTTCCCACGTCCCCCGCACGCACGCACCTCTTTGCTGCAGGAATCCCGGGCTGCCCC | |
| | GACCTGGAGTAGGGGGGGTGGTGAGTGGGACTGAGTCCCTAGAAGCCTGGACCCTCACTT | |
| | CGTTCCTGTACATCCAGCTCGCCTGTAGACAGTGGGGGAGGATGAAGGGAAGAGGACTCA | |
| | AGCGCAACTTTGAATCATCACGCCTTCGACAGTCCGCGCACGTTTATTTCATTTATCTTT | |
| | GAAAACGAGGGAGGGGAAGCCTGGAGAAGGCGGGATGGGCCAAGGGTGAGTTGGCCCCCG | |
| | GGGAGCTGGTCCCTGTTCCTGGCTTTAGTCCCAGGGGCGCGGTCTGTGTGTAGGGCGGGC | |
| | TGGGCATAAAAGTCAGGGCAGAGCCATCTATTGCTTACATTTGCTTCTG | |
| CNS-3 | TGCTACCAGAGCCGGGAGAGCTGCTCGGAGACGCCTCCGGGGTGCGGGCTGGACATGAGC | |
| (SEQ | AGCGGCTGCCGGTCCTGGGACTAGGCCCCGCCATTTTGGATCCGCTGACAGGTCAGCGAA | |
| ID NO: | GTCTCTTCCTAGAGTTCCGGTGTCGTGAAGGCCGCCCTGACATCGCAATAGGGAATTAGT | |
| 30) | GGGAAGGGCCCTTAAATTGGGCGAGCCAAGGTGGGGGGAGGATTGGAACAGAGACAAAAG | |
| | GGAGGAGAGACGGACAGCGACAAGTGGAGAAAATCGGCGAAACTTGAGTGGCAGAGAAGT | |
| | CTGAGCGCTGAGACCCGGCGGCCCCGTGCGCCTTCCCACCTGGCGCCGATCCACTTTCCT | |
| | CGGGGTAGCGGCCCAACCCACTTCGCTGCCAGCCGATCCCTTTTACCCGTGGCTACCGGG | |
| | ACCACTCTACTCTCGCCCACTTGGCTCTGCCTAAGCGTCCTAGCCGGAGCGCGGTCTCTG | |
| | CCACGTGGGGAGGGGCGCGGCCGAGTTGCTGAAGAGCGCTTCTGATTGGCCAGAGGGCGG | |
| | GGTTCTTGGCGTCTCGCCGGCCAGACCCCTCCCTCAAAGGCGGGGCCTGGAGATCCACAG | |
| | CTGGAAAGGGCGGAGCCCCAGCAGGGCAGCTGGAAAGGGGCGGGGCCTGACGCGCGCGGC | |
| | TCGCCGCGGCGGGCTGGGGGCGCCCTGGTCTGCCATAAAGTGAATGGGCGCCGGCTGGGG | |
| | GTGGCAGTACGCGGTGAGGCTCACTCCCTCCGAGAGTCCAGGAGCGCC | |
| CNS-4 | AAGGAGAATGGTAAACAGCAGGAGCGAAGCGGCTGAGGAGAAAGAAGAGGAAAGAAAGGC | |
| (SEQ | GAGACGTGGGAGGATTGGAACAGAGACAAAAGGGAGGAGAGACGGACAGCGACAAGTGGA | |
| ID NO: | GAAAATCGGCGAAACTTGAGTGGCAGAGAAGTCTGAGCGCTGAGACCCGGCGGCCCCGTG | |
| 31) | CGCCTTCCCACCTGGCGCCGATCCACTTTCCTCGGGGTAGCGGCCCAACCCACTTCGCTG | |
| | CCAGCCGATCCCTTTTACCCGTGGCTACCGGGACCACTCTACTCTCGCCCACTTGGCTCT | |
| | GCCTAAGCGTCCTAGCCGGAGCGCGGTCTCTGCCACGTGGGGAGGGGCGCGGCCGAGTTG | |
| | CTGAAGAGCGCTTCTGATTGGCCAGAGGGCGGGGTTCTTGGCGTCTCGCCGGCCAGACCC | |
| | CTCCCTCAAAGGCGGGGCCTGGAGATCCACAGCTGGAAAGGGCGGAGCCCCAGCAGGGCA | |
| | GCTGGAAAGGGGCGGGGCCTGACGCGCGCGGCTCGCCGCGGCGGGCTGGGGGCGCCCTGG | |
| | TCTGCCATAAAGTGAATGGGCGCCGGCTGGGGGTGGCAGTACGCGGTGAGGCTCACTCCC | |
| | TCCGAGAGTCCAGGAGCGCCCGAGCGGAGAGGCGGCCCGGGAGCAGGGGGGCGGCCCCCA | |
| | CTCCGGCCGGGTGCCCGGCCCCTGGCCCCTGCCTGCCCTCTAGATCGCCGCCGCAGCCGC | |
| | CGCTACTGGGAGTCTG | |
| CNS-5_ | ttgtAATGGGAATAAGGGCAGGACtcctgggtataagtagctcagctgatcccaccctgc | 1744 |
| v2 | ttctatgtgttaattcatttattcattcattcaacaagcatttgttgaaatgctctttgt | |
| (SEQ | gtcaggctcagcaggaagcagtggcaataaaatggtgaacaagaaagactcggggtttct | |
| ID NO: | tcatctatgttgatgtctgcagagaacagtatcagccttctaggaagtttgtaatcagat | |
| 32) | acattgttagagagatacttatctagtaaattcctactcatcctataaggctcaaaacaa | |
| | atgcctctatgaaaccttccgtgattccctcaggcagagttaagagcttcctttcctggg | |
| | cctctatctccttccattagtattataactgtttaccagtttcccctctagactaaattt | |
| | ctcaaaagagagaatgaggtctctttcagtcttctttgcatctttaaactagcctgggtc | |
| | ccagcctgtttgatgaaagaaacaagaacactgatacaagccacagccccttggcaaaaa | |
| | agatacccaatagcaatggcaatgtaaaatcagttttagtaaatgaatcaagaattctga | |
| | tgctttagggaaagtaatgtgaacctggcaccattaacaaattcagaactcttcttctta | |
| | ggagctctctaactgaacagacagagggatgtcaacccctaattcagcttgatcgtatct | |
| | cagcaactacatttaatgagacagtgggaaaaagagagctgtccacttttaaatcagcat | |
| | atttctaactaaacaatggcaatggctaaatctttaaaatgcctatttctctcaagaaca | |
| | ctgcaatggaacatttagactttgggaaagagattagtgatttacattgctatctcactg | |
| | atttaatttaaatgctcttccaaaccaaacacacatgtgccgaagaggctactaagaaac | |
| | ccaacatgcagagttctctataagtgcagccgacagtgttgactgaaactaaacttggaa | |
| | atccagggcactaatgcacaatatcaagcaataaaacggcatctctttggcaatatttaa | |
| | tttaaaaaagaagaaagagacaggcgaagatcaggcactgtctgttttggaggatcaacc | |
| | attctgcatttcaaagcattggtccctgcaatatccaggttactgtgctagaatctcgac | |
| | tattatatcgcagttgtgagagggagggcaaagatgtgtttactcagtgattaggccctt | |
| | agaataagcctctagctcctagagagacagctcaccacttattcatttgggccaattcac | |
| | aaagcctaggaagattaaacatccatgctgagaagacaagcgaatgcagacggtgaaaaa | |
| | gaaataaaaattctttaaaaactctgagatgacttcattatttttccacaaggaaacttt | |
| | aggaaagtgtttagttagagaaaaacccacattgacctctctctaaacccttaatctttc | |
| | ctttgtggtggcactgctttgtggtaagcgactggctcgcctcgcccctcttttcactgg | |
| | aagctgagagaaaaaagactctggagaaacagttttcgttccagggacacaaacccctga | |
| | cactgttaaacatgagatgccaggaaaacacacttaaaaaaaaaattcccactttaagct | |
| | ttagactgaatgtgagaaaggagatgataaaaagagtatcacaaGAGAATCTTCAGGCTG | |
| | TGGG | |
| CNS-6_ | TTTGGCACTGTGAGCAGTTTACttgacaaattctgtcaaatatttgctttctgaaatctc | 1104 |
| v2 | gagaattggttgaatataattgtacttaatgtttgcaaaataaataaatatgggactaag | |
| (SEQ | gacgttctatcattaatttgtcagaaaagagagttgtcatttctgaaaatttaatgtcat | |
| ID NO: | tgaagctctatttccaatagcaaaggagcactattgctaatagacttcagagcttgaaat | |
| 33) | aaataaatctttggaatcctgttgcatctcttggggtgtgacatttgacagtcttttata | |
| | gcacagaacgaaacaagtttgtgagctggaattcaattgtggcgtattgattccttgcat | |
| | cagtcattattccctgctgattgacaggtgaaaattggttacgttaagtatttcatatgt | |
| | tatattggctgacatttgcttgcctgctcttgtgtcaatattgttgtaaagatctccagc | |
| | tttatgagatagcaatagacactgactgtggcttttgtgtgatgttccagtgtttttcct | |
| | gacataatttaagacatattaaaaaccagcagcatcttccctcttgagaagcttaatgcc | |
| | aatattattgtcttccaggggaagatcatgtatgctcataatcgggtgctaatttccacc | |
| | agtacgctcatgtttaggcattaggcactataactgtaaaattgagccttcttgattgat | |
| | tcatgtcaagcctcatctcggctcctgcaggggaagtcatccggctgaccctttttacac | |
| | taaaagaagagatttgtgttcctttctttcacctggaaccatcaaattgactgaataatc | |
| | tgtaatacattagtgctgacatttgttagggagaattaaacaagacacagtaatcattcc | |
| | ccagaataaaaattgtgtttgatttccagcagagttctattaaagggaggacagaatctg | |
| | tctcttccaaggtggaaaatcgtgaatattccctgcattaatgaaccaagttaacacttt | |
| | aattgcttatagaaccgagttctccaatgacagcattaaaagatagggaggctctgattt | |
| | aTGGTCAACACAGATTTGTAACCC | |
| CNS-7_ | TCAACATGGATAACCAAAGTTCTtaaaactacgctttcaatgaacacatatcctttgagc | 1941 |
| v2 | aagactaataatgaggaatgggagccagctcctgtgatatttatgcaactactaaattct | |
| (SEQ | cactgaagtcaatgggagtttgcttacgtaagggctgcaaactttagcctccagagatta | |
| ID NO: | aaggggaaaaaaatccttaaactctttcaacattaatattgcctgtaaggaatccagcca | |
| 34) | tgacctaagccatggagctttctgaacctagcaagtagaagggtaaacagtaaacaccag | |
| | ttattttaagcacaatctaatcagagttcaatgagaagcaatattatatttgatctctaa | |
| | ggtattaatacttgtatatcactattagacatctttatgtagtccattatccaaacaatg | |
| | gcttaagtctgtggtatttaataaatcaagtttccatggccgtgagactgagtgggagtg | |
| | gggatgaagccttttttcttcatttttttttcctcaggtgcaattctgtgttaatataag | |
| | agaagtgtggccttccttctcatagcactaaaagtgagataatccctgtgtaagaaatca | |
| | gtaagtacggtctgcttaatctagtcccagtgtgaaactgttgacatttgttcttttttc | |
| | tatcattatgtgactgggcctgttttgtgctggattaggcacaaatctcctatgcagcac | |
| | atttggcatgttactagtagtttaacttcattaataatgtatgaagaaaatgtaatccat | |
| | gacaaggaagcaaagaaaagtatttttttttttttttgcttctcccaaatcctttggaat | |
| | gagtaattattcaacattttatgtttgatgttatattttacaattcaacttccatagtga | |
| | tatttaaaaaagaaactttggcaaatgcttgcaaaaaacacaccttttacaattttaaat | |
| | gtgatttactgatggccagaacttgttaaacatagtaggaaattaaatatttattcatct | |
| | tatttcattttcagggccgtaaacgctccttctgagtcattcccaataacaagaatttct | |
| | accagtaaagctattaacaggcatcaaaataggggagtgctaaattaagatgagattgta | |
| | aaagcaaataagaacatacgcagactcgcataggagtgcaaatgatcgtttctgattgaa | |
| | atgtttatagctaaatgagtttggctgaattaaacacaaatgttccaaaagataagccgt | |
| | agctggtgcttcttttttctgttttttaagctgctttacagacgaaaatggaactatatt | |
| | tggaacaatgctttctgtttttccatactattgatatttgtggaaagtcacaaaatggcc | |
| | taaggaagctaagctcgccccaagcagtggtcacttacaagtacttttgtactctgtact | |
| | cctgtcacatttgggcgatcagagcaacagctggggagactttttcaacaaagatgagtg | |
| | tcagataatcctgatgagattccacatccaacatcttttgtaattatgtcacattcagct | |
| | gtaatggaataattcaagctgaaagaacaagctttgatcctttcttaaacctttccctgt | |
| | ggactggctatctaaaagatttaaagatatttctgttacaagatctagtgtttcctcaga | |
| | gaagtcatgcttctgaagcatcgtgatctacaagaacaatatcaagtttgccaaacacat | |
| | ttctgaaagcatcgtgttttggggggaggggttgtatttaatgaagatatcaataatatg | |
| | ctatgcttcaattttcatctaggtgatcaagattcattttcttgttctgtcatccaaata | |
| | ggcagacagaaaagtgattgaaatacattaTGGAGATGTGTCATTGCACA | |
| CNS-8_ | GCTGGTGCTTCTTTTTTCTGTTTTTTAAGCTGCTTTACAGACGAAAATGGAACTATATTT | 540 |
| v2 | GGAACAATGCTTTCTGTTTTTCCATACTATTGATATTTGTGGAAAGTCACAAAATGGCCT | |
| (SEQ | AAGGAAGCTAAGCTCGCCCCAAGCAGTGGTCACTTACAAGTACTTTTGTACTCTGTACTC | |
| ID NO: | CTGTCACATTTGGGCGATCAGAGCAACAGCTGGGGAGACTTTTTCAACAAAGATGAGTGT | |
| 35) | CAGATAATCCTGATGAGATTCCACATCCAACATCTTTTGTAATTATGTCACATTCAGCTG | |
| | TAATGGAATAATTCAAGCTGAAAGAACAAGCTTTGATCCTTTCTTAAACCTTTCCCTGTG | |
| | GACTGGCTATCTAAAAGATTTAAAGATATTTCTGTTACAAGATCTAGTGTTTCCTCAGAG | |
| | AAGTCATGCTTCTGAAGCATCGTGATCTACAAGAACAATATCAAGTTTGCCAAACACATT | |
| | TCTGAAAGCATCGTGTTTTGGGGGGAGGGGTTGTATTTAATGAAGATATCAATAATATGC | |
| CNS-1 + | CTGGGCAGAGAGGGGGCATCGGGGGCATGGCTAGGGGCCAGCACTGTGCTTCCTGGGCGC | 982 |
| CMV- | CTCACCTCCTCCCTGACTCCTGGAGACTCCCAGCCCCTGTCTGGGAGATGAGCATTTAGG | |
| IE UTR | AATCTGCTTGTGCAGGGGTGGTGGGAGGGGCCGGGGTGGAGGGCGCATCCCCACGGGGAG | |
| and | ATTGGATGGAAATGGCCTGCCAGTGTGTGTGTGAGTGTGCGCCTGTGGCAGCAGCAGAGT | |
| intron | AAACAGCCGCTGCCCTGTCCTCTCTGCGGCCGTGGCCAGGTACACAGGCCTGTTTGGACA | |
| (SEQ | GCTGCCTTGTCTGTCCGTCTGTTTGGGAGATGCTGGCTGATAGATGGGGATGGGCGGACT | |
| ID NO: | GTTAACCCCTCGTTGCCTGCACTGCTATGTGCTTCCTGCCTCATCCATGGGGTAGAAGGT | |
| 36) | AGCCAGAAGGTGGTCCTGGCTGTGCCCCCAGCTCCTCTCTAGGGGGGAAACCTCTAGTTC | |
| | TGAGTCAGGGACAGAGTGAGGAGGGCTCCAGGGCATCAAGAGCTTGCTCCTCCCCGCACC | |
| | AGGGAGCCAAGGACAGAGGAGAAGGGGGTCTTCCCCAGTGGTGACTAGGGGCAGAATATG | |
| | TCTCTGAGTGAGTGTCTGGAGCCCTCCTCACCCCAACACCATGGGGCTGGGCATAAAAGT | |
| | CAGGGCAGAGCCATCTATTGCTTACATTTGTCAGATCGCCTGGAGACGCCATCCACGCTG | |
| | TTTTGACCTCCATAGAAGACACCGGGACCGATCCAGCCTCCGCGGCCGGGAACGGTGCAT | |
| | TGGAACGCGGATTCCCCGTGCCAAGAGTGACGTAAGTACCGCCTATAGACTCTATAGGCA | |
| | CACCCCTTTGGCTCTTATGCATGAACGGTGGAGGGCAGTGTAGTCTGAGCAGTACTCGTT | |
| | GCTGCCGCGCGCGCCACCAGACATAATAGCTGACAGACTAACAGACTGTTCCTTTCCATG | |
| | GGTCTTTTCTGCAGATGCCACC | |
| CNS-4 + | AAGGAGAATGGTAAACAGCAGGAGCGAAGCGGCTGAGGAGAAAGAAGAGGAAAGAAAGGC | 876 |
| CMV- | GAGACGTGGGAGGATTGGAACAGAGACAAAAGGGAGGAGAGACGGACAGCGACAAGTGGA | |
| IE UTR | GAAAATCGGCGAAACTTGAGTGGCAGAGAAGTCTGAGCGCTGAGACCCGGCGGCCCCGTG | |
| and | CGCCTTCCCACCTGGCGCCGATCCACTTTCCTCGGGGTAGCGGCCCAACCCACTTCGCTG | |
| intron | CCAGCCGATCCCTTTTACCCGTGGCTACCGGGACCACTCTACTCTCGCCCACTTGGCTCT | |
| (SEQ | GCCTAAGCGTCCTAGCCGGAGCGCGGTCTCTGCCACGTGGGGAGGGGCGCGGCCGAGTTG | |
| ID NO: | CTGAAGAGCGCTTCTGATTGGCCAGAGGGCGGGGTTCTTGGCGTCTCGCCGGCCAGACCC | |
| 37) | CTCCCTCAAAGGcGGGGCCTGGAGATCCACAGCTGGAAAGGGCGGAGCCCCAGCAGGGCA | |
| | GCTGGAAAGGGGCGGGGCCTGACGCGCGCGGCTCGCCGCGGCGGGCTGGGGGCGCCCTGG | |
| | TCTGCCATAAAGTGAATGGGCGCCGGCTGGGGGTGGCAGTACGCTCAGATCGCCTGGAGA | |
| | CGCCATCCACGCTGTTTTGACCTCCATAGAAGACACCGGGACCGATCCAGCCTCCGCGGC | |
| | CGGGAACGGTGCATTGGAACGCGGATTCCCCGTGCCAAGAGTGACGTAAGTACCGCCTAT | |
| | AGACTCTATAGGCACACCCCTTTGGCTCTTATGCATGAACGGTGGAGGGCAGTGTAGTCT | |
| | GAGCAGTACTCGTTGCTGCCGCGCGCGCCACCAGACATAATAGCTGACAGACTAACAGAC | |
| | TGTTCCTTTCCATGGGTCTTTTCTGCAGATGCCACC | |
| CNS-5 | GGAACATTTAGACTTTGGGAAAGAGATTAGTGATTTACATTGCTATCTCACTGATTTAAT | 835 |
| (SEQ | TTAAATGCTCTTCCAAACCAAACACACATGTGCCGAAGAGGCTACTAAGAAACCCAACAT | |
| ID NO: | GCAGAGTTCTCTATAAGTGCAGCCGACAGTGTTGACTGAAACTAAACTTGGAAATCCAGG | |
| 38) | GCACTAATGCACAATATCAAGCAATAAAACGGCATCTCTTTGGCAATATTTAATTTAAAA | |
| | AAGAAGAAAGAGACAGGCGAAGATCAGGCACTGTCTGTTTTGGAGGATCAACCATTCTGC | |
| | ATTTCAAAGCATTGGTCCCTGCAATATCCAGGTTACTGTGCTAGAATCTCGACTATTATA | |
| | TCGCAGTTGTGAGAGGGAGGGCAAAGATGTGTTTACTCAGTGATTAGGCCCTTAGAATAA | |
| | GCCTCTAGCTCCTAGAGAGACAGCTCACCACTTATTCATTTGGGCCAATTCACAAAGCCT | |
| | AGGAAGATTAAACATCCATGCTGAGAAGACAAGCGAATGCAGACGGTGAAAAAGAAATAA | |
| | AAATTCTTTAAAAACTCTGAGATGACTTCATTATTTTTCCACAAGGAAACTTTAGGAAAG | |
| | TGTTTAGTTAGAGAAAAACCCACATTGACCTCTCTCTAAACCCTTAATCTTTCCTTTGTG | |
| | GTGGCACTGCTTTGTGGTAAGCGACTGGCTCGCCTCGCCCCTCTTTTCACTGGAAGCTGA | |
| | GAGAAAAAAGACTCTGGAGAAACAGTTTTCGTTCCAGGGACACAAACCCCTGACACTGTT | |
| | AAGGGCTGGGCATAAAAGTCAGGGCAGAGCCATCTATTGCTTACATTTGCTTCTG | |
| CNS-6 | GAAAATTTAATGTCATTGAAGCTCTATTTCCAATAGCAAAGGAGCACTATTGCTAATAGA | 812 |
| (SEQ | CTTCAGAGCTTGAAATAAATAAATCTTTGGAATCCTGTTGCATCTCTTGGGGTGTGACAT | |
| ID NO: | TTGACAGTCTTTTATAGCACAGAACGAAACAAGTTTGTGAGCTGGAATTCAATTGTGGCG | |
| 39) | TATTGATTCCTTGCATCAGTCATTATTCCCTGCTGATTGACAGGTGAAAATTGGTTACGT | |
| | TAAGTATTTCATATGTTATATTGGCTGACATTTGCTTGCCTGCTCTTGTGTCAATATTGT | |
| | TGTAAAGATCTCCAGCTTTATGAGATAGCAATAGACACTGACTGTGGCTTTTGTGTGATG | |
| | TTCCAGTGTTTTTCCTGACATAATTTAAGACATATTAAAAACCAGCAGCATCTTCCCTCT | |
| | TGAGAAGCTTAATGCCAATATTATTGTCTTCCAGGGGAAGATCATGTATGCTCATAATCG | |
| | GGTGCTAATTTCCACCAGTACGCTCATGTTTAGGCATTAGGCACTATAACTGTAAAATTG | |
| | AGCCTTCTTGATTGATTCATGTCAAGCCTCATCTCGGCTCCTGCAGGGGAAGTCATCCGG | |
| | CTGACCCTTTTTACACTAAAAGAAGAGATTTGTGTTCCTTTCTTTCACCTGGAACCATCA | |
| | AATTGACTGAATAATCTGTAATACATTAGTGCTGACATTTGTTAGGGAGAATTAAACAAG | |
| | ACACAGTAATCATTCCCCAGAATAAAAATTGTGTTTGATGGGCTGGGCATAAAAGTCAGG | |
| | GCAGAGCCATCTATTGCTTACATTTGCTTCTG | |
| CNS-7 | TGAGACTGAGTGGGAGTGGGGATGAAGCCTTTTTTCTTCATTTTTTTTTCCTCAGGTGCA | 487 |
| (SEQ | ATTCTGTGTTAATATAAGAGAAGTGTGGCCTTCCTTCTCATAGCACTAAAAGTGAGATAA | |
| ID NO: | TCCCTGTGTAAGAAATCAGTAAGTACGGTCTGCTTAATCTAGTCCCAGTGTGAAACTGTT | |
| 40) | GACATTTGTTCTTTTTTCTATCATTATGTGACTGGGCCTGTTTTGTGCTGGATTAGGCAC | |
| | AAATCTCCTATGCAGCACATTTGGCATGTTACTAGTAGTTTAACTTCATTAATAATGTAT | |
| | GAAGAAAATGTAATCCATGACAAGGAAGCAAAGAAAAGTATTTTTTTTTTTTTTTGCTTC | |
| | TCCCAAATCCTTTGGAATGAGTAATTATTCAACATTTTATGTTTGATGTTATATTTTACA | |
| | ATTCAACTTCCATAGGGCTGGGCATAAAAGTCAGGGCAGAGCCATCTATTGCTTACATTT | |
| | GCTTCTG | |
| CNS-8 | GCTGGTGCTTCTTTTTTCTGTTTTTTAAGCTGCTTTACAGACGAAAATGGAACTATATTT | 593 |
| (SEQ | GGAACAATGCTTTCTGTTTTTCCATACTATTGATATTTGTGGAAAGTCACAAAATGGCCT | |
| ID NO: | AAGGAAGCTAAGCTCGCCCCAAGCAGTGGTCACTTACAAGTACTTTTGTACTCTGTACTC | |
| 41) | CTGTCACATTTGGGCGATCAGAGCAACAGCTGGGGAGACTTTTTCAACAAAGATGAGTGT | |
| | CAGATAATCCTGATGAGATTCCACATCCAACATCTTTTGTAATTATGTCACATTCAGCTG | |
| | TAATGGAATAATTCAAGCTGAAAGAACAAGCTTTGATCCTTTCTTAAACCTTTCCCTGTG | |
| | GACTGGCTATCTAAAAGATTTAAAGATATTTCTGTTACAAGATCTAGTGTTTCCTCAGAG | |
| | AAGTCATGCTTCTGAAGCATCGTGATCTACAAGAACAATATCAAGTTTGCCAAACACATT | |
| | TCTGAAAGCATCGTGTTTTGGGGGGAGGGGTTGTATTTAATGAAGATATCAATAATATGC | |
| | GGGCTGGGCATAAAAGTCAGGGCAGAGCCATCTATTGCTTACATTTGCTTCTG | |

TABLE 3
Minimal/Proximal Promoters comprised in the promoters of Table 2

| Name | SEQUENCE |
|---|---|
| SYNP_CRE151 | GGGCTGGGCATAAAAGTCAGGGCAGAGCCATCTATTGCTTACATTTGCTTCTG |
| (SEQ ID NO: 42) | |
| CRE0001v1_Pitx3 | GGGAGGATTGGAACAGAGACAAAAGGGAGGAGAGACGGACAGCGACAAGTGGAGAAAATCG |
| (SEQ ID NO: 43) | GCGAAACTTGAGTGGCAGAGAAGTCTGAGCGCTGAGACCCGGCGGCCCCGTGCGCCTTCCC |
| | ACCTGGCGCCGATCCACTTTCCTCGGGGTAGCGGCCCAACCCACTTCGCTGCCAGCCGATC |
| | CCTTTTACCCGTGGCTACCGGGACCACTCTACTCTCGCCCACTTGGCTCTGCCTAAGCGTC |
| | CTAGCCGGAGCGCGGTCTCTGCCACGTGGGGAGGGGCGCGGCCGAGTTGCTGAAGAGCGCT |
| | TCTGATTGGCCAGAGGGCGGGGTTCTTGGCGTCTCGCCGGCCAGACCCCTCCCTCAAAGGC |
| | GGGGCCTGGAGATCCACAGCTGGAAAGGGCGGAGCCCCAGCAGGGCAGCTGGAAAGGGGCG |
| | GGGCCTGACGCGCGCGGCTCGCCGCGGCGGGCTGGGGGCGCCCTGGTCTGCCATAAAGTGA |
| | ATGGGCGCCGGCTGGGGGTGGCAGTACGCGGTGAGGCTCACTCCCTCCGAGAGTCCAGGAG |
| | CGCC |

TABLE 4
Synthetic CNS-specific promoter overview

| Promoter | Minimal/proximal | | |
|---|---|---|---|
| name | promoter | CRE | UTR |
| CNS-1 | SYNP_CRE151 | CRE0004_Lmx1b | |
| CNS-2 | SYNP_CRE151 | CRE0003_Pitx3 | |
| CNS-3 | CRE0001v1_Pitx3 | CRE0002_Gbf1 | |
| CNS-4 | CRE0001_Pitx3 | | |
| CNS-5_v2 | CRE0005_faf1 | | |
| CNS-6_v2 | CRE0006_Pitx2 | | |
| CNS-7_v2 | CRE0007_Pitx2 | | |
| CNS-8_v2 | CRE0008_Pitx2 | | |
| CNS-1 + | SYNP_CRE151 | CRE0004_Lmx1b | CMV-IE |
| CMV-IE | | | UTR and |
| UTR and | | | intron |
| intron | | | |
| CNS-4 + | CRE0001_Pitx3 | | CMV-IE |
| CMV-IE | | | UTR and |
| UTR and | | | intron |
| intron | | | |
| CNS-5 | SYNP_CRE151 | CRE0005_faf1_short | |
| CNS-6 | SYNP_CRE151 | CRE0006_Pitx2_short | |
| CNS-7 | SYNP_CRE151 | CRE0007_Pitx2_short | |
| CNS-8 | SYNP_CRE151 | CRE0008_Pitx2_short | |

TABLE 5
Exemplary CREs

| Name | SEQUENCE |
|---|---|
| CRE0006_ | AACATATCCTGGTGTGGAGTAGGGGACGCTGCTCTGACAGAGGCTCGGGGGCCTGAGCTG |
| GFAP | GCTCTGTGAGCTGGGGAGGAGGCAGACAGCCAGGCCTTGTCTGCAAGCAGACCTGGCAGC |
| (SEQ ID | ATTGGGCTGGCCGCCCCCCAGGGCCTCCTCTTCATGCCCAGTGAATGACTCACCTTGGCA |
| NO: 44) | CAGACACAATGTTCGGGGTGGGCACAGTGCCTGCTTCCCGCCGCACCCCAGCCCCCCTCA |
| | AATGCCTTCCGAGAAGCCCATTGAGCAGGGGGCTTGCATTGCACCCCAGCCTGACAGCCT |
| | GGCATCTTGGGATAAAAGCAGCACAGCCCCCTAGGGGCTGCCCTTGCTGTGTGGCGCCA |
| CRE0008_ | AACATATCCTGGTGTGGAGTAGGGGACGCTGCTCTGACAGAGGCTCGGGGGCCTGAGCTG |
| GFAP | GCTCTGTGAGCTGGGGAGGAGGCAGACAGCCAGGCCTTGTCTGCAAGCAGACCTGGCAGC |
| (SEQ ID | ATTGGGCTGGCCGCCCCCCAGGGCCTCCTCTTCATGCCCAGTGAATGACTCACCTTGGCA |
| NO: 45) | CAGACACAATGTTCGGGGTGGGCACAGTGCCTGCTTCCCGCCGCACCCCAGCCCCCCTCA |
| | AATGCCTTCCGAGAAGCCCATTGAGCAGGGGGCTTGCATTGCACCCCAGCCTGACAGCCT |
| | GGCATCTTGGGATAAAAGCACCGGCGGTGGAGAACAAGGCTCTATTCAGCCTGTGCCCAG |
| | GAAAGGGGATCAGGGGATGCCCAGGCATGGACAGTGGGTGGCAGGGGGGGAGAGGAGGGC |
| | TGTCTGCTTCCCAGAAGTCCAAGGACACAAATGGGTGAGGGGA |
| CRE0006_ | CTAGCCCACAGGAAATGTCTGTCTATATCCAGGCAAGTACCTTGCTCATTGGACCAACCC |
| AQP4 | GAAACTGTTCAGGGAAGATCAGGGAAATCAACTCAGTTACAAATGGGATAATCATGCCCA |
| (SEQ ID | GTAAAAACTACCTGTGGTGAATAAAGAGTTAACCCCTGTTCCATCTTAGGTCACTATGCA |
| NO: 46) | GAGTACCAATGAGTACAAGAGATGGTGCCAAAGAGGGTGGCCCCTCCCTAGCTGGGAACA |
| | GTCAACCCTTAGGAACTAGACTGTCAACACATCAGCCAGCCAGAGACAAGGGAAACCGTG |
| | GCAACCAAGTGTTGCTGGCACATTGTGAGGTGGTGATGGGAACTGCAGAGGCCCTGCACA |
| | GCATGCTAATGAGCCCAGGCAAACAT |
| CRE0008_ | TCCAAAGAAAAGCCAGATAAGTAGCTGATTATTGCATAGAGCTGACAGTATCACAGGAAG |
| AQP4 | ATCAGTAGTAGCAGCTCAAGTACAAAAAGGTTAATTAGCAATACTTAATAAGAAAAACTA |
| (SEQ ID | CCTCTGGCAGGTGAAGAGTTAATCCCTGGTCAATTTTAAGCTACTCTGCTGAGAGTACTA |
| NO: 47) | ATAAGTGTAGGGGTTGGAGCCAATGAGGGTGACCCCTTCCTTGATGGGAACAGTCATCCC |
| | TTAGGAACTGCCCTGGAAAGCATCAGCCAGCCAGAAAACAGGGAAAGAGGCTGAGAAACC |
| | GTGGTAACCAAGTTTTGCTGGCACTTTGTAAAATGGTAACTGCAACTGCCGAGGCTGTGC |
| | AGAGAATGCTAATAAGCCTAGGACAACCTGTAAAGAGTGGACCTAGAAAATGTCCACC |
| CRE0005_ | AACATATCCTGGTGTGGAGTAGGGGACGCTGCTCTGACAGAGGCTCGGGGGCCTGAGCTG |
| GFAP | GCTCTGTGAGCTGGGGAGGAGGCAGACAGCCAGGCCTTGTCTGCAAGCAGACCTGGCAGC |
| (SEQ ID | ATTGGGCTGGCCGCCCCCCAGGGCCTCCTCTTCATGCCCAGTGAATGACTCACCTTGGCA |
| NO: 48) | CAGACACAATGTTCGGGGTGGGCACAGTGCCTGCTTCCCGCCGCACCCCAGCCCCCCTCA |
| | AATGCCTTCCGAGAAGCCCATTGAGCAGGGGGCTTGCATTGCACCCCAGCCTGACAGCCT |
| | GGCATCTTGGGATAAAAGCAGCACAGCCCCCTAGGGGCTGCCCTTGCTGTGTGGCGCCAC |
| | CGGCGGTGGAGAACAAGGCTCTATTCAGCCTGTGCCCAGGAAAGGGGATCAGGGGATGCC |
| | CAGGCATGGACAGTGGGTGGCAGGGGGGGAGAGGAGGGCTGTCTGCTTCCCAGAAGTCCA |
| | AGGACACAAATGGGTGAGGGGA |
| CRE0007_ | AACATATCCTGGTGTGGAGTAGGGGACGCTGCTCTGACAGAGGCTCGGGGGCCTGAGCTG |
| GFAP | GCTCTGTGAGCTGGGGAGGAGGCAGACAGCCAGGCCTTGTCTGCAAGCAGACCTGGCAGC |
| (SEQ ID | ATTGGGCTGGCCGCCCCCCAGGGCCTCCTCTTCATGCCCAGTGAATGACTCACCTTGGCA |
| NO: 49) | CAGACACAATGTTCGGGGTGGGCACAGTGCCTGCTTCCCGCCGCACCCCAGCCCCCCTCA |
| | AATGCCTTCCGAGAAGCCCATTGAGCAGGGGGCTTGCATTGCACCCCAGCCTGACAGCCT |
| | GGCATCTTGGGATAAAAGCAGCAC |
| CRE0012_ | AGCGCGCAGAGTCTGCATGCGTGAGGAAGCTCCTGGGCGCGTCACAGCCGCGCTATTCTC |
| Arc | AGCGTCTCTCCTTTTATGGCTCCGGAAGTGAGCTGGGGTTGCTGGCAGCCTGGCTGGCAC |
| (SEQ ID | T |
| NO: 50) | |
| CRE0001_ | CTTCCTCTTATATTTCACCAAGACTCAGTTCCTGAGCAAGAAACCACAGGCACAGCAAGT |
| S100B | GCCATGAAAAGCGGCTTTGTGTGGGGTGGGCTCTTCACACTCCAATCTCCACTTCCTTCT |
| (SEQ ID | CAAGGCCTCAAAAAAAGTTGAAAAATGAAAACAAAAGCCCTGCTGTGTTGAGCTGGGCTC |
| NO: 51) | TGGCGTTGCCATGGACCCAGGGCAAACAGCGGTGCTCCTGCTCTGCCCCCGGCTCAGCTC |
| | ATGCTGGGCCTGCACTTCTGGAAGGGAGCATGGACTTTGGAATGACTGGTTAGAACCCAA |
| | ATGAATTAATGGAATTTGACATAGTTCAAAAATAATAAAATGTGATACCCATGAAATGCT |
| | GATATTCTGCCTTAATTTGCCAGATTGGGGGCC |
| CRE0009_ | TTCAGAGGTCGGATCTGAATCCAGCTCCAAGGCCCCAGCACCCAAGCCCTGACCCCATGG |
| S100B | CTGCCTGCTGGCTGGGAGTGGCATTCTTTAGAACAGGAAAGGCAGTGAGTTCTCATTGCA |
| (SEQ ID | TCAATACTTGCATTTGCTACAACAGAAGCTTTTTGTCTCTTCCTCACATTCCTTTAGAAC |
| NO: 52) | ACAAGCCTCCTTTTCTGCCTGTGGCCATCGGTCACTGGAGTCAGCCTCGTGAGTGGCTTG |
| | GTGGCGGATGGCACCGTCCTTTGTGGGAGAAAACAATGTTGCTGCCCAGGCCTTTCTGGA |
| | ATGACCCCTTCCCACCCCCTGACCAGCCCCAGCAAGGCCCGGGGCTGGCTGCCTAGTTGA |
| | TTGGAGCCAAGAGTTTGCTGAATGGATGAAGGGAGAAGGGACATCCAA |
| CRE0002_ | TCAGGGGTGCAGCTTTTTTTCTGTCTTTTACTCAGCCTGAGAAAGGTTGTCGTTTGACAA |
| S100B | GGTTTGTTCAGAGGTCGGATCTGAATCCAGCTCCAAGGCCCCAGCACCCAAGCCCTGACC |
| (SEQ ID | CCATGGCTGCCTGCTGGCTGGGAGTGGCATTCTTTAGAACAGGAAAGGCAGTGAGTTCTC |
| NO: 53) | ATTGCATCAATACTTGCATTTGCTACAACAGAAGCTTTTTGTCTCTTCCTCACATTCCTT |
| | TAGAACACAAGCCTCCTTTTCTGCCTGTGGCCATCGGTCACTGGAGTCAGCCTCGTGAGT |
| | GGCTTGGTGGCGGATGGCACCGTCCTTTGTGGGAGAAAACAATGTTGCTGCCCAGGCCTT |
| | TCTGGAATGACCCCTTCCCACCCCCTGACCAGCCCCAGCAAGGCCCGGGGCTGGCTGCCT |
| | AGTTGATTGGAGCCAAGAGTTTGCTGAATGGATGAAGGGAGAAGGGACATCCAAGTGACT |
| | GAAGAAGATCTTAACAGAA |

TABLE 6
Cis-regulatory elements (CRE) comprised in the promoters of Table 2

| Name | SEQUENCE |
|---|---|
| CRE0004_Lmx1b | CTGGGCAGAGAGGGGGCATCGGGGGCATGGCTAGGGGCCAGCACTGTGCTTCCTGGGCGC |
| (SEQ ID NO: 54) | CTCACCTCCTCCCTGACTCCTGGAGACTCCCAGCCCCTGTCTGGGAGATGAGCATTTAGG |
| | AATCTGCTTGTGCAGGGGTGGTGGGAGGGGCCGGGGTGGAGGGCGCATCCCCACGGGGAG |
| | ATTGGATGGAAATGGCCTGCCAGTGTGTGTGTGAGTGTGCGCCTGTGGCAGCAGCAGAGT |
| | AAACAGCCGCTGCCCTGTCCTCTCTGCGGCCGTGGCCAGGTACACAGGCCTGTTTGGACA |
| | GCTGCCTTGTCTGTCCGTCTGTTTGGGAGATGCTGGCTGATAGATGGGGATGGGCGGACT |
| | GTTAACCCCTCGTTGCCTGCACTGCTATGTGCTTCCTGCCTCATCCATGGGGTAGAAGGT |
| | AGCCAGAAGGTGGTCCTGGCTGTGCCCCCAGCTCCTCTCTAGGGGGGAAACCTCTAGTTC |
| | TGAGTCAGGGACAGAGTGAGGAGGGCTCCAGGGCATCAAGAGCTTGCTCCTCCCCGCACC |
| | AGGGAGCCAAGGACAGAGGAGAAGGGGGTCTTCCCCAGTGGTGACTAGGGGCAGAATATG |
| | TCTCTGAGTGAGTGTCTGGAGCCCTCCTCACCCCAACACCATG |
| CRE0003_Pitx3 | GGTGTGTGGAAGGGTGAGAGGCACACACACAGACACTGAAAGAATCCTAGGCCTGGTAGG |
| (SEQ ID NO: 55) | CACTTAACAAATGTCTGTTACAGACCAGAATTTTATTGCTGTTAGAGACCCAAGCCCCTC |
| | ATAGGAACAGTGAGAAACAGGTGCAGAAAGGCGGAGTAACTTTATCTAAAGTCATAGGCT |
| | CCCTGAATAGCAGAGCTGACACCTACAAGGAAGCGTTGGAGACCAGATCTACCAGCTAGC |
| | CTCCCTGAGACCACGAGGTGGCGCCGCAGCACCGGCTGTGGCCGATGCCAGCCAGGTAGC |
| | CGGTTTCCCACGTCCCCCGCACGCACGCACCTCTTTGCTGCAGGAATCCCGGGCTGCCCC |
| | GACCTGGAGTAGGGGGGGTGGTGAGTGGGACTGAGTCCCTAGAAGCCTGGACCCTCACTT |
| | CGTTCCTGTACATCCAGCTCGCCTGTAGACAGTGGGGGAGGATGAAGGGAAGAGGACTCA |
| | AGCGCAACTTTGAATCATCACGCCTTCGACAGTCCGCGCACGTTTATTTCATTTATCTTT |
| | GAAAACGAGGGAGGGGAAGCCTGGAGAAGGCGGGATGGGCCAAGGGTGAGTTGGCCCCCG |
| | GGGAGCTGGTCCCTGTTCCTGGCTTTAGTCCCAGGGGCGCGGTCTGTGTGTAGGGC |
| CRE0002_Gbf1 | TGCTACCAGAGCCGGGAGAGCTGCTCGGAGACGCCTCCGGGGTGCGGGCTGGACATGAGC |
| (SEQ ID NO: 56) | AGCGGCTGCCGGTCCTGGGACTAGGCCCCGCCATTTTGGATCCGCTGACAGGTCAGCGAA |
| | GTCTCTTCCTAGAGTTCCGGTGTCGTGAAGGCCGCCCTGACATCGCAATAGGGAATTAGT |
| | GGGAAGGGCCCTTAAATTGGGCGAGCCAAGGTGGG |
| CRE0005_faf1_ | GGAACATTTAGACTTTGGGAAAGAGATTAGTGATTTACATTGCTATCTCACTGATTTAAT |
| short | TTAAATGCTCTTCCAAACCAAACACACATGTGCCGAAGAGGCTACTAAGAAACCCAACAT |
| (SEQ ID NO: 57) | GCAGAGTTCTCTATAAGTGCAGCCGACAGTGTTGACTGAAACTAAACTTGGAAATCCAGG |
| | GCACTAATGCACAATATCAAGCAATAAAACGGCATCTCTTTGGCAATATTTAATTTAAAA |
| | AAGAAGAAAGAGACAGGCGAAGATCAGGCACTGTCTGTTTTGGAGGATCAACCATTCTGC |
| | ATTTCAAAGCATTGGTCCCTGCAATATCCAGGTTACTGTGCTAGAATCTCGACTATTATA |
| | TCGCAGTTGTGAGAGGGAGGGCAAAGATGTGTTTACTCAGTGATTAGGCCCTTAGAATAA |
| | GCCTCTAGCTCCTAGAGAGACAGCTCACCACTTATTCATTTGGGCCAATTCACAAAGCCT |
| | AGGAAGATTAAACATCCATGCTGAGAAGACAAGCGAATGCAGACGGTGAAAAAGAAATAA |
| | AAATTCTTTAAAAACTCTGAGATGACTTCATTATTTTTCCACAAGGAAACTTTAGGAAAG |
| | TGTTTAGTTAGAGAAAAACCCACATTGACCTCTCTCTAAACCCTTAATCTTTCCTTTGTG |
| | GTGGCACTGCTTTGTGGTAAGCGACTGGCTCGCCTCGCCCCTCTTTTCACTGGAAGCTGA |
| | GAGAAAAAAGACTCTGGAGAAACAGTTTTCGTTCCAGGGACACAAACCCCTGACACTGTT |
| | AA |
| CRE0006_Pitx2_ | GAAAATTTAATGTCATTGAAGCTCTATTTCCAATAGCAAAGGAGCACTATTGCTAATAGA |
| short (SEQ ID | CTTCAGAGCTTGAAATAAATAAATCTTTGGAATCCTGTTGCATCTCTTGGGGTGTGACAT |
| NO: 58) | TTGACAGTCTTTTATAGCACAGAACGAAACAAGTTTGTGAGCTGGAATTCAATTGTGGCG |
| | TATTGATTCCTTGCATCAGTCATTATTCCCTGCTGATTGACAGGTGAAAATTGGTTACGT |
| | TAAGTATTTCATATGTTATATTGGCTGACATTTGCTTGCCTGCTCTTGTGTCAATATTGT |
| | TGTAAAGATCTCCAGCTTTATGAGATAGCAATAGACACTGACTGTGGCTTTTGTGTGATG |
| | TTCCAGTGTTTTTCCTGACATAATTTAAGACATATTAAAAACCAGCAGCATCTTCCCTCT |
| | TGAGAAGCTTAATGCCAATATTATTGTCTTCCAGGGGAAGATCATGTATGCTCATAATCG |
| | GGTGCTAATTTCCACCAGTACGCTCATGTTTAGGCATTAGGCACTATAACTGTAAAATTG |
| | AGCCTTCTTGATTGATTCATGTCAAGCCTCATCTCGGCTCCTGCAGGGGAAGTCATCCGG |
| | CTGACCCTTTTTACACTAAAAGAAGAGATTTGTGTTCCTTTCTTTCACCTGGAACCATCA |
| | AATTGACTGAATAATCTGTAATACATTAGTGCTGACATTTGTTAGGGAGAATTAAACAAG |
| | ACACAGTAATCATTCCCCAGAATAAAAATTGTGTTTGAT |
| CRE0007_Pitx2_ | TGAGACTGAGTGGGAGTGGGGATGAAGCCTTTTTTCTTCATTTTTTTTTCCTCAGGTGCA |
| short (SEQ ID | ATTCTGTGTTAATATAAGAGAAGTGTGGCCTTCCTTCTCATAGCACTAAAAGTGAGATAA |
| NO: 59) | TCCCTGTGTAAGAAATCAGTAAGTACGGTCTGCTTAATCTAGTCCCAGTGTGAAACTGTT |
| | GACATTTGTTCTTTTTTCTATCATTATGTGACTGGGCCTGTTTTGTGCTGGATTAGGCAC |
| | AAATCTCCTATGCAGCACATTTGGCATGTTACTAGTAGTTTAACTTCATTAATAATGTAT |
| | GAAGAAAATGTAATCCATGACAAGGAAGCAAAGAAAAGTATTTTTTTTTTTTTTTGCTTC |
| | TCCCAAATCCTTTGGAATGAGTAATTATTCAACATTTTATGTTTGATGTTATATTTTACA |
| | ATTCAACTTCCATA |
| CRE0008_Pitx2_ | GCTGGTGCTTCTTTTTTCTGTTTTTTAAGCTGCTTTACAGACGAAAATGGAACTATATTT |
| short (SEQ ID | GGAACAATGCTTTCTGTTTTTCCATACTATTGATATTTGTGGAAAGTCACAAAATGGCCT |
| NO: 60) | AAGGAAGCTAAGCTCGCCCCAAGCAGTGGTCACTTACAAGTACTTTTGTACTCTGTACTC |
| | CTGTCACATTTGGGCGATCAGAGCAACAGCTGGGGAGACTTTTTCAACAAAGATGAGTGT |
| | CAGATAATCCTGATGAGATTCCACATCCAACATCTTTTGTAATTATGTCACATTCAGCTG |
| | TAATGGAATAATTCAAGCTGAAAGAACAAGCTTTGATCCTTTCTTAAACCTTTCCCTGTG |
| | GACTGGCTATCTAAAAGATTTAAAGATATTTCTGTTACAAGATCTAGTGTTTCCTCAGAG |
| | AAGTCATGCTTCTGAAGCATCGTGATCTACAAGAACAATATCAAGTTTGCCAAACACATT |
| | TCTGAAAGCATCGTGTTTTGGGGGGAGGGGTTGTATTTAATGAAGATATCAATAATATGC |

In some embodiments, the promoter is a synapsin (Syn1) promoter (see e.g., SEQ ID NO: 61). In one aspect, the promoter comprises a nucleic acid sequence at least 80% identical, e.g., at least 850%, at least 900%, at least 950%, at least 980%, at least 990% or 10000 identical, to SEQ ID NO: 61. In one aspect, provided herein is a composition comprising a recombinant viral vector comprising a promoter comprising a nucleic acid sequence at least 80% identical, e.g., at least 85%, at least 90%, at least 95%, at least 98%, at least 99% or 1000% identical, to SEQ ID NO: 61.

Synapsin-1 (SEQ ID NO: 61)
(SEQ ID NO: 61)
GAGGGCCCTGCGTATGAGTGCAAGTGGGTTTTAGGACCAGGATGAGGCGGGGTGGGGGTGCC
TACCTGACGACCGACCCCGACCCACTGGACAAGCACCCAACCCCCATTCCCCAAATTGCGCA
TCCCCTATCAGAGAGGGGGAGGGGAAACAGGATGCGGCGAGGCGCGTGCGCACTGCCAGCTT
CAGCACCGCGGACAGTGCCTTCGCCCCCGCCTGGCGGCGCGCGCCACCGCCGCCTCAGCACT
GAAGGCGCGCTGACGTCACTCGCCGGTCCCCCGCAAACTCCCCTTCCCGGCCACCTTGGTCG
CGTCCGCGCCGCCGCCGGCCCAGCCGGACCGCACCACGCGAGGCGCGAGATAGGGGGGCACG
GGCGCGACCATCTGCGCTGCGGCGCCGGCGACTCAGCGCTGCCTCAGTCTGCGGTGGGCAGC
GGAGGAGTCGTGTCGTGCCTGAGAGCGCAGTCG

In one embodiment, the nucleic acid further includes an enhancer sequence helpful in driving expression to the CNS, for example, to specified CNS tissues or cell types. Exemplary enhancer sequences are described in, e.g., U.S. patent application Ser. Nos. 17/283,232; 17/291,584; or International Patent Publication Nos WO2020168279A2; WO2021195591A2; WO2021248085A2; WO2021216778A2; the contents of each are incorporated herein by reference in their entireties.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the protein is GDNF. In some embodiments, the protein is human GDNF. In some embodiments, the GDNF gene encodes SEQ ID NO: 2 or a protein comprising SEQ ID NO: 2. In some embodiments, the GDNF gene encodes a protein with a sequence identity of at least 80%, at least 85%, at least 90%, at least 95%, at least 98% to SEQ ID NO: 2. In some embodiments, the therapeutic protein, and gene encoding such protein, is useful for treatment or slowing of the progression of PD.

A nucleic acid described herein may further comprise a reporter sequence (e.g., nucleic acid sequences encoding a reporter protein). Reporter sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid.

### GDNF

Glial cell line-derived neurotrophic factor (GDNF; NCBI Gene ID: 2668), also known as ATF, ATF1, ATF2, HSCR3, or HFB1-GDNF, is a neurotrophic factor that supports the development and survival of peripheral sympathetic, parasympathetic, enteric and sensory neurons as well as midbrain dopamine neurons and motoneurons. In various animal models of Parkinson's disease (PD) GDNF can prevent the neurotoxin-induced death of dopamine neurons and can promote axonal sprouting leading to functional recovery. Two GDNF splice variants, called pre-(α)pro-GDNF (previously called GDNFα) and pre-(β)pro-GDNF (previously called GDNFβ), have been described (Suter-Crazzolara and Unsicker, Neuroreport, 5:2486-2488 (1994)). These splice variants are produced by alternative splicing of the GDNF mRNA.

Many secreted proteins, including neurotrophic factors, are synthesized in the forms of precursors, pre-pro-mature proteins. The pre-region, consisting of the ER signal peptide, is clipped off during translation by a signal peptidase, and the pro-mature protein is released into the lumen of the ER immediately after being synthesized. The proteolytic cleavage of the mature protein can occur either inside the cell or in the extracellular matrix, or both.

The pro-mature protein can also remain uncleaved and have different function than the cleaved mature protein. For example, both mature brain-derived neurotrophic factor (BDNF) and pro-BDNF are secreted from neuronal cells. Mature BDNF binds to TrkB receptor inducing neuronal survival, differentiation and synaptic modulation, whereas pro-BDNF binds to p75^{NTR} and sortilin receptors inducing apoptosis (to review, see Thomas and Davies, Curr. Biol., 15:262-264 (2005); Teng et al., J. Neurosci., 25:5455-5463 (2005)).

In the scientific text, the names GDNF mRNA and GDNF protein have been used for the full-length pre-(α)pro-GDNF mRNA and for the mature GDNF protein that is produced by proteolytic cleavage of the (α)pro-GDNF protein. This mature GDNF protein has been extensively studied, and in PubMed more than 2500 citations are available for GDNF. GDNF was identified based on its ability to increase neurite length, cell size, and the number of dopaminergic neurons as well as their high affinity dopamine uptake in culture (Lin et al., Science, 260:1130-1132 (1993)). GDNF is a potent factor for the protection of nigral dopaminergic neurons against their toxin-induced degeneration in animal models of PD and also in the treatment of patients with PD (reviewed in Airaksinen and Saarma, Nat. Rev. Neurosci. 3:383-394 (2002) and Bespalov and Saarma, Trends Pharmacol. Sci. 28:68-74 (2007)). In addition, GDNF has a therapeutic role in the treatment of animal models of amyotrophic lateral sclerosis (ALS), addiction, alcoholism and depression (reviewed in Bohn, Exp. Neurol., 190:263-275 (2004); Messer et al., Neuron, 26:247-257 (2000); He et al., J. Neurosci., 25:619-628 (2005); Angelucci et al., Int. J. Neuropsychopharmacol., 6:225-231 (2003)). GDNF has important roles also outside the nervous system. It acts as a morphogen in kidney development and regulates the differentiation of spermatogonia (reviewed in Sariola and Saarma, J. Cell Sci. 116:3855-3862 (2003)).

In some embodiments, described herein is a viral vector for slowing or inhibiting progression of PD, wherein the vector comprises a GDNF encoding nucleic acid. In some embodiments, the viral vector is an Adeno-Associated Virus (AAV) vector (e.g., an rAAV).

In some embodiments, the viral vector comprises a nucleic acid sequence that encodes the amino acid sequence SEQ ID NO: 2. In some embodiments, the viral vector comprises a nucleic acid sequence that encodes an amino acid sequence with at least 80%, at least 85%, at least 90%, at least 95%, at least 98%, or more sequence identity to SEQ ID NO: 2.

In some embodiments, the viral vector comprises a sequence with at least 80%, at least 85%, at least 90%, at least 95%, at least 98%, or more sequence identity to SEQ ID NO: 1. In some embodiments, the viral vector comprises the sequence of SEQ ID NO: 1.

The term “gene” refers to a polynucleotide containing at least one open reading frame that is capable of encoding a particular polypeptide or protein after being transcribed or translated.

The terms “coding sequence” or “a sequence which encodes a particular protein”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

An exemplary cDNA sequence for GDNF is disclosed in Genbank Access NM_000514.4 (SEQ ID NO: 1). The amino acid sequence is shown in SEQ ID NO: 2. Methods described herein makes use of a nucleic acid construct comprising sequence SEQ ID NO: 1 or a variant thereof for slowing or inhibiting the progression of PD. The variants include, for instance, naturally-occurring variants due to allelic variations between individuals (e.g., polymorphisms), alternative splicing forms, etc. The term variant also includes GDNF gene sequences from other sources or organisms. Variants are preferably substantially homologous to SEQ ID NO: 1 and/or 2, i.e., exhibit a nucleotide sequence identity of typically at least about 75%, preferably at least about 85%, more preferably at least about 90%, more preferably at least about 95% with SEQ ID NO: 1 or 2. In some embodiments, the nucleic acid construct comprises a sequence with at least 95% sequence identity to SEQ ID NO: 1 and which retains the activity of SEQ ID NO: 1 or 2. Variants of a GDNF gene also include nucleic acid sequences, which hybridize to a sequence as defined above (or a complementary strand thereof) under stringent hybridization conditions. Typical stringent hybridization conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

There are reports of variants at nucleotide 277, 633, and 1389 of GDNF. For example, a C to T point mutation at nucleotide 277 (see, e.g., SEQ ID NO: 62), a C to G point mutation at nucleotide 633 (see, e.g., SEQ ID NO: 63), and a A to G point mutation at nucleotide 1389. Other variants are possible including codon optimized sequences, and conservative changes. Conservative substitutions are well known in the art.

In one embodiment, GDNF gene is codon optimized. In one embodiment, the GDNF nucleic acid sequence is codon optimized, for example, for any one or more of (1) enhanced expression in vivo, (2) to reduce CpG islands or (3) reduce the innate immune response. A skilled artisan can codon-optimize GDNF using standard techniques in the art.

In some embodiments, the viral vector comprises a nucleic acid sequence that encodes the amino acid sequence SEQ ID NO: 2, or variant thereof. In some embodiments, the viral vector comprises a nucleic acid sequence that encodes an amino acid sequence with at least 80%, at least 85%, at least 90%, at least 95%, at least 98%, or more sequence identity to SEQ ID NO: 2.

SEQ ID NO: 1 GDNF mRNA
(SEQ ID NO: 1)
atgaagtta tgggatgtcg tggctgtctg cctggtgctg ctccacaccg
cgtccgcctt cccgctgccc gccggtaaga ggcctcccga ggcgcccgcc
gaagaccgct ccctcggccg ccgccgcgcg cccttcgcgc tgagcagtga
ctcaaatatg ccagaggatt atcctgatca gttcgatgat gtcatggatt
ttattcaagc caccattaaa agactgaaaa ggtcaccaga taaacaaatg
gcagtgcttc ctagaagaga gcggaatcgg caggctgcag ctgccaaccc
agagaattcc agaggaaaag gtcggagagg ccagaggggc aaaaaccggg
gttgtgtctt aactgcaata catttaaatg tcactgactt gggtctgggc
tatgaaacca aggaggaact gatttttagg tactgcagcg gctcttgcga
tgcagctgag acaacgtacg acaaaatatt gaaaaactta tccagaaata
gaaggctggt gagtgacaaa gtagggcagg catgttgcag acccatcgcc
tttgatgatg acctgtcgtt tttagatgat aacctggttt accatattct
aagaaagcat tccgctaaaa ggtgtggatg tatctga
SEQ ID NO: 2 GDNF amino acid sequence
(SEQ ID NO: 2)
MKLWDVVAVCLVLLHTASAFPLPAGKRPPEAPAEDRSLGRRRAPFALSSDSNMPEDYPDQFDD
VMDFIQATIKRLKRSPDKQMAVLPRRERNRQAAAANPENSRGKGRRGQRGKNRGCVLTAIHLN
VTDLGLGYETKEELIFRYCSGSCDAAETTYDKILKNLSRNRRLVSDKVGQACCRPIAFDDDLS
FLDDNLVYHILRKHSAKRCGCI[stop]
SEQ ID NO: 62
variant GDNF mRNA (C277T)
(SEQ ID NO: 1)
atgaagtta tgggatgtcg tggctgtctg cctggtgctg ctccacaccg
cgtccgcctt cccgctgccc gccggtaaga ggcctcccga ggcgcccgcc
gaagaccgct ccctcggccg ccgccgcgcg cccttcgcgc tgagcagtga
ctcaaatatg ccagaggatt atcctgatca gttcgatgat gtcatggatt
ttattcaagc caccattaaa agactgaaaa ggtcaccaga taaacaaatg
gcagtgcttc ctagaagaga gcggaattgg caggctgcag ctgccaaccc
agagaattcc agaggaaaag gtcggagagg ccagaggggc aaaaaccggg
gttgtgtctt aactgcaata catttaaatg tcactgactt gggtctgggc
tatgaaacca aggaggaact gatttttagg tactgcagcg gctcttgcga
tgcagctgag acaacgtacg acaaaatatt gaaaaactta tccagaaata
gaaggctggt gagtgacaaa gtagggcagg catgttgcag acccatcgcc
tttgatgatg acctgtcgtt tttagatgat aacctggttt accatattct
aagaaagcat tccgctaaaa ggtgtggatg tatctga
variant GDNF mRNA (C633G)
SEQ ID NO: 63
atgaagtta tgggatgtcg tggctgtctg cctggtgctg ctccacaccg
cgtccgcctt cccgctgccc gccggtaaga ggcctcccga ggcgcccgcc
gaagaccgct ccctcggccg ccgccgcgcg cccttcgcgc tgagcagtga
ctcaaatatg ccagaggatt atcctgatca gttcgatgat gtcatggatt
ttattcaagc caccattaaa agactgaaaa ggtcaccaga taaacaaatg
gcagtgcttc ctagaagaga gcggaatcgg caggctgcag ctgccaaccc
agagaattcc agaggaaaag gtcggagagg ccagaggggc aaaaaccggg
gttgtgtctt aactgcaata catttaaatg tcactgactt gggtctgggc
tatgaaacca aggaggaact gatttttagg tactgcagcg gctcttgcga
tgcagctgag acaacgtacg acaaaatatt gaaaaactta tccagaaata
gaaggctggt gagtgacaaa gtagggcagg catgttgcag acccatcgcc
tttgatgatg acctgtcgtt tttagatgat aacctggttt accatattct

### Vectors

In some embodiments, the vector is adeno-associated virus (AAV) or recombinant AAV. In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around AAV genome. In some embodiments, the functions of the capsid proteins are to protect the viral genome, deliver the genome and interact with the host. In some aspects, capsid proteins deliver the viral genome to a host in a tissue specific manner.

In some embodiments, a recombinant AAV (rAAV) capsid protein is of an AAV serotype selected from the group consisting of AAV2, AAV3, AAV4, AAV5, AAV6, AAV8, AAVrh8, AAVrh10, AAV 2G9, AAV 2.5G9, AAV9, and AAV10. In some embodiments, an AAV capsid protein is of a serotype derived from a non-human primate, for example AAVrh10 serotype. In some embodiments, an AAV capsid protein is of an AAV9 serotype. In some embodiments, the capsid protein is an AAV1, AAV2, AAV3b, AAV4, AAV5, AAV6, AAV7, AAV8, AAV9, AAV10, AAV11, AAV12, or AAV13 capsid protein or, a chimera thereof. In some embodiments, the rAAV comprises a capsid protein from serotype AAV1, AAV2, AAV3a, AAV3b, AAV4, AAV5, AAV6, AAV7, AAV8, AAV9, AAV 2G9, AAV 2.5G9, AAV rh8, AAV rh10, AAV rh74, AAV10, or, AAV11 or, a chimera thereof.

In one embodiment, the AAV serotype and/or capsid described herein is selected from Table 7.

TABLE 7

| Serotype and where capsid sequence is published | Serotype and where capsid sequence is published |
|---|---|
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO: 10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in US20030138772) |
| | and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in |
| | US20030138772) |
| AAV42.2 (See SEQ ID NO: 102 in US20030138772) | AAV42.3b (See SEQ ID NO: 36 in |
| | US20030138772) |
| AAV42.3B (See SEQ ID NO: 107 in | AAV42.4 (See SEQ ID NO: 33 in |
| US20030138772) | US20030138772) |
| AAV42.4 (See SEQ ID NO: 88 in US20030138772) | AAV42.8 (See SEQ ID NO: 27 in |
| | US20030138772) |
| AAV42.8 (See SEQ ID NO: 85 in US20030138772) | AAV43.1 (See SEQ ID NO: 39 in |
| | US20030138772) |
| AAV43.1 (See SEQ ID NO: 92 in US20030138772) | AAV43.12 (See SEQ ID NO: 41 in |
| | US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in |
| | US20030138772; US20150315612 SEQ |
| ID NO: 182 | AAV8 (See SEQ ID NO: 95 in US20030138772), |
| | US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | U.S. Pat. No. 7,198,951, or SEQ ID NO: 223 in |
| | US20150315612) |
| AAV8 (See SEQ ID NO: 8 in US20150376240) | AAV8 (See SEQ ID NO: 214 in |
| | US20150315612) |
| AAV-8b (See SEQ ID NO: 5 in US20150376240) | AAV-8b (See SEQ ID NO: 3 in |
| | US20150376240) |
| AAV-8h (See SEQ ID NO: 6 in US20150376240) | AAV-8h (See SEQ ID NO: 4 in |
| | US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in |
| | US20030138772), U.S. Pat. No. 7,198,951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in US20030138772) | AAVA3.3 (See SEQ ID NO: 57 in |
| | US20030138772) |
| AAVA3.3 (See SEQ ID NO: 66 in US20030138772) | AAVA3.4 (See SEQ ID NO: 54 in |
| | US20030138772) |
| AAVA3.4 (See SEQ ID NO: 68 in US20030138772) | AAVA3.5 (See SEQ ID NO: 55 in |
| | US20030138772) |
| AAVA3.5 (See SEQ ID NO: 69 in US20030138772) | AAVA3.7 (See SEQ ID NO: 56 in |
| | US20030138772) |
| AAVA3.7 (See SEQ ID NO: 67 in US20030138772) | AAV29. (See SEQ ID NO: 11 in (AAVbb. l) 161 |
| | US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in US20030138772) | AAVcy.3 (AAV24.1) (See SEQ ID NO: 16 in |
| | US20030138772) |
| AAV27.3 (See SEQ ID NO: 104 in US20030138772) | AAVcy.4 (AAV27.3) (See SEQ ID NO: 17 in |
| | US20030138772) |
| AAVcy.5 (See SEQ ID NO: 227 in US20150315612) | AAV7.2 (See SEQ ID NO: 103 in |
| | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in US20150159173) | AAVCy.5Rl (See SEQ ID NO: in |
| | US20150159173 |
| AAVCy.5R2 (See SEQ ID NO: in US20150159173) | AAVCy.5R3 (See SEQ ID NO: in |
| | US20150159173 |
| AAVCy.5R4 (See SEQ ID NO: in US20150159173) | AAVDJ (See SEQ ID NO: 3 in |
| | US20140359799) and SEQ ID NO: 2 in |
| | U.S. Pat. No. 7,588,772) |
| | AAVDJ (See SEQ ID NO: 2 in |
| | US20140359799; and SEQ ID NO: 1 in |
| | U.S. Pat. No. 7,588,772) |
| | AAVDJ-8 (See SEQ ID NO: in U.S. Pat. No. 7,588,772; |
| | Grimm et al 2008 |
| AAVDJ-8 (See SEQ ID NO: in U.S. Pat. No. 7,588,772; Grimm | AAVF5 (See SEQ ID NO: 110 in |
| et al 2008 | US20030138772) |
| AAVH2 (See SEQ ID NO: 26 in US20030138772) | AAVH6 (See SEQ ID NO: 25 in |
| | US20030138772) |
| AAVhEl. l (See SEQ ID NO: 44 in U.S. Pat. No. 9,233,131) | AAVhErl.14 (See SEQ ID NO: 46 in |
| | U.S. Pat. No. 9,233,131) |
| AAVhErl.16 (See SEQ ID NO: 48 in U.S. Pat. No. 9,233,131) | AAVhErl.18 (See SEQ ID NO: 49 in |
| | U.S. Pat. No. 9,233,131) |
| AAVhErl.23 (AAVhEr2.29) (See SEQ ID NO: 53 in | AAVhErl.35 (See SEQ ID NO: 50 in |
| U.S. Pat. No. 9,233,131) | U.S. Pat. No. 9,233,131) |
| AAVhErl.36 (See SEQ ID NO: 52 in U.S. Pat. No. 9,233,131) | AAVhErl.5 (See SEQ ID NO: 45 in U.S. Pat. No. 9,233,131) |
| AAVhErl.7 (See SEQ ID NO: 51 in U.S. Pat. No. 9,233,131) | AAVhErl.8 (See SEQ ID NO: 47 in U.S. Pat. No. 9,233,131) |
| AAVhEr2.16 (See SEQ ID NO: 55 in U.S. Pat. No. 9,233,131) | AAVhEr2.30 (See SEQ ID NO: 56 in |
| | U.S. Pat. No. 9,233,131) |
| AAVhEr2.31 (See SEQ ID NO: 58 in U.S. Pat. No. 9,233,131) | AAVhEr2.36 (See SEQ ID NO: 57 in |
| | U.S. Pat. No. 9,233,131) |
| AAVhEr2.4 (See SEQ ID NO: 54 in U.S. Pat. No. 9,233,131) | AAVhEr3.1 (See SEQ ID NO: 59 in |
| | U.S. Pat. No. 9,233,131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 in |
| US20150315612) | US20150315612) |
| AAVhu.l l (AAV16.12) (See SEQ ID NO: 57 in | AAVhu.l l (AAV16.12) (See SEQ ID NO: 153 in |
| US20150315612) | US20150315612) |
| AAVhu.12 (See SEQ ID NO: 59 in US20150315612) | AAVhu.12 (See SEQ ID NO: 154 in |
| | US20150315612) |
| AAVhu.13 (See SEQ ID NO: 16 in US2015015917 | |
| and ID NO: 71 in US20150315612) | |
| AAVhu.13 (See SEQ ID NO: 32 in US20150159173 | |
| and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in US20150315612) | AAVhu.20 (See SEQ ID NO: 63 in |
| | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in US20150159173 | |
| and SEQ ID NO: 132 in US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in US20150315612) | AAVhu.3 (See SEQ ID NO: 145 in |
| | US20150315612) |
| AAVhu.30 (See SEQ ID NO: 70 in US20150315612) | AAVhu.30 (See SEQ ID NO: 131 in |
| | US20150315612) |
| AAVhu.31 (See SEQ ID NO: 1 in US20150315612) | AAVhu.31 (See SEQ ID NO: 121 in |
| | US20150315612) |
| AAVhu.32 (See SEQ ID NO: 2 in US20150315612) | AAVhu.32 (See SEQ ID NO: 122 in |
| | US20150315612) |
| AAVhu.33 (See SEQ ID NO: 75 in US20150315612) | AAVhu.33 (See SEQ ID NO: 124 in |
| | US20150315612) |
| AAVhu.34 (See SEQ ID NO: 72 in US20150315612) | AAVhu.34 (See SEQ ID NO: 125 in |
| | US20150315612) |
| AAVhu.35 (See SEQ ID NO: 73 in US20150315612) | AAVhu.35 (See SEQ ID NO: 164 in |
| | US20150315612) |
| AAVhu.36 (See SEQ ID NO: 74 in US20150315612) | AAVhu.36 (See SEQ ID NO: 126 in |
| | US20150315612) |
| AAVhu.37 (See SEQ ID NO: 34 in US20150159173 | |
| and SEQ ID NO: 88 in US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in US20150315612) | AAVhu.40 (See SEQ ID NO: 87 in |
| | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 in |
| US20150315612) | US20150315612) |
| AAVhu.44 (See SEQ ID NO: 45 in US20150159173 | |
| and SEQ ID NO: 158 in US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in US20150159173 | AAVhu.44R3 (See SEQ ID NO: in |
| | US20150159173 |
| AAVhu.45 (See SEQ ID NO: 76 in US20150315612) | AAVhu.45 (See SEQ ID NO: 127 in |
| | US20150315612) |
| AAVhu.46 (See SEQ ID NO: 82 in US20150315612) | AAVhu.46 (See SEQ ID NO: 159 in |
| | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 in |
| US20150315612) | US20150315612) |
| AAVhu.48Rl (See SEQ ID NO: in US20150159173 | AAVhu.48R2 (See SEQ ID NO: in |
| | US20150159173 |
| AAVhu.48R3 (See SEQ ID NO: in US20150159173 | AAVhu.49 (See SEQ ID NO: 209 in |
| | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in US20150315612) | AAVhu.51 (See SEQ ID NO: 208 in |
| | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in US20150159173) | AAVhu.53 (AAV145.1) (See SEQ ID NO: 176 in |
| | US20150315612) |
| AAVhu.54 (See SEQ ID NO: 188 in | AAVhu.54 (AAV145.5) (See SEQ ID NO: 177 in |
| US20150315612) | US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 in |
| US20150315612) | US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in US20150315612) | AAVhu.7 (See SEQ ID NO: 150 in |
| | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in US20150315612) | AAVhu.8 (See SEQ ID NO: 12 in |
| | US20150315612) |
| AAVhu.8 (See SEQ ID NO: 151 in US20150315612) | AAVhu.9 (AAV3.1) (See SEQ ID NO: 58 in |
| | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in WO2015121501 | |
| and SEQ ID NO: 31 in US20150376607) | |
| AAV-LK04 (See SEQ ID NO: 5 in US20150376607) | AAV-LK04 (See SEQ ID NO: 32 in |
| | US20150376607) |
| AAV-LK05 (See SEQ ID NO: 6 in US20150376607) | AAV-LK05 (See SEQ ID NO: 33 in |
| | US20150376607) |
| AAV-LK06 (See SEQ ID NO: 7 in US20150376607) | AAV-LK06 (See SEQ ID NO: 34 in |
| | US20150376607) |
| AAV-LK07 (See SEQ ID NO: 8 in US20150376607) | AAV-LK07 (See SEQ ID NO: 35 in |
| | US20150376607) |
| AAV-LK08 (See SEQ ID NO: 9 in US20150376607) | AAV-LK08 (See SEQ ID NO: 36 in |
| | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in US20150315612) | AAVpi.l (See SEQ ID NO: 93 in |
| | US20150315612; AAVpi.2 408, see SEQ ID NO: |
| | 30 in US20150315612) |
| AAVpi.2 (See SEQ ID NO: 95 in US20150315612) | AAVpi.3 (See SEQ ID NO: 29 in |
| | US20150315612) |
| AAVpi.3 (See SEQ ID NO: 94 in US20150315612) | AAVrh.10 (See SEQ ID NO: 9 in |
| | US20150159173) |
| AAVrh.10 (See SEQ ID NO: 25 in US20150159173) | AAV44.2 (See SEQ ID NO: 59 in |
| | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in US20150159173) | AAVrh.13 (See SEQ ID NO: 228 in |
| | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in US20150315612) | AAVrh.20 (See SEQ ID NO: 1 in |
| | US20150159173) |
| AAV42.10 (See SEQ ID NO: 106 in | AAVrh.21 (AAV42.10) (See SEQ ID NO: 35 in |
| US20030138772) | US20030138772) |
| AAV42.11 (See SEQ ID NO: 108 in | AAVrh.22 (AAV42.11) (See SEQ ID NO: 37 in |
| US20030138772) | US20030138772) |
| AAV42.12 (See SEQ ID NO: 113 in | AAVrh.23 (AAV42.12) (See SEQ ID NO: 58 in |
| US20030138772) | US20030138772) |
| AAV42.13 (See SEQ ID NO: 86 in US20030138772) | AAVrh.24 (AAV42.13) (See SEQ ID NO: 31 in |
| | US20030138772) |
| AAV42.15 (See SEQ ID NO: 84 in US20030138772) | AAVrh.25 (AAV42.15) (See SEQ ID NO: 28 in |
| | US20030138772) |
| AAVrh.2R (See SEQ ID NO: in US20150159173 | AAVrh.31 (AAV223.1) (See SEQ ID NO: 48 in |
| | US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in US20150159173 | |
| and SEQ ID NO: 116 in US20150315612) | |
| AAVrh.55 (See SEQ ID NO: 37 in US20150315612) | AAVrh.55 (AAV4-19) (See SEQ ID NO: 117 in |
| | US20150315612) |
| AAVrh.56 (See SEQ ID NO: 54 in US20150315612) | AAVrh.56 (See SEQ ID NO: 152 in |
| | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in US20150315612) | AAVrh.58 (See SEQ ID NO: 48 in |
| | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in US20150315612) | AAVrh.59 (See SEQ ID NO: 110 in |
| | US20150315612) |
| AAVrh.60 (See SEQ ID NO: 31 in US20150315612) | AAVrh.60 (See SEQ ID NO: 120 in |
| | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 in |
| US20150315612) | US20150315612) |
| AAVrh.64 (See SEQ ID NO: 15 in US20150315612) | AAVrh.64 (See SEQ ID NO: 43 in |
| | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in US20150159173 | AAVRh.64R2 (See SEQ ID NO: in |
| | US20150159173 |
| AAVrh.65 (See SEQ ID NO: 35 in US20150315612) | AAVrh.65 (See SEQ ID NO: 112 in |
| | US20150315612) |
| AAVrh.67 (See SEQ ID NO: 36 in US20150315612) | AAVrh.67 (See SEQ ID NO: 230 in |
| | US20150315612) |
| AAVrh.67 (See SEQ ID NO: 47 in US20150159173 | |
| and SEQ ID NO: 47 in US20150315612) | |
| AAVrh.68 (See SEQ ID NO: 16 in US20150315612) | AAVrh.68 (See SEQ ID NO: 100 in |
| | US20150315612) |
| AAVrh.69 (See SEQ ID NO: 39 in US20150315612) | AAVrh.69 (See SEQ ID NO: 119 in |
| | US20150315612) |
| AAVrh.70 (See SEQ ID NO: 20 in US20150315612) | AAVrh.70 (See SEQ ID NO: 98 in |
| | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in US20150159173) | AAVrh.74 (See SEQ ID NO: 6 in |
| | US20150159173) |
| AAVrh.8 (See SEQ ID NO: 41 in US20150159173) | AAVrh.8 (See SEQ ID NO: 235 in |
| | US20150315612) |
| AAVrh.8R (See SEQ ID NO: 9 in US20150159173, | AAVrh.8R A586R mutant (See SEQ ID NO: 10 |
| WO2015168666) | in WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in U.S. Pat. No. 7,427,396) | caprine AAV (See SEQ ID NO: 4 in |
| | U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | U.S. Pat. No. 8,734,809) |
| AAV CKd-1 (See SEQ ID NO: 131 in U.S. Pat. No. 8,734,809) | AAV CKd-10 (See SEQ ID NO: 58 in |
| | U.S. Pat. No. 8,734,809) |
| AAV CKd-10 (See SEQ ID NO: 132 in U.S. Pat. No. 8,734,809) | AAV CKd-2 (See SEQ ID NO: 59 in |
| | U.S. Pat. No. 8,734,809) |
| AAV CKd-2 (See SEQ ID NO: 133 in U.S. Pat. No. 8,734,809) | AAV CKd-3 (See SEQ ID NO: 60 in |
| | U.S. Pat. No. 8,734,809) |
| AAV CKd-3 (See SEQ ID NO: 134 in U.S. Pat. No. 8,734,809) | AAV CKd-4 (See SEQ ID NO: 61 in |
| | U.S. Pat. No. 8,734,809) |
| AAV CKd-4 (See SEQ ID NO: 135 in U.S. Pat. No. 8,734,809) | AAV CKd-6 (See SEQ ID NO: 62 in |
| | U.S. Pat. No. 8,734,809) |
| AAV CKd-6 (See SEQ ID NO: 136 in U.S. Pat. No. 8,734,809) | AAV CKd-7 (See SEQ ID NO: 63 in |
| | U.S. Pat. No. 8,734,809) |
| AAV CKd-7 (See SEQ ID NO: 137 in U.S. Pat. No. 8,734,809) | AAV CKd-8 (See SEQ ID NO: 64 in |
| | U.S. Pat. No. 8,734,809) |
| AAV CKd-8 (See SEQ ID NO: 138 in U.S. Pat. No. 8,734,809) | AAV CKd-B 1 (See SEQ ID NO: 73 in |
| | U.S. Pat. No. 8,734,809) |
| AAV CKd-B 1 (See SEQ ID NO: 147 in | AAV CKd-B2 (See SEQ ID NO: 74 in |
| U.S. Pat. No. 8,734,809) | U.S. Pat. No. 8,734,809) |
| AAV CKd-B2 (See SEQ ID NO: 148 in U.S. Pat. No. 8,734,809) | AAV CKd-B3 (See SEQ ID NO: 75 in |
| | U.S. Pat. No. 8,734,809) |
| AAV CKd-B3 (See SEQ ID NO: in U.S. Pat. No. 8,734,809 | AAV CKd-B3 (See SEQ ID NO: 149 in |
| | U.S. Pat. No. 8,734,809) |
| AAV CLv-1 (See SEQ ID NO: 65 in U.S. Pat. No. 8,734,809) | AAV CLv-1 (See SEQ ID NO: 139 in |
| | U.S. Pat. No. 8,734,809) |
| AAV CLvl-1 (See SEQ ID NO: 171 in U.S. Pat. No. 8,734,809) | AAV Civ 1-10 (See SEQ ID NO: 178 in |
| | U.S. Pat. No. 8,734,809) |
| AAV CLvl-2 (See SEQ ID NO: 172 in U.S. Pat. No. 8,734,809) | AAV CLv-12 (See SEQ ID NO: 66 in |
| | U.S. Pat. No. 8,734,809) |
| AAV CLv-12 (See SEQ ID NO: 140 in U.S. Pat. No. 8,734,809) | AAV CLvl-3 (See SEQ ID NO: 173 in |
| | U.S. Pat. No. 8,734,809) |
| AAV CLv-13 (See SEQ ID NO: 67 in U.S. Pat. No. 8,734,809) | AAV CLv-13 (See SEQ ID NO: 141 in |
| | U.S. Pat. No. 8,734,809) |
| AAV CLvl-4 (See SEQ ID NO: 174 in U.S. Pat. No. 8,734,809) | AAV Civ 1-7 (See SEQ ID NO: 175 in |
| | U.S. Pat. No. 8,734,809) |
| AAV Civ 1-8 (See SEQ ID NO: 176 in U.S. Pat. No. 8,734,809) | AAV Civ 1-9 (See SEQ ID NO: 177 in |
| | U.S. Pat. No. 8,734,809) |
| AAV CLv-2 (See SEQ ID NO: 68 in U.S. Pat. No. 8,734,809) | AAV CLv-2 (See SEQ ID NO: 142 in |
| | U.S. Pat. No. 8,734,809) |
| AAV CLv-3 (See SEQ ID NO: 69 in U.S. Pat. No. 8,734,809) | AAV CLv-3 (See SEQ ID NO: 143 in |
| | U.S. Pat. No. 8,734,809) |
| AAV CLv-4 (See SEQ ID NO: 70 in U.S. Pat. No. 8,734,809) | AAV CLv-4 (See SEQ ID NO: 144 in |
| | U.S. Pat. No. 8,734,809) |
| AAV CLv-6 (See SEQ ID NO: 71 in U.S. Pat. No. 8,734,809) | AAV CLv-6 (See SEQ ID NO: 145 in |
| | U.S. Pat. No. 8,734,809) |
| AAV CLv-8 (See SEQ ID NO: 72 in U.S. Pat. No. 8,734,809) | AAV CLv-8 (See SEQ ID NO: 146 in |
| | U.S. Pat. No. 8,734,809) |
| AAV CLv-Dl (See SEQ ID NO: 22 in U.S. Pat. No. 8,734,809) | AAV CLv-Dl (See SEQ ID NO: 96 in |
| | U.S. Pat. No. 8,734,809) |
| AAV CLv-D2 (See SEQ ID NO: 23 in U.S. Pat. No. 8,734,809) | AAV CLv-D2 (See SEQ ID NO: 97 in |
| | U.S. Pat. No. 8,734,809) |
| AAV CLv-D3 (See SEQ ID NO: 24 in U.S. Pat. No. 8,734,809) | AAV CLv-D3 (See SEQ ID NO: 98 in |
| | U.S. Pat. No. 8,734,809) |
| AAV CLv-D4 (See SEQ ID NO: 25 in U.S. Pat. No. 8,734,809) | AAV CLv-D4 (See SEQ ID NO: 99 in |
| | U.S. Pat. No. 8,734,809) |
| AAV CLv-D5 (See SEQ ID NO: 26 in U.S. Pat. No. 8,734,809) | AAV CLv-D5 (See SEQ ID NO: 100 in |
| | U.S. Pat. No. 8,734,809) |
| AAV CLv-D6 (See SEQ ID NO: 27 in U.S. Pat. No. 8,734,809) | AAV CLv-D6 (See SEQ ID NO: 101 in |
| | U.S. Pat. No. 8,734,809) |
| AAV CLv-D7 (See SEQ ID NO: 28 in U.S. Pat. No. 8,734,809) | AAV CLv-D7 (See SEQ ID NO: 102 in |
| | U.S. Pat. No. 8,734,809) |
| AAV CLv-D8 (See SEQ ID NO: 29 in U.S. Pat. No. 8,734,809) | AAV CLv-D8 (See SEQ ID NO: 103 in |
| | U.S. Pat. No. 8,734,809); AAV CLv-Kl 762, see SEQ ID NO: |
| | 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | U.S. Pat. No. 8,734,809) |
| AAV CLv-Rl (See SEQ ID NO: 104 in U.S. Pat. No. 8,734,809) | AAV CLv-R2 (See SEQ ID NO: 31 in |
| | U.S. Pat. No. 8,734,809) |
| AAV CLv-R2 (See SEQ ID NO: 105 in U.S. Pat. No. 8,734,809) | AAV CLv-R3 (See SEQ ID NO: 32 in |
| | U.S. Pat. No. 8,734,809) |
| AAV CLv-R3 (See SEQ ID NO: 106 in U.S. Pat. No. 8,734,809) | AAV CLv-R4 (See SEQ ID NO: 33 in |
| | U.S. Pat. No. 8,734,809) |
| AAV CLv-R4 (See SEQ ID NO: 107 in U.S. Pat. No. 8,734,809) | AAV CLv-R5 (See SEQ ID NO: 34 in |
| | U.S. Pat. No. 8,734,809) |
| AAV CLv-R5 (See SEQ ID NO: 108 in U.S. Pat. No. 8,734,809) | AAV CLv-R6 (See SEQ ID NO: 35 in |
| | U.S. Pat. No. 8,734,809) |
| AAV CLv-R6 (See SEQ ID NO: 109 in | AAV CLv-R7 (See SEQ ID NO: 110 in |
| U.S. Pat. No. 8,734,809); AAV CLv-R7 802 (see SEQ ID NO: 36 | U.S. Pat. No. 8,734,809) |
| in U.S. Pat. No. 8,734,809) | |
| AAV CLv-R8 (See SEQ ID NO: 37 in U.S. Pat. No. 8,734,809) | AAV CLv-R8 (See SEQ ID NO: 111 in |
| | U.S. Pat. No. 8,734,809) |
| AAV CLv-R9 (See SEQ ID NO: 38 in U.S. Pat. No. 8,734,809) | AAV CLv-R9 (See SEQ ID NO: 112 in |
| | U.S. Pat. No. 8,734,809) |
| AAV CSp-1 (See SEQ ID NO: 45 in U.S. Pat. No. 8,734,809) | AAV CSp-1 (See SEQ ID NO: 119 in |
| | U.S. Pat. No. 8,734,809) |
| AAV CSp-10 (See SEQ ID NO: 46 in U.S. Pat. No. 8,734,809) | AAV CSp-10 (See SEQ ID NO: 120 in |
| | U.S. Pat. No. 8,734,809) |
| AAV CSp-11 (See SEQ ID NO: 47 in U.S. Pat. No. 8,734,809) | AAV CSp-11 (See SEQ ID NO: 121 in |
| | U.S. Pat. No. 8,734,809) |
| AAV CSp-2 (See SEQ ID NO: 48 in U.S. Pat. No. 8,734,809) | AAV CSp-2 (See SEQ ID NO: 122 in |
| | U.S. Pat. No. 8,734,809) |
| AAV CSp-3 (See SEQ ID NO: 49 in U.S. Pat. No. 8,734,809) | AAV CSp-3 (See SEQ ID NO: 123 in |
| | U.S. Pat. No. 8,734,809) |
| AAV CSp-4 (See SEQ ID NO: 50 in U.S. Pat. No. 8,734,809) | AAV CSp-4 (See SEQ ID NO: 124 in |
| | U.S. Pat. No. 8,734,809) |
| AAV CSp-6 (See SEQ ID NO: 51 in U.S. Pat. No. 8,734,809) | AAV CSp-6 (See SEQ ID NO: 125 in |
| | U.S. Pat. No. 8,734,809) |
| AAV CSp-7 (See SEQ ID NO: 52 in U.S. Pat. No. 8,734,809) | AAV CSp-7 (See SEQ ID NO: 126 in |
| | U.S. Pat. No. 8,734,809) |
| AAV CSp-8 (See SEQ ID NO: 53 in U.S. Pat. No. 8,734,809) | AAV CSp-8 (See SEQ ID NO: 127 in |
| | U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| U.S. Pat. No. 8,734,809) | U.S. Pat. No. 8,734,809) |
| AAV.hu.48R3 (See SEQ ID NO: 183 in U.S. Pat. No. 8,734,809) | AAV.VR-355 (See SEQ ID NO: 181 in |
| | U.S. Pat. No. 8,734,809) |
| AAV3B (See SEQ ID NO: 48 in WO2016065001) | AAV3B (See SEQ ID NO: 98 in |
| | WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in |
| | WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

In certain embodiments, the rAAV comprises a chemically modified capsid as disclosed in WO 2017/212019 e.g., mannose ligand is chemically coupled to AAV2. The rAAVs with chemically modified capsids disclosed in WO 2017/212019 is incorporated herein by reference in its entirety. As a further embodiment, the AAV capsid proteins and virus capsids used herein can be polyploid (also referred to as rational haploid) in that they can comprise different combinations of VP1, VP2 and VP3 AAV serotypes in a single AAV capsid as described in PCT/US18/22725, PCT/US2018/044632, or U.S. Pat. No. 10,550,405 which are incorporated by reference.

The components to be cultured in the host cell to package a rAAV vector in an AAV capsid may be provided to the host cell in trans. Alternatively, any one or more of the required components {e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art. In some embodiments, the instant disclosure relates to a host cell containing a nucleic acid that comprises a coding sequence encoding a protein (e.g., wild-type huntingtin protein, optionally “hardened” wild-type huntingtin protein). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13: 197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term “vector” includes cloning and expression vehicles, as well as viral vectors. One type of vector is a “plasmid,” which refers to a circular double stranded DNA loop into which additional DNA segments are ligated. Another type of vector is a viral vector, wherein DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the technology described herein is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

In some aspects of the invention, the recombinant AAV comprising a nucleic acid encoding GDNF (AAV2-GDNF) is produced by the triple transfection method that uses close ended linear duplexed DNA molecules that lack bacterial backbone sequences, for example, as described in PCT/US2021/013689, published as WO/2021/146591, which is incorporated herein by reference in its entirety.

In some aspects of the invention, the recombinant AAV comprising a nucleic acid encoding GDNF (AAV2-GDNF) is produced by the method as described in PCT/US2022/013279, published as WO2022159679, which is incorporated herein by reference in its entirety.

In some embodiments, useful vectors are contemplated to be those vectors in which the nucleic acid segment to be transcribed is positioned under the transcriptional control of a promoter. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a coding sequence if the promoter region were capable of effecting transcription of that DNA sequence such that the resulting transcript can be translated into the desired protein or polypeptide.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of described herein may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

The phrases “operatively linked,” “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the ordinarily skilled artisan.

In some embodiments, one or more of the recombinantly expressed gene can be integrated into the genome of the cell.

A nucleic acid molecule described herein can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule described herein also may be accomplished by integrating the nucleic acid molecule into the genome.

In one embodiment, the genome packaged within AAV2-GDNF comprises a sequence of SEQ ID NO: 64. In one embodiment, the genome packaged within AAV2-GDNF consists of or consists essentially of the sequence of SEQ ID NO: 64. In one embodiment, the genome packaged within AAV2-GDNF comprises, consists of, or consist essentially of a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical to the sequence of SEQ ID NO: 64.

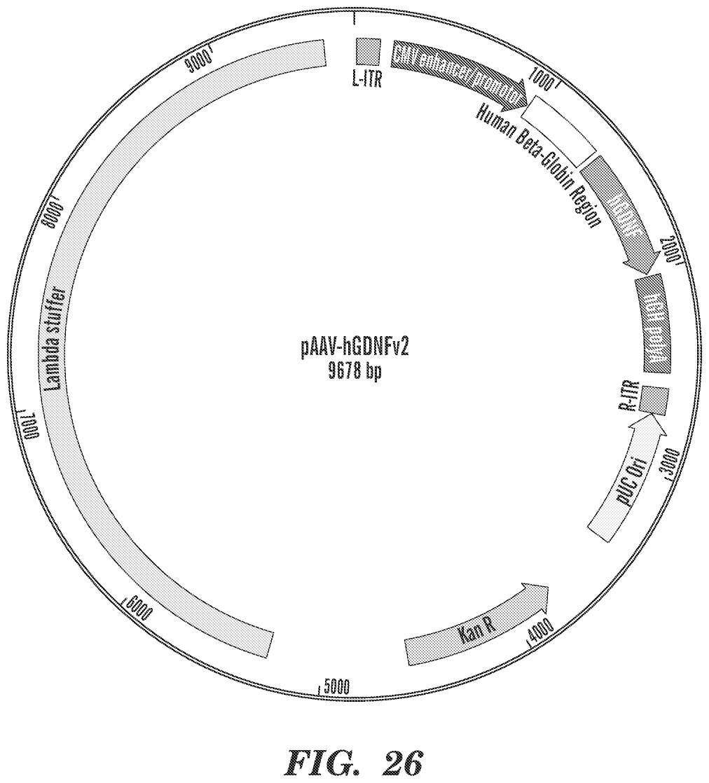

In one embodiment, the AAV2-GDNF vector comprises the ITR to ITR portion of sequence SEQ ID NO: 64 (i.e., base pairs 12-2,716 of SEQ ID NO: 64). In one embodiment, the AAV2-GDNF vector consists of or consists essentially of the ITR to ITR portion of sequence SEQ ID NO: 64 (i.e., base pairs 12-2,716 of SEQ ID NO: 64). In one embodiment, the AAV2-GDNF vector comprises, consists of, or consists essentially of the ITR to ITR portion of sequence SEQ ID NO: 64 (i.e., base pairs 12-2,716 of SEQ ID NO: 64) and is generated form a plasmid comprising, consisting of, or consisting essentially of SEQ ID NO: 64. In one embodiment, the AAV2-GDNF vector comprises, consists of, or consist essentially of a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical to the sequence of the ITR to ITR portion of sequence SEQ ID NO: 64 (i.e., base pairs 12-2,716 of SEQ ID NO: 64). In some embodiments, rAAV is manufactured using plasmid DNA as set forth in SEQ ID NO: 64, which is depicted in FIG. 26. In some embodiments, rAAV is manufactured using close ended linear duplexed DNA. In various embodiments, AAV2-GDNF includes a plasmid comprising the ITR to ITR portion of sequence SEQ ID NO: 64 (i.e., base pairs 12-2,716 of SEQ ID NO: 64). In other embodiments, AAV2-GDNF includes close ended linear duplexed DNA comprising the ITR to ITR portion of sequence SEQ ID NO: 64 (i.e., base pairs 12-2,716 of SEQ ID NO: 64), a non-limiting example of which is Doggybone DNA (dbDNA™), as disclosed in US Application 2018/0037943 and Karbowniczek et al., Bioinsights, 2017, which is incorporated herein in its entirety by reference.

SEQ ID NO: 64
cctgcaggcagctgcgcgctcgctcgctcactgaggccgcccgggcgtcgggcgacctttggtcgcccggcct
cagtgagcgagcgagcgcgcagagagggagtggccaactccatcactaggggttccttgtagttaatgattaa
cccgccatgctacttatctacgtagccatgctctagagcggccgcacgcgtctagttattaatagtaatcaat
tacggggtcattagttcatagcccatatatggagttccgcgttacataacttacggtaaatggcccgcctggc
tgaccgcccaacgacccccgcccattgacgtcaataatgacgtatgttcccatagtaacgccaatagggactt
tccattgacgtcaatgggtggagtatttacggtaaactgcccacttggcagtacatcaagtgtatcatatgcc
aagtacgccccctattgacgtcaatgacggtaaatggcccgcctggcattatgcccagtacatgaccttatgg
gactttcctacttggcagtacatctagtattagtcatcgctattaccatggtgatgcggttttggcagtacat
caatgggcgtggatagcggtttgactcacggggatttccaagtctccaccccattgacgtcaatgggagtttg
ttttggcaccaaaatcaacgggactttccaaaatgtcgtaacaactccgccccattgacgcaaatgggcggta
ggcgtgtacggtgggaggtctatataagcagagctcgtttagtgaaccgtcagatcgcctggagacgccatcc
acgctgttttgacctccatagaagacaccgggaccgatccagcctccgcggattcgaatcccggccgggaacg
gtgcattggaacgcggattccccgtgccaagagtgacgtaagtaccgcctatagagtctataggcccacaaaa
aatgctttcttcttttaatatacttttttgtttatcttatttctaatactttccctaatctctttctttcagg
gcaataatgatacaatgtatcatgcctctttgcaccattctaaagaataacagtgataatttctgggttaagg
caatagcaatatttctgcatataaatatttctgcatataaattgtaactgatgtaagaggtttcatattgcta
atagcagctacaatccagctaccattctgcttttattttatggttgggataaggctggattattctgagtcca
agctaggcccttttgctaatcatgttcatacctcttatcttcctcccacagctcctgggcaacgtgctggtct
gtgtgctggcccatcactttggcaaagaattgggattcgaacatcgattgaattgcaccatgaagttatggga
tgtcgtggctgtctgcctggtgctgctccacaccgcgtccgccttcccgctgcccgccggtaagaggcctccc
gaggcgcccgccgaagaccgctccctcggccgccgccgcgcgcccttcgcgctgagcagtgactcaaatatgc
cagaggattatcctgatcagttcgatgatgtcatggattttattcaagccaccattaaaagactgaaaaggtc
accagataaacaaatggcagtgcttcctagaagagagcggaatcggcaggctgcagctgccaacccagagaat
tccagaggaaaaggtcggagaggccagaggggcaaaaaccggggttgtgtcttaactgcaatacatttaaatg
tcactgacttgggtctgggctatgaaaccaaggaggaactgatttttaggtactgcagcggctcttgcgatgc
agctgagacaacgtacgacaaaatattgaaaaacttatccagaaatagaaggctggtgagtgacaaagtaggg
caggcatgttgcagacccatcgcctttgatgatgacctgtcgtttttagatgataacctggtttaccatattc

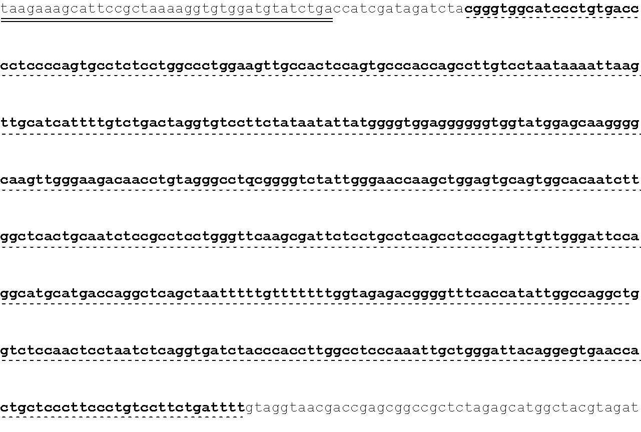

aagtagcatggcgggttaatcattaactacaaggaacccctagtgatggagttggccactccctctctgcgcg
ctcgctcgctcactgaggccgggcgaccaaaggtcgcccgacgcccgggctttgcccgggcggcctcagtgag
cgagcgagcgcgcagctgcctgcaggacatgtgagcaaaaggccagcaaaaggccaggaaccgtaaaaaggcc
gcgttgctggcgtttttccataggctccgcccccctgacgagcatcacaaaaatcgacgctcaagtcagaggt
ggcgaaacccgacaggactataaagataccaggcgtttccccctggaagctccctcgtgcgctctcctgttcc
gaccctgccgcttaccggatacctgtccgcctttctcccttcgggaagcgtggcgctttctcatagctcacgc
tgtaggtatctcagttcggtgtaggtcgttcgctccaagctgggctgtgtgcacgaaccccccgttcagcccg
accgctgcgccttatccggtaactatcgtcttgagtccaacccggtaagacacgacttatcgccactggcagc
agccactggtaacaggattagcagagcgaggtatgtaggcggtgctacagagttcttgaagtggtggcctaac
tacggctacactagaagaacagtatttggtatctgcgctctgctgaagccagttaccttcggaaaaagagttg
gtagctcttgatccggcaaacaaaccaccgctggtagccggtggttttttgtttgcaagcagcagattacgcg
cagaaaaaaaggatctcaagaagatcctttgatcttttctacggggtctgacgctcagtggaactccgtcgag
aggtctgcctcgtgaagaaggtgttgctgactcataccaggcctgaatcgccccatcatccagccagaaagtg
agggagccacggttgatgagagctttgttgtaggtggaccagttggtgattttgaacttttgctttgccacgg
aacggtctgcgttgtcgggaagatgcgtgatctgatccttcaactcagcaaaagttcgatttattcaacaaag
ccacgttgtgtctcaaaaatctctgatgttacattgcacaagataaaaatatatcatcatgaacaataaaactg

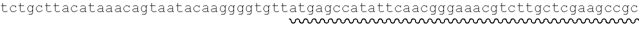

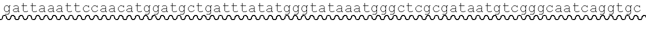

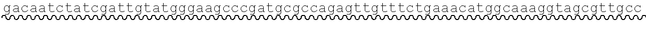

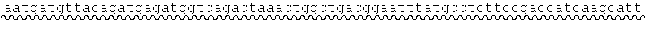

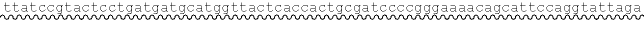

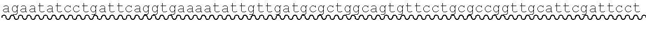

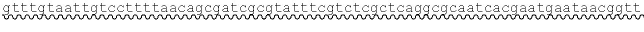

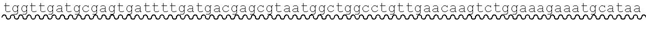

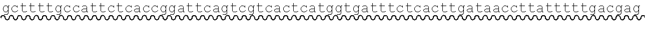

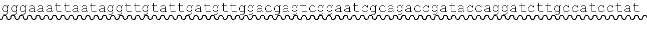

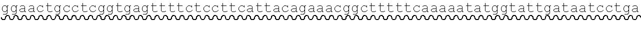

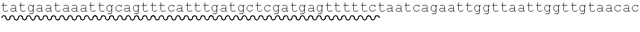

tggcagagcattacgctgacttgacgggacggcggctttgttgaataaatcgcattcgccattcaggctgcgc
aactgttgggaagggcgatcggtgcgggcctcttcgctattacgccagctggcgaaagggggatgtgctgcaa
ggcgattaagttgggtaacgccagggttttcccagtcacgacgttgtaaaacgacggccagtgccaagcttgc
atgcctgcaggtctaaatcaaaagaatagcccgagatagagttgagtgttgttccagtttggaacaagagtcc
actattaaagaacgtggactccaacgtcaaagggcgaaaaaccgtctatcagggcgatggcccactacgtgaa
ccatcacccaaatcaagttttttggggtcgaggtgccgtaaagcactaaatcggaaccctaaagggagccccc
gatttagagcttgacggggaaagccggcgaacgtggcgagaaaggaagggaagaaagcgaaaggagcgggcgc
taaggcgctggcaagtgtagcggtcacgctgcgcgtaaccaccacacccgccgcgcttaatgcgccgctacag
ggcgcgtactatggttgctttgacgtatgcggtgtgaaataccgcacagatgcgtaaggagaaaataccgcat
caggcaggtcgccgccccgtaacctgtcggatcaccggaaaggacccgtaaagtgataatgattatcatctac
atatcacaacgtgcgtggaggccatcaaaccacgtcaaataatcaattatgacgcaggtatcgtattaattga
tctgcatcaacttaacgtaaaaacaacttcagacaatacaaatcagcgacactgaatacggggcaacctcatg
tcaacgaagaacagaacccgcagaacaacaacccgcaacatccgctttcctaaccaaatgattgaacaaatta
acatcgctcttgagcaaaaagggtccgggaatttctcagcctgggtcattgaagcctgccgtcggagactaac
gtcagaaaagagagcatatacatcaattaaaagtgatgaagaatgaacatcccgcgttcttccctccgaacag
gacgatattgtaaattcacttaattacgagggcattgcagtaattgagttgcagttttaccactttcctgaca
gtgacagactgcgtgttggctctgtcacagactaaatagtttgaatgattagcagttatggtgatcagtcaac
caccagggaataatccttcatattattatcgtgcttcaccaacgctgcctcaattgctctgaatgcttccaga
gacaccttatgttctatacatgcaattacaacatcagggtaactcatagaaatggtgctattaagcatatttt
ttacacgaatcagatccacggagggatcatcagcagattgttctttattcattttgtcgctccatgcgcttgc
tcttcatctagcggttaaaatattacttcaaatctttctgtatgaagatttgagcacgttggccttacataca
tctgtcggttgtatttccctccagaatgccagcaggaccgcactttgttacgcaaccaatactattaagtgaa
aacattcctaatatttgacataaatcatcaacaaaacacaaggaggtcagaccagattgaaacgataaaaacg
ataatgcaaactacgcgccctcgtatcacatggaaggttttaccaatggctcaggttgccatttttaaagaaa
tattcgatcaagtgcgaaaagatttagactgtgaattgttttattctgaactaaaacgtcacaacgtctcaca
ttatatttactatctagccacagataatattcacatcgtgttagaaaacgataacaccgtgttaataaaagga
cttaaaaaggttgtaaatgttaaattctcaagaaacacgcatcttatagaaacgtcctatgataggttgaaat
caagagaaatcacatttcagcaatacagggaaaatcttgctaaagcaggagttttccgatgggttacaaatat
ccatgaacataaaagatattactatacctttgataattcattactatttactgagagcattcagaacactaca
caaatctttccacgctaaatcataacgtccggtttcttccgtgtcagcaccggggcgttggcataatgcaata
cgtgtacgcgctaaaccctgtgtgcatcgttttaattattcccggacactcccgcagagaagttccccgtcag
ggctgtggacatagttaatccgggaatacaatgacgattcatcgcacctgacatacattaataaatattaaca
atatgaaatttcaactcattgtttagggtttgtttaattttctacacatacgattctgcgaacttcaaaaagc
atcgggaataacaccatgaaaaaaatgctactcgctactgcgctggccctgcttattacaggatgtgctcaac
agacgtttactgttcaaaacaaaccggcagcagtagcaccaaaggaaaccatcacccatcatttcttcgtttc
tggaattgggcagaagaaaactgtcgatgcagccaaaatttgtggcggcgcagaaaatgttgttaaaacagaa
acccagcaaacattcgtaaatggattgctcggttttattactttaggcatttatactccgctggaagcgcgtg
tgtattgctcacaataattgcatgagttgcccatcgatatgggcaactctatctgcactgctcattaatatac
ttctgggttccttccagttgtttttgcatagtgatcagcctctctctgagggtgaaataatcccgttcagcgg
tgtctgccagtcggggggaggctgcattatccacgccggaggcggtggtggcttcacgcactgactgacagac
tgctttgatgtgcaaccgacgacgaccagcggcaacatcatcacgcagagcatcattttcagctttagcatca
gctaactccttcgtgtattttgcatcgagcgcagcaacatcacgctgacgcatctgcatgtcagtaattgccg
cgttcgccagcttcagttctctggcatttttgtcgcgctgggctttgtaggtaatggcgttatcacggtaatg
attaacagcccatgacaggcagacgatgatgcagataaccagagcggagataatcgcggtgactctgctcata
catcaatctctctgaccgttccgcccgcttctttgaattttgcaatcaggctgtcagccttatgctcgaacta
accataaccagcgcccggcagtgaagcccagatattgctgcaacggtcgattgcctgacggatatcaccacga
tcaatcataggtaaagcgccacgctccttaatctgctgcaatgccacagcgtcctgacttttcggagagaagt
ctttcaggccaagctgcttgcggtaggcatcccaccaacgggaaagaagctggtagcgtccggcgcctgttga
tttgagttttgggtttagcgtgacaagtttgcgagggtgatcggagtaatcagtaaatagctctccgcctaca
atgacgtcataaccatgatttctggttttctgacgtccgttatcagttccctccgaccacgccagcatatcga
ggaacgccttacgttgattattgatttctaccatcttctactccggcttttttagcagcgaagcgtttgataa
gcgaaccaatcgagtcagtaccgatgtagccgataaacacgctcgttatataagcgagattgctacttagtcc
ggcgaagtcgagaaggtcacgaatgaactaggcgataatggcgcacatcgttgcgtcgattactgtttttgta
aacgcaccgccattatatctgccgcgaaggtacgccattgcaaacgcaaggattgccccgatgccttgttcct
ttgccgcgagaatggcggccaacaggtcatgtttttctggcatcttcatgtcttacccccaataaggggattt
gctctatttaattaggaataaggtcgattactgatagaacaaatccaggctactgtgtttagtaatcagattt
gttcgtgaccgatatgcacgggcaaaacggcaggaggttgttagcgcgacctcctgccacccgctttcacgaa
ggtcatgtgtaaaaggccgcagcgtaactattactaatgaattcaggacagacagtggctacggctcagtttg
ggttgtgctgttgctgggcggcgatgacgcctgtacgcatttggtgatccggttctgcttccggtattcgctt
aattcagcacaacggaaagagcactggctaaccaggctcgccgactcttcacgattatcgactcaatgctctt
acctgttgtgcagatataaaaaatcccgaaaccgttatgcaggctctaactattacctgcgaactgtttcggg
attgcattttgcagacctctctgcctgcgatggttggagttccagacgatacgtcgaagtgaccaactaggcg
gaatcggtagtaagcgccgcctcttttcatctcactaccacaacgagcgaattaacccatcgttgagtcaaat
ttacccaattttattcaataagtcaatatcatgccgttaatatgttgccatccgtggcaatcatgctgctaac
gtgtgaccgcattcaaaatgttgtctgcgattgactcttctttgtggcattgcaccaccagagcgtcatacag
cggcttaacagtgcgtgaccaggtgggttgggtaaggtttgggattagcatcgtcacagcgcgatatgctgcg
cttgctggcatccttgaatagccgacgcctttgcatcttccgcactctttctcgacaactctcccccacagct
ctgttttggcaatatcaaccgcacggcctgtaccatggcaactgttgggaagggcgatcggtgcgggcctctt
cgctattacgccagctggcgaaagggggatgtgctgcaaggcgattaagttgggtaacgccagggttttccca
gtcacgacgttgtaaaacgacggccagtgccaagcttgcatg (SEQ ID NO: 64)

In one embodiment, the plasmid depicted in FIG. 26 is used to generate the AAV2-GDNF genome.

In one embodiment, genome packaged within AAV2-GDNF is depicted in FIG. 26.

In one embodiment, the AAV2-GDNF is manufactured using the plasmid depicted in FIG. 26.

In one embodiment, AAV2-GDNF comprises at least one component listed in Table 8.

TABLE 8

| | SEQ | | |
|---|---|---|---|
| Component of | ID | Denoted in | Base Pairs of |
| AAV2-GDNF | NO: | SEQ ID NO: 34 | SEQ ID NO: 64 |
| Left ITR sequence | 65 | denoted by underlined | 12-130 |
| | | text in SEQ ID NO: 64 | |
| Right ITR sequence | 66 | denoted by bolded, | 2,587-2,716 |
| | | double underlined text | |
| | | in SEQ ID NO: 64 | |
| CMV | 67 | denoted by bolded text | 198-944 |
| enhancer/promoter | | in SEQ ID NO: 64 | |
| sequence | | | |
| Human Beta-Globin | 68 | denoted by bolded, | 946-1,345 |
| Region sequence | | underlined text in | |
| | | SEQ ID NO: 64 | |
| hGDNF transgene | 69 | denoted by double- | 1,374-2009 |
| sequence | | underlined text in | |
| | | SEQ ID NO: 64 | |
| hGH polyA tail | 70 | denoted by dotted- | 2,025-2,510 |
| sequence | | underlined text in | |
| | | SEQ ID NO: 64 | |
| pUC Ori sequence | 71 | denoted by bolded, | 2,734-3,399 |
| | | italicized, underlined | |
| | | text in SEQ ID NO: 64 | |
| Kanamycin | 72 | denoted by squiggled- | 3,757-4,569 |
| resistance sequence | | underlined text in | |
| | | SEQ ID NO: 64 | |
| Lambda stuffer | 73 | denoted by italicized, | 5,274-9,529 |
| sequence | | underlined text in | |
| | | SEQ ID NO: 64 | |

### Modified Capsids

In one embodiment, the capsid described herein is further modified to increase tropism for the CNS. In one embodiment, tropism of the capsid, and therefore the AAV, is increased by at least 1%; 2%; 3%; 4%; 5%; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 500%; 510%; 52%; 53%; 54%; 550%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%%; 89%; 90%; 91%; 92%; 93%; 94%; 95%; 96%; 97%; 98%; 99% or greater, or at least 1×, 2×, 3×, 4×, 0×; 5×; 10×; 15×; 20×; 25×; 30×; 35×; 40×; 45×; 50×; 55×; 60×; 65×; 70×; 75×; 80×; 85×; 90×; 95×; 100×; 250×; 500×, 750×, or 1,000× or greater as compared to an unmodified AAV.

In one embodiment, a capsid is modified such that its tropism for a non-CNS tissue is decreased. For example, a capsid having a liver-specific tropism can be modified such that it no longer has such tropism. In one embodiment, a capsid is modified such that its tropism for a non-CNS tissue is decreased by at least 1%; 2%; 3%; 4%; 500; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or greater as compared to the unmodified capsid.

In yet another embodiment, the modified capsid is modified such that its tropism for CNS tissue is increased and its tropism for a non-CNS tissue is decreased. For example, a capsid having liver-specific tropism can be modified such that it exhibits CNS-specific tropism and has decreased liver-specific tropism. In one embodiment, CNS-tropism of the capsid is increased by at least 1%; 2%; 3%; 4%; 5%; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or greater, or at least 1×, 2×, 3×, 4×, 0×; 5×; 10×; 15×; 20×; 25×; 30×; 35×; 40×; 45×; 50×; 55×; 60×; 65×; 70×; 75×; 80×; 85×; 90×; 95×; 100×; 250×; 500×, 750×, or 1,000× or greater as compared to an unmodified capsid, and tropism for a non-CNS tissue is decreased by at least 1%; 2%; 3%; 4%; 5%; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or greater as compared to the unmodified capsid.

Provided herein is a composition comprising a modified viral capsid comprising a payload, wherein the payload comprises a regulatory sequence and a nucleic sequence flanked by inverted terminal repeats (ITRs) that target a central nervous system disorder, and wherein the modification is a chemical, non-chemical or amino acid modification. In some embodiments, the nucleic acid sequence of the payload comprises an isolated nucleic acid encoding a transgene, e.g., GDNF. In some embodiments, the nucleic acid sequence of the payload comprises an isolated nucleic acid encoding a GDNF protein.

In certain embodiments, the modified viral capsid comprises modification that results in its preferential targeting of the CNS. For example, the modified viral capsid has increased tropism for the CNS, and/or decreased tropism for at least a second location, e.g., the liver. Preferential targeting of the CNS does not exclude targeting to other sites, but rather indicates that it is more highly targeted to the CNS as compared to another site.

In one embodiment, the modified viral capsid comprises modification that results in its targeting of the CNS. For example, a modification to a capsid that typically targets a non-CNS site (e.g., the liver) can redirect the capsid to now target both the CNS and the non-CNS site. In such embodiment, the CNS-targeting does not need to be preferential.

In one embodiment, the modification to the capsid is an amino acid modification, e.g., an amino acid deletion, insertion, or substitute. In one embodiment, the amino acid modification increases tropism for the CNS. In one embodiment, the amino acid modification targets the modified capsid to the CNS.

In one embodiment, the modified viral capsid has or consists of, or consists essentially of a nucleic acid sequence that is 90% identical to SEQ ID NOs 1-4 of U.S. patent application Ser. No. 16/511,913, the contents of which are incorporated herein by references in its entirety. This US patent application describes chimeric AAV capsid sequences that exhibit a dominant tropism for oligodendrocytes, and can be used to create AAV vectors that transduce oligodendrocytes in the CNS of subject.

In one embodiment, the modified viral capsid is an AAV capsid protein comprising one or more amino acids substitutions, wherein the substitutions introduce a new glycan binding site into the AAV capsid protein. In some embodiments, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV8 or AAV10. Such AAV capsid protein is further described in, e.g., U.S. patent application Ser. No. 16/110,773; the contents of which are incorporated herein by references in its entirety.

In one embodiment, the modified viral capsid is an AAV capsid protein that comprises, consists of, or consists essentially of an AAV 2.5 capsid protein (SEQ ID NO: 1 of International Patent Application No. PCT/US2020/029493; the contents of which are incorporated herein by references in its entirety) comprising one or more amino acid substitutions that introduce a new glycan binding site. Such amino acid substitutions can target the capsid to neurons and glial cells, such as astrocytes. In embodiments of the capsid proteins, capsids, viral vectors and methods described in the International Patent Application No. WO/2020/219656, the one or more amino acid substitutions comprise A267S, SQAGASDIRDQSR464-476SX₁AGX₂SX₃X₄X₅X₆QX₇R (SEQ ID NOS 153 and 154, respectively), wherein X₁₋₇ can be any amino acid, and EYSW 500-503 (SEQ ID NO: 155) EX₈X₉W, wherein X₈₋₉ can be any amino acid. In embodiments of the capsid proteins, capsids, viral vectors and methods described herein, X₁ is V or a conservative substitution thereof, X₂ is P or a conservative substitution thereof, X₃ is N or a conservative substitution thereof, X₄ is M or a conservative substitution thereof, X₅ is A or a conservative substitution thereof; X₆ is V or a conservative substitution thereof, X₇ is G or a conservative substitution thereof, X₈ is F or a conservative substitution thereof, and/or X₉ is A or a conservative substitution thereof. In embodiments of the capsid proteins, capsids, viral vectors and methods described herein, X₁ is V, X₂ is P, X₃ is N, X₄ is M, X₅ is A, X₆ is V, X₇ is G, X₈ is F, and X₉ is A, wherein the new glycan binding site is a galactose binding site. Such AAV capsid protein is further described in, e.g., International Patent Application No. WO/2020/219656; the contents of which are incorporated herein by references in its entirety.

In one embodiment, the modified viral capsid is an AAV capsid protein particle comprising a surface-bound peptide, wherein the peptide bound to the surface of the AAV particle is Angiopep-2, GSH, HIV-1 TAT (48-60), ApoE (159-167)2, Leptin 30 (61-90), THR, PB5-3, PB5-5, PB5-14, or any combination thereof, as described in, e.g., U.S. patent application Ser. No. 16/956,306; the contents of which are incorporated herein by references in its entirety. Such AAV capsid permits delivery, e.g., of a payload, across the blood brain barrier.

In one embodiment, the modified viral capsid comprises a AAV capsid protein (e.g., an AAV1, AAV5, or AAV6 capsid protein), wherein the VP3 region of the capsid protein comprises modifications (e.g., replacement of a tyrosine residue with a non-tyrosine residue and/or a threonine residue with a non-threonine residue) at positions corresponding to: one or more of, or each of Y705, Y731, and T492 of a wild-type AAV1 capsid protein (e.g., SEQ ID NO: 1 of U.S. patent application Ser. No. 16/565,191; the contents of which are incorporated herein by references in its entirety); one or more of, or each of Y436, Y693, and Y719 of a wild-type AAV5 capsid protein (e.g., SEQ ID NO: 2 of U.S. patent application Ser. No. 16/565,191); or one or more of, or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid comprises a AAV capsid protein (e.g., an AAV1, AAV5, or AAV6 capsid protein) comprising Y to F (tyrosine to phenylalanine) modifications or T to V (threonine to valine) modifications in the VP3 region of the capsid at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the modified viral capsid comprises AAV capsid protein (e.g., an AAV1, AAV5, or AAV6 capsid protein), wherein a VP3 region of the capsid protein comprises modifications (e.g., replacement of a tyrosine residue with a non-tyrosine residue and/or a threonine residue with a non-threonine residue) at positions corresponding to: one or more of or each of Y705, Y731, and T492 of a wild-type AAV1 capsid protein (e.g., SEQ ID NO: 1 of U.S. patent application Ser. No. 16/565,191); one or more of or each of Y436, Y693, and Y719 of a wild-type AAV5 capsid protein (e.g., SEQ ID NO: 2 of U.S. patent application Ser. No. 16/565,191); or one or more of or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein) comprising Y to F (tyrosine to phenylalanine) modifications or T to V (threonine to valine) modifications in the VP3 region of the capsid protein at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the amino acid modification permits the modified capsid to evade neutralizing antibodies, for example, that are generated against a viral vector, e.g., of the same serotype. In one embodiment, the amino acid modification permits the modified capsid to be used for repeat administration, for example, the modification will enable the capsid to have a therapeutic effect upon re-administration.

In one embodiment, the modified viral capsid is a chimeric capsid. A “chimeric” capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein. Production of a chimeric capsid protein can be carried out according to protocols well known in the art and a significant number of chimeric capsid proteins are described in the literature as well as herein that can be included in the capsid.

In one embodiment, the modified viral capsid is a haploid capsid. As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric protein is present.

In some embodiments of the technology described herein, a modified viral capsid comprises one or more modifications, e.g., a chemical modification, a non-chemical modification, or an amino acid modification to the capsid. Such modifications can, for example, modify the tissue-type tropism or cell-type tropism of the modified capsid, among other things.

Modifications can alter the properties of the capsid, including biochemical properties such as receptor binding, directly, such that the modification itself alters the behavior of the capsid, or can permit further modification, such as the attachment of a ligand which in turn modifies behavior of the capsid in a desired manner.

In one embodiment, chemical modification of cysteine residues, which may be naturally present or introduced by genetic modification of a capsid polypeptide coding sequence, permits the covalent attachment of a ligand via disulfide bond formation (see, e.g., WO 2005/106046, the contents of which are incorporated herein by reference).

Various ligands are contemplated, including but not limited to antibodies or antigen-binding fragments thereof that, for example, target a cell-surface protein expressed by a target cell (see, e.g., WO 2000/002654, which is incorporated herein by reference).

WO2015/062516, the contents of which are also incorporated herein by reference, describes the insertion of an amino acid comprising an azido group by genetic modification of the capsid gene, followed by chemical conjugation of a ligand via the azido group.

The modification of AAV capsid tropism by glycation, or chemical conjugation of sugar moieties, is described by Horowitz et al., Bioconjugate Chem. 22: 529-532 (2011). That approach, and similar approaches are contemplated for modification of capsids as described herein.

In other embodiments, the coating of a viral capsid with a polymer, such as polyethylene glycol (PEG) or poly-(N-hydroxypropyl)methacrylamide (pHPMA) is specifically contemplated. Such modification can, for example, reduce specific and nonspecific interactions with non-target tissues.

In other embodiments, carbodiimide coupling is specifically contemplated. See, e.g., Joo et al. ACS Nano 5, titled “Enhanced Real-time Monitoring of Adeno-Associated Virus Trafficking by Virus-Quantum Dot Conjugates” (2011).

In other embodiments, the viral capsid can be modified, e.g., as described in WO 2017/212019, see also U.S. National Phase U.S. Ser. No. 16/308,740, the contents of which are each incorporated herein by reference. The approach described therein couples a viral capsid to a ligand via bonds comprising —CSNH— and an aromatic moiety. While genetically modified viral capsids can be further modified by this approach, the modifications described therein do not require genetic modification of the viral capsid. Ligands described therein include, for example, a targeting agent, a steric shielding agent for avoiding neutralizing antibody interactions, a labeling agent or a magnetic agent. Targeting ligands described therein include, for example, a cell-type specific ligand, a protein, a mono- or polysaccharide, a steroid hormone, an RGD motif peptide (e.g., Arg-Gly-Asp, a cell adhesion motif which can mimic cell adhesion proteins and bind to integrins), a vitamin, and a small molecule.

In one embodiment, the chemical modification described herein is a modification described in International patent application WO/2017/212019, the content of which is incorporated herein by reference in its entirety.

In one embodiment, the chemical modification described herein is a modification described in International patent application WO/2021/005210, the content of which is incorporated herein by reference in its entirety.

In one embodiment, the capsid has at least one chemically-modified tyrosine residue in its capsid, wherein said chemically-modified tyrosine residue is of formula (I):

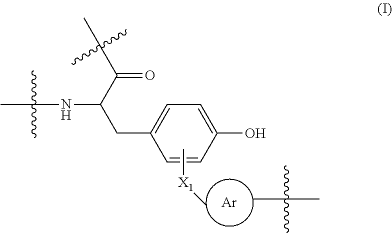

wherein:
X1 is selected from the group consisting of:

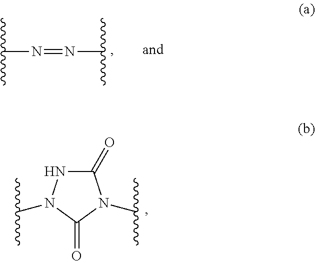

and
Ar is an aryl or a heteroaryl moiety optionally substituted.

In one embodiment, the capsid has at least one chemically-modified tyrosine residue is of formula (Ia):

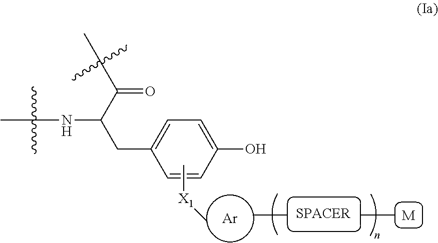

wherein:
Xi, and Ar are as defined herein above,
Spacer is a group for linking the “Ar” group to the functional moiety “M” which preferably comprises up to 1000 carbon atoms and which is preferably in the form of a chemical chain which optionally comprises heteroatoms and/or cyclic moieties,
n is 0 or 1; and
M is a functional moiety comprising a steric agent, a labelling agent, cell-types specific ligand or a drug moiety.

In one embodiment, Xi is of formula (a) and/or “Ar” is selected from substituted or unsubstituted phenyl, pyridyl, naphthyl, and anthracenyl.

In one embodiment, the capsid has at least one chemically-modified tyrosine is of formula (Ic):

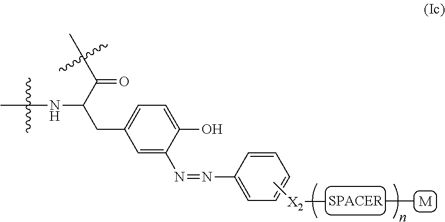

wherein:
X2 is —C(═O)—NH, —C(═O)—O, —C(═O)—O—C(═O)—, O—(C═O)—, NH—C(═O)—, NH—C(═O)—NH, —O—C═O—O—, O, NH, —NH(C═S)—, or —(C═S)—NH—, preferably —(C═O)—NH— or —(C═O)—O—
X2 is at position para, meta or ortho, preferably at position para of the phenyl group,
Spacer, n and M are as defined herein above.

In one embodiment, “Spacer”, when present, is selected from the group consisting of saturated or unsaturated, linear or branched C2-C40 hydrocarbon chains, optionally substituted, polyethylene glycol, polypropylene glycol, pHPMA (polymer of N-(2-Hydroxypropyl)methacrylamide), Poly Lactic-co-Glycolic Acid (PLGA), polymers of alkyl diamines and combinations thereof, and/or

“M” comprises, or consists of, cell-type targeting ligand, preferably selected from a mono- or a polysaccharide, a hormone, including a steroid hormone, a peptide such as RGD peptide (e.g., Arg-Gly-Asp, a cell adhesion motif which can mimic cell adhesion proteins and bind to integrins), a muscle targeting peptide (MTP) or Angiopep-2, a protein or a fragment thereof, a membrane receptor or a fragment thereof, an aptamer, an antibody including heavy-chain antibody, and fragments thereof such as antigen-binding fragment (Fab), Fab′ (which is the antigen-binding fragment further comprising a free sulfhydryl group), and VHH, a single-chain fragment variable (ScFv), a spiegelmer, a peptide aptamer, vitamins and drugs such as Cannabinoid receptor 1 (CB1) and/or Cannabinoid receptor 2 (CB2) ligands.

In one embodiment, “Spacer” (when present) is selected from the group consisting of linear or branched C2-C20 alkyl chains, polyethylene glycol, polypropylene glycol, pHPMA, PLGA, polymer of alkyl diamine and combinations thereof, said polymers having from 2 to 20 monomers and/or “M” comprises, or consists of, a cell-type specific ligand derived from a protein selected from transferrin, Epidermal Growth Factor (EGF), and basic Fibroblast Growth Factor 13FGF, a mono- or a polysaccharide comprising one or several galactose, mannose, N-acetylgalactosamine residues, bridge GalNac, or mannose-6-phosphate, MTP selected from SEQ ID NO:1 to SEQ ID NO:7, and vitamins such as folic acid.

In one embodiment, the capsid further has at least one additional chemically modified amino acid residue in the capsid, which is different from a tyrosine residue, said amino acid residue preferably bearing an amino group chemically modified with a group of formula (V):

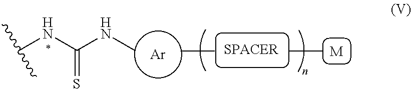

wherein:
N* being the nitrogen of the amino group of an amino acid residue, e.g. of a lysine residue or arginine residue, and
Ar, Spacer, n and M has the same definition as Ar, Spacer, n and M of formula (II) of claim 2.

In one embodiment, the capsid is incubated a chemical reagent bearing a reactive group selected from an aryl diazonium, and a 4-phenyl-1,2,4-triazole-3,5-dione (PTAD) moiety in conditions conducive for reacting said reactive group with a tyrosine residue present in the capsid so as to form a covalent bound.

In one embodiment, the capsid is incubated with a chemical reagent of formula VId to obtain the at least one chemically-modified tyrosine residue in the capsid of formula Ic.

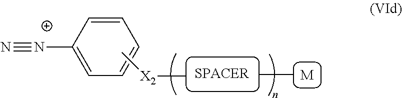

### Pharmaceutical Compositions

The expression cassettes, vectors or virions of the present invention may be formulated in a pharmaceutical composition with a pharmaceutically acceptable excipient i.e., one or more pharmaceutically acceptable carrier substances and/or additives, e.g., buffers, carriers, excipients, stabilizers, etc. The pharmaceutical composition may be provided in the form of a kit.

Accordingly, a further aspect of the present invention provides a pharmaceutical composition comprising an expression cassette, a vector or virion as described herein.

In various aspects, the pharmaceutical composition comprises a phosphate buffer. The phosphate buffer comprises from about 1 mM to about 50 mM phosphate, such as phosphate at a concentration of about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, or about 50 mM. The phosphate is prepared from a combination of dibasic phosphate (e.g., Na₂HPO₄, K₂HPO₄) and monobasic phosphate (e.g., NaH₂PO₄, KH₂PO₄) at a dibasic phosphate: monobasic phosphate molar ratio of from about 1:10 to about 10:1. For example, in various exemplary embodiments, the 10 mM phosphate comprises 9.5 mM dibasic phosphate and 0.5 mM monobasic phosphate, 9 mM dibasic phosphate and 1 mM monobasic phosphate, 8.5 mM dibasic phosphate and 1.5 mM monobasic phosphate, 8 mM dibasic phosphate and 2 mM monobasic phosphate, 7.5 mM dibasic phosphate and 2.5 mM monobasic phosphate, or 7 mM dibasic phosphate and 3 mM monobasic phosphate. The pH of the phosphate buffer is from about 6.5 to about 7.5, such as a pH of 6.5, 6.6, 6.7, 6.8, 6.9, 7.0, 7.1, 7.2, 7.3, 7.4, or 7.5. In one embodiment, the pH of the phosphate buffer is 7.2-7.3. In one embodiment, the pH of the phosphate buffer is 7.22. The phosphate buffer can also include NaCl at a concentration of from about 50 mM to about 200 mM, such as at a concentration of about 50 mM, about 60 mM, about 70 mM, about 80 mM, about 90 mM, about 100 mM, about 110 mM, about 120 mM, about 130 mM, about 135 mM, about 136 mM, about 137 mM, about 138 mM, about 139 mM, about 140 mM, about 150 mM, about 160 mM, about 170 mM, about 180 mM, about 190 mM, or about 200 mM. The phosphate buffer can also include KCl at a concentration of from about 0.5 mM to about 10 mM, such as at a concentration of about 0.5 mM, about 0.6 mM, about 0.7 mM, about 0.8 mM, about 0.9 mM, about 1 mM, about 2 mM, about 2.5 mM, about 2.6 mM, about 2.7 mM, about 2.8 mM, about 2.9 mM, about 3 mM, about 4 mM, about 5 mM, about 6 mM, about 7 mM, about 8 mM, about 9 mM, or about 10 mM. The phosphate buffer can also include CaCl₂ at a concentration of from about 0.20 mM to about 10 mM, such as at a concentration of about 0.2 mM, about 0.3 mM, about 0.4 mM, about 0.5 mM, about 0.6 mM, about 0.7 mM, about 0.8 mM, about 0.81 mM, about 0.82 mM, about 0.83 mM, about 0.84 mM, about 0.85 mM, about 0.86 mM, about 0.87 mM, about 0.88 mM, about 0.89 mM, about 0.9 mM, about 1 mM, about 2 mM, about 3 mM, about 4 mM, about 5 mM, about 6 mM, about 7 mM, about 8 mM, about 9 mM, or about 10 mM. The phosphate buffer can also include MgCl₂ at a concentration of from about 0.10 mM to about 1 mM, such as at a concentration of about 0.1 mM, about 0.2 mM, about 0.3 mM, about 0.4 mM, 0.41 mM, 0.42 mM, 0.43 mM, 0.44 mM, 0.45 mM, 0.46 mM, 0.47 mM, 0.48 mM, 0.49 mM, about 0.5 mM, about 0.6 mM, about 0.7 mM, about 0.8 mM, about 0.9 mM, or about 1 mM. The phosphate buffer can also include Poloxamer 188 (e.g., Pluronic™ F-68 non-ionic surfactant) at a concentration of from about 0.0001 wt. % to about 0.005 wt. %, such as at a concentration of about 0.0001 wt. %, about 0.0002 wt. %, about 0.0003 wt. %, about 0.0004 wt. %, about 0.0005 wt. %, about 0.0006 wt. %, about 0.0007 wt. %, about 0.0008 wt. %, about 0.0009 wt. %, about 0.001 wt. %, about 0.0015 wt. %, about 0.002 wt. %, about 0.0025 wt. %, about 0.003 wt. %, about 0.0035 wt. %, about 0.004 wt. %, about 0.0045 wt. %, or about 0.005 wt. %. The phosphate buffer can also include sorbitol at a concentration of from about 0.005 wt. % to about 10 wt. %, such as at a concentration of about 0.005 wt. %, about 0.075 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %. The rAAV comprising the nucleic acid (for example, AAV2 comprising the CMV promoter and the nucleic acid comprising a sequence at least 80% identical, e.g., at least 85%, at least 90%, at least 95%, at least 98%, at least 99% or 100% identical, to SEQ ID NO: 1; AAV2-GDNF) can have a titer in the phosphate buffer of from about 1×10¹² vg/mL to about 4×10¹² vg/mL; 2×10¹² vg/mL to about 4×10¹² vg/mL; 1×10¹² vg/mL to about 3×10¹² vg/mL; 1×10¹¹ vg/mL to about 2×10¹² vg/mL; 2×10¹² vg/mL to about 4×10¹² vg/mL; 8×10¹¹ vg/mL to about 9×10¹² vg/mL; 9×10¹¹ vg/mL to about 9×10¹² vg/mL; 1×10¹² vg/mL to about 9×10¹² vg/mL; 2×10¹² vg/mL to about 9×10¹² vg/mL; 3×10¹² vg/mL to about 9×10¹² vg/mL; 4×10¹² vg/mL to about 9×10¹² vg/mL; 5×10¹¹ vg/mL to about 9×10¹² vg/mL; 6×10¹² vg/mL to about 9×10¹² vg/mL; 7×10¹² vg/mL to about 9×10¹² vg/mL; 8×10¹² vg/mL to about 9×10¹² vg/mL; 8×10¹¹ vg/mL to about 8×10¹² vg/mL; 8×10¹¹ vg/mL to about 7×10¹² vg/mL; 8×10¹¹ vg/mL to about 6×10¹² vg/mL; 8×10¹¹ vg/mL to about 5×10¹² vg/mL; 8×10¹¹ vg/mL to about 4×10¹² vg/mL; 8×10¹¹ vg/mL to about 3×10¹² vg/mL; 8×10¹¹ vg/mL to about 2×10¹² vg/mL; 8×10¹¹ vg/mL to about 1×10¹² vg/mL; 8×10¹¹ vg/mL to about 9×10¹¹ vg/mL; 1×10¹² vg/mL to about 7×10¹² vg/mL; 3×10¹² vg/mL to about 6×10¹² vg/mL; 4×10¹² vg/mL to about 5×10¹² vg/mL; 3.1×10¹² vg/mL to about 4×10¹² vg/mL; 3.2×10¹² vg/mL to about 4×10¹² vg/mL; 3.3×10¹² vg/mL to about 4×10¹² vg/mL; 3.4×10¹² vg/mL to about 4×10¹² vg/mL; 3.5×10¹² vg/mL to about 4×10¹² vg/mL; 3.6×10¹² vg/mL to about 4×10¹² vg/mL; 3.7×10¹² vg/mL to about 4×10¹² vg/mL; 3.8×10¹² vg/mL to about 4×10¹² vg/mL; 3.9×10¹² vg/mL to about 4×10¹² vg/mL; 3×10¹² vg/mL to about 3.9×10¹² vg/mL; 3×10¹² vg/mL to about 3.8×10¹² vg/mL; 3×10¹² vg/mL to about 3.7×10¹² vg/mL; 3×10¹² vg/mL to about 3.6×10¹² vg/mL; 3×10¹² vg/mL to about 3.5×10¹² vg/mL; 3×10¹² vg/mL to about 3.4×10¹² vg/mL; 3×10¹² vg/mL to about 3.3×10¹² vg/mL; 3×10¹² vg/mL to about 3.2×10¹² vg/mL; and 3×10¹² vg/mL to about 3.1×10¹² vg/mL.

In one embodiment, the pharmaceutical composition comprises, consists essentially of, or consists the composition described in Table 9.

TABLE 9

| Component | Concentration | |
|---|---|---|
| Sodium Chloride | 180 | mM |
| Sodium Phosphate Monobasic Monohydrate | 2 | mM |
| Sodium Phosphate Dibasic Dihydrate | 8 | mM |

| | |
|---|---|
| Poloxamer P188 | 0.001% |
| Water for injection q.s. | NA |
| pH | 7.22 |

In some embodiments, the pharmaceutical composition comprises, consists essentially of, or consists of phosphate (monobasic and dibasic phosphate), NaCl, and poloxamer; pH 7.2-7.3.

In some embodiments, the pharmaceutical composition comprises, consists essentially of, or consists of about 10 mM phosphate (monobasic and dibasic phosphate), about 180 mM NaCl, and about 0.0010% poloxamer; pH 7.2-7.3.

In some embodiments, the pharmaceutical composition comprises, consists essentially of, or consists of about 8 mM dibasic phosphate, about 2 mM monobasic phosphate, about 180 mM NaCl, and about 0.0010% poloxamer; pH 7.2-7.3.

In some embodiments, the pharmaceutical composition comprises, consists essentially of, or consists of about 8 mM Na₂HPO₄, about 2 mM NaH₂PO₄, about 180 mM NaCl, and about 0.001% poloxamer; pH 7.2-7.3.

In some embodiments, the pharmaceutical composition comprises, consists essentially of, or consists of about 8 mM dibasic phosphate, about 2 mM monobasic phosphate, about 180 mM NaCl, and about 0.001% poloxamer; pH 7.2-7.3; and about 1×10¹² vg/mL to about 3.1×10¹³ vg/mL AAV2-GDNF.

In some embodiments, the pharmaceutical composition comprises, consists essentially of, or consists of about 8 mM dibasic phosphate, about 2 mM monobasic phosphate, about 180 mM NaCl, and about 0.001% poloxamer; pH 7.2-7.3; and at least 1×10¹² vg/mL AAV2-GDNF.

In some embodiments, the pharmaceutical composition comprises, consists essentially of, or consists of about 8 mM dibasic phosphate, about 2 mM monobasic phosphate, about 180 mM NaCl, and about 0.001% poloxamer; pH 7.2-7.3; and at least 5×10¹² vg/mL vg/mL AAV2-GDNF.

In some embodiments, the pharmaceutical composition comprises, consists essentially of, or consists of about 8 mM dibasic phosphate, about 2 mM monobasic phosphate, about 180 mM NaCl, and about 0.001% poloxamer; pH 7.2-7.3; and at least 1×10¹³ vg/mL AAV2-GDNF.

In some embodiments, the pharmaceutical composition comprises, consists essentially of, or consists of about 8 mM dibasic phosphate, about 2 mM monobasic phosphate, about 180 mM NaCl, and about 0.001% poloxamer; pH 7.2-7.3; and at least 3×10¹³ vg/mL AAV2-GDNF.

In some embodiments, the pharmaceutical composition comprises, consists essentially of, or consists of about 8 mM K₂HPO₄, about 2 mM KH₂PO₄, about 180 mM NaCl, and about 0.001% poloxamer; pH 7.2-7.3; and about 1×10¹² vg/mL to about 3.1×10¹³ vg/mL AAV2-GDNF.

In some embodiments, the pharmaceutical composition comprises, consists essentially of, or consists of about 8 mM K₂HPO₄, about 2 mM KH₂PO₄, about 180 mM NaCl, and about 0.001% poloxamer; pH 7.2-7.3; and at least 1×10¹² vg/mL AAV2-GDNF.

In some embodiments, the pharmaceutical composition comprises, consists essentially of, or consists of about 8 mM K₂HPO₄, about 2 mM KH₂PO₄, about 180 mM NaCl, and about 0.001% poloxamer; pH 7.2-7.3; and at least 5×10¹² vg/mL vg/mL AAV2-GDNF.

In some embodiments, the pharmaceutical composition comprises, consists essentially of, or consists of about 8 mM K₂HPO₄, about 2 mM KH₂PO₄, about 180 mM NaCl, and about 0.001% poloxamer; pH 7.2-7.3; and at least 1×10¹³ vg/mL AAV2-GDNF.

In some embodiments, the pharmaceutical composition comprises, consists essentially of, or consists of about 8 mM K₂HPO₄, about 2 mM KH₂PO₄, about 180 mM NaCl, and about 0.001% poloxamer; pH 7.2-7.3; and at least 3×10¹³ vg/mL AAV2-GDNF.

### Administration

The rAAVs of the disclosure may be delivered to a subject in compositions according to any appropriate methods known in the art. For example, an rAAV, preferably suspended in a physiologically compatible carrier (i.e., in a composition), may be administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

It may be desirable to deliver the rAAVs described herein directly to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

In one embodiment, the rAAVs or compositions thereof are delivered locally to the CNS, e.g., directly to the putamen, via a stepped cannula, e.g., as described in U.S. Pat. Nos. 7,815,623; 8,337,458; and 9,302,070, the contents of each of which are incorporated herein by reference in their entireties.

In one embodiment, the rAAVs or compositions thereof are delivered locally to the CNS, e.g., directly to the putamen, via SmartFlow cannula connected to MRI-compatible infusion pumps (e.g. Medfusion syringe pump, Smiths Medical Inc.

In one embodiment, the compostions described herein are locally administered, e.g., to the putamen, at a flow rate of 1-30 μL/min via a cannula. In one embodiment, the flow rate is about 1-25 μL/min; 1-20 μL/min; 1-15 μL/min; 1-10 μL/min; 1-5 μL/min; 5-30 μL/min; 10-30 μL/min; 15-30 μL/min; 20-30 μL/min; 25-30 μL/min; 5-25 μL/min; 10-20 μL/min; 15-25 μL/min; 5-15 μL/min; 5-25 μL/min; or 10-15 μL/min.

In one embodiment, the flow rate is about 1 μL/min; 2 μL/min; 3 μL/min; 4 μL/min; 5 μL/min; 6 μL/min; 7 μL/min; 8 μL/min; 9 μL/min; 10 μL/min; 11 μL/min; 12 μL/min; 13 μL/min; 14 μL/min; 15 μL/min; 16 μL/min; 17 μL/min; 18 μL/min; 19 μL/min; 20 μL/min; 21 μL/min; 22 μL/min; 23 μL/min; 24 μL/min; 25 μL/min; 26 μL/min; 27 μL/min; 28 μL/min; 29 μL/min; or 30 μL/min.

Moreover, in certain instances, it may be desirable to deliver the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

In one embodiment, the rAAV or composition thereof is administered during the subject “off” period.

In one embodiment, the rAAV or composition thereof is administered during the subject “on” period.

Aspects of the instant disclosure relate to compositions for slowing or inhibiting a progression of PD in a subject comprising any of the recombinant adeno-associated virus (rAAV) comprising a genome comprising a glial cell line-derived neurotrophic factor (GDNF) gene operably linked to a promoter described herein and a pharmaceutically acceptable carrier. In one aspect, the composition comprises any of the viral vectors described herein, and optionally, a pharmaceutically acceptable carrier. The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

The compositions of the disclosure may further comprise a second therapeutic, e.g., an anti-Parkinson's therapeutic described herein. The compositions of the disclosure may further comprise any immune modulator described herein. The compositions of the disclosure may further comprise a second therapeutic, e.g., an anti-Parkinson's therapeutic described herein and any immune modulator described herein.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., delivery to the putamen), oral, inhalation (including intranasal and intratracheal delivery), intraocular, intravenous, intramuscular, subcutaneous, intradermal, intratumoral, and other parental routes of administration. Routes of administration may be combined, if desired. In some embodiments, all or, at least one of the nucleic acid sequences disclosed herein are delivered via non-viral DNA constructs comprising at least one DD-ITR. For example, the non viral DNA constructs as described in WO 2019/246554 can be utilized to deliver one or more of the nucleic acids described herein. WO 2019/246554 is incorporated herein by reference in its entirety.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

In one embodiment, the rAAV is administered at a total dose within the range of 5×10¹² vg to about 1.5×10¹³ vg. In another embodiment the rAAV is administered at a total dose within the range of 1×10¹² vg to about 6.5×10¹³ vg; 2×10¹² vg to about 6.5×10¹³ vg; 3×10¹² vg to about 6.5×10¹³ vg 4×10¹² vg to about 6.5×10¹³ vg; 6×10¹² vg to about 6.5×10¹³ vg; 7×10¹² vg to about 6.5×10¹³ vg 8×10¹² vg to about 6.5×10¹³ vg; 9×10¹² vg to about 6.5×10¹³ vg; 1×10¹³ vg to about 6.5×10¹³ vg; 1×10¹² vg to about 1.5×10¹³ vg; 2×10¹² vg to about 1.5×10¹³ vg; 3×10¹³ vg to about 1.5×10¹³ vg 4×10¹² vg to about 1.5×10¹³ vg; 6×10¹² vg to about 1.5×10¹³ vg; 7×10¹² vg to about 1.5×10¹³ g; 8×10¹² vg to about 1.5×10¹³ vg; 9×10¹² vg to about 1.5×10¹³ vg; 1×10¹³ vg to about 1.5×10¹³ vg; 1×10¹² vg to about 6.5×10¹³ vg; 2×10¹² vg to about 6.5×10¹³ vg; 3×10¹² vg to about 6.5×10¹³ vg; 4×10¹² vg to about 6.5×10¹³ vg; 6×10¹² vg to about 6.5×10¹³ vg; 7×10¹³ vg to about 6.5×10¹³ vg; 8×10¹² vg to about 6.5×10¹³ vg; 9×10¹² vg to about 6.5×10¹³ vg; 7×10¹³ vg to about 6.5×10¹³ vg 1×10¹² vg to about 1×10¹³ vg; 1×10¹² vg to about 9×10¹³ vg; 1×10¹² vg to about 8×10¹³ vg; 1×10¹² vg to about 7×10¹² vg; 1×10¹² vg to about 6×10¹² vg; 1×10¹² vg to about 5×10¹² vg; 1×10¹² vg to about 4×10¹² vg; 1×10¹² vg to about 3×10¹² vg; 1×10¹² vg to about 2×10¹² vg; 5×10¹² vg to about 1×10¹³ vg; 5×10¹² vg to about 9×10¹² vg; 5×10¹² vg to about 8×10¹² vg; 5×10¹² vg to about 7×10¹² vg; 5×10¹² vg to about 6×10¹² vg; 5×10¹² vg to about 5.5×10¹³ vg; 5×10¹² vg to about 4.5×10¹³ vg; 5×10¹² vg to about 3.5×10¹³ vg; and 5×10¹² vg to about 2.5×10¹³ vg.

In one embodiment, the rAAV is administered at a total of at least 5.1×10¹² vg; 5.2×10¹² vg; 5.3×10¹² vg; 5.4×10¹² vg; 5.5×10¹² vg; 5.6×10¹² vg; 5.7×10¹² vg; 5.8×10¹² vg; 5.9×10¹² vg; 6×10¹² vg; 6.1×10¹² vg; 6.2×10¹² vg; 6.3×10¹² vg; 6.4×10¹² vg; 6.5×10¹² vg; 6.6×10¹² vg; 6.7×10¹² vg; 6.8×10¹² vg; 6.9×10¹² vg; 7×10¹² vg; 7.1×10¹² vg; 7.2×10¹² vg; 7.3×10¹² vg; 7.4×10¹² vg; 7.5×10¹² vg; 7.6×10¹² vg; 7.7×10¹² vg; 7.8×10¹² vg; 7.9×10¹² vg; 8×10¹² vg; 8.1×10¹² vg; 8.2×10¹² vg; 8.3×10¹² vg; 8.4×10¹² vg; 8.5×10¹² vg; 8.6×10¹² vg; 8.7×10¹² vg; 8.8×10¹² vg; 8.9×10¹² vg; 9×10¹² vg; 9.1×10¹² vg; 9.2×10¹² vg; 9.3×10¹² vg; 9.4×10¹² vg; 9.5×10¹² vg; 9.6×10¹² vg; 9.7×10¹² vg; 9.8×10¹² vg; 9.9×10¹² vg; 1.1×10¹³ vg; 1.2×10¹³ vg; 1.3×10¹³ vg; and 1.4×10¹³ vg. In one embodiment, the rAAV is administered at a total of at least 1×10¹² vg; at least 2×10¹² vg; at least 3×10¹² vg; at least 4×10¹² vg; at least 5×10¹² vg; at least 6×10¹² vg; at least 7×10¹² vg; at least 8×10¹² vg; at least 9×10¹² vg; at least 1×10¹³ vg; at least 2×10¹³ vg; at least 3×10¹³ vg; at least 4×10¹³ vg; at least 5×10¹³ vg; at least 6×10¹³ vg; and at least 7×10¹³ vg or more.

In one embodiment, wherein administration or inducing is performed locally to the subject's putamen, one half of the total dose is administered to each of the subject's putamen. Said another way, the total dose is divided substantially evenly between the subject's left putamen and right putamen.

An effective amount of an rAAV is an amount sufficient to target an infection in an animal, or to target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue (i.e., the putamen). In some cases, stable transgenic animals are produced by multiple doses of an rAAV.

In one embodiment, the rAAV is introduced or administered in a liquid composition. In one embodiment, the liquid composition has an rAAV concentration of from about 3×10¹² vg/mL to about 4×10¹² vg/mL. In one embodiment, the liquid composition has an rAAV concentration of from about 1×10¹² vg/mL to about 4×10¹² vg/mL; 2×10¹² vg/mL to about 4×10¹² vg/mL; 1×10¹² vg/mL to about 3×10¹² vg/mL; 1×10¹² vg/mL to about 2×10¹² vg/mL; 2×10¹² vg/mL to about 4×10¹² vg/mL; 8×10¹¹ vg/mL to about 9×10¹² vg/mL; 9×10¹¹ vg/mL to about 9×10¹² vg/mL; 1×10¹² vg/mL to about 9×10¹² vg/mL; 2×10¹² vg/mL to about 9×10¹² vg/mL; 3×10¹² vg/mL to about 9×10¹² vg/mL; 4×10¹² vg/mL to about 9×10¹² vg/mL; 5×10¹² vg/mL to about 9×10¹² vg/mL; 6×10¹² vg/mL to about 9×10¹² vg/mL; 7×10¹² vg/mL to about 9×10¹² vg/mL; 8×10¹² vg/mL to about 9×10¹² vg/mL; 8×10¹¹ vg/mL to about 8×10¹² vg/mL; 8×10¹¹ vg/mL to about 7×10¹² vg/mL; 8×10¹¹ vg/mL to about 6×10¹² vg/mL; 8×10¹¹ vg/mL to about 5×10¹² vg/mL; 8×10¹¹ vg/mL to about 4×10¹² vg/mL; 8×10¹¹ vg/mL to about 3×10¹² vg/mL; 8×10¹¹ vg/mL to about 2×10¹² vg/mL; 8×10¹¹ vg/mL to about 1×10¹² vg/mL; 8×10¹¹ vg/mL to about 9×10¹ vg/mL; 1×10¹² vg/mL to about 7×10¹² vg/mL; 3×10¹² vg/mL to about 6×10¹² vg/mL; 4×10¹¹ vg/mL to about 5×10¹² vg/mL; 3.1×10¹² vg/mL to about 4×10¹² vg/mL; 3.2×10¹² vg/mL to about 4×10¹² vg/mL; 3.3×10¹² vg/mL to about 4×10¹² vg/mL; 3.4×10¹² vg/mL to about 4×10¹² vg/mL; 3.5×10¹² vg/mL to about 4×10¹² vg/mL; 3.6×10¹² vg/mL to about 4×10¹² vg/mL; 3.7×10¹² vg/mL to about 4×10¹² vg/mL; 3.8×10¹² vg/mL to about 4×10¹² vg/mL; 3.9×10¹² vg/mL to about 4×10¹² vg/mL; 3×10¹² vg/mL to about 3.9×10¹² vg/mL; 3×10¹² vg/mL to about 3.8×10¹² vg/mL; 3×10¹² vg/mL to about 3.7×10¹² vg/mL; 3×10¹² vg/mL to about 3.6×10¹² vg/mL; 3×10¹² vg/mL to about 3.5×10¹² vg/mL; 3×10¹² vg/mL to about 3.4×10¹² vg/mL; 3×10¹² vg/mL to about 3.3×10¹² vg/mL; 3×10¹² vg/mL to about 3.2×10¹² vg/mL; and 3×10¹² vg/mL to about 3.1×10¹² vg/mL.

In one embodiment, the liquid composition has an rAAV concentration of about 8×10¹¹ vg/mL; 9×10¹¹ vg/mL; 1×10¹² vg/mL; 2×10¹² vg/mL; 3×10¹² vg/mL; 3.1×10¹² vg/mL; 3.2×10¹² vg/mL; 3.3×10¹² vg/mL; 3.4×10¹² vg/mL; 3.5×10¹² vg/mL; 3.6×10¹² vg/mL; 3.7×10¹² vg/mL; 3.8×10¹² vg/mL; 3.9×10¹² vg/mL; 4×10¹² vg/mL; 5×10¹² vg/mL; 6×10¹² vg/mL; 7×10¹² vg/mL; 8×10¹² vg/mL; and 9×10¹² vg/mL.

In some embodiments, a dose of rAAV is administered to a subject no more than once, e.g., it is administered to each putamen no more than once. In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In one embodiment, the rAAV or composition thereof is administered as an infusion via a trans-frontal (e.g., bi-frontal”) trajectory. The trans-frontal trajectory is at least one trajectory that is substantially perpendicular to the A-P axis of each putamen and accessed through the frontal bone of a skull. The trans-frontal administration can include infusing the rAAV through more than one trajectory that are substantially perpendicular the A-P axis of each putamen. Each trajectory may be accessed through a single burr hole through the frontal bone of the skull or through separate individual burr holes. Thus, at least one burr hole in the frontal bone is required for each of the left and right putamen. For example, the administration can be performed using a trans-frontal trajectory, such as a bi-frontal trajectory, in which a cannula is guided from the frontal bone at a first trajectory that is substantially perpendicular to the A-P axis of a first putamen and the rAAV is infused using the first trajectory. The cannula is then positioned at a second trajectory that is substantially perpendicular to the A-P axis of the first putamen (the second trajectory being different from the first trajectory) and the rAAV is infused using the second trajectory. The process is then repeated at a second putamen. It is understood that each trajectory results in the cannula contacting the putamen at different locations. FIG. 13A shows exemplary trans-frontal trajectories.

In one embodiment, the rAAV or composition thereof is administered as an infusion via a bi-occipital trajectory. An occipital trajectory is a single posterior trajectory that is substantially parallel to the A-P axis of a putamen and accessed through the occipital bone of a skull. Accordingly, a single burr hole in the occipital bone is needed per putamen. For example, the administration can be performed using the bi-occipital trajectory in which a cannula is guided from the occipital bone using a trajectory that is substantially parallel to the A-P axis of a first putamen and the rAAV is infused while the cannula is being advanced toward a rostral end of the first putamen. The process is then repeated at a second putamen. In a variation, the rAAV is infused into each putamen while the cannula is stationary and not being advanced to the rostral ends of each putamen. FIG. 13B shows an exemplary occipital trajectory.

In one embodiment, each putamen is infused using the same trajectory technique, i.e., each putamen is infused via a bi-frontal trajectory. In one embodiment, each putamen is infused using a different trajectory technique, i.e., one putamen is infused via a bi-frontal trajectory and the other putamen is infused via a bi-occipital trajectory.

In one embodiment, the infusion volume into a single putamen is less than or equal to 2000 μl, and greater than 1,800 μl. For example, the single putamen can be infused with a total volume that is 1,800 ul; 1,810 μl; 1,820 μl; 1,830 μl; 1,840 μl; 1,850 μl; 1,860 μl; 1,870 μl; 1,880 μl; 1,890 μl; 1,900 μl; 1,910 μl; 1,920 μl; 1,930 μl; 1,940 μl; 1,950 μl; 1,960 μl; 1,970 μl; 1,980 μl; 1,990 μl; or 2,000 μl. In one embodiment, the infusion volume for each putamen is the same. In one embodiment, the infusion volume for each putamen is different (e.g., one putamen is infused with 1,800 ul volume and the other putamen is infused with 2,000 um volume).

In some embodiments, rAAV compositions are formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

One aspect described herein is a formulation for slowing or inhibiting a progression of PD in a subject comprising any viral vector (e.g., an AAV) described herein at a concentration of 3×10¹² vg to 4×10¹² vg per mL of a pharmaceutically acceptable carrier. In one embodiment, the concentration of the viral vector is from about 1×10¹² vg/mL to about 4×10¹² vg/mL; 2×10¹² vg/mL to about 4×10¹² vg/mL; 1×10¹² vg/mL to about 3×10¹² vg/mL; 1×10¹² vg/mL to about 2×10¹² vg/mL; 2×10¹² vg/mL to about 4×10¹² vg/mL; 8×10¹¹ vg/mL to about 9×10¹² vg/mL; 9×10¹¹ vg/mL to about 9×10¹² vg/mL; 1×10¹² vg/mL to about 9×10¹² vg/mL; 2×10¹² vg/mL to about 9×10¹² vg/mL; 3×10¹² vg/mL to about 9×10¹² vg/mL; 4×10¹² vg/mL to about 9×10¹² vg/mL; 5×10¹² vg/mL to about 9×10¹² vg/mL; 6×10¹² vg/mL to about 9×10¹² vg/mL; 7×10¹² vg/mL to about 9×10¹² vg/mL; 8×10¹² vg/mL to about 9×10¹² vg/mL; 8×10¹¹ vg/mL to about 8×10¹² vg/mL; 8×10¹¹ vg/mL to about 7×10¹² vg/mL; 8×10¹¹ vg/mL to about 6×10¹² vg/mL; 8×10¹¹ vg/mL to about 5×10¹² vg/mL; 8×10¹¹ vg/mL to about 4×10¹² vg/mL; 8×10¹¹ vg/mL to about 3×10¹² vg/mL; 8×10¹¹ vg/mL to about 2×10¹² vg/mL; 8×10¹¹ vg/mL to about 1×10¹² vg/mL; 8×10¹¹ vg/mL to about 9×10¹ vg/mL; 1×10¹² vg/mL to about 7×10¹² vg/mL; 3×10¹² vg/mL to about 6×10¹² vg/mL; 4×10¹² vg/mL to about 5×10¹² vg/mL; 3.1×10¹² vg/mL to about 4×10¹² vg/mL; 3.2×10¹² vg/mL to about 4×10¹² vg/mL; 3.3×10¹² vg/mL to about 4×10¹² vg/mL; 3.4×10¹² vg/mL to about 4×10¹² vg/mL; 3.5×10¹² vg/mL to about 4×10¹² vg/mL; 3.6×10¹² vg/mL to about 4×10¹² vg/mL; 3.7×10¹² vg/mL to about 4×10¹² vg/mL; 3.8×10¹² vg/mL to about 4×10¹² vg/mL; 3.9×10¹² vg/mL to about 4×10¹² vg/mL; 3×10¹² vg/mL to about 3.9×10¹² vg/mL; 3×10¹² vg/mL to about 3.8×10¹² vg/mL; 3×10¹² vg/mL to about 3.7×10¹² vg/mL; 3×10¹² vg/mL to about 3.6×10¹² vg/mL; 3×10¹² vg/mL to about 3.5×10¹² vg/mL; 3×10¹² vg/mL to about 3.4×10¹² vg/mL; 3×10¹² vg/mL to about 3.3×10¹² vg/mL; 3×10¹² vg/mL to about 3.2×10¹² vg/mL; and 3×10¹² vg/mL to about 3.1×10¹² vg/mL. In one embodiment, the concentration of the viral vector is from about 8×10¹¹ vg/mL; 9×10¹¹ vg/mL; 1×10¹² vg/mL; 2×10¹² vg/mL; 3×10¹² vg/mL; 3.1×10¹² vg/mL; 3.2×10¹² vg/mL; 3.3×10¹² vg/mL; 3.4×10¹² vg/mL; 3.5×10¹² vg/mL; 3.6×10¹² vg/mL; 3.7×10¹² vg/mL; 3.8×10¹² vg/mL; 3.9×10¹² vg/mL; 4×10¹² vg/mL; 5×10¹² vg/mL; 6×10¹² vg/mL; 7×10¹² vg/mL; 8×10¹² vg/mL; and 9×10¹² vg/mL.

Typically, these formulations may contain at least about 0.10% of the active compound or more, although the percentage of the active ingredient(s) may, of course, be varied and may conveniently be between about 1% or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, lipid nanoparticles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the methods described herein relate to treating a subject having or diagnosed as having a PD with a nucleic acid described herein. Subjects having a PD can be identified by a clinician using current methods of diagnosing such diseases and disorders, for example, as described herein above. Symptoms and/or complications of PD which characterize this conditions and will aid in diagnosis, as well as clinical test to do the same are well known in the art and are described herein above. A family history of PD can also aid in determining if a subject is likely to have PD or in making a diagnosis of PD.

In one embodiment, the subject has been diagnosed as having PD prior to receiving a treatment as described herein. In one embodiment, the subject was diagnosed as having PD at least 1 year; 2 years; 3 years; 4 years; 5 years; 6 years; 7 years; 8 years; 9 years; 10 years or more prior to receiving a treatment as described herein.

In one embodiment, the subject has been diagnosed as being at risk of having PD prior to receiving a treatment as described herein.

In one embodiment, the subject has not been diagnosed as having, or being at risk of having PD prior to receiving a treatment as described herein.

In one embodiment, the subject is diagnosed as having PD prior to receiving a treatment as described herein. In one embodiment, the subject is diagnosed as being at risk of having PD prior to receiving a treatment as described herein.

In one embodiment, prior to administering treatment, the person administering the treatment receives results of a diagnostic assay(s) that diagnoses the subject as having PD. In one embodiment, prior to administering treatment, the person administering the treatment receives results of an assay(s) that diagnoses the subject as being at risk of having PD.

The compositions and methods described herein can be administered to a subject having or diagnosed as having PD. In some embodiments, the methods described herein comprise administering an effective amount of compositions described herein, e.g. a nucleic acid described herein to a subject in order to alleviate a symptom of a PD. As used herein, “alleviating a symptom” is ameliorating any condition or symptom associated with PD. As compared with an equivalent untreated control, such reduction is by at least 5%, 10%, 20%, 40%, 50%, 60%, 80%, 90%, 95%, 99% or more as measured by any standard technique.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for neuronal degradation or functionality among others. The dosage can be determined by a physician and adjusted, as necessary, to suit observed effects of the treatment.

### Combination Therapy

In one embodiment, the subject is administered at least one anti-PD therapeutic prior to instruction or administration of any of the rAAVs described herein. In on

In one embodiment, the subject is administered at least one anti-PD therapeutic following to instruction or administration of any of the rAAVs described herein.

In one embodiment, the at least one anti-PD therapeutic excludes Duopa.

In one embodiment, the rAAV described herein is used as a monotherapy. In one embodiment, the rAAV described herein can be used in combination with other known agents and therapies for PD. Administered “in combination,” as used herein, means that two (or more) different treatments are delivered to the subject during the course of the subject's affliction with the disease, e.g., the two or more treatments are delivered after the subject has been diagnosed with PD and before the disease has been cured or eliminated, or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disease is greater than what would be observed with one treatment delivered in the absence of the other. The effect of the two treatments can be partially additive, wholly additive, or greater than additive. The delivery can be such that an effect of the first treatment delivered is still detectable when the second is delivered. The rAAV described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat PD are described herein above.

When administered in combination with the rAAV, and the additional therapeutic (e.g., second or third anti-PD therapeutic) can be administered in an amount or dose that is higher, lower or the same as the amount or dosage of each therapeutic used individually, e.g., as a monotherapy. In certain embodiments, the administered amount or dosage of additional therapeutic is lower (e.g., at least 5%; 10%; 15%; 20%; 25%; 30%; 35%; 40%; 45%; 50%; 55%; 60%; 65%; 70%; 75%; 80%; 85%; 90%; 95% or more lower) than the amount or dosage of each additional therapeutic used individually.

In other embodiments, the amount or dosage of additional therapeutic that results in a desired effect (e.g., inhibiting or slowing the progression of PD) is lower (e.g., at least 5%; 10%; 15%; 20%; 25%; 30%; 35%; 40%; 45%; 50%; 55%; 60%; 65%; 70%; 75%; 80%; 85%; 90%; 95% or more lower) than the amount or dosage of each additional therapeutic individually required to achieve the same therapeutic effect.

In one embodiment, the subject maintains the same amount or dosage of the at least one anti-PD therapeutic following introduction or administration of any of the rAAVs described herein.

In one embodiment, the subject decreases the amount or dosage of the at least one anti-PD therapeutic following introduction or administration of any of the rAAVs described herein. In one embodiment, the amount or dosage of the at least one anti-PD therapeutic is decreased by at least 5%; 10%; 15%; 20%; 25%; 30%; 35%; 40%; 45%; 50%; 55%; 60%; 65%; 70%; 75%; 80%; 85%; 90%; 95% or more as compared to the amount or dosage taken prior to introduction or administration of any of the rAAVs.

In one embodiment, the subject is no longer administered an anti-PD therapeutic following introduction or administration of any of the rAAVs described herein.

### Immune Modulators

In some embodiments, the compositions described herein include an immune modulator, and the methods further comprise administering the immune modulator. The immune modulator can be administered at the time of administration, before the administration or, after the administration. In the case in which a subject is re-administered at least a second composition, the immune modulator can be administered prior to, with, or after the at least second administration.

In a preferred embodiment, the immune modulator is administered prior to administration of a recombinant viral vector. In various embodiments, the immune modulator is administered at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more prior to administration of a recombinant viral vector. In one embodiment, the immune modulator is administered no more than 24 hours prior to administration of a recombinant viral vector.

In one embodiment, the immune modulator is administered at substantially the same time as the recombinant viral vector, e.g., slightly before administration of the recombinant viral vector as disclosed herein (i.e., within 6-hours, or 5-hours, or 4-hours, or 3-hours, or 2-hours, or 1-hour). In some embodiments, the immune modulator is administered simultaneously, or within 6 hours after, administration of the viral vector, (i.e., within 1-hour, or within 2-hours, or within 3-hours, or within 4-hours, or within 5-hours or within 6-hours, or about 6-hours after administration of a viral vector composition as disclosed.

In some embodiments, the immune modulator allows for the administration of a recombinant viral vector to a subject who would otherwise not be a good candidate to receive such vector. A subject who would otherwise not be a good candidate to receive such a vector is, for example, a subject who has previously received administration of a recombinant viral vector and/or who was previously exposed to the recombinant viral vector and has subsequently developed an antibody response to the vector. Typically, a subject is considered to be a candidate, i.e., a good candidate, for administration of a recombinant viral vector when they have a titer for viral vector binding antibodies that is less than 1:5 (e.g., 1:1, 1:2, 1:3, or 1:4). In contrast, a subject is considered not to be a suitable candidate for administration of a recombinant viral vector when they have a titer for viral vector binding antibodies that is 1:5 or greater (e.g., 1:6, 1:6, 1:7, 1:8, 1:9, 1:10, 1:20, 1:30, 1:50, 1:100, 1:1,000 or more). One skilled in the art can assess the antibody titer of a subject using standard techniques in the art, e.g., by taking a biological sample from a subject, e.g., the subject's blood, challenging the biological sample with known antigens, and detecting the presence of the viral binding antibodies to the known antigens. An antibody titer is a measure of how much a sample can be diluted before a 50% viral vector neutralization can be detected in the sample. Antibody titers are usually expressed as ratios, such as 1:100, meaning that one-part serum to 100 parts saline solution (i.e., dilutant) results in 50% antibody, neutralization in the sample, i.e., a reciprocal dilution of serum required to inhibit viral infection by 50% can be designated as neutralizing antibody titer at 50% inhibition. A titer of 1:10 of viral vector antibody is, therefore, an indication of lower level of viral vector antibodies than a 1:100 titer.

Accordingly, in one embodiment, the subject is assessed for the presence of anti-AAV antibodies to the AAV vector of a gene therapy prior to administration of the gene therapy. In one embodiment, the subject is assessed for the presence of neutralizing anti-AAV antibodies to the AAV vector of a gene therapy prior to administration of the gene therapy. Methods for detecting neutralizing anti-AAV antibodies is further described in, e.g., Kasprzyk T., et al. Mol Therapy. Methods & Clinical Dev. Jan. 6, 2022, the contents of which are incorporated herein in its entirety by reference.

In one embodiment, the immune modulator is administered to a subject having a titer of viral vector binding antibodies present in the biological sample, e.g., a blood sample, from the subject that is less than about 1:5 (e.g., 1:1, 1:2, 1:3, or 1:4), where 1 part of the biological sample diluted in 10,000 parts of buffer results in 50% viral vector neutralization.

In one embodiment, the immune modulator is administered to a subject having a titer of viral vector binding antibodies present in the biological sample or blood product from the subject that is greater than or equal to 1:5 and less than about 1:10 (e.g., 1:6, 1:7, 1:8, or 1:9), where 1 part of the biological sample or blood product diluted in 10,000 parts of buffer results in 50% viral vector neutralization, to enlarge the pool of subjects that can effectively be treated with AAV gene therapy. Currently, prospective patients with viral neutralizing antibody levels 1:5 or higher are excluded from such treatment, i.e., they are not good candidates. Administration of the immune modulator to a subject having: an antibody titer greater than or equal to 1:5 but less than 1:10 is expected to decrease the antibody titer present in the subject to less that 1:5, thereby qualifying the subject as a candidate for administration of the recombinant viral vector (e.g., a gene therapy vector).

In one embodiments, the immune modulator is administered to a subject that was found to have a titer of viral vector binding antibodies present in the biological sample, e.g., a blood sample, from the subject that is greater than or equal to 1:5 and less than about 1:25 (e.g., 1:6, 1:7, 1:8, 1:9, 1:10, 1:11, 1:12, 1:13, 1:14, 1:15, 1:16, 1:17, 1:18, 1:19, 1:20, 1:21, 1:22, 1:23 and 1:24), where 1 part of the biological sample or blood product diluted in 10,000 parts of buffer results in 50% viral vector neutralization. For example, administration of the immune modulator to a subject having a titer greater than or equal to 1:5 but less than 1:15 is expected to decrease the antibody titer present in the subject to less that 1:5, thereby qualifying the subject as a candidate for administration of the recombinant viral vector (e.g., a gene therapy vector). In one embodiment, the immune modulator is administered to a subject having an antibody titer of viral vector binding antibodies present in the biological sample from the subject that is greater than or equal to 1:5 and less than about 1:100 (e.g., 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70, 1:75, 1:80, 1:85, 1:90, 1:95, and 1:99), where 1 part of the biological sample or blood product diluted in 10,000 parts of buffer results in 50% viral vector neutralization. Administration of the immune modulator to a subject having an antibody titer greater than or equal to 1:5 but less than 1:25 is expected to decrease the antibody titer present in the subject to less that 1:5, thereby qualifying the subject as a candidate for administration of the recombinant viral vector (e.g. a gene therapy vector).

In some embodiments, the immune modulator enables repeated dosages, or repeat administration of an AAV vector as disclosed herein. For example, administration of the viral vector, e.g., a AAV vector disclosed herein, with an immune modulator (at substantially the same time, or before or after the administration of the AAV vector) can be administered multiple times (i.e., greater than one time) over a defined time period. For exemplary purposes, the AAV vector can be administered several times, i.e., more than once, over a several weeks (e.g., 2-weeks) to several months (e.g., 2-months). Without wishing to be limited to theory, administration of the AAV vector with the immune modulator according to the methods as disclosed herein can be, as non-limiting examples, every month over a period of 6-months, 3-4 times over a period of 6-weeks, every week over a period of 1-month (or about 4 weeks) or 2-months (or about 8-weeks). In some embodiments, where a AAV vector as disclosed herein is administered with an immune modulator (at substantially the same time, or before, or after the administration of the AAV vector) multiple times (e.g., a repeat dose), the dose of the viral vector, e.g., AAV vector is lower than typically used in a single-dose regimen, for example, at a dose lower than a single-dose regimen as described herein. For example, the dose of the AAV vector can be less than or equal to about 10¹², or lower than about 10¹², for example, the dose can be about 10⁷, 10⁸, 10⁹, 10¹⁰, 10¹¹, or 10¹², or any dose between 10⁷ and 10¹². In some embodiments, where repeat doses of a AAV vector as disclosed herein are administered to a subject according to the methods as disclosed herein with an immune modulator, the immune modulator can be changed between the doses, i.e., the same or different immune modulators can be used in repeat doses. For example, when the first dose of AAV is co-administered with an immune modulator A, a second immune modulator administered with the second or third dose of AAV is different than immune modulator A, e.g., the second immune modulator is immune modulator B. For exemplary purposes, a dosing regimen according to the methods as disclosed can be administration of a AAV vector at a concentration of 10¹² or less than 10¹², where the immune modulator is administered as A-B-C-D, or A-A-B-C, or A-B-A-C, where A, B, C and D are different immune modulators as disclosed herein. Put another way, the dosing regimen can include a plurality of doses of immune modulator over the time period, wherein each dose of the plurality includes an immune modulator independently selected from immune modulator A, immune modulator B, immune modulator C, immune modulator D, and combinations thereof (i.e., each dose can include more than one immune modulator). Such varying immune modulators enables repeated doses of the AAV vector as disclosed herein. In some embodiments, for example, an immune modulator “A” can be such as IdeS, and an immune modulator “B” can be ImmTOR™, as disclosed herein. In some embodiments, an AAV vector as disclosed herein? is administered at a first timepoint with an IdeS immune modulator, and an AAV vector as disclosed herein is administered at a second timepoint with a different immune modulator, such as immunoglobulin degrading protein or a small molecule, e.g., ImmTOR™, or vice versa. For example, an AAV vector as disclosed here? can be administered at a first timepoint with an ImmTOR™ immune modulator, and an AAV vector as disclosed herein can be administered at a second timepoint with an IdeS.

One aspect herein provides a method for administering a recombinant viral vector (e.g., a gene therapy vector) to a subject who has previously received a recombinant viral vector, for example, the same recombinant viral vector or another viral vector having a similar serotype, the method comprising, prior to administering the recombinant viral vector, administering to the subject an immune modulator. In one embodiment, the previously received recombinant viral vector elicits an immune response resulting in anti-AAV antibodies that target (i.e., recognizes and binds) to the recombinant viral vector administered.

Another aspect herein provides a method for administering a recombinant viral vector (e.g., a gene therapy vector) to a subject who was previously exposed to a viral vector, wherein the exposure elicits an immune response resulting in anti-AAV antibodies that target the recombinant viral vector to be administered, and wherein the subject has anti-AAV antibody titer of at least 1:5-1:15, at least 1:5-1:25, at least 1:5-1:50, or at least 1:5-1:100, the method comprising the steps of, prior to administering the recombinant viral vector, administering to the subject an immune modulator.

In one embodiment, the immune modulator is administered systemically.

In some embodiments, the immune modulator crosses the blood brain barrier. In alternative embodiments, the immune modulator does not cross the blood brain barrier.

In one embodiment, the immune modulator is administered locally. For example, when the recombinant viral vector is to be administer locally to the brain tissue and the immune modulator does not cross the blood brain barrier, it is preferred to administer the immune modulator locally to the brain tissue, e.g., via an appropriate catheter, either directly to the brain tissue or indirectly to the brain tissue through cerebrospinal fluid circulating about the spinal cord (i.e., a spinal tap).

In one embodiment, the immune modulator is administered locally to central nervous system (CNS) tissue (e.g., brain tissue, spinal cord tissue, cerebrospinal fluid (CSF)). CNS tissue also includes, but is not limited to, neuronal cells, glial cells, astrocytes, cerebrospinal fluid (CSF), interstitial spaces, bone, cartilage and the like. Any composition described herein may be delivered directly to the CNS or brain by injection into, e.g., the ventricular region, as well as to the striatum (e.g., the caudate nucleus or putamen of the striatum), spinal cord and neuromuscular junction, or cerebellar lobule.

In one embodiment, the immune modulator is administered locally to any of the following: neural pathways, somatosensory systems, visual systems, auditory systems, nerves, neuro endocrine systems, neuro vascular systems, brain neurotransmitter systems, dural meningeal system, or combinations thereof.

In one embodiment, the immune modulator is administered locally to the eye, e.g., the vitreous, the retina, or the sclera.

In one embodiment, the immune modulator is administered systemically.

In some embodiments, the immune modulator is an immunoglobulin degrading enzyme such as IdeS, IdeZ, IdeS/Z, Endo S, or, their functional variant. Non-limiting examples of such immunoglobulin degrading enzymes and their uses are described in U.S. Pat. Nos. 7,666,582, 8,133,483, US 20180037962, US 20180023070, US 20170209550, U.S. Pat. No. 8,889,128, WO 2010057626, U.S. Pat. Nos. 9,707,279, 8,323,908, US 20190345533, US 20190262434, US 20210246469 and WO 2020016318, each of which are incorporated in their entirety herein by reference.

In some embodiments, an immune modulator disclosed herein can be administered to a subject at any suitable dose, such as a suitable dose determined by a medical professional. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., IdeZ, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., IdeS, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., EndoS, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

Further, reference to a numerical range, such as “0.01 to 10” includes 0.011, 0.012, 0.013, etc., as well as 9.5, 9.6, 9.7, 9.8, 9.9, 10, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

In various embodiments, administration of a recombinant viral vector to a subject is preceded by administration of a protease and/or glycosidase to inhibit, reduce, or prevent an immune response (e.g., a humoral immune response) against the recombinant viral vector or antibodies that bind to the heterologous polynucleotide or a protein or peptide encoded by the heterologous polynucleotide encapsidated by the viral vector. For example, administration of the viral vector can be preceded by administration of a protease and/or glycosidase by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours; or at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days; or by at least 1 month, 2 months, 3 months, 4 months, 5 months, 6 months, 7 months, 8 months, 9 months, 10 months, 11 months; or by at least 1 year, 2 years, 3 years, 4 years, 5 years, or more.

In one embodiments, administration of a recombinant viral vector to a subject is performed concurrently with administration of a protease and/or glycosidase to inhibit, reduce, or prevent an immune response (e.g., a humoral immune response) against the recombinant viral vector or antibodies that bind to the heterologous polynucleotide or a protein or peptide encoded by the heterologous polynucleotide encapsidated by the viral vector.

In certain embodiments, a protease and/or glycosidase is administered to a subject before an immune response (e.g., a humoral immune response), such as before development of neutralizing antibodies or development of antibodies that bind to the heterologous polynucleotide, protein, or peptide encoded by the heterologous polynucleotide encapsidated by the viral vector. In one embodiment, an immune response occurs within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours; or within 1 day, 2 days, 3 days, 4 days, 5 days, or more following administration of a recombinant viral vector.

In some embodiments, the immune modulator is a proteasome inhibitor. In some embodiments, the immune modulator is a protease or glycosidase. In certain aspects, the proteasome inhibitor is Bortezomib. In some aspects of the embodiment, the immune modulator comprises bortezomib and an anti-CD20 antibody, such as Rituximab. In other aspects of the embodiment, the immune modulator comprises bortezomib, Rituximab, methotrexate, and intravenous gamma globulin. Non-limiting examples of proteasome inhibitors and their combinations with Rituximab, methotrexate and intravenous gamma globulin are described in U.S. Pat. Nos. 10,028,993, 9,592,247, and 8,809,282, each of which is incorporated in its entirety herein by reference.

In alternative embodiments, the immune modulator is an inhibitor of the NF-kB pathway. In certain aspects of the embodiment, the immune modulator is Rapamycin or a functional variant thereof. Non-limiting examples of uses of rapamycin are described in U.S. Pat. No. 10,071,114, US 20160067228, US 20160074531, US 20160074532, US 20190076458, U.S. Pat. No. 10,046,064, which are each incorporated herein by reference in their entirety. In other aspects of the embodiment, the immune modulator is synthetic nanocarriers comprising an immunosuppressant. Non limiting examples of immunosuppressants, immunosuppressants coupled to synthetic nanocarriers, synthetic nanocarriers comprising rapamycin, and/or, tolerogenic synthetic nanocarriers, their doses, administration and use are described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which is incorporated in its entirety herein by reference.

In some embodiments, the immune modulator comprises synthetic nanocarriers comprising rapamycin (i.e., ImmTOR™ nanoparticles) as disclosed in Kishimoto, et al., 2016, Nat Nanotechnol, 11(10): 890-899; Maldonado, et al., 2015, PNAS, 112(2): E156-165) and in US20200038463 and U.S. Pat. No. 9,006,254, each of which is incorporated herein by reference in its entirety. In some embodiments, the immune modulator is an engineered cell, e.g., an immune cell that has been modified using SQZ technology as described in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, Aquila's QS21 stimulon, and combinations thereof. In another further embodiment, the immunomodulator or poly-ICLC.

In some embodiments, the immune modulator is a small molecule that inhibits the innate immune response in cells, such as chloroquine (a TLR signaling inhibitor) and/or 2-aminopurine (a PKR inhibitor), which can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, the immune modulator is photopheresis, also known as extracorporeal photochemotherapy, or ECP. Photopheresis treatment is performed on a subject's blood. Using either an IV or a catheter, blood is routed from the subject through a device which separates out a portion of white blood cells (leukocytes). The separated white blood cells are treated with naturally occurring photosensitizing chemicals called 8-methoxypsoralen (8-MOP) and then exposed to specific wavelengths of ultraviolet (UVA) light. Following exposure to the UVA light, the blood is administered back to the subject. Photopheresis can be performed at least once daily. In one embodiment, photopheresis is performed at least 1, 2, 3, 4, 5, 6, 7 times a week prior to administration of the recombinant viral vector. In one embodiment, photopheresis is performed at least 1, 2, 3, 4, 5, 6, 7 times a week for at least 1, 2, 3, 4, 5, 6, 7, 8, 9, 10, 11, 12 or more weeks, or for at least 1, 2, 3, 4, 5, 6, 7, 8, 9, 10, 11, or 12 months prior to administration of the recombinant viral vector. Therefore, the administering the immune modulator to the subject can include performing photopheresis on the subject. It is understood that the photopheresis can be performed in conjunction with administration of a second immune modulator selected from the enzymes, nanoparticles, and chemical compositions described herein and/or as a portion of a multiple dosing regimen.

In some embodiments, a rAAV vector having the modified viral capsid can also encode a negative regulator of innate immunity such as NLRX1. Accordingly, in some embodiments, a rAAV vector can also optionally encode one or more of NLRX1, NS1, NS3/4A, or A46R. Additionally, in some embodiments, a composition comprising at least one rAAV vector as disclosed herein can also comprise a synthetic, modified-RNA encoding inhibitor of the innate immune system to avoid the innate immune response generated by the tissue or the subject.

In some embodiments, an immune modulator for use in the administration methods as disclosed herein is an immunosuppressive drug or agent. As used herein, the term “immunosuppressive drug or agent” refers to pharmaceutical agents that inhibit or interfere with normal immune function. Examples of immunosuppressive drugs or agents suitable for the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211, which is incorporated herein by reference in its entirety. In one embodiment, an immunosuppressive agent is cyclosporine A. Other examples of immunosuppressive agents include myophenylate mofetil, rapamicin, and anti-thymocyte globulin. In various embodiments, the immunosuppressive drug is administered in a composition comprising at least one rAAV vector as disclosed herein, or in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a composition disclosed herein is also administered an immunosuppressive agent. Various methods are known for achieving immunosuppression of an immune response in a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference in their entireties, is bortezomib. In some embodiments, the immunosuppressive agent is an antibody, including polyclonal, monoclonal, scfv or other antibody-derived molecule that is capable of suppressing the immune response, for instance, through the elimination or suppression of antibody producing cells. In a further embodiment, the immunosuppressive element is a short hairpin RNA (shRNA). In this embodiment, the coding region of the shRNA is included in the rAAV cassette and is generally located downstream, i.e., 3′, of the poly-A tail. The shRNA can be targeted to reduce, reduce, or eliminate expression of immunostimulatory agents, such as cytokines, growth factors (including transforming growth factors 31 and 02, TNF and others that are known in the art).

The use of such immune modulating agents facilitates the ability to use multiple doses (e.g., multiple administration) over a plurality of months and/or years. This permits using multiple agents as discussed below, e.g., a rAAV vector encoding multiple genes, or multiple administrations to the subject.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology described herein, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

For convenience, certain terms employed herein, in the specification, examples and appended claims are collected here.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to decrease or stabilize a subject's MDS-UPDRS score, to extend the lifespan of a subject, to improve in the subject one or more symptoms of disease (e.g., a symptom of PD), etc. The effective amount will depend on a variety of factors such as, for example, the species, age, weight, health of the subject, and the tissue to be targeted, and may thus vary among subject and tissue as described elsewhere in the disclosure.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or non-human animal. Usually the non-human animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of PD. A subject can be male or female.

A subject can be one who has been previously diagnosed with or identified as suffering from or having a condition in need of treatment (e.g. PD) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition (e.g. PD) can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, miRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector described herein include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. PD. The term “treating” includes reducing or alleviating at least one adverse effect or symptom of a condition disease or disorder, e.g., as assessed by decreasing or stabilizing the subject's initial (i.e., prior to administration) MDS-UPDRS score. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” or “introducing” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

As used herein, “on” refers to a period of time in which the subject is administered an anti-Parkinson's therapeutic (e.g., levodopa) and wherein the subject has a beneficial response to the therapeutic.

As used herein, “off” refers to a period of time (e.g., up to 12 hours) in which the subject is not administered an anti-Parkinson's therapeutic (e.g., levodopa), or a period of time in which the subject is administered an anti-Parkinson's therapeutic (e.g., levodopa) but exhibits a low beneficial response to the therapeutic.

As used herein, “cDNA” or a “cDNA molecule” refers to “complementary DNA” that is synthesized by RNA-dependent DNA polymerase- or reverse transcriptase-catalyzed extension of a primer that anneals to an RNA molecule of interest using at least a portion of the RNA molecule of interest as a template (which process is also called “reverse transcription”). The cDNA molecules synthesized are “homologous to” or “base pair with” or “form a complex with” at least a portion of the template.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology described herein.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the technology disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this technology is not limited to the particular methodology, protocols, and reagents, etc., described herein and as such can vary. The terminology used herein is for the purpose of describing particular embodiments only, and is not intended to limit the scope of the present technology, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), WO 2018/057855A, U.S. Pat. No. 10,457,940, the contents of each of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the technology described herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

The technology described herein can further be described in the following numbered paragraphs:
1. A method of slowing or inhibiting progression of Parkinson's disease (PD) in a subject in need thereof, the method comprising:
   introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the nucleic acid and/or wherein at least 30% of the subject's putamen volume is covered by the rAAV, and
   wherein the subject does not exhibit an increase in PD-associated symptoms for a least 6 months following the introducing as compared to prior to introducing. As is known in the art, one can use the recombinant AAV comprising a nucleic acid sequence encoding GDNF operably linked to a promoter to prepare a medicament for carrying out the various methods disclosed here and in the following paragraphs.
2. The method of paragraph 1, wherein the rAAV is introduced via systemic introduction.
3. The method of any preceding paragraph, wherein the rAAV is introduced via local introduction.
4. The method of any preceding paragraph, wherein local introduction is introduction directly to the subject's putamen.
5. The method of any preceding paragraph, wherein the local introduction comprises directly introducing the rAAV to each of the subject's putamen.
6. The method of any preceding paragraph, wherein the local introduction is performed in simultaneously with non-invasive imaging.
7. The method of any preceding paragraph, wherein the non-invasive imaging is selected from the group consisting of intraoperative magnetic resonance image (iMRI)-guided convection enhanced delivery (CED), ultrasound, computed tomography (CT); functional magnetic resonance imaging (fMRI); positron emission tomography (PET); electroencephalography (EEG); magnetoencephalography (MEG); functional near-infrared spectroscopy (fNIRS); and combinations thereof.
8. The method of any preceding paragraph, wherein the local introduction comprises introducing about half of the rAAV vector to each putamen via intraoperative magnetic resonance image (iMRI)-guided convection enhanced delivery (CED).
9. The method of any preceding paragraph, wherein local introduction further comprises introducing an MRI contrast agent at substantially the same time as the AAV vector.
10. The method of any preceding paragraph, wherein the MRI contrast agent is gadoteridol.
11. The method of any preceding paragraph, wherein the MRI contrast agent is introduced to the subject in the same composition as the rAAV.
12. The method of any preceding paragraph, wherein the MRI contrast agent is introduced to the subject in a different composition as the rAAV.
13. The method of any preceding paragraph, wherein the rAAV is introduced via systemic introduction.
14. The method of any preceding paragraph, wherein the transduction and/or coverage of the putamen is assessed via Magnetic-resonance imaging.
15. The method of any preceding paragraph, wherein at least 40%, 50%, 60%, 70%, 80%, 90%, 95% or more of the volume of the subject's putamen is transduced with the GDNF gene.
16. The method of any preceding paragraph, wherein the subject does not exhibit a substantial increase in PD-associated symptoms for at least 12 months immediately following the introducing as compared to prior to the introducing.
17. The method of any preceding paragraph, wherein the subject exhibits a decrease in PD-associated symptoms for at least 6 months or more immediately following the introducing as compared to prior to introducing.
18. The method of any preceding paragraph, wherein the subject exhibits a decrease in PD-associated symptoms for a least 12 months or more immediately following the introducing as compared to prior to introducing.
19. The method of any preceding paragraph, wherein the subject has an initial Movement Disorder Society-Unified Parkinson Disease Rating Scale (MDS-UPDRS) score, prior to introduction, that is less than 32.
20. The method of any preceding paragraph, wherein the slowing or inhibiting the progression of Parkinson's disease in the subject is characterized by a second MDS-UPDRS score 6 months immediately following the introducing that is not substantially higher than the initial MDS-UPDRS score.
21. The method of any preceding paragraph, wherein the slowing or inhibiting the progression of Parkinson's disease in the subject is characterized by a second MDS-UPDRS score about 12 months immediately following the introducing that is not substantially higher than the initial MDS-UPDRS score.
22. The method of any preceding paragraph, wherein the subject has an initial MDS-UPDRS score, prior to introduction, that is greater than or equal to 32.
23. The method of any preceding paragraph, wherein the subject exhibits a decrease in the initial MDS-UPDRS score for at least 6 months immediately following the introducing as compared to prior to introducing.
24. The method of any preceding paragraph, wherein the slowing or inhibiting the progression of Parkinson's disease in the subject is characterized by a second MDS-UPDRS score about 6 months immediately following the introducing that is at least about 20% lower than the initial MDS-UPDRS score.
25. The method of any preceding paragraph, wherein the slowing or inhibiting the progression of Parkinson's disease in the subject is characterized by a second MDS-UPDRS score about 12 months immediately following the introducing that is at least about 30% lower than the initial MDS-UPDRS score
26. The method of any preceding paragraph, further comprising, prior to introducing, determining an initial MDS-UPDRS score for the subject.
27. The method of any preceding paragraph, further comprising, prior to introducing, receiving results of an assay that provides an initial MDS-UPDRS score for the subject.
28. The method of any preceding paragraph, wherein slowing or inhibiting the progression of PD in the subject is characterized by a reduction of an initial MDS-UPDRS score following introduction.
29. The method of any preceding paragraph, wherein the reduction is an at least 20%, 30%, 40%, 50%, 60%, 70%, 80%, 90%, 95% or greater reduction of the initial MDS-UPDRS score 6 months following introduction.
30. The method of any preceding paragraph, wherein the reduction is an at least 20%, 30%, 40%, 50%, 60%, 70%, 80%, 90%, 95% or greater reduction of the initial MDS-UPDRS score 12 months following introduction.
31. The method of any preceding paragraph, wherein slowing or inhibiting the progression of PD in the subject is characterized stabilization of an initial MDS-UPDRS score following introduction.
32. The method of any preceding paragraph, wherein the stabilization is characterized by no more than a 10% increase or decrease of the initial MDS-UPDRS score.
33. The method of any preceding paragraph, wherein stabilization occurs for at least 6 months or longer.
34. The method of any preceding paragraph, wherein the subject is mildly affected by PD.
35. The method of any preceding paragraph, wherein the subject mildly affected by PD has an initial MDS-UPDRS score less than 32 prior to the introduction of rAAV and was diagnosed with PD less than 5 years prior to the introduction.
36. The method of any preceding paragraph, further comprising, prior to the introduction, diagnosing the subject as being mildly affected by PD.
37. The method of any preceding paragraph, further comprising, prior to the introduction, receiving the results of an assay that diagnoses the subject as being mildly affected by PD.
38. The method of any preceding paragraph, wherein the subject is moderately affected by PD.
39. The method of any preceding paragraph, wherein the subject moderately affected by PD has an initial MDS-UPDRS score equal to or greater than 32 prior to the introduction of rAAV and was diagnosed with PD less than 4 years prior to the introduction.
40. The method of any preceding paragraph, further comprising, prior to the introduction, diagnosing the subject as being moderately affected by PD.
41. The method of any preceding paragraph, further comprising, prior to introduction, receiving the results of an assay that diagnoses the subject as being moderately affected by PD.
42. The method of any preceding paragraph, wherein the promoter is a cytomegalovirus (CMV) promoter.
43. The method of any preceding paragraph, wherein the promoter is a nervous system (NS) or central nervous system (CNS) specific promoter.
44. The method of any preceding paragraph, wherein the NS specific promoter is selected from the NS specific promoters in Table 1.
45. The method of any preceding paragraph, wherein the CNS specific promoter is selected from the CNS specific promoters in Table 2.
46. The method of any preceding paragraph, wherein the nucleic acid comprises a sequence of SEQ ID NO: 1, or a functional variant that is at least 85, 86, 87, 88, 89, 90, 91, 92, 93, 94, 95, 96, 97, 98, 99% or more identical to SEQ ID NO: 1.
47. The method of any preceding paragraph, wherein the rAAV is AAV1, AAV2, AAV3, AAV3b, AAV4, AAV5, AAV6, AAV7, AAV8, AAV9, AAV10, AAV11, AAV12, Rh10, or a rational haploid thereof.
48. The method of any preceding paragraph, wherein the rAAV is AAV2.
49. The method of any preceding paragraph, wherein the rAAV exhibits brain-specific tropism.
50. The method of any preceding paragraph, wherein the rAAV comprises a modification that increases its brain-specific tropism.
51. The method of any preceding paragraph, wherein brain-specific tropism is increased by at least 20%, 30%, 40%, 50%, 60%, 70%, 80%, 90%, 95% or greater as compared to an unmodified AAV.
52. The method of any preceding paragraph, wherein the rAAV is introduced at a total dose within the range of 5×10¹² vg to about 1.5×10¹³ vg.
53. The method of any preceding paragraph, wherein about one half of the total dose is administered to each of the subject's putamen.
54. The method of any preceding paragraph, wherein introducing is performed at a flow rate of from about 1 μL/min to about 30 μL/min.
55. The method of any preceding paragraph, wherein the rAAV is introduced as a liquid composition comprising the rAAV and a pharmaceutically acceptable carrier.
56. The method of any preceding paragraph, wherein the liquid composition has an rAAV concentration of from about 3×10¹² vg/mL to about 4×10¹² vg/mL.
57. The method of any preceding paragraph, wherein the subject is administered at least one anti-PD therapeutic prior to the introduction of the rAAV.
58. The method of any preceding paragraph, wherein the subject is administered at least one anti-PD therapeutic prior to and following the introduction of the rAAV.
59. The method of any preceding paragraph, wherein the at least one anti-PD therapeutic is selected from the group consisting of levodopa, Sinemet, Rytary, Stalevo, amantadine, pramipexole, rotigotine, ropinirole, apomorphine, entacapone.
60. The method of any preceding paragraph, wherein the subject maintains or decreases the dose of the at least one anti-PD therapeutic following introduction.
61. The method of any preceding paragraph, wherein the dose of the at least one anti-PD therapeutic is decreased by at least 5%, 10%, 15%, 20%, 25%, 30%, 35%, 40%, 45%, 50% or more.
62. A method of slowing or inhibiting a progression of Parkinson's disease (PD) in a subject in need thereof, the method comprising:
   locally introducing to the subject's putamen a recombinant adeno-associated virus (rAAV) vector comprising a nucleic acid encoding glial cell line-derived neurotrophic factor (GDNF) operably linked to a promoter,
   wherein at least 30% of the volume of the subject's putamen is transduced with the GDNF gene.
63. A method of slowing or inhibiting a progression of PD in a subject in need thereof, the method comprising:
   transducing greater than or equal to about 30% of the volume of the subject's putamen with a glial cell line-derived neurotrophic factor (GDNF) gene,
   wherein the subject does not exhibit a substantial increase in PD-associated symptoms for a least 6 months following the transducing.
64. The method of any preceding paragraph, wherein the transducing is performed by administering a rAAV comprising the GDNF gene to each of the subject's putamen.
65. A method of slowing or inhibiting a progression of PD in a subject in need thereof, the method comprising:
   covering greater than or equal to about 30% of the volume of the subject's putamen with a glial cell line-derived neurotrophic factor (GDNF) gene,
   Wherein the subject does not exhibit a substantial increase in PD-associated symptoms for at least 6 months following the covering.
66. The method of any preceding paragraph, wherein the transducing is performed by administering a rAAV comprising the GDNF gene to each of the subject's putamen.
67. A method of reducing or stabilizing an initial Movement Disorder Society-Unified Parkinson's Disease Rating Scale Part (MDS-UPDRS) score in a subject having Parkinson's disease (PD), the method comprising:
   administering to the subject's putamen a recombinant adeno-associated virus (rAAV) comprising a nucleic acid encoding glial cell line-derived neurotrophic factor (GDNF) operably linked to a promoter,
   wherein the subject has a second MDS-UPDRS score at 6 months following the administration is decreased or stabilized as compared to the initial MDS-UPDRS score of the subject prior to administering.
68. The method of any preceding paragraph, further comprising the step of, prior to administering, obtaining or receiving an initial MDS-UPDRS score from the subject.
69. The method of any preceding paragraph, wherein the second MDS-UPDRS score is decreased by at least 20%, 30%, 40%, 50%, 60%, 70%, 80%, 90%, 95% or greater as compared to the initial MDS-UPDRS score 12 months following administering.
70. The method of any preceding paragraph, wherein stabilization is no more than a 10% increase or decrease of the initial MDS-UPDRS score.
71. A method of treating a subject mildly affected by Parkinson's disease (PD), the method comprising:
   administering to each of the subject's putamen a recombinant adeno-associated virus (rAAV) comprising a nucleic acid encoding glial cell line-derived neurotrophic factor (GDNF) operably linked to a promoter,
   wherein at least 30% of the subject's putamen is transduced with GDNF, and
   wherein the subject has a second MDS-UPDRS score at 6 months post-administering that is stabilized as compared to the initial MDS-UPDRS score.
72. The method of any preceding paragraph, wherein the subject has a MDS-UPDRS score at 12 month post-administering that is stabilized as compared to the initial MDS-UPDRS score prior to administering.
73. The method of any preceding paragraph, wherein stabilization is no more than a 10% increase or decrease of the initial MDS-UPDRS score.
74. A method of treating a subject a subject mildly affected by Parkinson's disease (PD), the method comprising:
   administering to each of the subject's putamen a recombinant adeno-associated virus (rAAV) comprising a nucleic acid encoding glial cell line-derived neurotrophic factor (GDNF) operably linked to a promoter,
   wherein at least 30% of the subject's putamen is covered by the rAAV, and
   wherein the subject has a second MDS-UPDRS score at 6 months post-administering that is stabilized as compared to the initial MDS-UPDRS score
75. The method of any preceding paragraph, wherein the subject has a MDS-UPDRS score at 12 month post-administering that is stabilized as compared to the initial MDS-UPDRS score prior to administering.
76. The method of any preceding paragraph, wherein stabilization is no more than a 10% increase or decrease of the initial MDS-UPDRS score
77. A method of treating a subject moderately affected by Parkinson's disease (PD), the method comprising:
   administering to each of the subject's putamen a recombinant adeno-associated virus (AAV) comprising a nucleic acid encoding glial cell line-derived neurotrophic factor (GDNF) operably linked to a promoter,
   wherein at least 30% of the subject's putamen is transduced with the nucleic acid, and
   wherein the subject has a second MDS-UPDRS score at 6 months post-administering that is at least about 20% lower than the initial MDS-UPDRS score.
78. The method of any preceding paragraph, wherein the reduction is an at least 30%, 40%, 50%, 60%, 70%, 80%, 90%, 95% or greater as compared to the initial MDS-UPDRS score.
79. A method of treating a subject moderately affected by Parkinson's disease (PD), the method comprising:
   administering to each of the subject's putamen a recombinant adeno-associated virus (AAV) comprising a nucleic acid encoding glial cell line-derived neurotrophic factor (GDNF) operably linked to a promoter,
   wherein at least 30% of the subject's putamen is covered with the rAAV, and
   wherein the subject has a second MDS-UPDRS score at 6 months post-administering that is at least about 20% lower than the initial MDS-UPDRS score
80. The method of any preceding paragraph, wherein the reduction is an at least 30%, 40%, 50%, 60%, 70%, 80%, 90%, 95% or greater as compared to the initial MDS-UPDRS score.
81. A method of slowing or inhibiting progression of Parkinson's disease (PD) in a subject in need thereof, the method comprising:
   locally introducing to each of the subject's putamen a recombinant adeno-associated virus (rAAV) vector comprising a nucleic acid encoding glial cell line-derived neurotrophic factor (GDNF) operably linked to a promoter; and
   locally introducing an MRI contrast agent to each of the subject's putamen at substantially the same time as the rAAV,
   wherein at least 30% of the volume of the subject's putamen is transduced with the nucleic acid, and
   wherein the subject does not exhibit a substantial increase in PD-associated symptoms for a least 6 months immediately following the introducing as compared to prior to introducing.
82. A method of slowing or inhibiting progression of Parkinson's disease (PD) in a subject in need thereof, the method comprising:
   locally introducing to each of the subject's putamen a recombinant adeno-associated virus (rAAV) vector comprising a nucleic acid encoding glial cell line-derived neurotrophic factor (GDNF) operably linked to a promoter; and
   locally introducing an MRI contrast agent to each of the subject's putamen at substantially the same time as the rAAV,
   wherein at least 30% of the volume of the subject's putamen is covered with the rAAV, and
   wherein the subject does not exhibit a substantial increase in PD-associated symptoms for a least 6 months immediately following the introducing as compared to prior to introducing.
83. A method of slowing or inhibiting progression of Parkinson's disease (PD) in a subject in need thereof, the method comprising:
   introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is transduced with the nucleic acid, and
   wherein the subject does not exhibit a substantial increase in PD-associated symptoms for a least 6 months immediately following the introducing as compared to prior to introducing.
84. A method of slowing or inhibiting progression of Parkinson's disease (PD) in a subject in need thereof, the method comprising:
   introducing to the subject a recombinant adeno-associated virus (rAAV) comprising a nucleic acid encoding glial cell line-derived neurotrophic factor (GDNF) operably linked to a promoter, wherein at least 30% of the volume of the subject's putamen is covered with the rAAV, and
   wherein the subject does not exhibit a substantial increase in PD-associated symptoms for a least 6 months immediately following the introducing as compared to prior to introducing
85. A composition for slowing or inhibiting a progression of Parkinson's disease (PD) in a subject, the composition comprising:
   a recombinant adeno-associated virus (rAAV) comprising a genome comprising a glial cell line-derived neurotrophic factor (GDNF) gene operably linked to a promoter; and
   a pharmaceutically acceptable carrier.
86. The composition of any preceding paragraph, wherein the composition has a rAAV concentration of 3×10¹² vg to 4×10¹² vg per mL.
87. The composition of any preceding paragraph, wherein the composition comprises an rAAV concentration of 3.3×10¹² vg per mL.
88. A formulation for slowing or inhibiting a progression of Parkinson's disease (PD) in a subject, the formulation comprising: an adeno-associated virus (AAV) at a concentration of 3×10¹² vg to 4×10¹² vg per mL of a pharmaceutically acceptable carrier,
   wherein the rAAV comprises a genome comprising a glial cell line-derived neurotrophic factor (GDNF) gene operably linked to a promoter.
89. The method of any preceding paragraph, wherein the subject does not exhibit any serious adverse event for a least 6 months immediately following the introducing or administering.
90. Use of a recombinant adeno-associated virus (rAAV) comprising a nucleic acid encoding glial cell line-derived neurotrophic factor (GDNF) operably linked to a promoter in the preparation of a medicament for a method of slowing or inhibiting progression of Parkinson's Disease (PD), or to stabilize progression of PD in a subject in need thereof, or treating a subject mildly or moderately affected by PD, comprising the methods of paragraphs 1-84 and 89.

### EXAMPLES

### Example 1—Production of Viral Vectors Comprising Nucleic Acid Encoding GDNF Polypeptide Operatively Linked to a CMV Promoter

Derivation of suspension HEK293 cells from an adherent HEK293 Qualified Master Cell Bank. The derivation of the suspension cell line from the parental HEK293 Master Cell Bank (MCB), is performed in a Class 10,000 clean room facility. The derivation of the suspension cell line is carried out in a two phase process that involved first weaning the cells off of media containing bovine serum and then adapting the cells to serum free suspension media compatible with HEK293 cells. The suspension cell line is created as follows. First, a vial of qualified Master Cell Bank (MCB) is thawed and placed into culture in DMEM media containing 10% fetal bovine serum (FBS) and cultured for several days to allow the cells to recover from the freeze/thaw cycle. The MCB cells are cultured and passaged over a 4 week period while the amount of FBS in the tissue culture media is gradually reduced from 10% to 2.5%. The cells are then transferred from DMEM 2.5% FBS into serum free suspension media and grown in shaker flasks. The cells are then cultured in the serum-free media for another 3 weeks while their growth rate and viability is monitored. The adapted cells are then expanded and frozen down. A number of vials from this cell bank are subsequently thawed and used during process development studies to create a scalable manufacturing process using shaker flasks and wave bioreactor systems to generate rAAV vectors. Suspension HEK293 cells are grown in serum-free suspension media that supports both growth and high transfection efficiency in shaker flasks and wave bioreactor bags. Multitron Shaker Incubators (ATR) are used for maintenance of the cells and generation of rAAV vectors at specific rpm shaking speeds (based on cell culture volumes), 80% humidity, and 5% CO₂.

Transfection of suspension HEK293 cells. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The plasmid DNA has a sequence comprising a heterologous nucleic acid sequence of a GDNF gene (i.e., the nucleic acid sequence encoding GDNF (SEQ ID NO: 1)) operatively linked to CMV promoter. The cocktail further comprises a Packaging plasmid encoding Rep2 and serotype-specific Cap2: AAV-Rep/Cap, and the Ad-Helper plasmid (XX680: encoding adenoviral helper sequences). The cocktail is inverted to mix prior to being incubated at room temperature. The transfection cocktail is then pipetted into the flasks and placed back in the shaker/incubator. All optimization studies are carried out at 30 mL culture volumes followed by validation at larger culture volumes. Cells are harvested 48 hours post-transfection.

Production of rAAV using wave bioreactor systems. Wave bags are seeded 2 days prior to transfection. Two days post-seeding the wave bag, cell culture counts are taken and the cell culture is then expanded/diluted before transfection. The wave bioreactor cell culture is then transfected. Cell culture is harvested from the wave bioreactor bag at least 48 hours post-induction.

Analyzing transfection efficiency using Flow Cytometry. Approximately 24 hours post-induction, 1 mL of cell culture is removed from each flask or wave bioreactor bag as well as an uninduced control. Samples are analyzed using a Dako Cyan flow cytometer to confirm that the plasmid DNA.

Harvesting suspension cells from shaker flasks and wave bioreactor bags. 48 hours post-induction, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet could either be stored in NLT-60° C. or continued through purification.

Titering rAAV from cell lysate using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl₂, 5 mM MgCl₂, 50 mM Tris-HCl pH 8.0) followed by sonication to lyse the cells efficiently. 300 ul is then removed and placed into a 1.5 mL microfuge tube. 140 units of DNase I is then added to each sample and incubated at 37° C. for 1 hour. To determine the effectiveness of the DNase digestion, 4-5 ug of plasmid DNA is spiked into a non-transfected cell lysate with and without the addition of DNase. 50 ul of EDTA/Sarkosyl solution (6.3% sarkosyl, 62.5 mM EDTA pH 8.0) is then added to each tube and incubated at 70° C. for 20 minutes. 50 ul of Proteinase K (10 mg/mL) is then added and incubated at 55° C. for at least 2 hours. Samples are then boiled for 15 minutes to inactivate the Proteinase K. An aliquot is removed from each sample to be analyzed by qPCR. Two qPCR reactions are carried out in order to effectively determine how much rAAV vector is generated per cell.

Purification of rAAV from crude lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art could use mechanical methods such as microfluidization or chemical methods such as detergents, etc., followed by a clarification step using depth filtration or Tangential Flow Filtration (TFF).

AAV vector purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc).

Titering rAAV using dot blot. 100 ul of DNase buffer (140 units DNase, 5 mM CaCl₂, 5 mM MgCl₂, 50 mM Tris-HCl pH 8.0) is added to each well of a 96-well microtiter plate. 1-3 ul or serial dilutions of virus is added to each well and incubated at 37° C. for 30 min. The samples are then supplemented with 15 ul Sarkosyl/EDTA solution (6.3% sarkosyl, 62.5 mM EDTA pH 8.0) and placed at 70° C. for 20 min. Next, 15 ul of Proteinase K (10 mg/mL) is added and incubated at 50° C. for at least 2 hours. 125 ul of NaOH buffer (80 mM NaOH, 4 mM EDTA pH 8.0) is added to each well. A series of transgene specific standards are created through a dilution series. NaOH buffer is then added and incubated. Nylon membrane is incubated at RT in 0.4 M Tris-HCl, pH 7.5 and then set up on dot blot apparatus. After a 10-15 minute incubation in NaOH buffer, the samples and standards are loaded into the dot blot apparatus onto the GeneScreen PlusR hybridization transfer membrane (PerkinElmer). The sample is then applied to the membrane using a vacuum. The nylon membrane is soaked in 0.4 M Tris-HCl, pH 7.5 and then cross linked using UV strata linker 1800 (Stratagene) at 600 ujouls×100. The membrane is then pre-hybridized in CHURCH buffer (1% BSA, 7% SDS, 1 mM EDTA, 0.5 M Na₃PO₄, pH 7.5). After pre-hybridization, the membrane is hybridized overnight with a ³²P-CTP labeled transgene probe (Roche Random Prime DNA labeling kit). The following day, the membrane is washed with low stringency SSC buffer (1×SSC, 0.1% SDS) and high stringency (0.1×SSC, 0.1% SDS). It is then exposed on a phosphorimager screen and analyzed for densitometry using a STORM840 scanner (GE).

Analyzing rAAV vector purity using silver stain method. Samples from purified vector are loaded onto NuPage 10% Bis-Tris gels (Invitrogen) and run using 1× NuPage running buffer. Typically, 1×10¹⁰ particles are loaded per well. The gels are treated with SilverXpress Silver staining kit #LC6100 (Invitrogen).

Analysis of self-complementary genomes using alkaline gel electrophoresis and southern blot. Briefly, purified self-complementary rAAV is added to 200 ul, of DNase I buffer (140 units DNase, 5 mM CaCl₂, 5 mM MgCl₂, 50 mM Tris-HCl pH 8.0) and incubated at 37° C. for 60 minutes, followed by inactivation of the DNase by adding 30 ul, of EDTA Sarkosyl/EDTA solution (6.3% sarkosyl, 62.5 mM EDTA pH 8.0) and placed at 70° C. for 20 min. 20 ul of Proteinase K (10 mg/mL) is then added to the sample and incubated for a minimum of 2 hours at 50° C. Phenol/Chloroform is added in a 1:1 ratio, followed by ethanol precipitation of the viral vector DNA. The pelleted DNA is then resuspended in alkaline buffer (50 mM NaOH, 1 mM EDTA) for denaturation, loaded onto a 1% alkaline agarose gel, and run at 25V overnight. The gel is then equilibrated in alkaline transfer buffer (0.4 M NaOH, 1 M NaCl) and a southern blot is performed via an overnight transfer of the vector DNA to a GeneScreen PlusR hybridization transfer membrane (PerkinElmer). The membrane is then neutralized using 0.5 M Tris pH 7.5 with 1 M NaCl, and is hybridized overnight with a ³²P-CTP labeled transgene probe. After washing the membrane as previously described, the membrane is exposed to a phosphorimager screen and analyzed using a STORM840 scanner.

Transduction Assays. HeLaRC-32 cells (Chadeuf et al., J Gene Med. 2:260 (2000)) are plated at 2×10⁵ cells/well of a 24 well plate and incubated at 37° C. overnight. The cells are observed for 90-100% confluence. 50 mL of DMEM with 2% FBS, 1% Pen/Strep is pre-warmed, and adenovirus (d1309) is added at a MOI of 10. The d1309 containing media is aliquoted in 900 ul fractions and used to dilute the rAAV in a series of ten-fold dilutions. The rAAV is then plated at 400 μl and allowed to incubate for 48 hours at 37° C.

Concentration Assays. The starting vector stock is sampled and loaded onto a vivaspin column and centrifuged at 470×g (Sorvall H1000B) in 10 minute intervals. Once the desired volume/concentration had been achieved, both sides of the membrane are rinsed with the retentate, which is then harvested. Samples of the pre-concentrated and concentrated rAAV are taken to determine physical titers and transducing units.

Transmission electron microscopy (TEM) of negatively stained rAAV particles. Electron microscopy allows a direct visualization of the viral particles. Purified dialyzed rAAV vectors are placed on a 400-mesh glow-discharged carbon grid by inversion of the grid on a 20 ul drop of virus. The grid is then washed 2 times by inversion on a 20 ul drop of ddH₂O followed by inversion of the grid onto a 20 ul drop of 2% uranyl acetate for 30 seconds. The grids are blotted dry by gently touching Whatman paper to the edges of the grids. Each vector is visualized using a Zeiss EM 910 electron microscope.

### Example 2—Local Administration of AAV2-GDNF for Treatment of Parkinson's Disease

GDNF is known to support the survival and promote differentiation of dopaminergic neurons and has long been evaluated as a putative therapeutic agent for Parkinson's disease. Nonclinical studies have demonstrated that local delivery of GDNF into the brain can both protect dopaminergic neurons against neurotoxic insults and stimulate anatomical and functional recovery in rodent and non-human primate models of PD (Kordower 2013).

Direct administration of recombinant GDNF protein has been investigated in multiple clinical studies since the 1990's (Kordower 1999, Nutt 2003, Gill 2003, Slevin 2005, Lang 2006, Whone 2019). Given the inability of GDNF to cross the blood brain barrier, these clinical investigations have utilized implanted devices intended to deliver continuous or intermittent doses of GDNF directly into the brain. To date, the direct delivery of GDNF protein has failed to demonstrate a significant clinical benefit compared to placebo-treated controls, despite intraputaminal delivery of GDNF protein being well-tolerated by PD patients. However, neuroimaging assessing putaminal dopamine function, via F-DOPA PET in the most recent study, revealed a significant restoration of activity in patients that received intermittent GDNF administration (Whone 2019). Those investigators proposed a potential lag between restoration of PET-determined brain dopamine function and clinical amelioration of PD clinical features, and that a longer duration of dosing and observation is warranted, beyond the 9-month assessments of their study.

The adoption of CED methodology for the injection of investigational agents into the brain has enabled targeting of large brain regions, not previously feasible by methods depending on fluid diffusion (Christine 2019). CED involves precise placement of a reflux-resistant delivery cannula into the target tissue, and CED displaces the interstitial fluid around the tip of cannula, enabling the safe, homogenous distribution of large molecules within the infusate, such as AAV vectors, over relatively large parenchymal volumes during a single administration procedure (Richardson 2011). Through the use of iMRI monitoring of the CED in real-time, larger putaminal CED infusion volumes than those previously delivered (50-450 μL), were delivered to more efficiently cover the putaminal volume.

The infusion volumes for the current investigation (up to 1.8 mL per putamen) have recently been safely and effectively used in another clinical PD study (ClinicalTrials.gov Identifier: NCT03065192).

Non-clinical studies have demonstrated that MRI visualization of gadoteridol co-administered with AAV2 vectors provides an accurate anatomical representation of the parenchymal volume histologically showing neuronal gene transfer, and that the delivered contrast agent provides no intrinsic local toxicity (Su 2010, Richardson 2011). Intraoperative MRI monitoring, therefore, provides real-time feedback to the surgical team as to the drug distribution, and provides an opportunity to tailor the CED based on the individual patient's anatomy, maximizing delivery to the target while limiting exposure of non-targeted regions from visualized reflux or leakage of infusate.

Recent nonclinical studies have documented the capacity for more effective distribution within the putamen using a single trajectory, occipital approach (Bankiewicz 2016), as opposed to the multiple trajectories required via the more traditional frontal approach used in previous studies. The occipital/parietal approach utilizes a single infusion trajectory along the long axis of the putamen and has been used effectively in previous PD gene therapy trial (ClinicalTrials.gov Identifier: NCT03065192).

### GDNF Gene Delivery for Parkinson's Disease

This current study described herein investigates GDNF gene delivery as a disease-modifying treatment for PD. AAV2-GDNF, the study drug, is an AAV serotype 2 vector containing a DNA expression cassette encoding the complementary sequence for human GDNF under the control of the cytomegalovirus immediate early promoter (CMV).

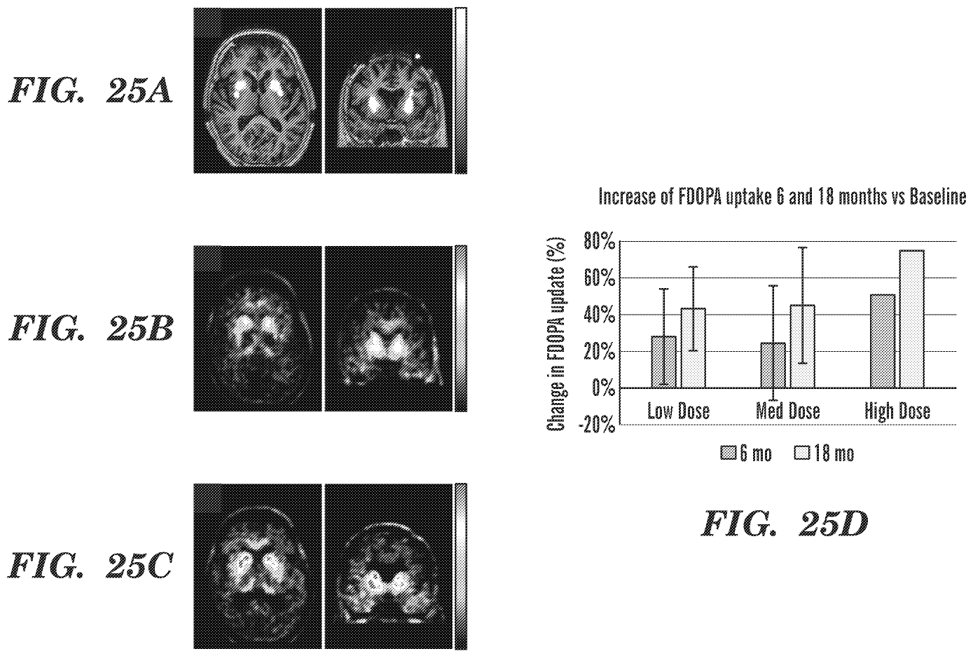

The ongoing observational follow-up of the dose-escalation safety study continues at the NIH Clinical Center (ClinicalTrials.gov Identifier: NCT01621581). A total of 13 participants with PD received the same study drug between 2013 and 2017. Three concentrations of study drug were evaluated (1.0×10¹¹, 3.3×10¹¹, and 1.0×10¹² vg/mL) with infusion volumes of 0.45 mL per putamen. The average putaminal coverage achieved in the previous study was 26% of the total putaminal volume. This lower than expected coverage noted on interim analysis, encouraged the elective stoppage of this previous study, after 13 treated subjects, in an effort to provide improved delivery options. An approved and planned higher dose of 3.3×10¹² vg/mL had not been tested in humans by the time the previous study was closed to recruitment. The 13 P study participants provided evidence that transfrontally-delivered, bilateral infusions of the study drug into the putamen is indeed safe and well-tolerated at the three doses administered (6 subjects each at the 9×10¹⁰ vg/mL and 3.3×10¹¹ vg/mL; 1 subject received the 9×10¹¹ vg/mL dose), with no SAE's related to the study drug. F-DOPA PET analysis of the putaminal drug delivery sites showed increased DA activity, indicative of a probable GDNF effect. An increase in F-DOPA uptake was observed at 6 and 18 months post administration as compared to baseline (FIG. 25A-25D).

The current study described herein features infusion of the study drug in study participants via intraoperative MRI-guided CED. A maximum of 1.8 mL of study drug was infused into each putamen with the anticipated distribution to 50-80% of the putaminal volume. The one-time delivery of the study drug is intended to result in the continuous expression of GDNF protein within >50% of the putaminal volume, primarily transducing intrinsic medium spiny neurons (MSNs). Additional anterograde transport of the study drug from the MSNs to the SN (via direct and indirect pathways) will provide GDNF expression and trophic support directly to dopaminergic neuronal somata within the SN.

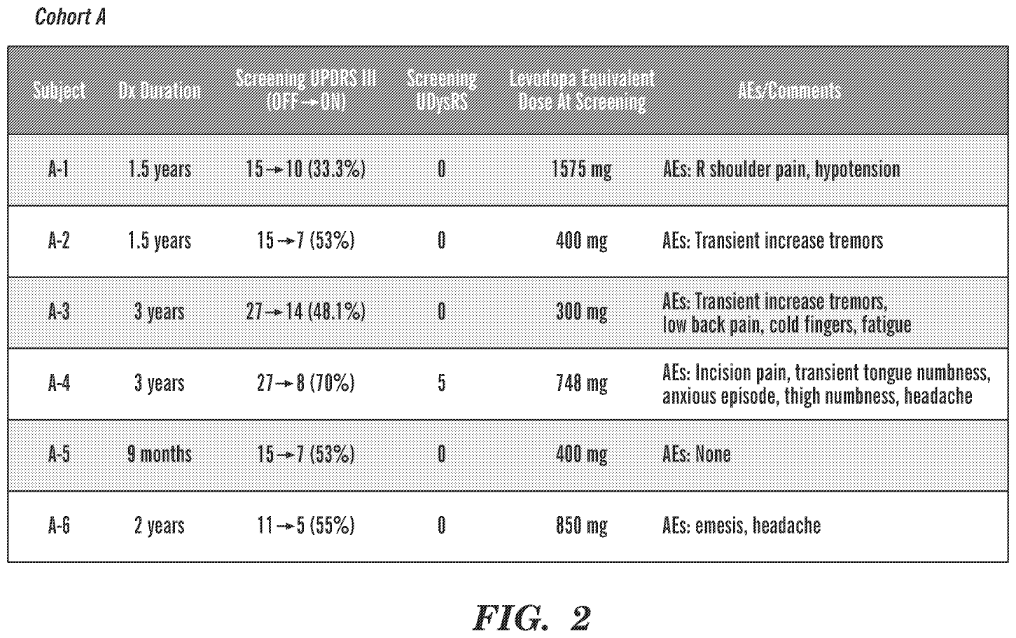

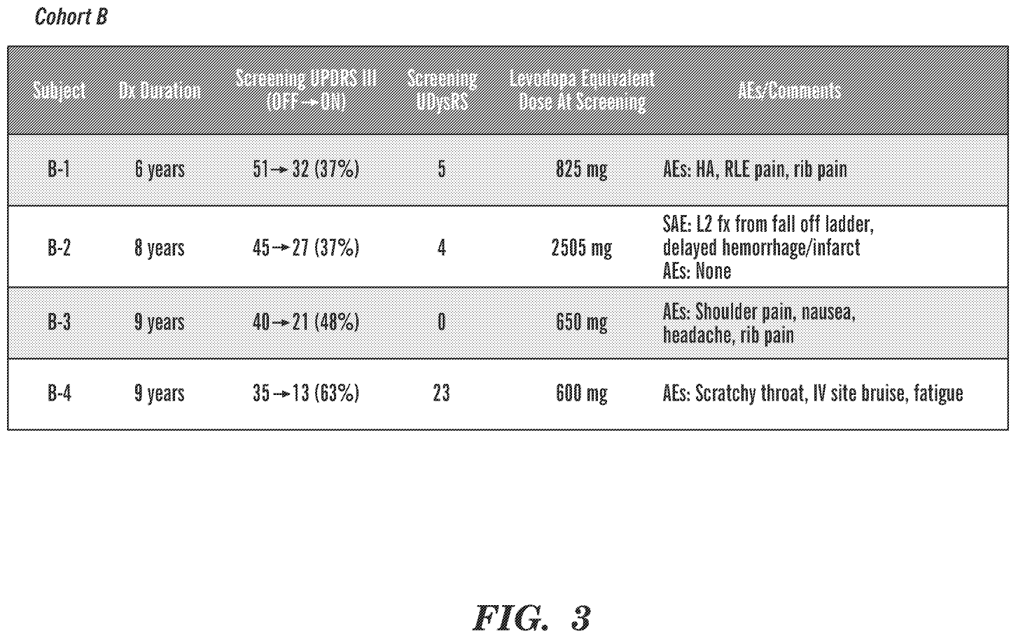

A total of 12 study participants have been administered the investigational product (i.e., AAV2-GDNF) in this study. Participants are enrolled into one of two parallel cohorts, based upon the duration and stage of their PD. Cohort A include subject that are mildly affected by PD (FIG. 2) and Cohort B include subject that are moderately affected by PD (FIG. 3).

### Study Inclusion Criteria

1. Male and female adults 35-75 years of age (inclusive) and able to provide Informed Consent
2. Diagnosed with Idiopathic PD
   a. At least 3 of the following clinical features: rest tremor, rigidity, bradykinesia, postural reflex impairment
   b. At least one of the above must be rest tremor or bradykinesia
The above clinical features must not be due to trauma, brain tumor, infection, cerebrovascular disease, other known neurological disease (e.g. multiple system atrophy, progressive supranuclear palsy, striatonigral degeneration, Huntington's disease, corticobasal syndrome, diffuse Lewy body disease, Wilson's disease), or secondary to drugs, chemicals or toxicants linked to PD diagnosis
3. Modified Hoehn and Yahr Stage I-III OFF medication
4. Time since receiving a clinical diagnosis of PD and disease severity consistent with one of the following:
EITHER: Less than 5 years from clinical diagnosis of PD and MDS-UPDRS III “OFF state” motor score ≤32 (Cohort A)
OR: At least 4 years since clinical diagnosis of PD and MDS-UPDRS III “OFF state” motor score of 33-60 (Cohort B)
5. Unequivocal responsiveness to levodopa, based on a 30% or greater improvement in the MDS-UPDRS III motor score after a levodopa challenge
6. Laboratory values at Screening visit
   a. Platelets >100,000/mm³ (transfusion independent)
   b. PT/PTT in normal range and INR<1.3 (and prior to surgery)
   c. Absolute neutrophil count (ANC)>1500/mm³
   d. Hemoglobin >10.0 g/dL (transfusion allowed)
   e. Aspartate aminotransferase or alanine aminotransferase <2.5×ULN
   f Total bilirubin <2.5 mg/dL
   g. Serum creatinine <1.5 mg/dL
   h. HbA1C<10%
7. Willingness to defer neurological surgery, including deep brain stimulation, until after completing the 18-month study visit (unless recommended by the Investigator)
8. Stable PD medication regimen for >5 weeks prior to screening

### Exclusion Criteria

1. Presence of prominent oculomotor palsy, cerebellar signs, vocal cord paresis, orthostatic hypotension (>20 mm Hg in systolic or of >10 mm Hg in diastolic pressure drop on standing), pyramidal tract signs or amyotrophy
2. PD related genetic factors (e.g. PRKN, PINK1, or LRKK2 mutations)
3. Severe dyskinesia (UDysRS>30)
4. Presence of dementia (Montreal Cognitive Assessment (MoCA)<25)
5. Presence or history of psychosis, including if induced by anti-PD medications at doses required to improve motor symptoms
6. Presence of untreated or suboptimally treated depression (BDI-II score >20) or a history of a serious mood disorder (i.e., requiring psychiatric hospitalization or a prior suicide attempt)
7. Presence of substance (drug, alcohol) abuse as defined by DSM-5 criteria and in the judgement of the Investigator. Note: Use of tetrahydrocannabinol or cannabidiol would not be exclusionary
8. Coagulopathy, anticoagulant therapy, low platelet count, or inability to temporarily stop any antithrombotic medication
9. History of stroke or poorly controlled cardiovascular disease
10. Uncontrolled hypertension or diabetes or any other acute or chronic medical condition that would significantly increase the risks of a neurosurgical procedure
11. History of malignancy (cerebral or systemic) other than treated cutaneous squamous or basal cell carcinomas
12. Clinically active infection, including acute or chronic scalp infection
13. Prior brain surgery or other brain imaging abnormalities
14. Contraindication to MRI and/or use of gadolinium
15. Pregnancy or lactation
16. Male or female with reproductive capacity that is unwilling to use barrier contraception for 3 months post-dosing
17. Chronic immunosuppressive therapy (e.g., chronic steroids, tumor necrosis factor, antagonists, chemotherapy)
18. Unwilling to defer any vaccination within 1 month before or after treatment
19. Received any investigational agent within 12 weeks prior to screening
20. Unable to comply with the protocol procedures, including frequent and prolonged follow-up assessments
21. History of cancer or poorly controlled medical condition that would increase surgical risk
22. Any significant issue raised by the neurologist, psychologist/psychiatrist or neurosurgeon, including concerning impulsive or compulsive behaviors

### Investigational Study Drug

The investigational product, AAV2-GDNF, comprises an adeno-associated virus, serotype 2 (AAV2) containing human GDNF complementary DNA (cDNA), human cytomegalovirus (CMV) promoter and 3′ UTR sequences. AAV2-GDNF is supplied in 1 mL aliquots at a concentration of 7.9×10¹² vg/mL.

Excipient for dilution of AAV2-GDNF were supplied in 1 mL aliquots. Gadoteridol (ProHance®) was added to the study drug preparation (to provide a 2 mM solution) prior to infusion within the brain. Gadoteridol (2 mM), a gadolinium-based contrast agent, was admixed with each study drug solution to provide enhanced real-time intraoperative MRI monitoring of the CED distribution.

### Overview of Study Design

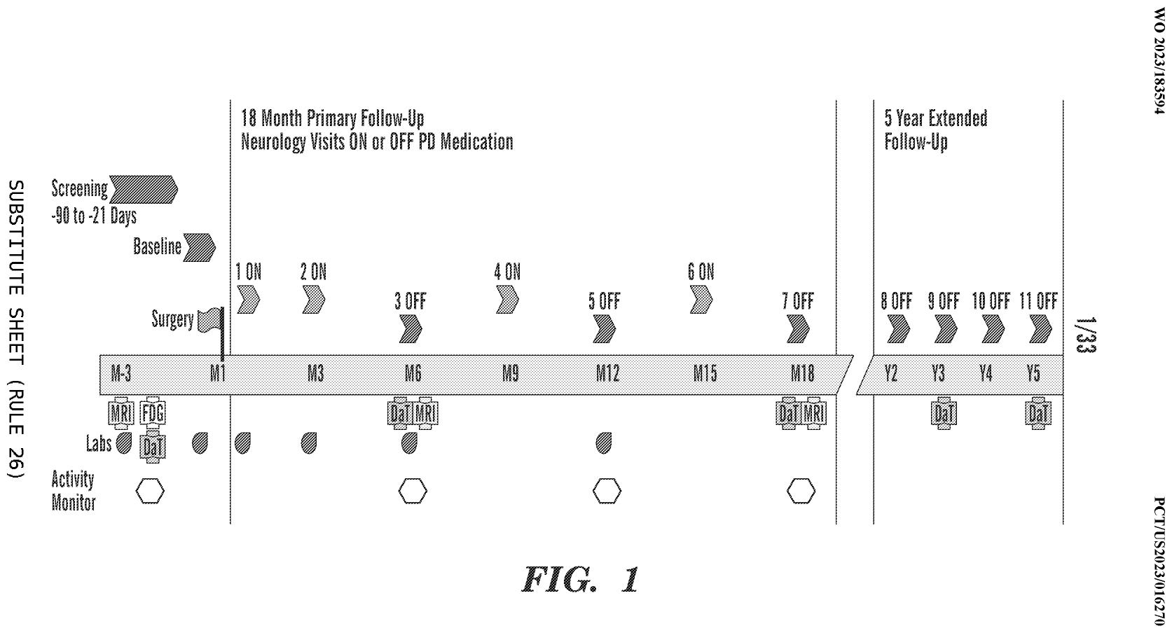

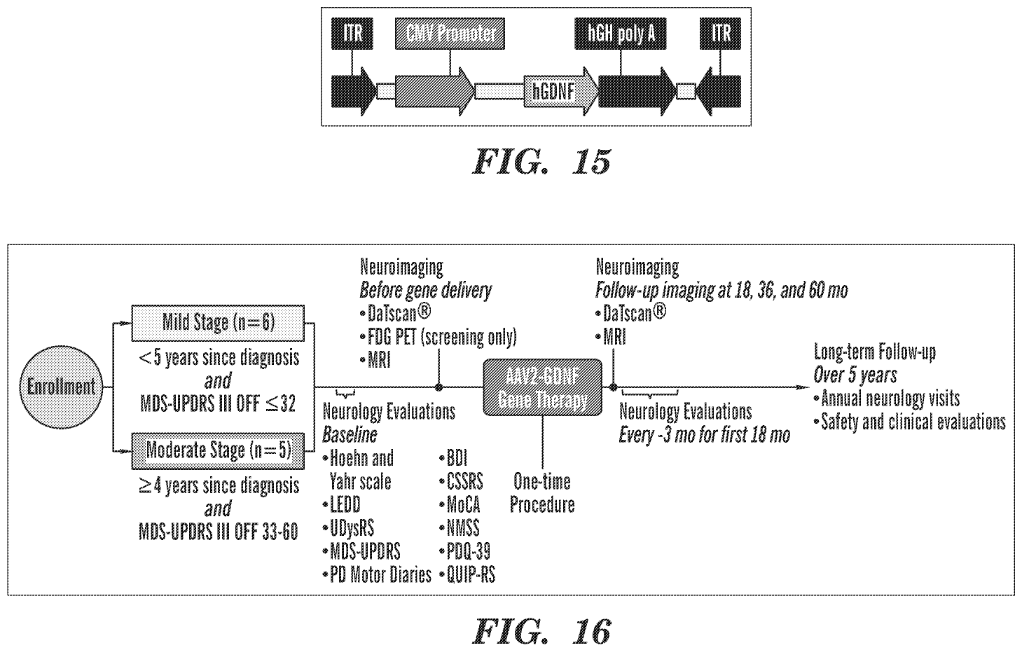

The current study described herein is an open-label safety study of AAV2-GDNF (see, e.g., FIG. 15) delivered by CED bilaterally into the putamen of patients with PD. The primary study objective was to confirm the safety of the delivered viral vector and subsequent expression of the GDNF transgene in both mild and moderately advanced PD patients. Primary safety and clinical outcome assessments are performed 18 months after administration of the study drug. Schematics of the current trial is presented in FIGS. 1 and 16.

Longer-term follow-up will be carried out to at least 5 years, if possible, to assess longer-term safety and durability of any preliminary clinical effects. The annual visits in the long-term follow-up are intended to be performed in-person and in the OFF/ON medication state; however, an abbreviated ON visit may be performed remotely via video call excluding the optional assessments listed in the Schedule of Assessments.

Baseline assessments and pre-operative evaluations prior to receiving the study drug are performed in study participants. The study drug is administered intracranially via iMRI-guided CED neurosurgical infusions into each putamen.

### Screening Procedures

Within 3 months of a tentative date for surgical administration of the study drug, study participants undergo a series of screening procedures, including
Medical History
Review of current PD and non-PD medications
Physical examination with vital signs; neurological examination
Activity monitor: Setup and review instructions for use
Labs:
   Hematology (CBC with platelets)
   Clinical Chemistries—Basic Metabolic Panel (sodium, potassium, chloride, total CO2, creatinine, glucose, urea nitrogen, eGFR), Hepatic Panel (alkaline phosphatase, ALT/GPT, AST/GOT, total & direct bilirubin), and HbA1C
   Coagulation panel (INR, PT, PTT)
   Pregnancy test (if applicable; serum or urine testing to be performed within 30 days of each neuroimaging session)
   PD genetic testing
   Pregnancy test (if applicable; serum or urine testing to be performed within 30 days of each neuroimaging session)
   Immunologic markers (serum): AAV2 neutralizing antibodies, GDNF antibodies
Neuroimaging:
   Brain MRI (ON or OFF state, with and without contrast for pre-surgical planning)
   FDG PET (OFF state, evaluate for PD-related pattern)
   DaT SPECT (OFF state, evaluate presynaptic terminal density)
Clinical assessments of PD motor symptoms and function:
   MDS-UPDRS Parts I-IV (Part III assessed OFF&ON) to evaluate levodopa responsiveness)
   Modified Hoehn & Yahr (OFF)
   Stand-Walk-Sit (OFF/ON)
   Dexterity and Balance testing (OFF/ON)
   Dyskinesia rating (UDysRS, ON)
PD Motor Diary with concordance testing
Neuropsychological assessments: MoCA, CANTAB, BDI-II, BAI, QUIP-RS
Quality of life measures (PDQ-39)
CGI and PGI
CSSRS (screening version to be used for this visit only)
PD non-motor symptom assessments: NMSS, BSIT, PDSS-2, SCOPA-AUT

Subjects can be rescreened up to 2 times at the discretion of the study investigator.

### Study Treatment and Dosing

Administration of the study drug was performed by study neurosurgeons with intraoperative MRI monitoring. Study drug was administered directly via MRI-compatible cannula (SmartFlow®; MRI Interventions, Irvine, CA USA) precisely guided along pre-planned trajectories. Surgeons preferentially plan parietal/occipital trajectories that traverse the long axis of each putamen, but can use alternative trajectories. The surgical procedure was performed within an interventional MRI suite with MRI-compatible trajectory guides (e.g. ClearPoint® neuronavigational system). The procedure was performed by neurosurgeons experienced with MR-based stereotactic placement of devices (e.g. biopsy needles, electrodes, ablation probes) into the brain. Subjects were secured in a head-holder and the planned entry points and target sites (bilateral putamen) located on the brain MRI images. Upon proper targeting of the delivery cannula, up to 1.8 mL of the study drug was infused into each putamen using CED. If the visualized distribution per MR imaging appears sub-optimal, reaches the target volume limits, or leaks (or refluxes) beyond the target, the surgeon can elect to reposition the cannula to optimize drug delivery to the putamen. The maximum volume of 3.6 mL (1.8 mL/putamen) was delivered using CED at variable flow rates of 1-30 L/min.

### Dosing

All participants were administered the study drug via a one-time surgical procedure. The concentration of the study drug was consistent for all subjects, at 3.3×10¹² vg/mL, with up to 1.8 mL infused into each putamen. Total drug dose per subject was up to 1.2×10¹³ vg (3.3×10¹² vg/mL×3.6 mL).

Study drug was delivered bilaterally into the putamen in a single surgical setting using SmartFlow cannula connected to MRI-compatible infusion pumps (e.g. Medfusion syringe pump, Smiths Medical Inc.). CED infusions with increasing rates of infusion (1-30 L/min) was used to deliver the drug volume into each putamen. Adjustments of the cannula depth and infusion rates was made by the surgical team to control distribution within the targeted volume, with a goal of covering 50-80% of each putaminal volume. Infusions were terminated when either target coverage is achieved, additional infusions were meaningfully increase target coverage or increases in infusion volume extends coverage beyond the target volume, or a maximum infusion volume of 1.8 mL per putamen is reached.

In the prior study, the volume of the study drug administered was 0.45 mL per putamen. Because of the lower dose volume utilized, and the transfrontal targeting approach, the mean putaminal volume coverage was limited to 26%. Enhancement of F-DOPA PET uptake in that study was limited to the infused region of the putamen, as documented via iMRI.

The objective of the MRI-monitored dosing procedure was to maximize coverage of each putamen while minimizing off-target delivery. Admixing a small quantity of gadoteridol with the study drug enabled the surgical team to visually monitor the distribution in real-time via iMRI, documenting coverage and allowing modification of cannula position, infusion rates, or infusion stoppage, as required. Given individual variability in the size, shape and anatomical characteristics of the brain, and in particular the putamen of PD patients, the actual volume of infusion and resulting volume of distribution, and the percentage of putaminal volume coverage was expected to vary between subjects. Monitoring the drug infusate distribution throughout the administration procedure optimally allowed for a 50 to 80% putaminal coverage. Infusion of the study drug was stopped when either (i) maximum target coverage of 80% is achieved, (ii) additional infusion will not meaningfully increase target coverage, (iii) significant reflux or leakage is documented beyond the target volume, or (iv) the maximum infusion volume of 1.8 mL per putamen is reached.

### Results

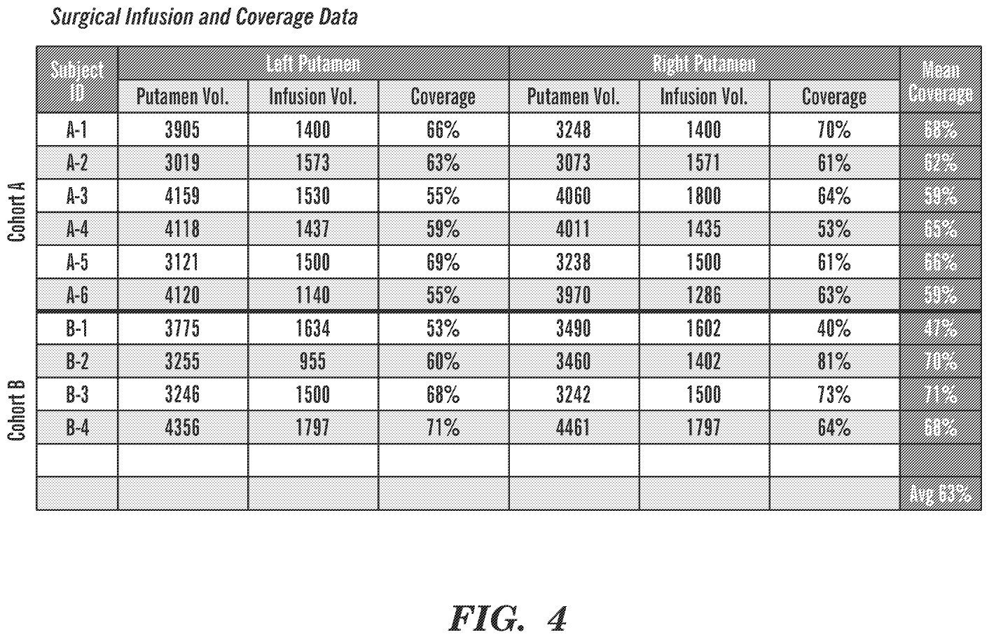

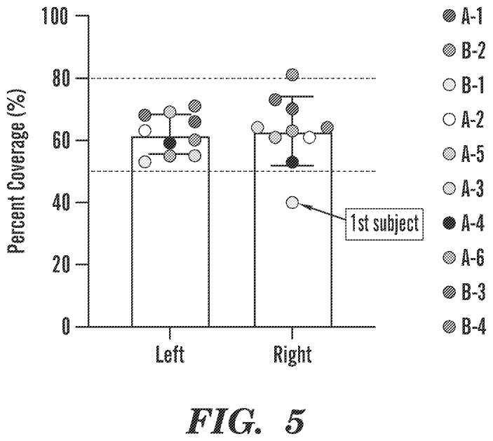

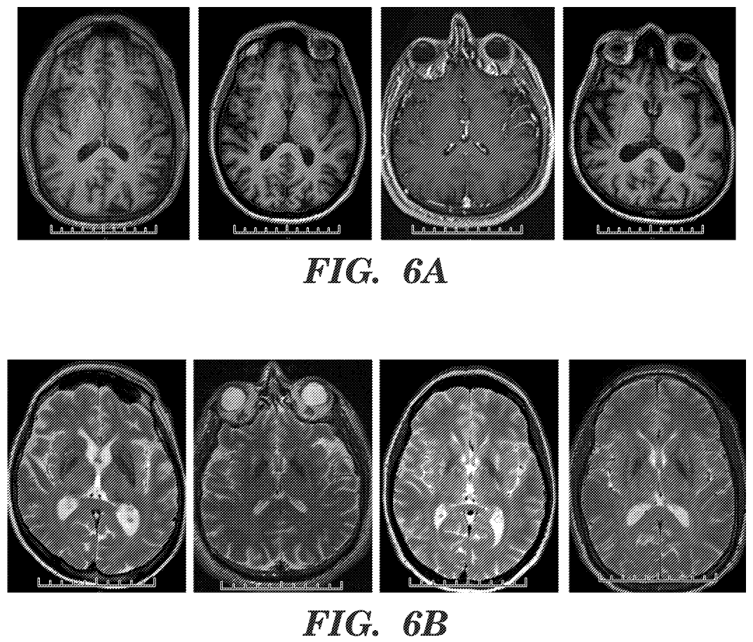

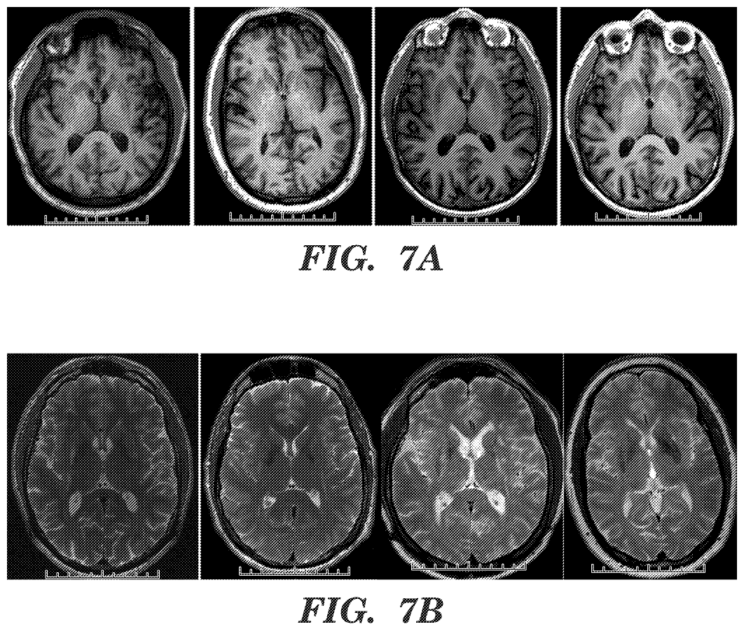

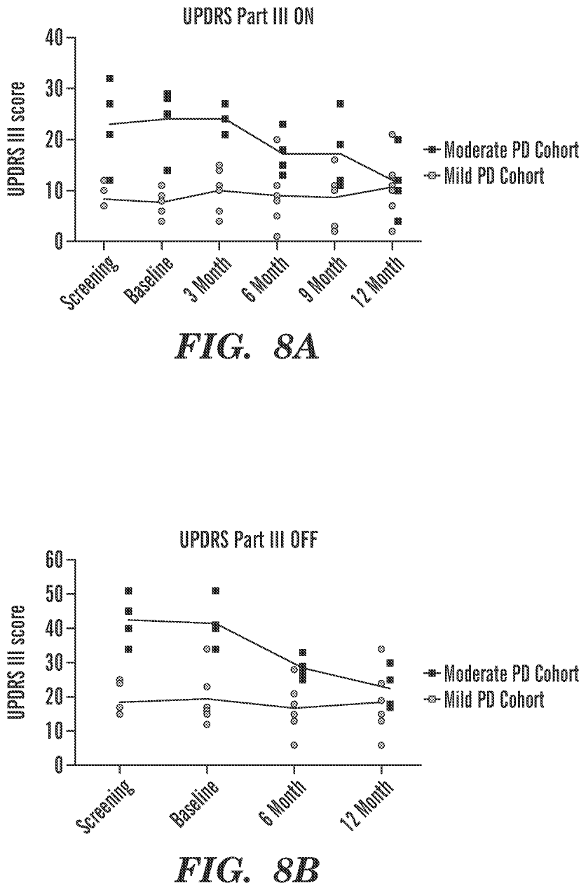

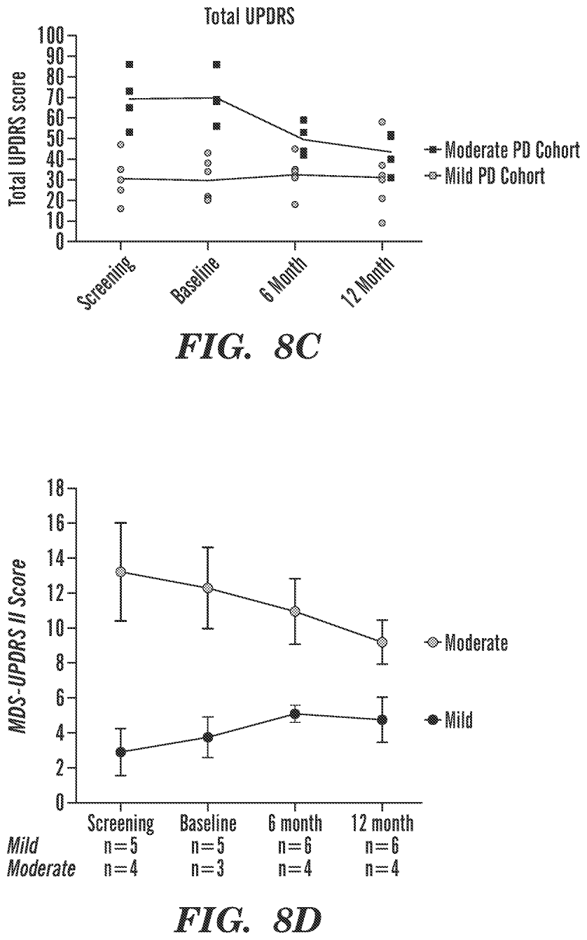

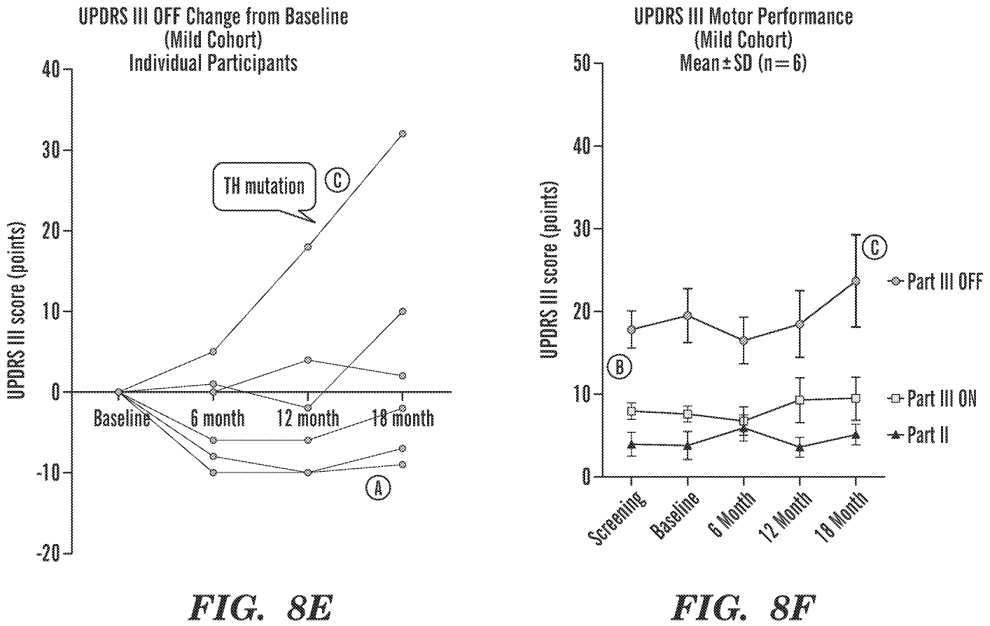

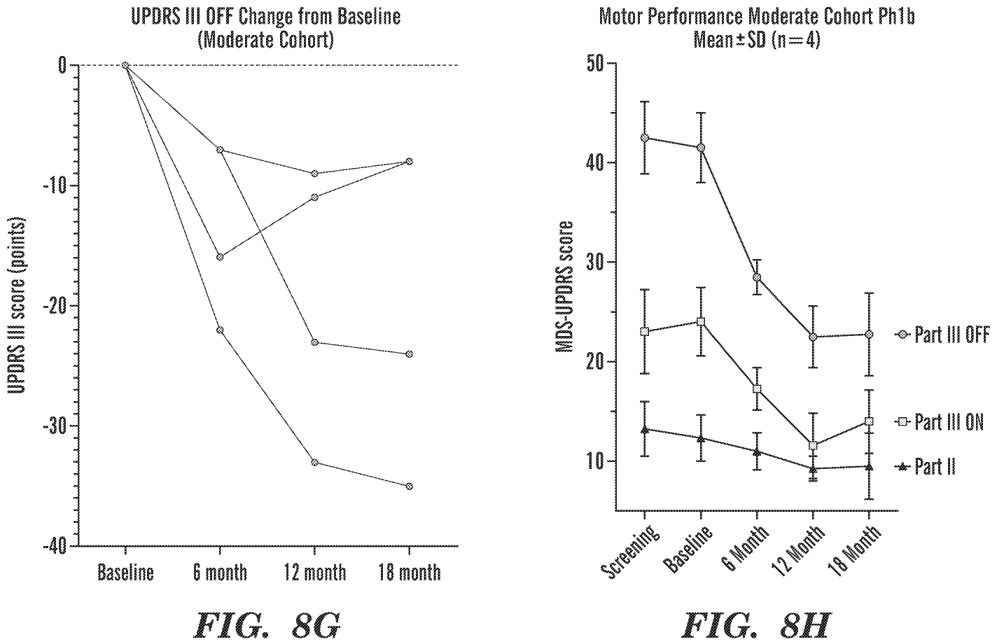

Utilizing infusion methods described herein results in an average coverage of 63% for a single putamen (i.e., the left or right putamen) for both cohort A and cohort B (see, e.g., FIGS. 4, 5, and 6A-7B). Subjects in cohort A exhibited a stabilization of their initial MDS-UPDRS III score for at least 12 months immediately following administration, whereas subject in cohort B exhibited a marked decline in their MDS-UPDRS III score for at least 12 months immediately following administration (FIG. 8A). These results were observed in both an “on” and “off” state (FIGS. 8A and 8B). Similar results were observed with the MDS-UPDRS II score (FIG. 8D)

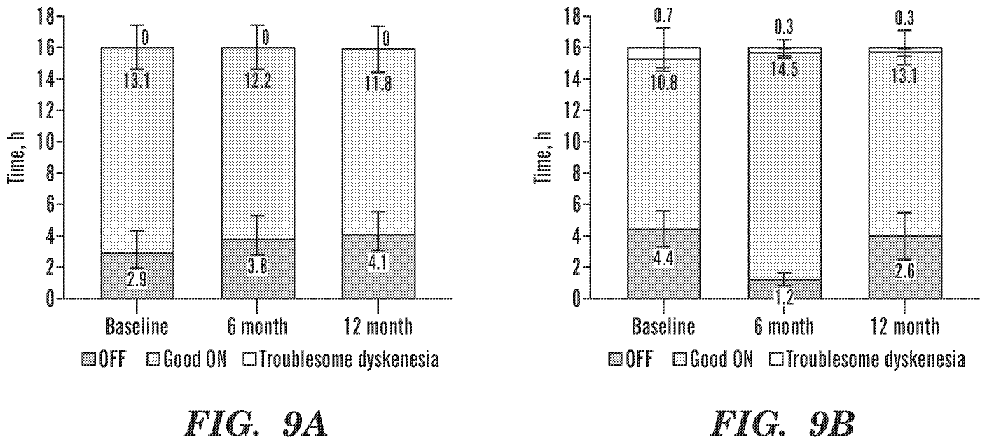

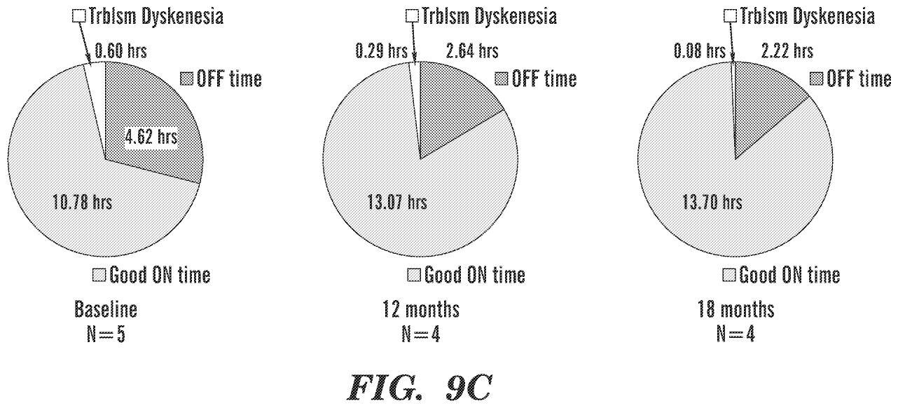

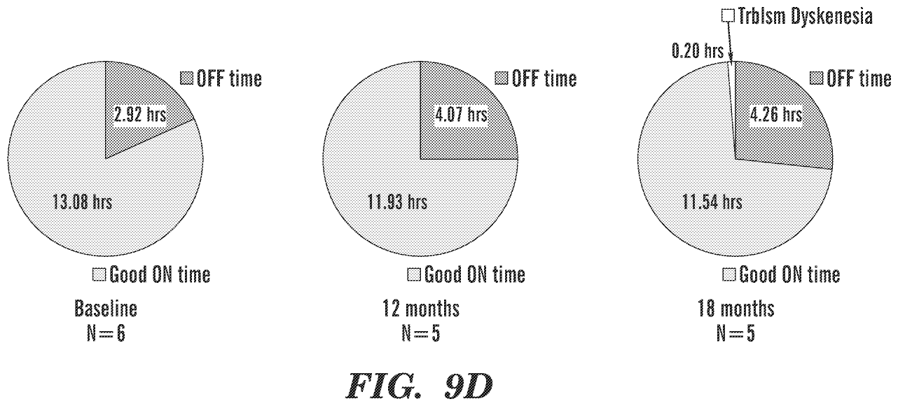

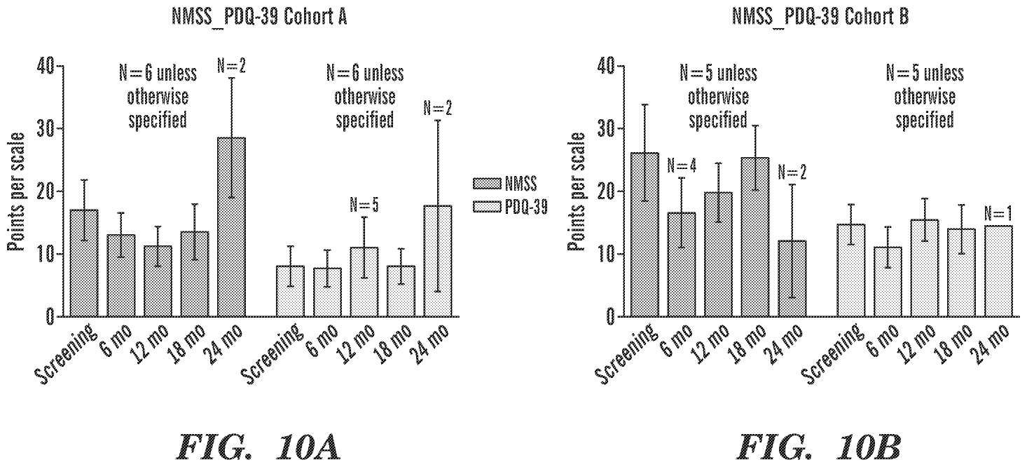

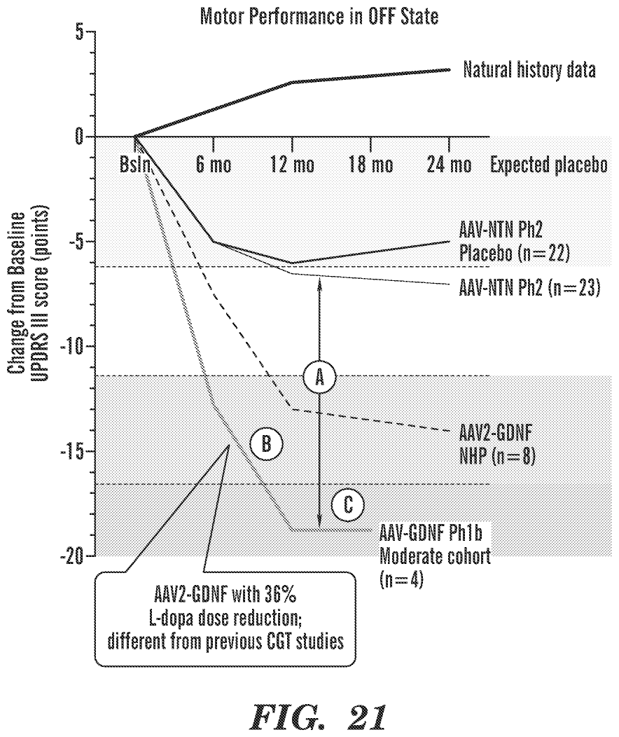

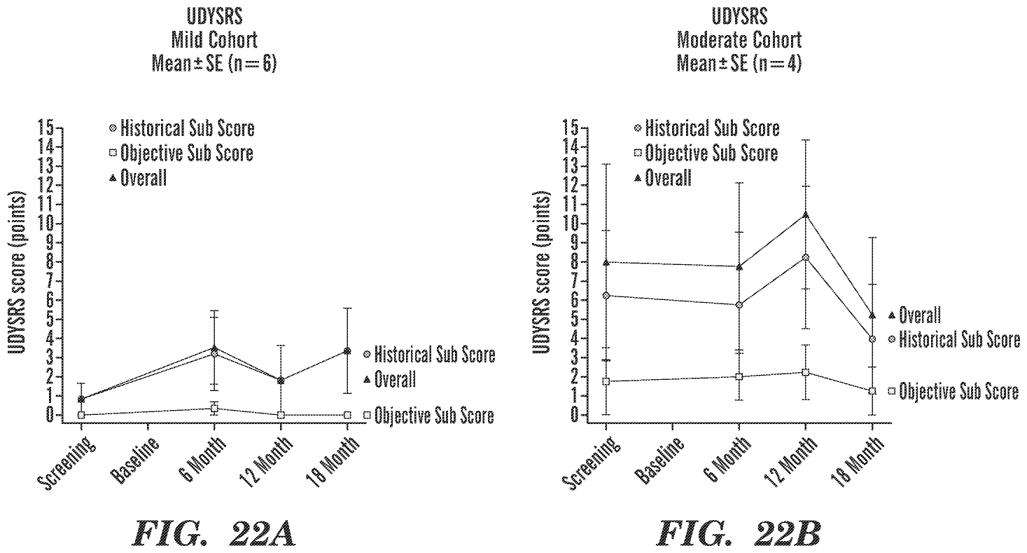

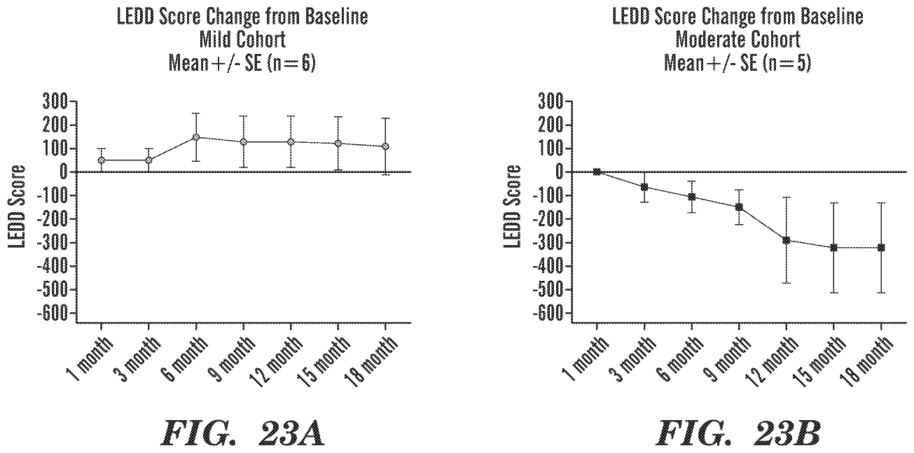

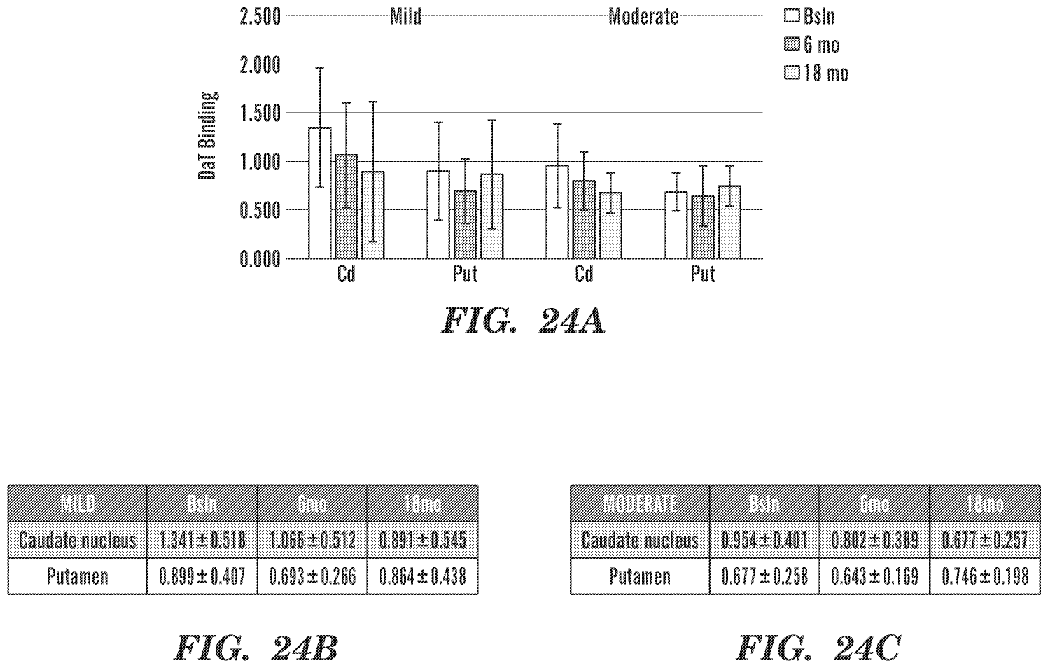

Similar results were observed when monitoring the subjects PD diary (see FIGS. 9A and 9B), and NMSS and PDQ-39 scores following administration. A stabilization of good “on” periods were observed in cohort A at 6 months immediately following administration and a marked increase of good on periods were observed in cohort B at 6 months immediately following administration (FIGS. (9A-9D). Further, a stabilization of the subjects' NMSS and PDQ-39 scores were observed in cohort A at 6, 12 and 18 months immediately following administration (FIG. 10A), and a marked decrease of the subjects' NMSS and PDQ-39 scores were observed in cohort B at 6 and 12 months immediately following administration (FIG. 10B). Finally, UDYSRS objective sub score was stable up to at least 18 months post administration in the mild cohort (FIG. 22A), and slightly decreased in the moderate cohort (FIG. 22B). In the moderate cohort, the UDYSRS overall and historical sub score is decreased at 18 months post administration (FIG. 22B). Moreover, the levadopa equivalent daily dose average values score is stabilized as compared to baseline at at least 18 months post administration in the mild cohort (FIG. 23A), and is decreased at at least 18 months post administration in the moderate cohort (FIG. 23B).

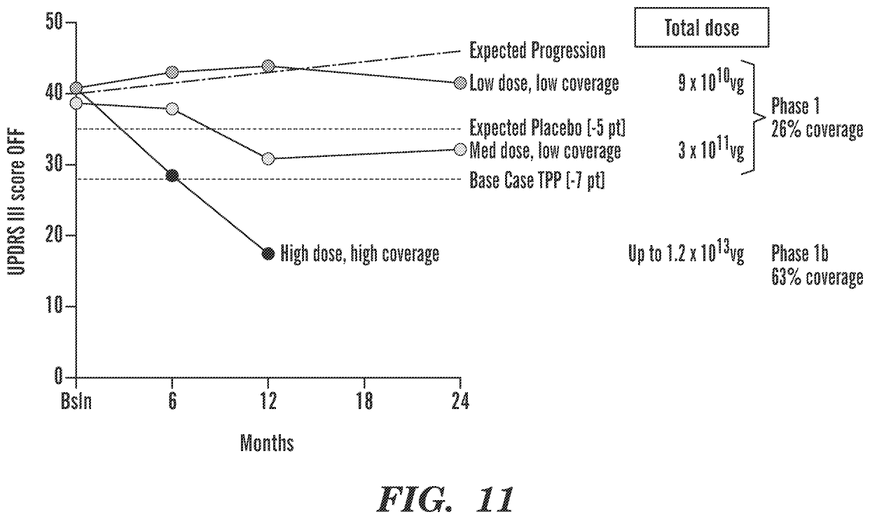

Finally, a dose-dependent decrease in the subjects' MDS-UPDRS III score was observed at 12 months post administration (FIG. 11) and 18 months post administration (FIG. 21). Previous clinical trials included administrations of a lower total dose than described herein (i.e., 9.0×10¹⁰ vg and 3.0×10¹¹ vg) and resulted in a lower total coverage of the putamen (i.e., 26%). This administration resulted in only a slight decrease in the subjects' initial MDS-UPDRS III score at 12 months post administration. In contrast, when a higher total dose (i.e., 1.2×10¹³ vg) was administered, resulting in a 63% coverage of the putamen, a marked decrease in the MDS-UPDRS III score was observed 12 months post administration. These data indicate that the methods described herein are superior than those previously utilized.

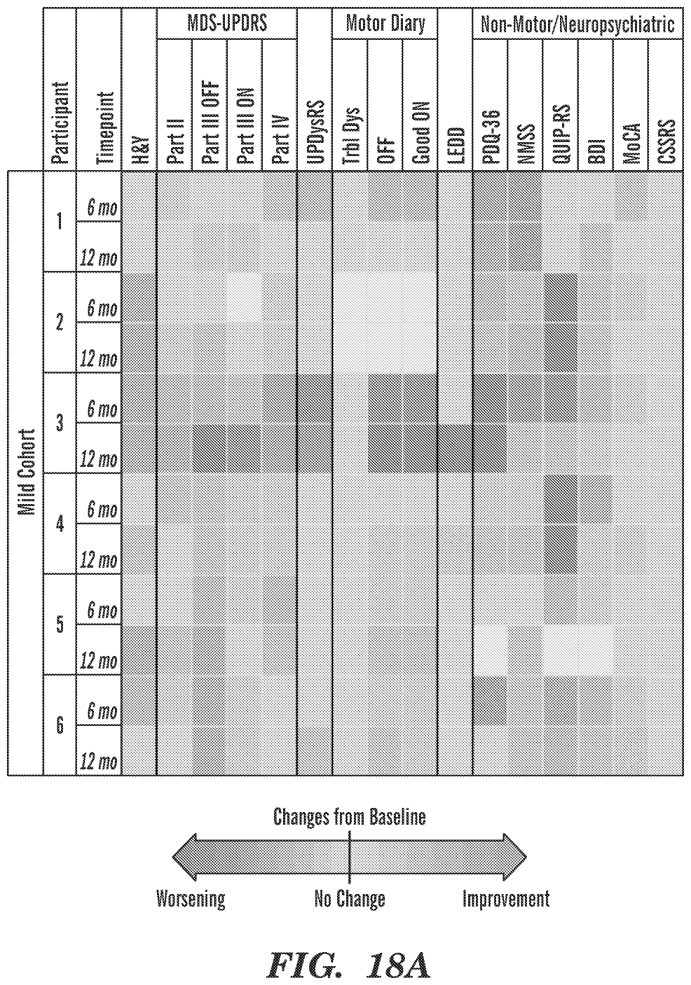

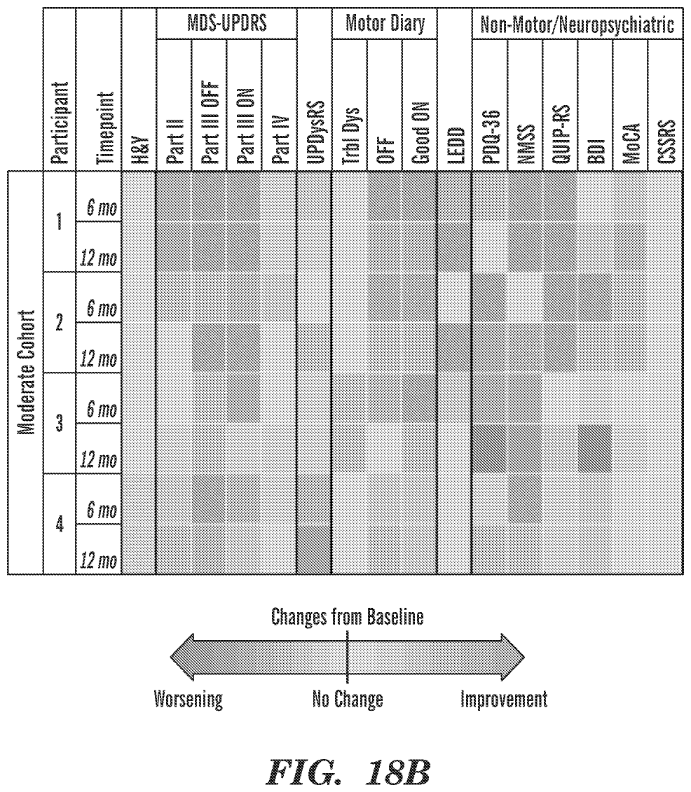

A summary of post-treatment changes for all motor and non-motor assessments for mild and moderate cohorts is provided in FIGS. 18A and 18B, respectively.

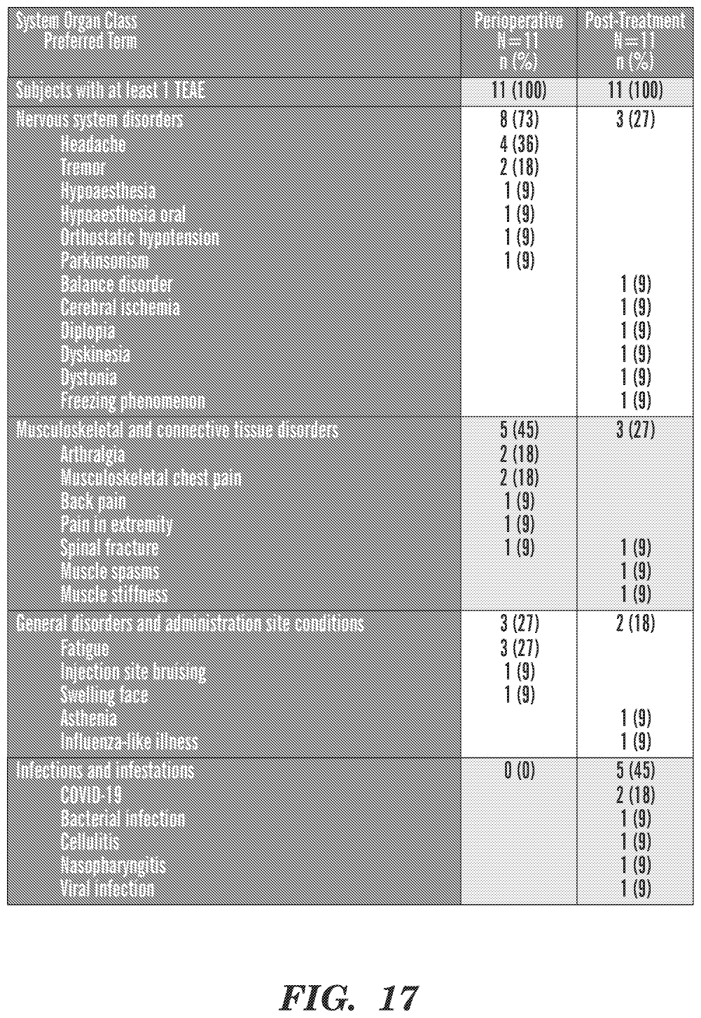

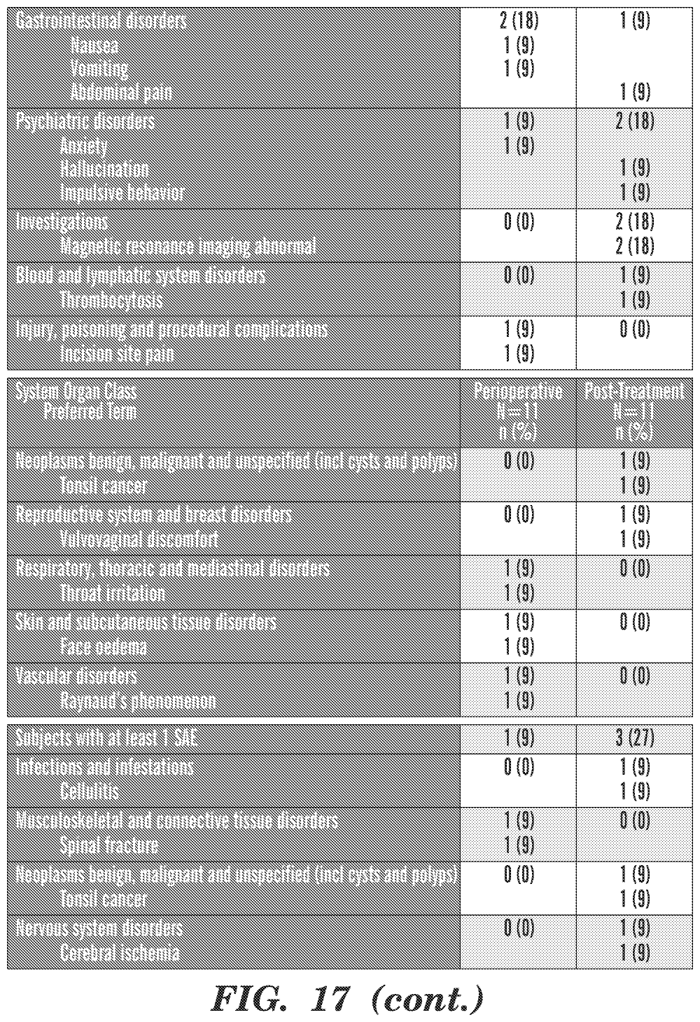

A total of fifty-six (56) TEAEs were observed in this study in 11 participants. The majority of these events were transient and expected perioperative events. These events are summarized in FIG. 17. No AAV2-GDNF-associated adverse events were reported. No subjects have discontinued the study for any reason (including TEAEs). No life-threatening TEAEs or deaths have occurred to date.

### Example 3—Highly-Reproducible and Safe Intraputaminal Delivery of AAV2-GDNF Via Bilateral Single Posterior Trajectories in Early and Moderate Stage Parkinson's Disease

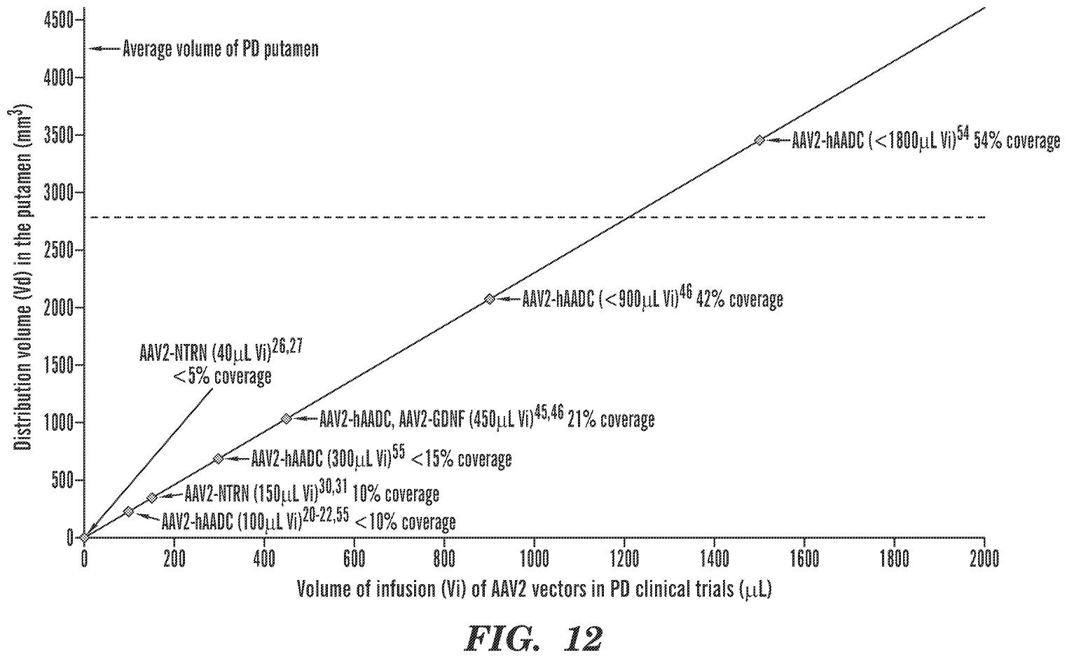

Introduction: Dose optimization for gene therapies (targeting specific CNS targets has presented a technical challengtease for direct intraparenchymal delivery but is critical to achieve meaningful outcomes (see, e.g., FIG. 12). A strong correlation between high putaminal coverage and improved clinical outcomes has been recently demonstrated in Parkinson's Disease (PD) gene therapy trials. However, trans-frontal trajectories used in earlier studies resulted in variable and low volumetric coverage (<5-42%). In the inventors' current PD clinical trial, bilateral single posterior trajectories provided improved AAV2-GDNF distribution within the elongated A-P axis of the putamen to evaluate the clinical effects of high-dose, high-coverage on PD symptomatology.

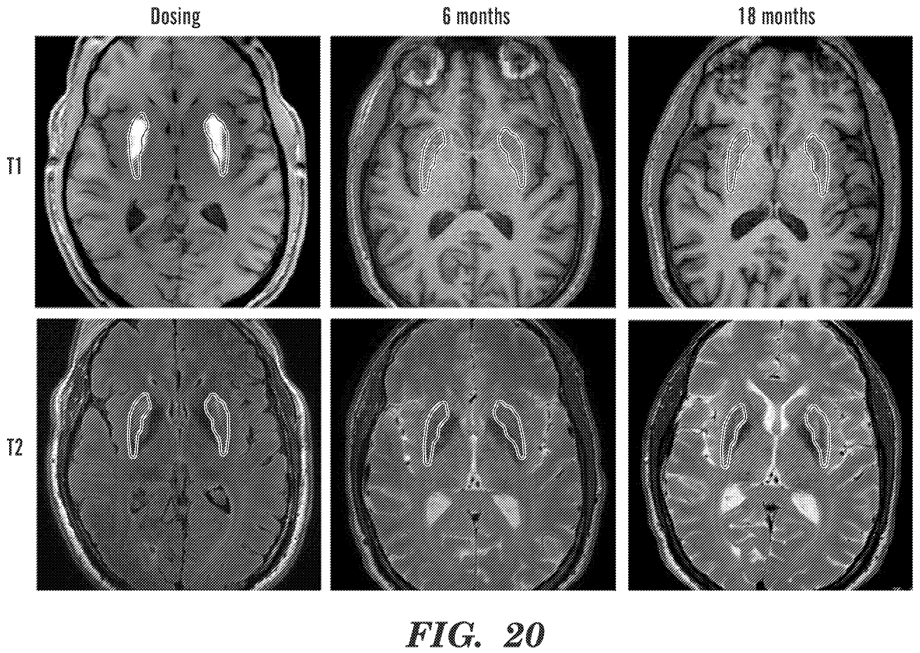

Methods: Eligible participants diagnosed with either Early stage (<5 years; MDS-UPDRS III OFF £32) or Moderate stage PD (>4 years; MDS-UPDRS 33-60) received up to 1.8 mL of AAV2-GDNF (3E¹² vg/ml) per putamen convected using intraoperative MRI (iMRI) monitoring. Participants (n=10) received AAV2-GDNF admixed with gadoteridol (2 mM), allowing infusions tailored to individual anatomical variations and defining putaminal coverage in near real-time by iMRI monitoring throughout the infusion. Gadoteridol signal was visible in the putamen immediately post administration via MRI, but was no longer present 6 months and 18 months post administration (FIG. 20).

Bi-frontal options to increase putaminal distribution, without additional trajectories, encouraged development of strategies for distributing the infusate parallel to the long axis of the putamen. Accordingly, a shape-conforming infusion methodology that evolved. Specifically designed for elongated brain targets, like putamen, the infuse-as-you-go method was published in 2022. Preclinical and recent clinical experiences have indicated significant parenchymal coverage is possible via a single trajectory, entering the tail of the putamen, where infusion begins, and slowly filling the putaminal volume while the cannula is being advanced toward the rostral pole. This infusion method allows consistent volumes (averaging 1500 microliters) to be delivered within the putamen, providing >50% coverage in the current PD clinical trials, while maintaining clinical safety.

Perivascular leakage can be commonly noted with infuse-as-you-go CED in the putamen, however, such leakage is difficult to predict preoperatively, in location and scope. Primary mitigation strategies for neurosurgeons include a) advancement of the delivery cannula and b) minor adjustments in the infusion flow rate. It is potentially advantageous perivascular extension into the caudate nucleus following perivascular channels.

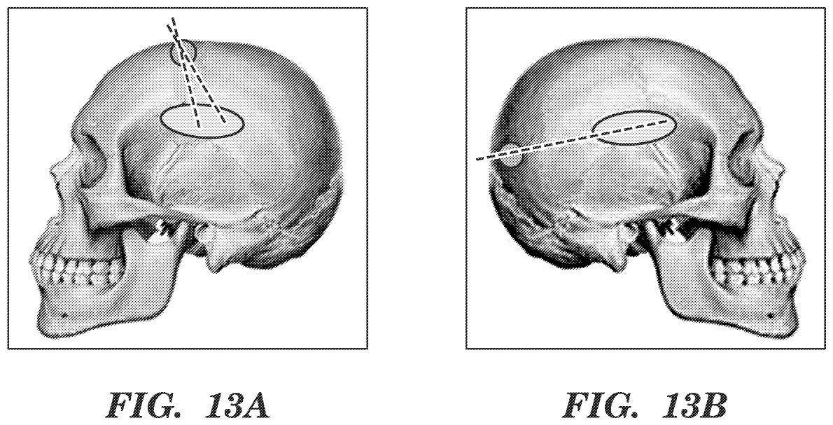

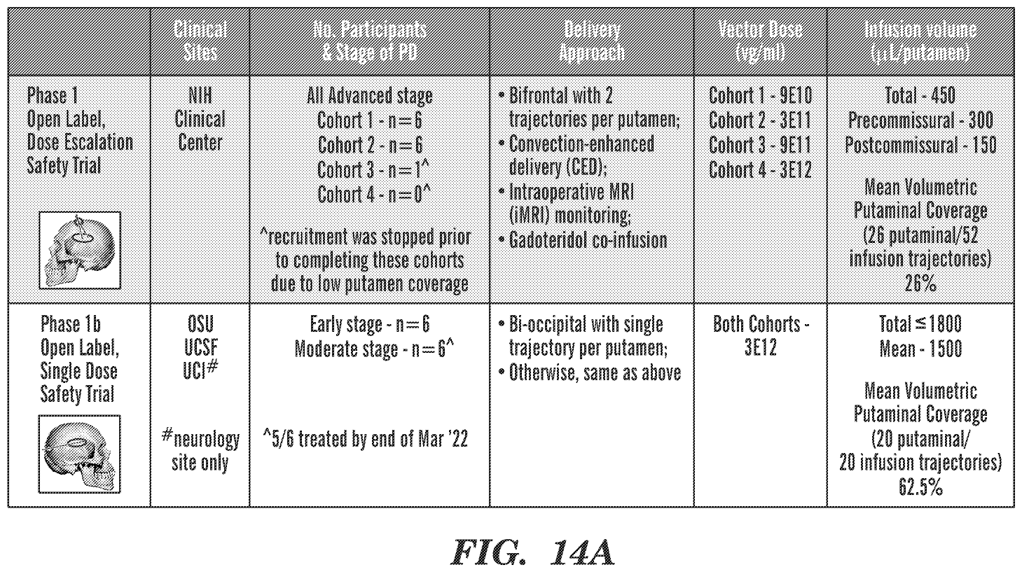

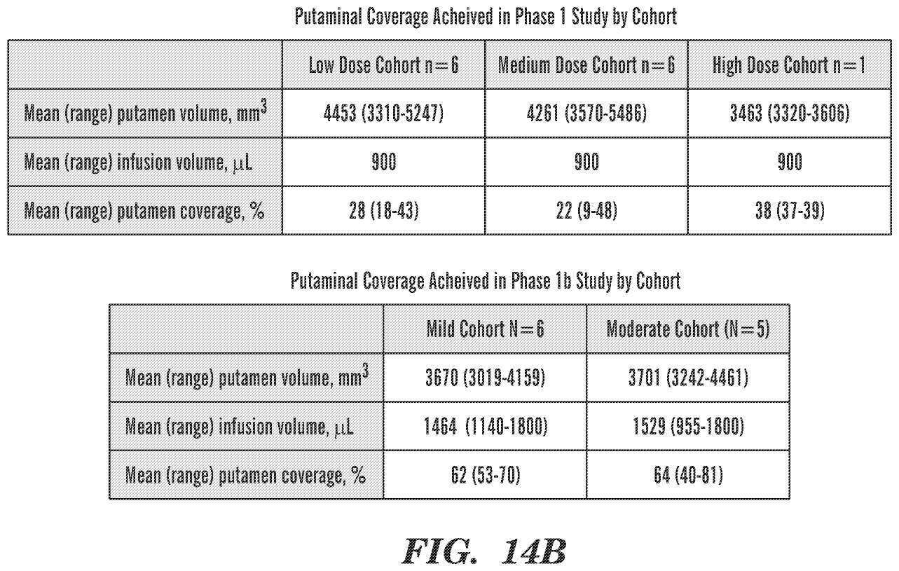

Results: Infusions were safely performed and well-tolerated by all participants. Evidence of a single, unilateral, asymptomatic cerebrovascular event was incidentally detected adjacent to the putamen on a scheduled 6-month MRI. Average putaminal volumetric coverage from 20 infusions was 62.5%, a marked improvement from the 26% average coverage in the initial AAV2-GDNF study and exceeds the >50% coverage goal (see, e.g., FIGS. 14A and 14B). Preliminary clinical findings indicate greater motor benefits and reductions in PD medications when compared to a previous Phase 1 study (Heiss 2019).

Conclusions: The delivery technique described herein is highly-reproducible despite anatomical heterogeneity and variable putaminal volumes. These results indicated that putaminal infusions utilizing posterior trajectories appear safe, well-tolerated, and consistently provide greater volumetric target coverage when compared to trans-frontal trajectories. This platform utilizes methods and technologies specifically designed to provide safe and effective intraparenchymal distribution of therapeutic infusion volumes. For putaminal gene therapies for Parkinson's disease, the following techniques are currently utilized: (i) A single infuse-as-you-go trajectory per putamen, utilizing an occipital approach, paralleling the long axis of the putamen; (ii) Gadoteridol co-infusion allowing direct, near-real time visualization of the infusate distribution within the intraoperative MR scanner; (iii) Convection enhanced delivery methods, that extend therapeutic distributions within the individualized target volume; (iv) A reflux-resistant cannula, that maximizes distribution of the infusate within the target; and (v) Intraoperative magnetic resonance imaging, for validation of preoperative trajectory planning, confirmation of intraoperative delivery cannula placement, and visualization of cannula advancement during infusion so as to tailor a “shape-fitting” distribution. This technique has enhanced dosing of AAV2-GDNF, and is for meaningful clinical improvement in PD patients.

### Example 4—AAV2-GDNF Expression Localized to Putamen Infusion Sites

The putamen of a Phase 1 safety trial participant was assessed 3.5 years post-administration to evaluate the level and location of GDNF transgene expression.

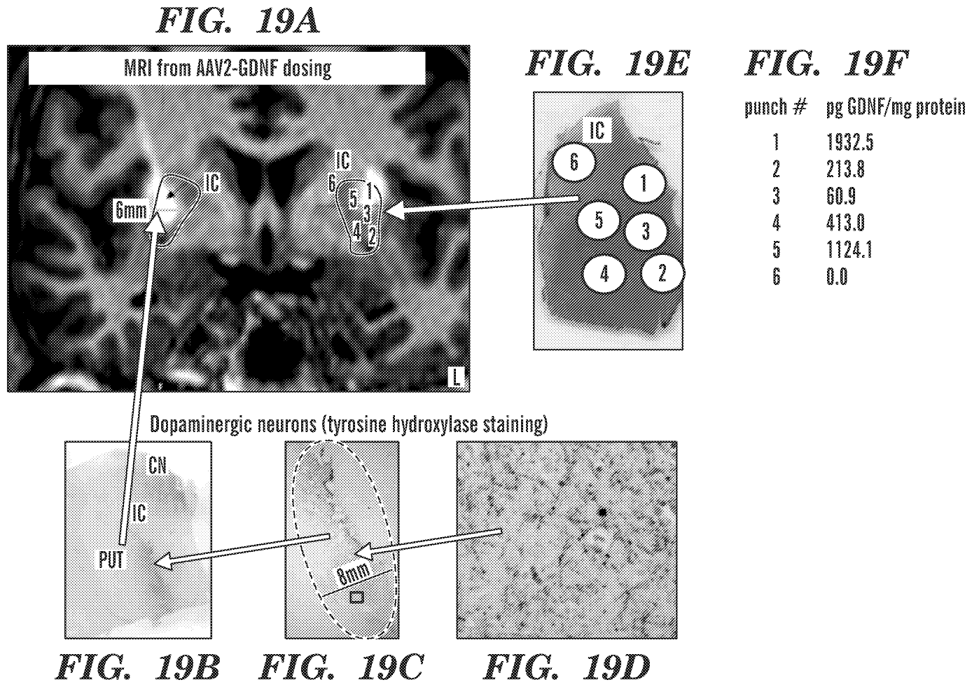

Trans-frontal administration of AAV2-GDNF in the Phase 1 trial resulted in ˜23% coverage of the putamen. Punch biopsies of the putamen obtained from the participant showed that local expression of AAV2-GNDF correlated with the local administration of gadoteridol to the putamen during surgery (FIGS. 19A-19E). Two infusions per putamen were administered during surgery; punch biopsies #1 and #5 correlate with the two putaminal infusions sites. The highest expression levels of GDNF were observed in those sites, indicating that the highest transgene expression of GDNF is sustained at the infusion sites (FIGS. 19E and 19F). These data indicate that AAV2-GDNF undergoes diffusion throughout the tissue following administration. Nonetheless, protocols that yield a higher percent coverage of the putamen following administration are desired. In further support of these data, no GDNF transgene expression was observed at punch biopsy #6, which was performed outside the putamen in the white matter tract (FIGS. 19E and 19F).

To assess if the observed GDNF transgenes levels were indicative of GDNF activity, the putamen sample was assessed for the presence of dopaminergic fibers. Tyrosine hydroxylase staining of the putamen sample revealed enhanced dopaminergic fibers in areas having high GDNF expression, indicating that GDNF is active in the putamen (FIGS. 19B-19D). These data demonstrate that wider distribution of GDNF in the putamen is provides for greater enhancement of dopaminergic fibers. In support of these data, a marked increase of F-DOPA was observed in study participants 6 and 18 months post administration. As described herein above, F-DOPA uptake was assessed via PET scan and compared to a baseline (e.g., prior to administration). A progressive increase was observed over time; an ˜25% increase in F-DOPA uptake over baseline was observed at 6 months and an ˜45% increase in F-DOPA uptake over baseline was observed at 18 months. Increased F-DOPA uptake suggests a positive neurotrophic effect on the dopaminergic neurons.

Although these data were obtained from the Phase 1 safety trial with 23% coverage of the putamen following infusion, these data confirmed that the administration protocol is safe and that the AAV2-GDNF is durable.

### Example 5—AAV2-GDNF

AAV2-GDNF will be administered to subjects having Parkinson's disease. The AAV2-GDNF will include close ended linear duplexed DNA encapsidated by AAV2. The close ended linear duplexed DNA will comprise the ITR to ITR portion of sequence SEQ ID NO: 64 (i.e., base pairs 12-2,716 of SEQ ID NO: 64). As an example, the close ended linear duplexed DNA can be manufactured from the plasmid DNA illustrated in FIG. 26. The AAV2-GDNF will be infused into the putamen of the subjects by MRI-guided CED using bi-occipital trajectories. The results are expected to be substantially similar to those described above.

1. A method of slowing or inhibiting progression of Parkinson's disease (PD) in a subject in need thereof, the method comprising:
introducing to the subject a recombinant adeno-associated virus (rAAV) comprising a glial cell line-derived neurotrophic factor (GDNF) transgene operably linked to a promoter,
wherein at least 30% of the volume of the subject's putamen is transduced with the GDNF transgene, and
wherein the subject does not exhibit an increase in PD-associated symptoms for a least 6 months following the introducing as compared to prior to introducing.

2. The method of claim 1, wherein the rAAV is introduced via systemic introduction.

3. The method of claim 1, wherein the rAAV is introduced via local introduction.

4. The method of claim 3, wherein local introduction is introduction directly to the subject's putamen.

5. The method of claim 3 or 4, wherein the local introduction comprises directly introducing the rAAV to each of the subject's putamen.

6. The method of any one of claims 3-5, wherein the local introduction is performed in simultaneously with non-invasive imaging.

7. The method of claim 6, wherein the non-invasive imaging is selected from the group consisting of intraoperative magnetic resonance image (iMRI)-guided convection enhanced delivery (CED), ultrasound, computed tomography (CT); functional magnetic resonance imaging (fMRI); positron emission tomography (PET); electroencephalography (EEG); magnetoencephalography (MEG); functional near-infrared spectroscopy (fNIRS); and combinations thereof.

8. The method of any one of claims 3-7, wherein the local introduction comprises introducing about half of the rAAV vector to each putamen via intraoperative magnetic resonance image (iMRI)-guided convection enhanced delivery (CED).

9. The method of any one of claims 3-8, wherein local introduction further comprises introducing an MRI contrast agent at substantially the same time as the AAV vector.

10. The method of claim 8, wherein the MRI contrast agent is gadoteridol.

11. The method of claim 9 or 10, wherein the MRI contrast agent is introduced to the subject in the same composition as the rAAV.

12. The method of claim 9 or 10, wherein the MRI contrast agent is introduced to the subject in a different composition as the rAAV.

13. The method of claim 1, wherein the rAAV is introduced via systemic introduction.

14. The method of any of claims 1-13, wherein the transduction and/or coverage of the putamen is assessed via Magnetic-resonance imaging.

15. The method of any of claims 1-14, wherein at least 40%, 50%, 60%, 70%, 80%, 90%, 95% or more of the volume of the subject's putamen is transduced with the GDNF transgene.

16. The method of any of claims 1-15, wherein the subject does not exhibit a substantial increase in PD-associated symptoms for at least 12 months immediately following the introducing as compared to prior to the introducing.

17. The method of any of claims 1-15, wherein the subject exhibits a decrease in PD-associated symptoms for at least 6 months or more immediately following the introducing as compared to prior to introducing.

18. The method of any of claims 1-15, wherein the subject exhibits a decrease in PD-associated symptoms for a least 12 months or more immediately following the introducing as compared to prior to introducing.

19. The method of claim 1, wherein the subject has an initial Movement Disorder Society-Unified Parkinson Disease Rating Scale (MDS-UPDRS) score, prior to introduction, that is less than 32.

20. The method according to claim 19, wherein the slowing or inhibiting the progression of Parkinson's disease in the subject is characterized by a second MDS-UPDRS score 6 months immediately following the introducing that is not substantially higher than the initial MDS-UPDRS score.

21. The method according to claim 19 or 20, wherein the slowing or inhibiting the progression of Parkinson's disease in the subject is characterized by a second MDS-UPDRS score about 12 months immediately following the introducing that is not substantially higher than the initial MDS-UPDRS score.

22. The method claim 1, wherein the subject has an initial MDS-UPDRS score, prior to introduction, that is greater than or equal to 32.

23. The method according to claim 22, wherein the subject exhibits a decrease in the initial MDS-UPDRS score for at least 6 months immediately following the introducing as compared to prior to introducing.

24. The method according to claim 22 or 23, wherein the slowing or inhibiting the progression of Parkinson's disease in the subject is characterized by a second MDS-UPDRS score about 6 months immediately following the introducing that is at least about 20% lower than the initial MDS-UPDRS score.

25. The method according to any one of claims 21-23, wherein the slowing or inhibiting the progression of Parkinson's disease in the subject is characterized by a second MDS-UPDRS score about 12 months immediately following the introducing that is at least about 30% lower than the initial MDS-UPDRS score

26. The method of claim 1, further comprising, prior to introducing, determining an initial MDS-UPDRS score for the subject.

27. The method of claim 1, further comprising, prior to introducing, receiving results of an assay that provides an initial MDS-UPDRS score for the subject.

28. The method according to claim 1, wherein slowing or inhibiting the progression of PD in the subject is characterized by a reduction of an initial MDS-UPDRS score following introduction.

29. The method according to claim 28, wherein the reduction is an at least 20%, 30%, 40%, 50%, 60%, 70%, 80%, 90%, 95% or greater reduction of the initial MDS-UPDRS score 6 months following introduction.

30. The method according to claim 28, wherein the reduction is an at least 20%, 30%, 40%, 50%, 60%, 70%, 80%, 90%, 95% or greater reduction of the initial MDS-UPDRS score 12 months following introduction.

31. The method according to claim 1, wherein slowing or inhibiting the progression of PD in the subject is characterized stabilization of an initial MDS-UPDRS score following introduction.

32. The method according to claim 31, wherein the stabilization is characterized by no more than a 10% increase or decrease of the initial MDS-UPDRS score.

33. The method of claim 31 or 32, wherein stabilization occurs for at least 6 months or longer.

34. The method of claim 1, wherein the subject is mildly affected by PD.

35. The method of claim 34, wherein the subject mildly affected by PD has an initial MDS-UPDRS score less than 32 prior to the introduction of rAAV and was diagnosed with PD less than 5 years prior to the introduction.

36. The method of claim 1, further comprising, prior to the introduction, diagnosing the subject as being mildly affected by PD.

37. The method of claim 1, further comprising, prior to the introduction, receiving the results of an assay that diagnoses the subject as being mildly affected by PD.

38. The method of claim 1, wherein the subject is moderately affected by PD.

39. The method of claim 38, wherein the subject moderately affected by PD has an initial MDS-UPDRS score equal to or greater than 32 prior to the introduction of rAAV and was diagnosed with PD less than 4 years prior to the introduction.

40. The method of claim 1, further comprising, prior to the introduction, diagnosing the subject as being moderately affected by PD.

41. The method of claim 1, further comprising, prior to introduction, receiving the results of an assay that diagnoses the subject as being moderately affected by PD.

42. The method of claim 1, wherein the promoter is a cytomegalovirus (CMV) promoter.

43. The method of claim 1, wherein the promoter is a nervous system (NS) or central nervous system (CNS) specific promoter.

44. The method of claim 43, wherein the NS specific promoter is selected from the NS specific promoters in Table 1.

45. The method of claim 43, wherein the CNS specific promoter is selected from the CNS specific promoters in Table 2.

46. The method of claim 1, wherein the GDNF transgene comprises a sequence of SEQ ID NO: 1, or a functional variant that is at least 85, 86, 87, 88, 89, 90, 91, 92, 93, 94, 95, 96, 97, 98, 99% or more identical to SEQ ID NO: 1.

47. The method of claim 1, wherein the rAAV is AAV1, AAV2, AAV3, AAV3b, AAV4, AAV5, AAV6, AAV7, AAV8, AAV9, AAV10, AAV11, AAV12, Rh10, or a rational haploid thereof.

48. The method of claim 47, wherein the rAAV is AAV2.

49. The method of claim 1, wherein the rAAV exhibits brain-specific tropism.

50. The method of claim 1, wherein the rAAV comprises a modification that increases its brain-specific tropism.

51. The method of claim 50, wherein brain-specific tropism is increased by at least 20%, 30%, 40%, 50%, 60%, 70%, 80%, 90%, 95% or greater as compared to an unmodified AAV.

52. The method of claim 1, wherein the rAAV is introduced at a total dose within the range of 5×10¹² vg to about 1.5×10¹³ vg.

53. The method of claim 52, wherein about one half of the total dose is administered to each of the subject's putamen.

54. The method of claim 3, wherein introducing is performed at a flow rate of from about 1 L/min to about 30 L/min.

55. The method of claim 1, wherein the rAAV is introduced as a liquid composition comprising the rAAV and a pharmaceutically acceptable carrier.

56. The method of claim 55, wherein the liquid composition has an rAAV concentration of from about 3×10¹² vg/mL to about 4×10¹² vg/mL.

57. The method of claim 1, wherein the subject is administered at least one anti-PD therapeutic prior to the introduction of the rAAV.

58. The method of claim 1, wherein the subject is administered at least one anti-PD therapeutic prior to and following the introduction of the rAAV.

59. The method of claim 57 or 58, wherein the at least one anti-PD therapeutic is selected from the group consisting of levodopa, Sinemet, Rytary, Stalevo, amantadine, pramipexole, rotigotine, ropinirole, apomorphine, entacapone.

60. The method of any one of claims 57-59, wherein the subject maintains or decreases the dose of the at least one anti-PD therapeutic following introduction.

61. The method of claim 60, wherein the dose of the at least one anti-PD therapeutic is decreased by at least 5%, 10, 1%, 20%, 25%, 30%, 35%, 40%, 45%, 50% or more.

62. A method of slowing or inhibiting a progression of Parkinson's disease (PD) in a subject in need thereof, the method comprising:
locally introducing to the subject's putamen a recombinant adeno-associated virus (rAAV) vector comprising a glial cell line-derived neurotrophic factor (GDNF) transgene operably linked to a promoter,
wherein at least 30% of the volume of the subject's putamen is transduced with the GDNF transgene.

63. A method of slowing or inhibiting a progression of PD in a subject in need thereof, the method comprising:
transducing greater than or equal to about 30% of the volume of the subject's putamen with a glial cell line-derived neurotrophic factor (GDNF) transgene,
wherein the subject does not exhibit a substantial increase in PD-associated symptoms for a least 6 months following the transducing.

64. The method according to claim 63, wherein the transducing is performed by administering a rAAV comprising the GDNF transgene to each of the subject's putamen.

65. A method of reducing or stabilizing an initial Movement Disorder Society-Unified Parkinson's Disease Rating Scale Part (MDS-UPDRS) score in a subject having Parkinson's disease (PD), the method comprising:
administering to the subject's putamen a recombinant adeno-associated virus (rAAV) comprising a glial cell line-derived neurotrophic factor (GDNF) transgene operably linked to a promoter,
wherein the subject has a second MDS-UPDRS score at 6 months following the administration is decreased or stabilized as compared to the initial MDS-UPDRS score of the subject prior to administering.

66. The method of claim 65, further comprising the step of, prior to administering, obtaining or receiving an initial MDS-UPDRS score from the subject.

67. The method according to claim 65, wherein the second MDS-UPDRS score is decreased by at least 20%, 30%, 40%, 50%, 60%, 70%, 80%, 90%, 95% or greater as compared to the initial MDS-UPDRS score 12 months following administering.

68. The method according to claim 65, wherein stabilization is no more than a 10% increase or decrease of the initial MDS-UPDRS score.

69. A method of treating a subject mildly affected by Parkinson's disease (PD), the method comprising:
administering to each of the subject's putamen a recombinant adeno-associated virus (rAAV) comprising a glial cell line-derived neurotrophic factor (GDNF) transgene operably linked to a promoter,
wherein at least 30% of the subject's putamen is transduced with the GDNF transgene, and
wherein the subject has a second MDS-UPDRS score at 6 months post-administering that is stabilized as compared to the initial MDS-UPDRS score.

70. The method of claim 69, wherein the subject has a MDS-UPDRS score at 12 month post-administering that is stabilized as compared to the initial MDS-UPDRS score prior to administering.

71. The method of claim 69 or 70, wherein stabilization is no more than a 10% increase or decrease of the initial MDS-UPDRS score.

72. A method of treating a subject moderately affected by Parkinson's disease (PD), the method comprising:
administering to each of the subject's putamen a recombinant adeno-associated virus (AAV) comprising a glial cell line-derived neurotrophic factor (GDNF) transgene operably linked to a promoter,
wherein at least 30% of the subject's putamen is transduced with the GDNF transgene, and
wherein the subject has a second MDS-UPDRS score at 6 months post-administering that is at least about 20% lower than the initial MDS-UPDRS score.

73. The method of claim 72, wherein the reduction is an at least 30%, 40%, 50%, 60%, 70%, 80%, 90%, 95% or greater as compared to the initial MDS-UPDRS score.

74. A method of slowing or inhibiting progression of Parkinson's disease (PD) in a subject in need thereof, the method comprising:
locally introducing to each of the subject's putamen a recombinant adeno-associated virus (rAAV) vector comprising a glial cell line-derived neurotrophic factor (GDNF) transgene operably linked to a promoter; and
locally introducing an MRI contrast agent to each of the subject's putamen at substantially the same time as the rAAV,
wherein at least 30% of the volume of the subject's putamen is transduced with the transgene, and
wherein the subject does not exhibit a substantial increase in PD-associated symptoms for a least 6 months immediately following the introducing as compared to prior to introducing.

75. A method of slowing or inhibiting progression of Parkinson's disease (PD) in a subject in need thereof, the method comprising:
introducing to the subject a recombinant adeno-associated virus (rAAV) comprising a glial cell line-derived neurotrophic factor (GDNF) transgene operably linked to a promoter,
wherein at least 30% of the volume of the subject's putamen is transduced with the GDNF transgene, and
wherein the subject does not exhibit a substantial increase in PD-associated symptoms for a least 6 months immediately following the introducing as compared to prior to introducing.

76. A composition for slowing or inhibiting a progression of Parkinson's disease (PD) in a subject, the composition comprising:
a recombinant adeno-associated virus (rAAV) comprising a genome comprising a glial cell line-derived neurotrophic factor (GDNF) transgene operably linked to a promoter; and
a pharmaceutically acceptable carrier.

77. The composition of claim 76, wherein the composition has a rAAV concentration of 3×10¹² vg to 4×10¹² vg per mL.

78. The composition of claim 76, wherein the composition comprises an rAAV concentration of 3.3×10¹² vg per mL.

79. A formulation for slowing or inhibiting a progression of Parkinson's disease (PD) in a subject, the formulation comprising: an adeno-associated virus (AAV) at a concentration of 3×10¹² vg to 4×10¹² vg per mL of a pharmaceutically acceptable carrier,
wherein the rAAV comprises a genome comprising a glial cell line-derived neurotrophic factor (GDNF) transgene operably linked to a promoter.

80. The method of any preceding claims, wherein the subject does not exhibit any serious adverse event for a least 6 months immediately following the introducing or administering.

81. Use of recombinant adeno-associated virus (rAAV) comprising a nucleic acid encoding glial cell line-derived neurotrophic factor (GDNF), hereinafter GDNF transgene, operably linked to a promoter in the preparation of a medicament for a method of slowing, inhibiting, or stabilizing progression of Parkinson's disease (PD) in a subject or treating a subject with a mild form of PD,
wherein at least 30% of the volume of the putamen is transduced with the GDNF transgene, and wherein the subject does not exhibit an increase in PD-associated symptoms for at least 6 months immediately following the introduction as compared to prior to introduction of the medicament.
